(19) United States
(12) Patent Application Publication
(10) Pub. No.: US 2004/0127501 A1
(43) Pub. Date: Jul. 1, 2004

(54) Therapeutic agents useful for treating pain

(76) Inventors: Zhengming Chen, Belle Mead, NJ (US); Laykea Tafesse, Robbinsville, NJ (US)

Correspondence Address: JONES DAY, 222 EAST 41ST ST, NEW YORK, NY, 10017, US

(21) Appl. No.: 10/669,875

(22) Filed: Sep. 23, 2003

Related U.S. Application Data
(60) Provisional application No. 60/413,193, filed on Sep. 24, 2002.
(60) Provisional application No. 60/456,042, filed on Mar. 19, 2003.

(51) Int. Cl. A61K 31/506; C 07D 4 3/14; C 07D 4 3/04

(52) U.S. Cl. 514/252.14; 544/295; 514/252.18; 514/252.19

(57) ABSTRACT

The invention provides a compound of formula:

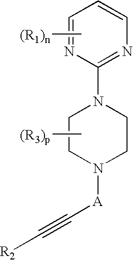

(where R₁, R₂, R₃, A, n, and p are disclosed herein) or a pharmaceutically acceptable salt thereof (a “2-Pyrimidinylpiperazine Compound”); pharmaceutical compositions comprising an effective amount of a 2-Pyrimidinylpiperazine Compound; and methods for treating or preventing a condition such as pain, urinary incontinence, an addictive disorder, Parkinson's disease, parkinsonism, anxiety, epilepsy, stroke, a seizure, a pruritic condition, psychosis, a cognitive disorder, a memory deficit, restricted brain function, Huntington's chorea, amyotrophic lateral sclerosis, dementia, retinopathy, a muscle spasm, a migraine, vomiting, dyskinesia, or depression in an animal comprising administering to an animal in need thereof an effective amount of a 2-Pyrimidinylpiperazine Compound.

This application claims the benefit of U.S. Provisional application No. 60/413,193, filed Sep. 24, 2002, and of U.S. Provisional application No. 60/456,042, filed Mar. 19, 2003, the disclosure of each of which is incorporated by reference herein in its entirety.

### 1. FIELD OF THE INVENTION

The present invention relates to 2-Pyrimidinylpiperazine Compounds, compositions comprising an effective amount of a 2-Pyrimidinylpiperazine Compound and methods for treating or preventing a condition such as pain, urinary incontinence (UI), an addictive disorder, Parkinson's disease, parkinsonism, anxiety, epilepsy, stroke, a seizure, a pruritic condition, psychosis, a cognitive disorder, a memory deficit, restricted brain function, Huntington's chorea, amyotrophic lateral sclerosis (ALS), dementia, retinopathy, a muscle spasm, a migraine, vomiting, dyskinesia, or depression, comprising administering to an animal in need thereof an effective amount of a 2-Pyrimidinylpiperazine Compound.

### 2. BACKGROUND OF THE INVENTION

Pain is the most common symptom for which patients seek medical advice and treatment. Pain can be acute or chronic. While acute pain is usually self-limited, chronic pain persists for 3 months or longer and can lead to significant changes in a patient's personality, lifestyle, functional ability and overall quality of life (K. M. Foley, Pain, in Cecil Textbook of Medicine 100-107 (J. C. Bennett and F. Plum eds., 20th ed. 1996)).

Moreover, chronic pain can be classified as either nociceptive or neuropathic. Nociceptive pain includes tissue injury-induced pain and inflammatory pain such as that associated with arthritis. Neuropathic pain is caused by damage to the peripheral or cental nervous system and is maintained by aberrant somatosensory processing. There is a large body of evidence relating activity at both Group I mGluRs (mGluR1 and mGluR5) (M. E. Fundytus, CNS Drugs 15:29-58 (2001)) and vanilloid receptors (VR1) (V. Di Marzo et al., Current Opinion in Neurobiology 12:372-379 (2002)) to pain processing. Inhibiting mGluR1 or mGluR5 reduces pain, as shown by in vivo treatment with antibodies selective for either mGluR1 or mGluR5, where neuropathic pain in rats was attenuated (M. E. Fundytus et al., NeuroReport 9:731-735 (1998)). It has also been shown that antisense oligonucleotide knockdown of mGluR1 alleviates both neuropathic and inflammatory pain (M. E. Fundytus et al., Brit. J. Pharmacol. 132:354-367 (2001); M. E. Fundytus et al., Pharmacol., Biochem. & Behavior 73:401-410 (2002)). Small molecule antagonists for mGluR5-attenuated pain in in vivo animal models are disclosed in, e.g., K. Walker et al., Neuropharmacology 40:1-9 (2000) and A. Dogrul et al., Neuroscience Let. 292:115-118 (2000)).

Nociceptive pain has been traditionally managed by administering non-opioid analgesics, such as acetylsalicylic acid, choline magnesium trisalicylate, acetaminophen, ibuprofen, fenoprofen, diflusinal, and naproxen; or opioid analgesics, including morphine, hydromorphone, methadone, levorphanol, fentanyl, oxycodone, and oxymorphone. Id. In addition to the above-listed treatments, neuropathic pain, which can be difficult to treat, has also been treated with anti-epileptics (e.g., gabapentin, carbamazepine, valproic acid, topiramate, phenyloin), NMDA antagonists (e.g., ketamine, dextromethorphan), topical lidocaine (for post-herpetic neuralgia), and tricyclic antidepressants (e.g., fluoxetine, sertraline and amitriptyline).

UI is uncontrollable urination, generally caused by bladder-detrusor-muscle instability. UI affects people of all ages and levels of physical health, both in health care settings and in the community at large. Physiologic bladder contraction results in large part from acetylcholine-induced stimulation of post-ganglionic muscarinic-receptor sites on bladder smooth muscle. Treatments for UI include the administration of drugs having bladder-relaxant properties, which help to control bladder-detrusor-muscle overactivity. For example, anticholinergics such as propantheline bromide and glycopyrrolate, and combinations of smooth-muscle relaxants such as a combination of racemic oxybutynin and dicyclomine or an anticholinergic, have been used to treat UI (See, e.g., A. J. Wein, Urol. Clin. N. Am. 22:557-577 (1995); Levin et al., J. Urol. 128:396-398 (1982); Cooke et al., S. Afr. Med. J. 63:3 (1983); R. K. Mirakhur et al., Anaesthesia 38:1195-1204 (1983)). These drugs are not effective, however, in all patients having uninhibited bladder contractions.

None of the existing commercial drug treatments for UI has achieved complete success in all classes of UI patients, nor has treatment occurred without significant adverse side effects. For example, drowsiness, dry mouth, constipation, blurred vision, headaches, tachycardia, and cardiac arrhythmia, which are related to the anticholinergic activity of traditional anti-UI drugs, can occur frequently and adversely affect patient compliance. Yet despite the prevalence of unwanted anticholinergic effects in many patients, anticholinergic drugs are currently prescribed for patients having UI. The Merck Manual of Medical Information 631-634 (R. Berkow ed., 1997).

Certain pharmaceutical agents have been administered for treating addiction. U.S. Pat. No. 5,556,838 to Mayer et al. discloses the use of nontoxic NMDA-blocking agents co-administered with an addictive substance to prevent the development of tolerance or withdrawal symptoms. U.S. Pat. No. 5,574,052 to Rose et al. discloses co-administration of an addictive substance with an antagonist to partially block the pharmacological effects of the addictive substance. U.S. Pat. No. 5,075,341 to Mendelson et al. discloses the use of a mixed opiate agonist/antagonist to treat cocaine and opiate addiction. U.S. Pat. No. 5,232,934 to Downs discloses administration of 3-phenoxypyridine to treat addiction. U.S. Pat. Nos. 5,039,680 and 5,198,459 to Imperato et al. disclose using a serotonin antagonist to treat chemical addiction. U.S. Pat. No. 5,556,837 to Nestler et. al. discloses infusing BDNF or NT-4 growth factors to inhibit or reverse neurological adaptive changes that correlate with behavioral changes in an addicted individual. U.S. Pat. No. 5,762,925 to Sagan discloses implanting encapsulated adrenal medullary cells into an animal's central nervous system to inhibit the development of opioid tolerance. U.S. Pat. No. 6,204,284 to Beer et al. discloses racemic (±)-1-(3,4-dichlorophenyl)-3-azabicyclo[3.1.0]hexane for use in the prevention or relief of a withdrawal syndrome resulting from addiction to drugs and for the treatment of chemical dependencies. Glutamate release is enhanced during opioid withdrawal (K. Jhamandas et al., J. Neurosience 16:2758-2766 (1996)). Recent evidence suggests a role for Group I mGluRs in opioid tolerance and dependence. An interaction between opioids and mGluRs was demonstrated when it was shown that an antagonist at Group I mGluRs significantly attenuated withdrawal symptoms in opioid-dependent rats (M. E. Fundytus et al., Brit. J. Pharmacol. 113:1215-1220 (1994)). More recent results show that antisense oligonucleotide knockdown of mGluR1 reduces protein kinase C activity (M. E. Fundytus et al., Brit. J. Pharmacol. 132:354-367 (2001)), which maybe associated in the development of opioid tolerance and dependence (see also M. E. Fundytus, CNS Drugs 15:29-58, (2001)). Very recently, it has been shown that antisense oligonucleotide knockdown of mGluR1 attenuates the development of opioid tolerance (R. N. Sharif et al., Brit. J. Pharmacol. 136:865-872 (2002)). Selective antagonists of the mGluR5 receptor have also been shown to exert anti-dependence activity in vivo (C. Chiamulera et al., Nature Neuroscience 4:873-874 (2001)).

Without treatment, Parkinson's disease progresses to a rigid akinetic state in which patients are incapable of caring for themselves. Death frequently results from complications of immobility, including aspiration pneumonia or pulmonary embolism. Drugs commonly used for the treatment of Parkinson's disease include carbidopa/levodopa, pergolide, bromocriptine, selegiline, amantadine, and trihexyphenidyl hydrochloride. There remains, however, a need for drugs useful for the treatment of Parkinson's disease and having an improved therapeutic profile.

Currently, benzodiazepines are the most commonly used anti-anxiety agents for generalized anxiety disorder. Benzodiazepines, however, carry the risk of producing impairment of cognition and skilled motor functions, particularly in the elderly, which can result in confusion, delerium, and falls with fractures. Sedatives are also commonly prescribed for treating anxiety. The azapirones, such as buspirone, are also used to treat moderate anxiety. The azapirones, however, are less useful for treating severe anxiety accompanied with panic attacks. Antagonists of the mGluR5 receptor have also been shown to exert anxiolytic and anti-depressant activity in in vivo animal models (E. Tatarczynska et al., Br. J. Pharmacol. 132(7):1423-1430 (2001) and P. J. M. Will et al., Trends in Pharmacological Sciences 22(7):331-37 (2001)).

Examples of drugs for treating a seizure and epilepsy include carbamazepine, ethosuximide, gabapentin, lamotrigine, phenobarbital, phenyloin, primidone, valproic acid, trimethadione, benzodiazepines, γ-vinyl GABA, acetazolamide, and felbamate. Anti-seizure drugs, however, can have side effects such as drowsiness; hyperactivity; hallucinations; inability to concentrate; central and peripheral nervous system toxicity, such as nystagmus, ataxia, diplopia, and vertigo; gingival hyperplasia; gastrointestinal disturbances such as nausea, vomiting, epigastric pain, and anorexia; endocrine effects such as inhibition of antidiuretic hormone, hyperglycemia, glycosuria, osteomalacia; and hypersensitivity such as scarlatiniform rash, morbilliform rash, Stevens-Johnson syndrome, systemic lupus erythematosus, and hepatic necrosis; and hematological reactions such as red-cell aplasia, agranulocytosis, thrombocytopenia, aplastic anemia, and megaloblastic anemia. The Merck Manual of Medical Information 345-350 (R. Berkow ed., 1997).

Symptoms of strokes vary depending on what part of the brain is affected. Symptoms include loss of or abnormal sensations in an arm or leg or one side of the body, weakness or paralysis of an arm or leg or one side of the body, partial loss of vison or hearing, double vision, dizziness, slurred speech, difficulty in thinking of the appropriate word or saying it, inability to recognize parts of the body, unusual movements, loss of bladder control, imbalance, and falling, and fainting. The symptoms can be permanent and can be associated with coma or stupor. Examples of drugs for treating strokes include anticoagulants such as heparin, drugs that break up clots such as streptokinase or tissue plasminogen activator, and drugs that reduce swelling such as mannitol or corticosteroids. The Merck Manual of Medical Information 352-355 (R. Berkow ed., 1997).

Pruritus is an unpleasant sensation that prompts scratching. Conventionally, pruritus is treated by phototherapy with ultraviolet B or PUVA or with therapeutic agents such as naltrexone, nalmefene, danazol, and tricyclic antidepressants.

Selective antagonists of the metabotropic glutamate receptor 5 (“mGluR5”) have been shown to exert analgesic activity in in vivo animal models (K. Walker et al., Neuropharmacology 40:1-9 (2000) and A. Dogrul et al., Neuroscience Let., 292(2):115-118 (2000)).

Selective antagonists of the mGluR5 receptor have also been shown to exert anti-Parkinson activity in vivo (K. J. Ossowska et al., Neuropharmacology 41(4):413-20 (2001) and P. J. M. Will et al., Trends in Pharmacological Sciences 22(7):331-37 (2001)).

Selective antagonists of the mGluR5 receptor have also been shown to exert anti-dependence activity in vivo (C. Chiamulera et al., Nature Neuroscience 4(9):873-74 (2001)).

International Publication No. WO 99/37304 by Rohne-Poulenc Rorer Pharmaceuticals, Inc. discloses oxoazaheterocyclic compounds useful for inhibiting factor Xa.

There remains, however, a clear need in the art for new drugs useful for treating or preventing pain, UI, an addictive disorder, Parkinson's disease, parkinsonism, anxiety, epilepsy, stroke, a seizure, a pruritic condition, psychosis, a cognitive disorder, a memory deficit, restricted brain function, Huntington's chorea, ALS, dementia, retinopathy, a muscle spasm, a migraine, vomiting, dyskinesia, or depression.

Citation of any reference in Section 2 of this application is not to be construed as an admission that such reference is prior art to the present application.

### 3. SUMMARY OF THE INVENTION

The present invention encompasses compounds of formula (I):

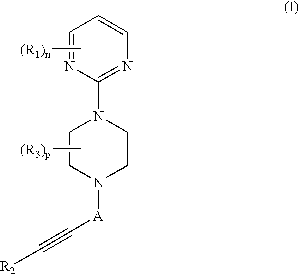

and pharmaceutically acceptable salts thereof, where:

A is —C(O)—, —C(S)—, —CH₂—, —CH(C₁-C₄ alkyl)-, or —C(C₁-C₄ alkyl)(C₁-C₄ alkyl)-;

n is an integer ranging from 0 to 3;

each R₁ is independently —(C₁-C₃)alkyl, —O—(C₁-C₃)alkyl, -halo, —C(halo)₃, —CH(halo)₂, —CH₂(halo), —NO₂, —OH, or —CN;

when A is —CH₂—, —CH(C₁-C₄ alkyl)-, or —C(C₁-C₄ alkyl)(C₁-C₄ alkyl)-, then R₂ is -phenyl, -naphthyl, or —(C₁₄)aryl, each of which is unsubstituted or substituted with one or more R₄ groups, or, when A is —C(O)— or —C(S)—, then R₂ is

(i) —H, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or

(ii)-phenyl, -naphthyl, -(C₁₄)aryl, or -(5- to 10-membered)heteroaryl, each of which is unsubstituted or substituted with one or more R₄ groups;

p is an integer ranging from 0 to 2;

each R₃ is independently —OH, -halo, —NO₂, —CN, —NH₂, —(C₁-C₃)alkyl, or —CH₂OH;

each R₄ is independently —(C₁-C₆)alkyl, —(C₂-C₆)alkenyl, —(C₂-C₆)alkynyl, -(C₃-C₈)cycloalkyl, —(C₅-C₈)cycloalkenyl, -phenyl, —(C₃-C₅)heterocycle, —C(halo)₃, —CH(halo)₂, —CH₂(halo), —CN, —OH, -halo, —N₃, —NO₂, —N(R₆)₂, —CH═NR₆, —NR₆OH, —COR₆, —C(O)OR₆, —OC(O)R₆, —OC(O)OR₆, —SR₆, —S(O)R₆, or —S(O)₂R₆;

each R₆ is independently —CN, —OH, -halo, —N₃, —NO₂, —N(R₆)₂, —CH═NR₆, —NR₆OH, —COR₆, —C(O)OR₆, —OC(O)R₆, —OC(O)OR₆, —SR₆, —S(O)R₆, or —S(O)₂R₆;

each R₆ is independently —H, —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, —(C₃-C₅)heterocycle, —C(halo)₃, —CH(halo)₂, or —CH₂(halo); and

each halo is independently —F, —Cl, —Br, or —I.

A compound of formula (I) or a pharmaceutically acceptable salt thereof (a “2-Pyrimidinylpiperazine Compound”) is useful for treating or preventing pain, UI, an addictive disorder, Parkinson's disease, parkinsonism, anxiety, epilepsy, stroke, a seizure, a pruritic condition, psychosis, a cognitive disorder, a memory deficit, restricted brain function, Huntington's chorea, ALS, dementia, retinopathy, a muscle spasm, a migraine, vomiting, dyskinesia, or depression (each being a “Condition”) in an animal.

The invention also relates to compositions comprising an effective amount of a 2-Pyrimidinylpiperazine Compound and a pharmaceutically acceptable carrier or excipient. The compositions are useful for treating or preventing a Condition in an animal.

The invention further relates to methods for treating a Condition comprising administering to an animal in need thereof an effective amount of a 2-Pyrimidinylpiperazine Compound.

The invention further relates to methods for preventing a Condition comprising administering to an animal in need thereof an effective amount of a 2-Pyrimidinylpiperazine Compound.

The invention still further relates to methods for inhibiting mGluR5 function in a cell, comprising contacting a cell capable of expressing mGluR5 with an effective amount of a 2-Pyrimidinylpiperazine Compound.

The invention still further relates to methods for inhibiting mGluR1 function in a cell, comprising contacting a cell capable of expressing mGluR1 with an effective amount of a 2-Pyrimidinylpiperazine Compound.

The invention still further relates to a method for preparing a composition comprising the step of admixing a 2-Pyrimidinylpiperazine Compound and a pharmaceutically acceptable carrier or excipient.

The invention still further relates to a kit comprising a container containing an effective amount of a 2-Pyrimidinylpiperazine Compound. The kit may further comprise printed instructions for using the 2-Pyrimidinylpiperazine Compound to treat any of the aforementioned Conditions.

The present invention can be understood more fully by reference to the following detailed description and illustrative examples, which are intended to exemplify non-limiting embodiments of the invention.

### 4. DETAILED DESCRIPTION OF THE INVENTION

### 4.1 Pyrimidinylpiperazine Compounds of Formula (I)

As stated above, the present invention encompasses 2-Pyrimidinylpiperazine Compounds of Formula (1):

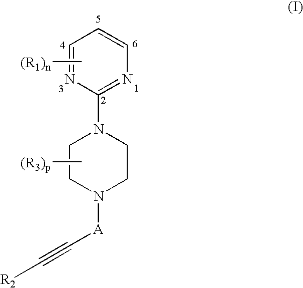

and pharmaceutically acceptable salts thereof, where R₁, R₂, R₃, A, n, and p are defined above.

In the 2-Pyrimidinylpiperazine Compounds, an R₁ group, when present, can be substituted at the 4-, 5-, or 6-position carbon atom of the pyrimidinyl ring. In one embodiment, n is 1 and R₁ is substituted at the 4-position of the pyrimidinyl ring. In another embodiment, n is 1 and R₁ is substituted at the 5-position of the pyrimidinyl ring. In another embodiment, n is 1 and R₁ is substituted at the 6-position of the pyrimidinyl ring.

In another embodiment p is 0 or 1.

In another embodiment n is 0 and p is 0.

In another embodiment A is —C(O)—.

In another embodiment A is —C(S)—.

In another embodiment A is —CH₂—.

In another embodiment A is —CH(C₁-C₄ alkyl)-.

In another embodiment A is —C(C₁-C₄ alkyl)(C₁-C₄ alkyl)-.

In another embodiment, when A is —C(O)—, R₂ is —H, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₁-C₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which is unsubstituted or substituted with one or more R₅ groups.

In another embodiment, when A is —C(O)—, R₂ is -phenyl, -naphthyl, —(C₁₋₄)aryl, or -(5- to 10-membered)heteroaryl, each of which is unsubstituted or substituted with one or more R₄ groups.

In another embodiment, when A is —C(O)—, R₂ is unsubstituted -phenyl.

In another embodiment, when A is —C(O)—, R₂ is -phenyl substituted with one or more R₄ groups.

In another embodiment, when A is —C(O)—, R₂ is -phenyl substituted in its 4-position with an R₄ group.

In another embodiment, when A is —C(O)—, R₂ is -phenyl substituted in its 4-position with a —(C₁-C₆)alkyl group.

In another embodiment, when A is —C(S)—, R₂ is —H, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which is unsubstituted or substituted with one or more R₅ groups.

In another embodiment, when A is —C(S)—, R₂ is -phenyl, -naphthyl, —(C₁₄)aryl, or -(5- to 10-membered)heteroaryl, each of which is unsubstituted or substituted with one or more R₄ groups.

In another embodiment, when A is —C(S)—, R₂ is unsubstituted -phenyl.

In another embodiment, when A is —C(S)—, R₂ is -phenyl substituted with one or more R₄ groups.

In another embodiment, when A is —C(S)—, R₂ is -phenyl substituted in its 4-position with an R₄ group.

In another embodiment, when A is —C(S)—, R₂ is -phenyl substituted in its 4-position with a —(C₁-C₆)alkyl group.

In another embodiment, when A is —CH₂—, R₂ is -phenyl, -naphthyl, or —(C₁₄)aryl, each of which is unsubstituted or substituted with one or more R₄ groups.

In another embodiment, when A is —CH₂—, R₂ is unsubstituted -phenyl.

In another embodiment, when A is —CH₂—, R₂ is -phenyl substituted with one or more R₄ groups.

In another embodiment, when A is —CH₂—, R₂ is -phenyl substituted in its 4-position with an R₄ group.

In another embodiment, when A is —CH₂—, R₂ is -phenyl substituted in its 4-position with a —(C₁-C₆)alkyl group.

In another embodiment, when A is —CH(C₁-C₄ alkyl)-, R₂ is -phenyl, -naphthyl, or —(C₁₄)aryl, each of which is unsubstituted or substituted with one or more R₄ groups.

In another embodiment, when A is —CH(C₁-C₄ alkyl)-, R₂ is unsubstituted -phenyl.

In another embodiment, when A is —CH(C₁-C₄ alkyl)-, R₂ is -phenyl substituted with one or more R₄ groups.

In another embodiment, when A is —CH(C₁-C₄ alkyl)-, R₂ is -phenyl substituted in its 4-position with an R₄ group.

In another embodiment, when A is —CH(C₁-C₄ alkyl)-, R₂ is -phenyl substituted in its 4-position with a —(C₁-C₆)alkyl group.

In another embodiment, when A is —C(C₁-C₄ alkyl)(C₁-C₄ alkyl)-, R₂ is -phenyl, -naphthyl, or —(C₁₋₄)aryl, each of which is unsubstituted or substituted with one or more R₄ groups.

In another embodiment, when A is —C(C₁-C₄ alkyl)(C₁-C₄ alkyl)-, R₂ is unsubstituted -phenyl.

In another embodiment, when A is —C(C₁-C₄ alkyl)(C₁-C₄ alkyl)-, R₂ is -phenyl substituted with one or more R₄ groups.

In another embodiment, when A is —C(C₁-C₄ alkyl)(C₁-C₄ alkyl)-, R₂ is -phenyl substituted in its 4-position with an R₄ group.

In another embodiment, when A is —C(C₁-C₄ alkyl)(C₁-C₄ alkyl)-, R₂ is -phenyl substituted in its 4-position with a —(C₁-C₆)alkyl group.

In another embodiment A is —C(O)—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is

(i) —H, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or

(ii)-phenyl, -naphthyl, —(C₁₄)aryl, or -(5- to 10-membered)heteroaryl, each of which is unsubstituted or substituted with one or more R₄ groups; and p is 0.

In another embodiment A is —C(O)—; n is 1; R₁ is substituted at the 4-position of the pyrimidinyl ring and is —CH₃, -halo, —C(halo)₃, —NO₂, —OH, or —CN; R₂ is

(i) —(C₃-C₁₀)cycloalkyl or -(3- to 7-membered)heterocycle, each of which is unsubstituted or substituted with one or more R₅ groups, or

(ii)-phenyl, -naphthyl, —(C₁₄)aryl, or -(5- to 10-membered)heteroaryl, each which is unsubstituted or substituted with one or more R₄ groups; and p is 0.

In another embodiment A is —C(O)—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is

(i) —H, —(C₁-C₁₀)alkyl, —(C₂-C₁₀)alkenyl, —(C₂-C₁₀)alkynyl —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or

(ii)-phenyl, -naphthyl, —(C₁₄)aryl, or -(5- to 10-membered)heteroaryl, each of which is unsubstituted or substituted with one or more R₄ groups;

p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —C(O)—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is

(i) —H, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or

(ii)-phenyl, -naphthyl, —(C₁₄)aryl, or -(5- to 10-membered)heteroaryl, each of which is unsubstituted or substituted with one or more R₄ groups;

p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —C(O)—; n is 1; R₁ is substituted at the 4-position of the pyrimidinyl ring and is —CH₃, -halo, —C(halo)₃, —NO₂, —OH, or —CN; R₂ is

(i) —(C₃-C₁₀)cycloalkyl or -(3- to 7-membered)heterocycle, each of which is unsubstituted or substituted with one or more R₅ groups, or

(ii) -phenyl, -naphthyl, —(C₁₄)aryl, or -(5- to 10-membered)heteroaryl, each of which is unsubstituted or substituted with one or more R₄ groups;

p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —C(O)—; n is 1; R₁ is substituted at the 4-position of the pyrimidinyl ring and is —CH₃, -halo, —C(halo)₃, —NO₂, —OH, or —CN; R₂ is

(i) —(C₃-C₁₀)cycloalkyl or -(3- to 7-membered)heterocycle, each of which is unsubstituted or substituted with one or more R₅ groups, or

(ii) -phenyl, -naphthyl, —(C₁₄)aryl, or -(5- to 10-membered)heteroaryl, each of which is unsubstituted or substituted with one or more R₄ groups;

p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —C(O)—; n is 1; R₁ is substituted at the 4-position of the pyrimidinyl ring and is —CH₃, -halo, —C(halo)₃, —NO₂, —OH, or —CN; R₂ is -phenyl, -naphthyl, —(C₄)aryl, or -(5- to 10-membered)heteroaryl, each which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —C(O)—; n is 1; R₁ is substituted at the 4-position of the pyrimidinyl ring and is —CH₃, -halo, —C(halo)₃, —NO₂, —OH, or —CN; R₂ is -phenyl, -naphthyl, —(C₁₄)aryl, or -(5- to 10-membered)heteroaryl, each which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —C(S)—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is

(i) —H, —(C₁-C₁₀)alkyl, —(C₂-C₁₀)alkenyl, —(C₂-C₁₀)alkynyl —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or

(ii)-phenyl, -naphthyl, —(C₁₋₄)aryl, or -(5- to 10-membered)heteroaryl, each of which is unsubstituted or substituted with one or more R₄ groups; and

p is 0.

In another embodiment A is —C(S)—; n is 1; R₁ is substituted at the 4-position of the pyrimidinyl ring and is —CH₃, -halo, —C(halo)₃, —NO₂, —OH, or —CN; R₂ is

(i) —(C₃-C₁₀)cycloalkyl or -(3- to 7-membered)heterocycle, each of which is unsubstituted or substituted with one or more R₅ groups, or

(ii)-phenyl, -naphthyl, —(C₁₋₄)aryl, or -(5- to 10-membered)heteroaryl, each which is unsubstituted or substituted with one or more R₄ groups; and

p is 0.

In another embodiment A is —C(S)—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is

(i) —H, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₈-C₁₄)tricycloalkyl, -(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or

(ii)-phenyl, -naphthyl, —(C₁₋₄)aryl, or -(5- to 10-membered)heteroaryl, each of which is unsubstituted or substituted with one or more R₄ groups;

p is 1; and R₃ is —OH, -halo, —NO₂, —CN, —NH2, —(C₁-C₃)alkyl, or —CH₂OH and is attached to a carbon atom adjacent to the piperazino nitrogen atom attached to the pyrimidinyl ring.

In another embodiment A is —C(S)—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is

(i) —H, —(C, —C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or

(ii) -phenyl, -naphthyl, —(C₁₄)aryl, or -(5- to 10-membered)heteroaryl, each of which is unsubstituted or substituted with one or more R₄ groups;

p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —C(S)—; n is 1; R₁ is substituted at the 4-position of the pyrimidinyl ring and is —CH₃, -halo, —C(halo)₃, —NO₂, —OH, or —CN; R₂ is

(i) —(C₃-C₁₀)cycloalkyl or -(3- to 7-membered)heterocycle, each of which is unsubstituted or substituted with one or more R₅ groups, or

(ii)-phenyl, -naphthyl, —(C₄)aryl, or -(5- to 10-membered)heteroaryl, each of which is unsubstituted or substituted with one or more R₄ groups;

p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —C(S)—; n is 1; R₁ is substituted at the 4-position of the pyrimidinyl ring and is —CH₃, -halo, —C(halo)₃, —NO₂, —OH, or —CN; R₂ is

(i) —(C₃-C₁₀)cycloalkyl or -(3- to 7-membered)heterocycle, each of which is unsubstituted or substituted with one or more R₅ groups, or

(ii) -phenyl, -naphthyl, —(C₁₄)aryl, or -(5- to 10-membered)heteroaryl, each of which is unsubstituted or substituted with one or more R₄ groups;

p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —C(S)—; n is 1; R₁ is substituted at the 4-position of the pyrimidinyl ring and is —CH₃, -halo, —C(halo)₃, —NO₂, —OH, or —CN; R₂ is -phenyl, -naphthyl, —(C₁₄)aryl, or -(5- to 10-membered)heteroaryl, each which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —C(S)—; n is 1; R₁ is substituted at the 4-position of the pyrimidinyl ring and is —CH₃, -halo, —C(halo)₃, —NO₂, —OH, or —CN; R₂ is -phenyl, -naphthyl, —(C₄)aryl, or -(5- to 10-membered)heteroaryl, each which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —CH₂—, —CH(C₁-C₄ alkyl)-, or —C(C₁-C₄ alkyl)(C₁-C₄ alkyl)-; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is -phenyl, -naphthyl, or —(C₁₄)aryl, each of which is unsubstituted or substituted with one or more R₄ groups; and p is 0.

In another embodiment A is —CH₂—, —CH(C₁-C₄ alkyl)-, or —C(C₁-C₄ alkyl)(C₁-C₄ alkyl)-; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl which is unsubstituted or substituted with one or more R₄ groups; and p is 0.

In another embodiment A is —CH₂—, —CH(C₁-C₄ alkyl)-, or —C(C₁-C₄ alkyl)(C₁-C₄ alkyl)-; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is -phenyl, -naphthyl, or —(C₁₄)aryl, each of which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —CH₂—, —CH(C₁-C₄ alkyl)-, or —C(C₁-C₄ alkyl)(C₁-C₄ alkyl)-; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is -phenyl, -naphthyl, or —(C₁₄)aryl, each of which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —CH₂—, —CH(C₁-C₄ alkyl)-, or —C(C₁-C₄ alkyl)(C₁-C₄ alkyl)-; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl, each of which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —CH₂—, —CH(C₁-C₄ alkyl)-, or —C(C₁-C₄ alkyl)(C₁-C₄ alkyl)-; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl of which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —CH₂—, —CH(C₁-C₄ alkyl)-, or —C(C₁-C₄ alkyl)(C₁-C₄ alkyl)-; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —CH₂—, —CH(C₁-C₄ alkyl)-, or —C(C₁-C₄ alkyl)(C₁-C₄ alkyl)-; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl, each which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —CH₂—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is -phenyl, -naphthyl, or —(C₁₄)aryl, each of which is unsubstituted or substituted with one or more R₄ groups; and p is 0.

In another embodiment A is —CH₂—; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl, each which is unsubstituted or substituted with one or more R₄ groups; and p is 0.

In another embodiment A is —CH₂—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is -phenyl, -naphthyl, or —(C₁₄)aryl, each of which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —CH₂—; n is 1; R₁ is substituted at the 4-position of the pyrimidinyl ring and is —CH₃, -halo, —C(halo)₃, —CH(halo)₂, —CH₂(halo), —NO₂, —OH, or —CN; R₂ is -phenyl, -naphthyl, or —(C₁₄)aryl, each of which is unsubstituted or substituted with one or more R₄ groups; p is 1; and R₃ is —OH, -halo, —NO₂, —CN, —N₁₂, —(C₁-C₃)alkyl, or —CH₂OH and is attached to a carbon atom adjacent to the piperazino nitrogen atom attached to the A group.

In another embodiment A is —CH₂—; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl, each of which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —CH₂—; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl, each of which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

In another embodiment A is —CH₂—; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl, each which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the pyrimidinyl ring.

In another embodiment A is —CH₂—; n is 1; R₁ is substituted at the 4-position of the pyrimidinyl ring and is —CH₃, -halo, —C(halo)₃, —NO₂, —OH, or —CN; R₂ is -phenyl, -naphthyl, or —(C₁₄)aryl, each which is unsubstituted or substituted with one or more R₄ groups; p is 1; and R₃ is —OH, -halo, —NO₂, —CN, —NH₂, —(C₁-C₃)alkyl, or —CH₂OH and is attached to a carbon atom adjacent to the piperazino nitrogen atom attached to the A group.

### 4.2 Pyrimidinylpiperazine Compounds of Formula (Ia)

In another embodiment, the 2-Pyrimidinylpiperazine Compounds of Formula (I) have the Formula (Ia):

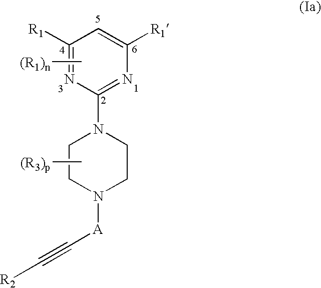

and pharmaceutically acceptable salts thereof, where:

A is —C(O)—, —C(S)—, —CH₂—, —CH(C₁-C₄ alkyl)-, or —C(C₁-C₄ alkyl)(C₁-C₄ alkyl)-;

R₁ and R₁′ are independently —H, —(C₁-C₃)alkyl, —O—(C₁-C₃)alkyl, -halo, —C(halo)₃, —CH(halo)₂, —CH₂(halo), —NO₂, —OH, or —CN;

when A is —CH₂—, —CH(C₁-C₄ alkyl)-, or —C(C₁-C₄ alkyl)(C₁-C₄ alkyl)-, then R₂ is -phenyl, -naphthyl, or —(C₁₄)aryl, each of which is unsubstituted or substituted with one or more R₄ groups, or, when A is —C(O)— or —C(S)—, then R₂ is

(i) —H, —(C₁-C₁₀)alkyl, —(C₂-C₁₀)alkenyl, —(C₂-C₁₀)alkynyl, —(C₃-C₁₀)cycloalkyl, —(C₈-C₈-C₁₄)bicycloalkyl, —(C₈-C₁₄)tricycloalkyl, —(C₅-C₁₀)cycloalkenyl, -(C₈-C₁₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or

(ii)-phenyl, -naphthyl, —(C₁₄)aryl, or -(5- to 10-membered)heteroaryl, each of which is unsubstituted or substituted with one or more R₄ groups;

p is an integer ranging from 0 to 2;

each R₃ is independently —OH, -halo, —NO₂, —CN, —NH₂, —(C₁-C₃)alkyl, or —CH₂OH;

each R₄ is independently —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, —(C₃-C₅)heterocycle, —C(halo)₃, —CH(halo)₂, —CH₂(halo), —CN, —OH, -halo, —N₃, —NO₂, —N(R₆)₂, —CH═NR₆, —NR₆OH, —COR₆, —C(O)OR₆, —OC(O)R₆, —OC(O)OR₆, —SR₆, —S(O)R₆, or —S(O)₂R₆;

each R₅ is independently —CN, —OH, -halo, —N₃, —NO₂, —N(R₆)₂, —CH═NR₆, —NROH, —COR₆, —C(O)OR₆, —OC(O)R₆, —OC(O)OR₆, —SR₆, —S(O)R₆, or —S(O)₂R₆; and

each R₆ is independently —H, —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, —(C₃-C₅)heterocycle, —C(halo)₃, —CH(halo)₂, or —CH₂(halo); and

each halo is independently —F, —Cl, —Br, or —I.

In one embodiment p is 0 or 1.

In another embodiment R₁ and R₁′ are —H.

In another embodiment R₁ and R₁′ are —CH₃.

In another embodiment R₁ is —OCH₃ and R₁′ is —CH₃.

In another embodiment R₁ is -halo and R₁′ is —CH₃.

In another embodiment R₁ is —Cl and R₁′ is —CH₃.

In another embodiment A is —C(O)—; R₁ is —CH₃, —OCH₃ or -halo; R₁′ is —H or —CH₃; R₂ is -phenyl or -pyridyl, each which is unsubstituted or substituted with one or more R₄ groups; and R₃ is —H, —CH₃ or —CH₂OH and is attached to a carbon atom adjacent to the piperazino nitrogen atom attached to the A group.

In another embodiment A is —C(O)—; R₁ is —CH₃, —OCH₃ or —Cl; R₁′ is —H or —CH₃; R₂ is -phenyl or -pyridyl, each which is unsubstituted or substituted with one or more R₄ groups; and R₃ is —H, —CH₃ or —CH₂OH and is attached to a carbon atom adjacent to the piperazino nitrogen atom attached to the A group.

In another embodiment A is —C(O)—; R₁ is —CH₃, —OCH₃ or -halo; R₁′ is —H or —CH₃; R₂ is -phenyl or -pyridyl, each which is unsubstituted or substituted with one or more R₄ groups selected from -halo and —OCH₃; and R₃ is —H, —CH₃ or —CH₂OH and is attached to a carbon atom adjacent to the piperazino nitrogen atom attached to the A group.

In another embodiment A is —C(O)—; R₁ is —CH₃, —OCH₃ or —Cl; R₁′ is —H or —CH₃; R₂ is -phenyl or -pyridyl, each which is unsubstituted or substituted with one or more R₄ groups selected from —F and —OCH₃; and R₃ is —H, —CH₃ or —CH₂OH and is attached to a carbon atom adjacent to the piperazino nitrogen atom attached to the A group.

In another embodiment A is —C(O)—; R₁ is —CH₃, —OCH₃ or —Cl; R₁′ is —H or —CH₃; R₂ is -phenyl which is unsubstituted or substituted with one R₄ group para to its point of attachment to (—C≡C-A-) and selected from —F and —OCH₃; and R₃ is —H, —CH₃ or —CH₂OH and is attached to a carbon atom adjacent to the piperazino nitrogen atom attached to the A group.

In another embodiment A is —C(O)—; R₁ is —CH₃, —OCH₃ or —Cl; R₁′ is —H or —CH₃; R₂ is 2-pyridyl which is unsubstituted or substituted with one R₄ group at the 5-position of the 2-pyridyl selected from —F and —OCH₃; and R₃ is —H, —CH₃ or —CH₂OH and is attached to a carbon atom adjacent to the piperazino nitrogen atom attached to the A group.

In another embodiment A is —C(O)—; R₁ is —CH₃, —OCH₃ or —Cl; R₁′ is —H or —CH₃; R₂ is 3-pyridyl which is unsubstituted or substituted with one R₄ group at the 6-position of the 3-pyridyl selected from —F and —OCH₃; and R₃ is —H, —CH₃ or —CH₂OH and is attached to a carbon atom adjacent to the piperazino nitrogen atom attached to the A group.

In the 2-Pyrimidinylpiperazine Compounds each R₃ group, if present, can be on any carbon of the piperazino ring. In one embodiment, the 2-Pyrimidinylpiperazine Compounds have only one R₃ group, and that R₃ group is attached to a carbon atom adjacent to the piperazino nitrogen atom attached to the pyrimidinyl group. In another embodiment, the 2-Pyrimidinylpiperazine Compound has only one R₃ group, and that R₃ group is attached to a carbon atom adjacent to the piperazino nitrogen atom attached to the A group.

In another embodiment, two R₃ groups are on a single atom of the piperazino ring. In another embodiment, an R₃ group is attached to a carbon atom adjacent to the piperazino nitrogen atom attached to the pyrimidinyl group and another R₃ group is attached to a carbon atom adjacent to the piperazino nitrogen atom attached to the A group.

In another embodiment, the 2-Pyrimidinylpiperazine Compound has two R₃ groups, each being attached to a different carbon atom adjacent to the piperazino nitrogen atom attached to the pyrimidinyl group. In another embodiment, the 2-Pyrimidinylpiperazine Compound has two R₃ groups, each being attached to a different carbon atom adjacent to the piperazino nitrogen atom attached to the A group.

In one embodiment, wherein the 2-Pyrimidinylpiperazine Compound has one or two R₃ groups, the carbon atom to which an R₃ group is attached has the (R) configuration. In another embodiment, wherein the 2-Pyrimidinylpiperazine Compound has one or two R₃ groups, the carbon atom to which the R₃ group is attached has the (S) configuration. In another embodiment, the 2-Pyrimidinylpiperazine Compound has one or two R₃ groups, and at least one of the carbon atoms to which an R₃ group is attached has the (R) configuration. In another embodiment, the 2-Pyrimidinylpiperazine Compound has one or two R₃ groups, and at least one of the carbon atoms to which an R₃ group is attached has the (S) configuration.

In another embodiment, the 2-Pyrimidinylpiperazine Compound has one or two R₃ groups, an R₃ group is attached to a carbon atom adjacent to the piperazino nitrogen atom attached to the pyrimidinyl group, and the carbon to which the R₃ group is attached is in the (R) configuration. In another embodiment, the 2-Pyrimidinylpiperazine Compound has one or two R₃ groups, an R₃ group is attached to a carbon atom adjacent to the piperazino nitrogen attached to the pyrimidinyl group, the carbon to which the R₃ group is attached is in the (R) configuration, and R₃ is —(C₁-C₃)alkyl. In another embodiment, the 2-Pyrimidinylpiperazine Compound has one or two R₃ groups, an R₃ group is attached to a carbon atom adjacent to the piperazino nitrogen attached to the pyrimidinyl group, the carbon to which the R₃ group is attached is in the (R) configuration, and R₃ is —CH₃. In another embodiment, the 2-Pyrimidinylpiperazine Compound has one or two R₃ groups, an R₃ group is attached to a carbon atom adjacent to the piperazino nitrogen attached to the pyrimidinyl group, the carbon to which the R₃ group is attached is in the (R) configuration, and R₃ is —CH₂OH. In another embodiment, the 2-Pyrimidinylpiperazine Compound has one or two R₃ groups, an R₃ group is attached to a carbon atom adjacent to the piperazino nitrogen attached to the pyrimidinyl group, the carbon to which the R₃ group is attached is in the (R) configuration, and R₃ is —CH₂CH₃.

In another embodiment, the 2-Pyrimidinylpiperazine Compound has one or two R₃ groups, an R₃ group is attached to a carbon atom adjacent to the piperazino nitrogen atom attached to the A group, and the carbon to which the R₃ group is attached is in the (R) configuration. In another embodiment, the 2-Pyrimidinylpiperazine Compound has one or two R₃ groups, an R₃ group is attached to a carbon atom adjacent to the piperazino nitrogen attached to the A group, the carbon to which the R₃ group is attached is in the (R) configuration, and R₃ is —(C₁-C₃)alkyl. In another embodiment, the 2-Pyrimidinylpiperazine Compound has one or two R₃ groups, an R₃ group is attached to a carbon atom adjacent to the piperazino nitrogen attached to the A group, the carbon to which the R₃ group is attached is in the (R) configuration, and R₃ is —CH₃. In another embodiment, the 2-Pyrimidinylpiperazine Compound has one or two R₃ groups, an R₃ group is attached to a carbon atom adjacent to the piperazino nitrogen attached to the A group, the carbon to which the R₃ group is attached is in the (R) configuration, and R₃ is —CH₂OH. In another embodiment, the 2-Pyrimidinylpiperazine Compound has one or two R₃ groups, an R₃ group is attached to a carbon atom adjacent to the piperazino nitrogen attached to the A group, the carbon to which the R₃ group is attached is in the (R) configuration, and R₃ is —CH₂CH₃.

In another embodiment, the 2-Pyrimidinylpiperazine Compound has one or two R₃ groups, an R₃ group is attached to a carbon atom adjacent to the piperazino nitrogen atom attached to the pyrimidinyl group, and the carbon to which the R₃ group is attached is in the (S) configuration. In another embodiment, the 2-Pyrimidinylpiperazine Compound has one or two R₃ groups, an R₃ group is attached to a carbon atom adjacent to the piperazino nitrogen attached to the pyrimidinyl group, the carbon to which the R₃ group is attached is in the (S) configuration, and R₃ is —(C₁-C₃)alkyl. In another embodiment, the 2-Pyrimidinylpiperazine Compound has one or two R₃ groups, an R₃ group is attached to a carbon atom adjacent to the piperazino nitrogen attached to the pyrimidinyl group, the carbon to which the R₃ group is attached is in the (S) configuration, and R₃ is —CH₃. In another embodiment, the 2-Pyrimidinylpiperazine Compound has one or two R₃ groups, an R₃ group is attached to a carbon atom adjacent to the piperazino nitrogen attached to the pyrimidinyl group, the carbon to which the R₃ group is attached is in the (S) configuration, and R₃ is —CH₂OH. In another embodiment, the 2-Pyrimidinylpiperazine Compound has one or two R₃ groups, an R₃ group is attached to a carbon atom adjacent to the piperazino nitrogen attached to the pyrimidinyl group, the carbon to which the R₃ group is attached is in the (S) configuration, and R₃ is —CH₂CH₃.

In another embodiment, the 2-Pyrimidinylpiperazine Compound has one or two R₃ groups, an R₃ group is attached to a carbon atom adjacent to the piperazino nitrogen atom attached to the A group, and the carbon to which the R₃ group is attached is in the (S) configuration. In another embodiment, the 2-Pyrimidinylpiperazine Compound has one or two R₃ groups, an R₃ group is attached to a carbon atom adjacent to the piperazino nitrogen attached to the A group, the carbon to which the R₃ group is attached is in the (S) configuration, and R₃ is —(C₁-C₃)alkyl. In another embodiment, the 2-Pyrimidinylpiperazine Compound has one or two R₃ groups, an R₃ group is attached to a carbon atom adjacent to the piperazino nitrogen attached to the A group, the carbon to which the R₃ group is attached is in the (S) configuration, and R₃ is —CH₃. In another embodiment, the 2-Pyrimidinylpiperazine Compound has one or two R₃ groups, an R₃ group is attached to a carbon atom adjacent to the piperazino nitrogen attached to the A group, the carbon to which the R₃ group is attached is in the (S) configuration, and R₃ is —CH₂OH. In another embodiment, the 2-Pyrimidinylpiperazine Compound has one or two R₃ groups, an R₃ group is attached to a carbon atom adjacent to the piperazino nitrogen attached to the A group, the carbon to which the R₃ group is attached is in the (S) configuration, and R₃ is —C₁H₂CH₃.

In another embodiment, the 2-Pyrimidinylpiperazine Compound has only one R₃ group, the R₃ group is attached to a carbon atom adjacent to the piperazino nitrogen atom attached to the pyrimidinyl group, and the carbon to which the R₃ group is attached is in the (R) configuration. In another embodiment, the 2-Pyrimidinylpiperazine Compound has only one R₃ group, the R₃ group is attached to a carbon atom adjacent to the piperazino nitrogen attached to the pyrimidinyl group, the carbon to which the R₃ group is attached is in the (R) configuration, and R₃ is —(C₁-C₃)alkyl. In another embodiment, the 2-Pyrimidinylpiperazine Compound has only one R₃ group, the R₃ group is attached to a carbon atom adjacent to the piperazino nitrogen attached to the pyrimidinyl group, the carbon to which the R₃ group is attached is in the (R) configuration, and R₃ is —CH₃. In another embodiment, the 2-Pyrimidinylpiperazine Compound has only one R₃ group, the R₃ group is attached to a carbon atom adjacent to the piperazino nitrogen attached to the pyrimidinyl group, the carbon to which the R³ group is attached is in the (R) configuration, and R₃ is —CH₂OH. In another embodiment, the 2-Pyrimidinylpiperazine Compound has only one R₃ group, the R₃ group is attached to a carbon atom adjacent to the piperazino nitrogen attached to the pyrimidinyl group, the carbon to which the R₃ group is attached is in the (R) configuration, and R₃ is —CH₂CH₃.

In another embodiment, the 2-Pyrimidinylpiperazine Compound has only one R₃ group, the R₃ group is attached to a carbon atom adjacent to the piperazino nitrogen atom attached to the A group, and the carbon to which the R₃ group is attached is in the (R) configuration. In another embodiment, the 2-Pyrimidinylpiperazine Compound has only one R₃ group, the R₃ group is attached to a carbon atom adjacent to the piperazino nitrogen attached to the A group, the carbon to which the R₃ group is attached is in the (R) configuration, and R₃ is —(C₁-C₃)alkyl. In another embodiment, the 2-Pyrimidinylpiperazine Compound has only one R₃ group, the R₃ group is attached to a carbon atom adjacent to the piperazino nitrogen attached to the A group, the carbon to which the R₃ group is attached is in the (R) configuration, and R₃ is —CH₃. In another embodiment, the 2-Pyrimidinylpiperazine Compound has only one R₃ group, the R₃ group is attached to a carbon atom adjacent to the piperazino nitrogen attached to the A group, the carbon to which the R₃ group is attached is in the (R) configuration, and R₃ is —CH₂OH. In another embodiment, the 2-Pyrimidinylpiperazine Compound has only one R₃ group, the R₃ group is attached to a carbon atom adjacent to the piperazino nitrogen attached to the A group, the carbon to which the R₃ group is attached is in the (R) configuration, and R₃ is —CH₂CH₃.

In another embodiment, the 2-Pyrimidinylpiperazine Compound has only one R₃ group, the R₃ group is attached to a carbon atom adjacent to the piperazino nitrogen atom attached to the pyrimidinyl group, and the carbon to which the R₃ group is attached is in the (S) configuration. In another embodiment, the 2-Pyrimidinylpiperazine Compound has only one R₃ group, the R₃ group is attached to a carbon atom adjacent to the piperazino nitrogen attached to the pyrimidinyl group, the carbon to which the R₃ group is attached is in the (S) configuration, and R₃ is —(C₁-C₃)alkyl. In another embodiment, the 2-Pyrimidinylpiperazine Compound has only one R₃ group, the R₃ group is attached to a carbon atom adjacent to the piperazino nitrogen attached to the pyrimidinyl group, the carbon to which the R₃ group is attached is in the (S) configuration, and R₃ is —CH₃. In another embodiment, the 2-Pyrimidinylpiperazine Compound has only one R₃ group, the R₃ group is attached to a carbon atom adjacent to the piperazino nitrogen attached to the pyrimidinyl group, the carbon to which the R₃ group is attached is in the (S) configuration, and R₃ is —CH₂OH. In another embodiment, the 2-Pyrimidinylpiperazine Compound has only one R₃ group, the R₃ group is attached to a carbon atom adjacent to the piperazino nitrogen attached to the pyrimidinyl group, the carbon to which the R₃ group is attached is in the (S) configuration, and R₃ is —CH₂CH₃.

In another embodiment, the 2-Pyrimidinylpiperazine Compound has only one R₃ group, the R₃ group is attached to a carbon atom adjacent to the piperazino nitrogen atom attached to the A group, and the carbon to which the R₃ group is attached is in the (S) configuration. In another embodiment, the 2-Pyrimidinylpiperazine Compound has only one R₃ group, the R₃ group is attached to a carbon atom adjacent to the piperazino nitrogen attached to the A group, the carbon to which the R₃ group is attached is in the (S) configuration, and R₃ is —(C₁-C₃)alkyl. In another embodiment, the 2-Pyrimidinylpiperazine Compound has only one R₃ group, the R₃ group is attached to a carbon atom adjacent to the piperazino nitrogen attached to the A group, the carbon to which the R₃ group is attached is in the (S) configuration, and R₃ is —CH₃. In another embodiment, the 2-Pyrimidinylpiperazine Compound has only one R₃ group, the R₃ group is attached to a carbon atom adjacent to the piperazino nitrogen attached to the A group, the carbon to which the R₃ group is attached is in the (S) configuration, and R₃ is —CH₂OH. In another embodiment, the 2-Pyrimidinylpiperazine Compound has only one R₃ group, the R₃ group is attached to a carbon atom adjacent to the piperazino nitrogen attached to the A group, the carbon to which the R₃ group is attached is in the (R) configuration, and R₃ is —CH₂CH₃.

In a preferred embodiment, the R₃ group is attached to a carbon atom adjacent to the piperazino nitrogen attached to the A group. In another preferred embodiment, the R₃ group is attached to a carbon atom adjacent to the piperazino nitrogen attached to the A group and the R₃ group is a —CH₃. In another preferred embodiment, the R₃ group is attached to a carbon atom adjacent to the piperazino nitrogen attached to the A group and the R₃ group is a —CF₃. In another preferred embodiment, the R₃ group is attached to a carbon atom adjacent to the piperazino nitrogen attached to the A group and the R₃ group is a —CH₂CH₃. In another preferred embodiment, the R₃ group is attached to a carbon atom adjacent to the piperazino nitrogen attached to the A group and the carbon to which the R₃ group is attached is in the (R) configuration. In another preferred embodiment, the R₃ group is attached to a carbon atom adjacent to the piperazino nitrogen attached to the A group, the carbon to which the R₃ group is attached is in the (R) configuration, and the R₃ group is a —CH₃. In another preferred embodiment, the R₃ group is attached to a carbon atom adjacent to the piperazino nitrogen attached to the A group, the carbon to which the R₃ group is attached is in the (R) configuration, and the R₃ group is a —CF₃. In another preferred embodiment, the R₃ group is attached to a carbon atom adjacent to the piperazino nitrogen attached to the A group, the carbon to which the R₃ group is attached is in the (R) configuration, and the R₃ group is a —CH₂CH₃.

In another embodiment A is —C(O)—; n is 2; an R₁ is substituted at the 4-position of the pyrimidinyl ring and is —CH₃, —OCH₃ or -halo; the other R₁ (denoted hereinafter for convenience as “R₁′” to distinguish it from the R₁ substituted at the 4-position) is substituted at the 6-position of the pyrimidinyl ring; and R₁′ is —H or —CH₃; R₂ is -phenyl or -pyridyl, each which is unsubstituted or substituted with one or more R₄ groups; and R₃ is —H, —CH₃ or —CH₂OH and is attached to a carbon atom adjacent to the piperazino nitrogen atom attached to the A group.

In another embodiment A is —C(O)—; n is 2; R₁ is substituted at the 4-position of the pyrimidinyl ring and is —CH₃, —OCH₃ or —Cl; R₁′ is substituted at the 6-position of the pyrimidinyl ring and is —H or —CH₃; R₂ is -phenyl or -pyridyl, each which is unsubstituted or substituted with one or more R₄ groups; and R₃ is —H, —CH₃ or —CH₂OH and is attached to a carbon atom adjacent to the piperazino nitrogen atom attached to the A group.

In another embodiment A is —C(O)—; n is 2; R₁ is substituted at the 4-position of the pyrimidinyl ring and is —CH₃, —OCH₃ or -halo; R₁′ is substituted at the 6-position of the pyrimidinyl ring and is —H or —CH₃; R₂ is -phenyl or -pyridyl, each which is unsubstituted or substituted with one or more R₄ groups selected from -halo and —OCH₃; and R₃ is —H, —CH₃ or —CH₂OH and is attached to a carbon atom adjacent to the piperazino nitrogen atom attached to the A group.

In another embodiment A is —C(O)—; n is 2; R₁ is substituted at the 4-position of the pyrimidinyl ring and is —CH₃, —OCH₃ or —Cl; R₁′ is substituted at the 6-position of the pyrimidinyl ring and is —H or —CH₃; R₂ is -phenyl or -pyridyl, each which is unsubstituted or substituted with one or more R₄ groups selected from —F and —OCH₃; and R₃ is —H, —CH₃ or —CH₂OH and is attached to a carbon atom adjacent to the piperazino nitrogen atom attached to the A group.

In another embodiment A is —C(O)—; n is 2; R₁ is substituted at the 4-position of the pyrimidinyl ring and is —CH₃, —OCH₃ or —Cl; R₁′ is substituted at the 6-position of the pyrimidinyl ring and is —H or —CH₃; R₂ is -phenyl which is unsubstituted or substituted with one R₄ group para to its point of attachment to (—C═C-A-) and selected from —F and —OCH₃; and R₃ is —H, —CH₃ or —CH₂OH and is attached to a carbon atom adjacent to the piperazino nitrogen atom attached to the A group.

In another embodiment A is —C(O)—; n is 2; R₁ is substituted at the 4-position of the pyrimidinyl ring and is —CH₃, —OCH₃ or —Cl; R₁′ is substituted at the 6-position of the pyrimidinyl ring and is —H or —CH₃; R₂ is 2-pyridyl and is unsubstituted or substituted with one R₄ group at the 5-position of the 2-pyridyl and selected from —F and —OCH₃; and R₃ is —H, —CH₃ or —CH₂OH and is attached to a carbon atom adjacent to the piperazino nitrogen atom attached to the A group.

In another embodiment A is —C(O)—; n is 2; R₁ is substituted at the 4-position of the pyrimidinyl ring and is —CH₃, —OCH₃ or —Cl; R₁′ is substituted at the 6-position of the pyrimidinyl ring and is —H or —CH₃; R₂ is 3-pyridyl and is unsubstituted or substituted with one R₄ group at the 6-position of the 3-pyridyl and selected from —F and —OCH₃; and R₃ is —H, —CH₃ or —CH₂OH and is attached to a carbon atom adjacent to the piperazino nitrogen atom attached to the A group.

Illustrative 2-Pyrimidinylpiperazine Compounds are listed below in Tables 1-4:

TABLE 1

| | |
|---|---|
| | (IIa) |
| 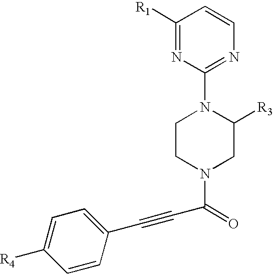 | |
| | (IIb) |
| 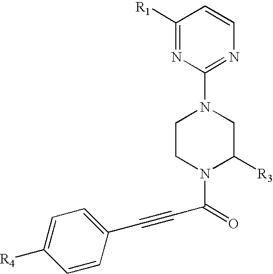 | |

and pharmaceutically acceptable salts thereof, where:

| Compound | R₁ | R₃ | R₄ |
|---|---|---|---|
| AAA(IIa) | —H | —H | —H |
| AAB(IIa) | —H | —H | —CH₃ |
| AAC(IIa) | —H | —H | -n-propyl |
| AAD(IIa) | —H | —H | -n-butyl |
| AAE(IIa) | —H | —H | -t-butyl |
| AAF(IIa) | —H | —H | -iso-butyl |
| AAG(IIa) | —H | —H | —OCH₃ |
| AAH(IIa) | —H | —H | —OC₂H₅ |
| AAI(IIa) | —H | —H | —OC₃H₇ |
| AAJ(IIa) | —H | —H | —CHF₂ |
| AAK(IIa) | —H | —H | —CF₃ |
| AAL(IIa) | —H | —H | —CHCl₂ |
| AAM(IIa) | —H | —H | —CCl₃ |
| AAN(IIa) | —H | —H | —F |
| AAO(IIa) | —H | —H | —Cl |
| AAP(IIa) | —H | —H | —Br |
| AAQ(IIa) | —H | —H | —I |
| AAR(IIa) or (IIb) | —H | —OH | —H |
| AAS(IIa) or (IIb) | —H | —OH | —CH₃ |
| AAT(IIa) or (IIb) | —H | —OH | -n-propyl |
| AAU(IIa) or (IIb) | —H | —OH | -n-butyl |
| AAV(IIa) or (IIb) | —H | —OH | -t-butyl |
| AAW(IIa) or (IIb) | —H | —OH | -iso-butyl |
| AAX(IIa) or (IIb) | —H | —OH | —OCH₃ |
| AAY(IIa) or (IIb) | —H | —OH | —OC₂H₅ |
| AAZ(IIa) or (IIb) | —H | —OH | —OC₃H₇ |
| ABA(IIa) or (IIb) | —H | —OH | —CHF₂ |
| ABB(IIa) or (IIb) | —H | —OH | —CF₃ |
| ABC(IIa) or (IIb) | —H | —OH | —CHCl₂ |
| ABD(IIa) or (IIb) | —H | —OH | —CCl₃ |
| ABE(IIa) or (IIb) | —H | —OH | —F |
| ABF(IIa) or (IIb) | —H | —OH | —Cl |
| ABG(IIa) or (IIb) | —H | —OH | —Br |
| ABH(IIa) or (IIb) | —H | —OH | —I |
| ABI(IIa) or (IIb) | —H | —F | —H |
| ABJ(IIa) or (IIb) | —H | —F | —CH₃ |
| ABK(IIa) or (IIb) | —H | —F | -n-propyl |
| ABL(IIa) or (IIb) | —H | —F | -n-butyl |
| ABM(IIa) or (IIb) | —H | —F | -t-butyl |
| ABN(IIa) or (IIb) | —H | —F | -iso-butyl |
| ABO(IIa) or (IIb) | —H | —F | —OCH₃ |
| ABP(IIa) or (IIb) | —H | —F | —OC₂H₅ |
| ABQ(IIa) or (IIb) | —H | —F | —OC₃H₇ |
| ABR(IIa) or (IIb) | —H | —F | —CHF₂ |
| ABS(IIa) or (IIb) | —H | —F | —CF₃ |
| ABT(IIa) or (IIb) | —H | —F | —CHCl₂ |
| ABU(IIa) or (IIb) | —H | —F | —CCl₃ |
| ABV(IIa) or (IIb) | —H | —F | —F |
| ABW(IIa) or (IIb) | —H | —F | —Cl |
| ABX(IIa) or (IIb) | —H | —F | —Br |
| ABY(IIa) or (IIb) | —H | —F | —I |
| ABZ(IIa) or (IIb) | —H | —Cl | —H |
| ACA(IIa) or (IIb) | —H | —Cl | —CH₃ |
| ACB(IIa) or (IIb) | —H | —Cl | -n-propyl |
| ACC(IIa) or (IIb) | —H | —Cl | -n-butyl |
| ACD(IIa) or (IIb) | —H | —Cl | -t-butyl |
| ACE(IIa) or (IIb) | —H | —Cl | -iso-butyl |
| ACF(IIa) or (IIb) | —H | —Cl | —OCH₃ |
| ACG(IIa) or (IIb) | —H | —Cl | —OC₂H₅ |
| ACH(IIa) or (IIb) | —H | —Cl | —OC₃H₇ |
| ACI(IIa) or (IIb) | —H | —Cl | —CHF₂ |
| ACJ(IIa) or (IIb) | —H | —Cl | —CF₃ |
| ACK(IIa) or (IIb) | —H | —Cl | —CHCl₂ |
| ACL(IIa) or (IIb) | —H | —Cl | —CCl₃ |
| ACM(IIa) or (IIb) | —H | —Cl | —F |
| ACN(IIa) or (IIb) | —H | —Cl | —Cl |
| ACO(IIa) or (IIb) | —H | —Cl | —Br |
| ACP(IIa) or (IIb) | —H | —Cl | —I |
| ACQ(IIa) or (IIb) | —H | —Br | —H |
| ACR(IIa) or (IIb) | —H | —Br | —CH₃ |
| ACS(IIa) or (IIb) | —H | —Br | -n-propyl |
| ACT(IIa) or (IIb) | —H | —Br | -n-butyl |
| ACU(IIa) or (IIb) | —H | —Br | -t-butyl |
| ACV(IIa) or (IIb) | —H | —Br | -iso-butyl |
| ACW(IIa) or (IIb) | —H | —Br | —OCH₃ |
| ACX(IIa) or (IIb) | —H | —Br | —OC₂H₅ |
| ACY(IIa) or (IIb) | —H | —Br | —OC₃H₇ |
| ACZ(IIa) or (IIb) | —H | —Br | —CHF₂ |
| ADA(IIa) or (IIb) | —H | —Br | —CF₃ |
| ADB(IIa) or (IIb) | —H | —Br | —CHCl₂ |
| ADC(IIa) or (IIb) | —H | —Br | —CCl₃ |
| ADD(IIa) or (IIb) | —H | —Br | —F |
| ADE(IIa) or (IIb) | —H | —Br | —Cl |
| ADF(IIa) or (IIb) | —H | —Br | —Br |
| ADG(IIa) or (IIb) | —H | —Br | —I |
| ADH(IIa) or (IIb) | —H | —I | —H |
| ADI(IIa) or (IIb) | —H | —I | —CH₃ |
| ADJ(IIa) or (IIb) | —H | —I | -n-propyl |
| ADK(IIa) or (IIb) | —H | —I | -n-butyl |
| ADL(IIa) or (IIb) | —H | —I | -t-butyl |
| ADM(IIa) or (IIb) | —H | —I | -iso-butyl |
| ADN(IIa) or (IIb) | —H | —I | —OCH₃ |
| ADO(IIa) or (IIb) | —H | —I | —OC₂H₅ |
| ADP(IIa) or (IIb) | —H | —I | —OC₃H₇ |
| ADQ(IIa) or (IIb) | —H | —I | —CHF₂ |
| ADR(IIa) or (IIb) | —H | —I | —CF₃ |
| ADS(IIa) or (IIb) | —H | —I | —CHCl₂ |
| ADT(IIa) or (IIb) | —H | —I | —CCl₃ |
| ADU(IIa) or (IIb) | —H | —I | —F |
| ADV(IIa) or (IIb) | —H | —I | —Cl |
| ADW(IIa) or (IIb) | —H | —I | —Br |
| ADX(IIa) or (IIb) | —H | —I | —I |
| ADY(IIa) or (IIb) | —H | —NO₂ | —H |
| ADZ(IIa) or (IIb) | —H | —NO₂ | —CH₃ |
| AEA(IIa) or (IIb) | —H | —NO₂ | -n-propyl |
| AEB(IIa) or (IIb) | —H | —NO₂ | -n-butyl |
| AEC(IIa) or (IIb) | —H | —NO₂ | -t-butyl |
| AED(IIa) or (IIb) | —H | —NO₂ | -iso-butyl |
| AEE(IIa) or (IIb) | —H | —NO₂ | —OCH₃ |
| AEF(IIa) or (IIb) | —H | —NO₂ | —OC₂H₅ |
| AEG(IIa) or (IIb) | —H | —NO₂ | —OC₃H₇ |
| AEH(IIa) or (IIb) | —H | —NO₂ | —CHF₂ |
| AEI(IIa) or (IIb) | —H | —NO₂ | —CF₃ |
| AEJ(IIa) or (IIb) | —H | —NO₂ | —CHCl₂ |
| AEK(IIa) or (IIb) | —H | —NO₂ | —CCl₃ |
| AEL(IIa) or (IIb) | —H | —NO₂ | —F |
| AEM(IIa) or (IIb) | —H | —NO₂ | —Cl |
| AEN(IIa) or (IIb) | —H | —NO₂ | —Br |
| AEO(IIa) or (IIb) | —H | —NO₂ | —I |
| AEP(IIa) or (IIb) | —H | —CN | —H |
| AEQ(IIa) or (IIb) | —H | —CN | —CH₃ |
| AER(IIa) or (IIb) | —H | —CN | -n-propyl |
| AES(IIa) or (IIb) | —H | —CN | -n-butyl |
| AET(IIa) or (IIb) | —H | —CN | -t-butyl |
| AEU(IIa) or (IIb) | —H | —CN | -iso-butyl |
| AEV(IIa) or (IIb) | —H | —CN | —OCH₃ |
| AEW(IIa) or (IIb) | —H | —CN | —OC₂H₅ |
| AEX(IIa) or (IIb) | —H | —CN | —OC₃H₇ |
| AEY(IIa) or (IIb) | —H | —CN | —CHF₂ |
| AEZ(IIa) or (IIb) | —H | —CN | —CF₃ |
| AFA(IIa) or (IIb) | —H | —CN | —CHCl₂ |
| AFB(IIa) or (IIb) | —H | —CN | —CCl₃ |
| AFC(IIa) or (IIb) | —H | —CN | —F |
| AFD(IIa) or (IIb) | —H | —CN | —Cl |
| AFE(IIa) or (IIb) | —H | —CN | —Br |
| AFF(IIa) or (IIb) | —H | —CN | —I |
| AFG(IIa) or (IIb) | —H | —NH₂ | —H |
| AFH(IIa) or (IIb) | —H | —NH₂ | —CH₃ |
| AFI(IIa) or (IIb) | —H | —NH₂ | -n-propyl |
| AFJ(IIa) or (IIb) | —H | —NH₂ | -n-butyl |
| AFK(IIa) or (IIb) | —H | —NH₂ | -t-butyl |
| AFL(IIa) or (IIb) | —H | —NH₂ | -iso-butyl |
| AFM(IIa) or (IIb) | —H | —NH₂ | —OCH₃ |
| AFN(IIa) or (IIb) | —H | —NH₂ | —OC₂H₅ |
| AFO(IIa) or (IIb) | —H | —NH₂ | —OC₃H₇ |
| AFP(IIa) or (IIb) | —H | —NH₂ | —CHF₂ |
| AFQ(IIa) or (IIb) | —H | —NH₂ | —CF₃ |
| AFR(IIa) or (IIb) | —H | —NH₂ | —CHCl₂ |
| AFS(IIa) or (IIb) | —H | —NH₂ | —CCl₃ |
| AFT(IIa) or (IIb) | —H | —NH₂ | —F |
| AFU(IIa) or (IIb) | —H | —NH₂ | —Cl |
| AFV(IIa) or (IIb) | —H | —NH₂ | —Br |
| AFW(IIa) or (IIb) | —H | —NH₂ | —I |
| AFX(IIa) or (IIb) | —H | —CH₃ | —H |
| AFY(IIa) or (IIb) | —H | —CH₃ | —CH₃ |
| AFZ(IIa) or (IIb) | —H | —CH₃ | -n-propyl |
| AGA(IIa) or (IIb) | —H | —CH₃ | -n-butyl |
| AGB(IIa) or (IIb) | —H | —CH₃ | -t-butyl |
| AGC(IIa) or (IIb) | —H | —CH₃ | -iso-butyl |
| AGD(IIa) or (IIb) | —H | —CH₃ | —OCH₃ |
| AGE(IIa) or (IIb) | —H | —CH₃ | —OC₂H₅ |
| AGF(IIa) or (IIb) | —H | —CH₃ | —OC₃H₇ |
| AGG(IIa) or (IIb) | —H | —CH₃ | —CHF₂ |
| AGH(IIa) or (IIb) | —H | —CH₃ | —CF₃ |
| AGI(IIa) or (IIb) | —H | —CH₃ | —CHCl₂ |
| AGJ(IIa) or (IIb) | —H | —CH₃ | —CCl₃ |
| AGK(IIa) or (IIb) | —H | —CH₃ | —F |
| AGL(IIa) or (IIb) | —H | —CH₃ | —Cl |
| AGM(IIa) or (IIb) | —H | —CH₃ | —Br |
| AGN(IIa) or (IIb) | —H | —CH₃ | —I |
| AGO(IIa) | —OH | —H | —H |
| AGP(IIa) | —OH | —H | —CH₃ |
| AGQ(IIa) | —OH | —H | -n-propyl |
| AGR(IIa) | —OH | —H | -n-butyl |
| AGS(IIa) | —OH | —H | -t-butyl |
| AGT(IIa) | —OH | —H | -iso-butyl |
| AGU(IIa) | —OH | —H | —OCH₃ |
| AGV(IIa) | —OH | —H | —OC₂H₅ |
| AGW(IIa) | —OH | —H | —OC₃H₇ |
| AGX(IIa) | —OH | —H | —CHF₂ |
| AGY(IIa) | —OH | —H | —CF₃ |
| AGZ(IIa) | —OH | —H | —CHCl₂ |
| AHA(IIa) | —OH | —H | —CCl₃ |
| AHB(IIa) | —OH | —H | —F |
| AHC(IIa) | —OH | —H | —Cl |
| AHD(IIa) | —OH | —H | —Br |
| AHE(IIa) | —OH | —H | —I |
| AHF(IIa) or (IIb) | —OH | —OH | —H |
| AHG(IIa) or (IIb) | —OH | —OH | —CH₃ |
| AHH(IIa) or (IIb) | —OH | —OH | -n-propyl |
| AHI(IIa) or (IIb) | —OH | —OH | -n-butyl |
| AHJ(IIa) or (IIb) | —OH | —OH | -t-butyl |
| AHK(IIa) or (IIb) | —OH | —OH | -iso-butyl |
| AHL(IIa) or (IIb) | —OH | —OH | —OCH₃ |
| AHM(IIa) or (IIb) | —OH | —OH | —OC₂H₅ |
| AHN(IIa) or (IIb) | —OH | —OH | —OC₃H₇ |
| AHO(IIa) or (IIb) | —OH | —OH | —CHF₂ |
| AHP(IIa) or (IIb) | —OH | —OH | —CF₃ |
| AHQ(IIa) or (IIb) | —OH | —OH | —CHCl₂ |
| AHR(IIa) or (IIb) | —OH | —OH | —CCl₃ |
| AHS(IIa) or (IIb) | —OH | —OH | —F |
| AHT(IIa) or (IIb) | —OH | —OH | —Cl |
| AHU(IIa) or (IIb) | —OH | —OH | —Br |
| AHV(IIa) or (IIb) | —OH | —OH | —I |
| AHW(IIa) or (IIb) | —OH | —F | —H |
| AHX(IIa) or (IIb) | —OH | —F | —CH₃ |
| AHY(IIa) or (IIb) | —OH | —F | -n-propyl |
| AHZ(IIa) or (IIb) | —OH | —F | -n-butyl |
| AIA(IIa) or (IIb) | —OH | —F | -t-butyl |
| AIB(IIa) or (IIb) | —OH | —F | -iso-butyl |
| AIC(IIa) or (IIb) | —OH | —F | —OCH₃ |
| AID(IIa) or (IIb) | —OH | —F | —OC₂H₅ |
| AIE(IIa) or (IIb) | —OH | —F | —OC₃H₇ |
| AIF(IIa) or (IIb) | —OH | —F | —CHF₂ |
| AIG(IIa) or (IIb) | —OH | —F | —CF₃ |
| AIH(IIa) or (IIb) | —OH | —F | —CHCl₂ |
| AII(IIa) or (IIb) | —OH | —F | —CCl₃ |
| AIJ(IIa) or (IIb) | —OH | —F | —F |
| AIK(IIa) or (IIb) | —OH | —F | —Cl |
| AIL(IIa) or (IIb) | —OH | —F | —Br |
| AIM(IIa) or (IIb) | —OH | —F | —I |
| AIN(IIa) or (IIb) | —OH | —Cl | —H |
| AIO(IIa) or (IIb) | —OH | —Cl | —CH₃ |
| AIP(IIa) or (IIb) | —OH | —Cl | -n-propyl |
| AIQ(IIa) or (IIb) | —OH | —Cl | -n-butyl |
| AIR(IIa) or (IIb) | —OH | —Cl | -t-butyl |
| AIS(IIa) or (IIb) | —OH | —Cl | -iso-butyl |
| AIT(IIa) or (IIb) | —OH | —Cl | —OCH₃ |
| AIU(IIa) or (IIb) | —OH | —Cl | —OC₂H₅ |
| AIV(IIa) or (IIb) | —OH | —Cl | —OC₃H₇ |
| AIW(IIa) or (IIb) | —OH | —Cl | —CHF₂ |
| AIX(IIa) or (IIb) | —OH | —Cl | —CF₃ |
| AIY(IIa) or (IIb) | —OH | —Cl | —CHCl₂ |
| AIZ(IIa) or (IIb) | —OH | —Cl | —CCl₃ |
| AJA(IIa) or (IIb) | —OH | —Cl | —F |
| AJB(IIa) or (IIb) | —OH | —Cl | —Cl |
| AJC(IIa) or (IIb) | —OH | —Cl | —Br |
| AJD(IIa) or (IIb) | —OH | —Cl | —I |
| AJE(IIa) or (IIb) | —OH | —Br | —H |
| AJF(IIa) or (IIb) | —OH | —Br | —CH₃ |
| AJG(IIa) or (IIb) | —OH | —Br | -n-propyl |
| AJH(IIa) or (IIb) | —OH | —Br | -n-butyl |
| AJI(IIa) or (IIb) | —OH | —Br | -t-butyl |
| AJJ(IIa) or (IIb) | —OH | —Br | -iso-butyl |
| AJK(IIa) or (IIb) | —OH | —Br | —OCH₃ |
| AJL(IIa) or (IIb) | —OH | —Br | —OC₂H₅ |
| AJM(IIa) or (IIb) | —OH | —Br | —OC₃H₇ |
| AJN(IIa) or (IIb) | —OH | —Br | —CHF₂ |
| AJO(IIa) or (IIb) | —OH | —Br | —CF₃ |
| AJP(IIa) or (IIb) | —OH | —Br | —CHCl₂ |
| AJQ(IIa) or (IIb) | —OH | —Br | —CCl₃ |
| AJR(IIa) or (IIb) | —OH | —Br | —F |
| AJS(IIa) or (IIb) | —OH | —Br | —Cl |
| AJT(IIa) or (IIb) | —OH | —Br | —Br |
| AJU(IIa) or (IIb) | —OH | —Br | —I |
| AJV(IIa) or (IIb) | —OH | —I | —H |
| AJW(IIa) or (IIb) | —OH | —I | —CH₃ |
| AJX(IIa) or (IIb) | —OH | —I | -n-propyl |
| AJY(IIa) or (IIb) | —OH | —I | -n-butyl |
| AJZ(IIa) or (IIb) | —OH | —I | -t-butyl |
| AKA(IIa) or (IIb) | —OH | —I | -iso-butyl |
| AKB(IIa) or (IIb) | —OH | —I | —OCH₃ |
| AKC(IIa) or (IIb) | —OH | —I | —OC₂H₅ |
| AKD(IIa) or (IIb) | —OH | —I | —OC₃H₇ |
| AKE(IIa) or (IIb) | —OH | —I | —CHF₂ |
| AKF(IIa) or (IIb) | —OH | —I | —CF₃ |
| AKG(IIa) or (IIb) | —OH | —I | —CHCl₂ |
| AKH(IIa) or (IIb) | —OH | —I | —CCl₃ |
| AKI(IIa) or (IIb) | —OH | —I | —F |
| AKJ(IIa) or (IIb) | —OH | —I | —Cl |
| AKK(IIa) or (IIb) | —OH | —I | —Br |
| AKL(IIa) or (IIb) | —OH | —I | —I |
| AKM(IIa) or (IIb) | —OH | —NO₂ | —H |
| AKN(IIa) or (IIb) | —OH | —NO₂ | —CH₃ |
| AKO(IIa) or (IIb) | —OH | —NO₂ | -n-propyl |
| AKP(IIa) or (IIb) | —OH | —NO₂ | -n-butyl |
| AKQ(IIa) or (IIb) | —OH | —NO₂ | -t-butyl |
| AKR(IIa) or (IIb) | —OH | —NO₂ | -iso-butyl |
| AKS(IIa) or (IIb) | —OH | —NO₂ | —OCH₃ |
| AKT(IIa) or (IIb) | —OH | —NO₂ | —OC₂H₅ |
| AKU(IIa) or (IIb) | —OH | —NO₂ | —OC₃H₇ |
| AKV(IIa) or (IIb) | —OH | —NO₂ | —CHF₂ |
| AKW(IIa) or (IIb) | —OH | —NO₂ | —CF₃ |
| AKX(IIa) or (IIb) | —OH | —NO₂ | —CHCl₂ |
| AKY(IIa) or (IIb) | —OH | —NO₂ | —CCl₃ |
| AKZ(IIa) or (IIb) | —OH | —NO₂ | —F |
| ALA(IIa) or (IIb) | —OH | —NO₂ | —Cl |
| ALB(IIa) or (IIb) | —OH | —NO₂ | —Br |
| ALC(IIa) or (IIb) | —OH | —NO₂ | —I |
| ALD(IIa) or (IIb) | —OH | —CN | —H |
| ALE(IIa) or (IIb) | —OH | —CN | —CH₃ |
| ALF(IIa) or (IIb) | —OH | —CN | -n-propyl |
| ALG(IIa) or (IIb) | —OH | —CN | -n-butyl |
| ALH(IIa) or (IIb) | —OH | —CN | -t-butyl |
| ALI(IIa) or (IIb) | —OH | —CN | -iso-butyl |
| ALJ(IIa) or (IIb) | —OH | —CN | —OCH₃ |
| ALK(IIa) or (IIb) | —OH | —CN | —OC₂H₅ |
| ALL(IIa) or (IIb) | —OH | —CN | —OC₃H₇ |
| ALM(IIa) or (IIb) | —OH | —CN | —CHF₂ |
| ALN(IIa) or (IIb) | —OH | —CN | —CF₃ |
| ALO(IIa) or (IIb) | —OH | —CN | —CHCl₂ |
| ALP(IIa) or (IIb) | —OH | —CN | —CCl₃ |
| ALQ(IIa) or (IIb) | —OH | —CN | —F |
| ALR(IIa) or (IIb) | —OH | —CN | —Cl |
| ALS(IIa) or (IIb) | —OH | —CN | —Br |
| ALT(IIa) or (IIb) | —OH | —CN | —I |
| ALU(IIa) or (IIb) | —OH | —NH₂ | —H |
| ALV(IIa) or (IIb) | —OH | —NH₂ | —CH₃ |
| ALW(IIa) or (IIb) | —OH | —NH₂ | -n-propyl |
| ALX(IIa) or (IIb) | —OH | —NH₂ | -n-butyl |
| ALY(IIa) or (IIb) | —OH | —NH₂ | -t-butyl |
| ALZ(IIa) or (IIb) | —OH | —NH₂ | -iso-butyl |
| AMA(IIa) or (IIb) | —OH | —NH₂ | —OCH₃ |
| AMB(IIa) or (IIb) | —OH | —NH₂ | —OC₂H₅ |
| AMC(IIa) or (IIb) | —OH | —NH₂ | —OC₃H₇ |
| AMD(IIa) or (IIb) | —OH | —NH₂ | —CHF₂ |
| AME(IIa) or (IIb) | —OH | —NH₂ | —CF₃ |
| AMF(IIa) or (IIb) | —OH | —NH₂ | —CHCl₂ |
| AMG(IIa) or (IIb) | —OH | —NH₂ | —CCl₃ |
| AMH(IIa) or (IIb) | —OH | —NH₂ | —F |
| AMI(IIa) or (IIb) | —OH | —NH₂ | —Cl |
| AMJ(IIa) or (IIb) | —OH | —NH₂ | —Br |
| AMK(IIa) or (IIb) | —OH | —NH₂ | —I |
| AML(IIa) or (IIb) | —OH | —CH₃ | —H |
| AMM(IIa) or (IIb) | —OH | —CH₃ | —CH₃ |
| AMN(IIa) or (IIb) | —OH | —CH₃ | -n-propyl |
| AMO(IIa) or (IIb) | —OH | —CH₃ | -n-butyl |
| AMP(IIa) or (IIb) | —OH | —CH₃ | -t-butyl |
| AMQ(IIa) or (IIb) | —OH | —CH₃ | -iso-butyl |
| AMR(IIa) or (IIb) | —OH | —CH₃ | —OCH₃ |
| AMS(IIa) or (IIb) | —OH | —CH₃ | —OC₂H₅ |
| AMT(IIa) or (IIb) | —OH | —CH₃ | —OC₃H₇ |
| AMU(IIa) or (IIb) | —OH | —CH₃ | —CHF₂ |
| AMV(IIa) or (IIb) | —OH | —CH₃ | —CF₃ |
| AMW(IIa) or (IIb) | —OH | —CH₃ | —CHCl₂ |
| AMX(IIa) or (IIb) | —OH | —CH₃ | —CCl₃ |
| AMY(IIa) or (IIb) | —OH | —CH₃ | —F |
| AMZ(IIa) or (IIb) | —OH | —CH₃ | —Cl |
| ANA(IIa) or (IIb) | —OH | —CH₃ | —Br |
| ANB(IIa) or (IIb) | —OH | —CH₃ | —I |
| ANC(IIa) | —F | —H | —H |
| AND(IIa) | —F | —H | —CH₃ |
| ANE(IIa) | —F | —H | -n-propyl |
| ANF(IIa) | —F | —H | -n-butyl |
| ANG(IIa) | —F | —H | -t-butyl |
| ANH(IIa) | —F | —H | -iso-butyl |
| ANI(IIa) | —F | —H | —OCH₃ |
| ANJ(IIa) | —F | —H | —OC₂H₅ |
| ANK(IIa) | —F | —H | —OC₃H₇ |
| ANL(IIa) | —F | —H | —CHF₂ |
| ANM(IIa) | —F | —H | —CF₃ |
| ANN(IIa) | —F | —H | —CHCl₂ |
| ANO(IIa) | —F | —H | —CCl₃ |
| ANP(IIa) | —F | —H | —F |
| ANQ(IIa) | —F | —H | —Cl |
| ANR(IIa) | —F | —H | —Br |
| ANS(IIa) | —F | —H | —I |
| ANT(IIa) or (IIb) | —F | —OH | —H |
| ANU(IIa) or (IIb) | —F | —OH | —CH₃ |
| ANV(IIa) or (IIb) | —F | —OH | -n-propyl |
| ANW(IIa) or (IIb) | —F | —OH | -n-butyl |
| ANX(IIa) or (IIb) | —F | —OH | -t-butyl |
| ANY(IIa) or (IIb) | —F | —OH | -iso-butyl |
| ANZ(IIa) or (IIb) | —F | —OH | —OCH₃ |
| AOA(IIa) or (IIb) | —F | —OH | —OC₂H₅ |
| AOB(IIa) or (IIb) | —F | —OH | —OC₃H₇ |
| AOC(IIa) or (IIb) | —F | —OH | —CHF₂ |
| AOD(IIa) or (IIb) | —F | —OH | —CF₃ |
| AOE(IIa) or (IIb) | —F | —OH | —CHCl₂ |
| AOF(IIa) or (IIb) | —F | —OH | —CCl₃ |
| AOG(IIa) or (IIb) | —F | —OH | —F |
| AOH(IIa) or (IIb) | —F | —OH | —Cl |
| AOI(IIa) or (IIb) | —F | —OH | —Br |
| AOJ(IIa) or (IIb) | —F | —OH | —I |
| AOK(IIa) or (IIb) | —F | —F | —H |
| AOL(IIa) or (IIb) | —F | —F | —CH₃ |
| AOM(IIa) or (IIb) | —F | —F | -n-propyl |
| AON(IIa) or (IIb) | —F | —F | -n-butyl |
| AOO(IIa) or (IIb) | —F | —F | -t-butyl |
| AOP(IIa) or (IIb) | —F | —F | -iso-butyl |
| AOQ(IIa) or (IIb) | —F | —F | —OCH₃ |
| AOR(IIa) or (IIb) | —F | —F | —OC₂H₅ |
| AOS(IIa) or (IIb) | —F | —F | —OC₃H₇ |
| AOT(IIa) or (IIb) | —F | —F | —CHF₂ |
| AOU(IIa) or (IIb) | —F | —F | —CF₃ |
| AOV(IIa) or (IIb) | —F | —F | —CHCl₂ |
| AOW(IIa) or (IIb) | —F | —F | —CCl₃ |
| AOX(IIa) or (IIb) | —F | —F | —F |
| AOY(IIa) or (IIb) | —F | —F | —Cl |
| AOZ(IIa) or (IIb) | —F | —F | —Br |
| APA(IIa) or (IIb) | —F | —F | —I |
| APB(IIa) or (IIb) | —F | —Cl | —H |
| APC(IIa) or (IIb) | —F | —Cl | —CH₃ |
| APD(IIa) or (IIb) | —F | —Cl | -n-propyl |
| APE(IIa) or (IIb) | —F | —Cl | -n-butyl |
| APF(IIa) or (IIb) | —F | —Cl | -t-butyl |
| APG(IIa) or (IIb) | —F | —Cl | -iso-butyl |
| APH(IIa) or (IIb) | —F | —Cl | —OCH₃ |
| API(IIa) or (IIb) | —F | —Cl | —OC₂H₅ |
| APJ(IIa) or (IIb) | —F | —Cl | —OC₃H₇ |
| APK(IIa) or (IIb) | —F | —Cl | —CHF₂ |
| APL(IIa) or (IIb) | —F | —Cl | —CF₃ |
| APM(IIa) or (IIb) | —F | —Cl | —CHCl₂ |
| APN(IIa) or (IIb) | —F | —Cl | —CCl₃ |
| APO(IIa) or (IIb) | —F | —Cl | —F |
| APP(IIa) or (IIb) | —F | —Cl | —Cl |
| APQ(IIa) or (IIb) | —F | —Cl | —Br |
| APR(IIa) or (IIb) | —F | —Cl | —I |
| APS(IIa) or (IIb) | —F | —Br | —H |
| APT(IIa) or (IIb) | —F | —Br | —CH₃ |
| APU(IIa) or (IIb) | —F | —Br | -n-propyl |
| APV(IIa) or (IIb) | —F | —Br | -n-butyl |
| APW(IIa) or (IIb) | —F | —Br | -t-butyl |
| APX(IIa) or (IIb) | —F | —Br | -iso-butyl |
| APY(IIa) or (IIb) | —F | —Br | —OCH₃ |
| APZ(IIa) or (IIb) | —F | —Br | —OC₂H₅ |
| AQA(IIa) or (IIb) | —F | —Br | —OC₃H₇ |
| AQB(IIa) or (IIb) | —F | —Br | —CHF₂ |
| AQC(IIa) or (IIb) | —F | —Br | —CF₃ |
| AQD(IIa) or (IIb) | —F | —Br | —CHCl₂ |
| AQE(IIa) or (IIb) | —F | —Br | —CCl₃ |
| AQF(IIa) or (IIb) | —F | —Br | —F |
| AQG(IIa) or (IIb) | —F | —Br | —Cl |
| AQH(IIa) or (IIb) | —F | —Br | —Br |
| AQI(IIa) or (IIb) | —F | —Br | —I |
| AQJ(IIa) or (IIb) | —F | —I | —H |
| AQK(IIa) or (IIb) | —F | —I | —CH₃ |
| AQL(IIa) or (IIb) | —F | —I | -n-propyl |
| AQM(IIa) or (IIb) | —F | —I | -n-butyl |
| AQN(IIa) or (IIb) | —F | —I | -t-butyl |
| AQO(IIa) or (IIb) | —F | —I | -iso-butyl |
| AQP(IIa) or (IIb) | —F | —I | —OCH₃ |
| AQQ(IIa) or (IIb) | —F | —I | —OC₂H₅ |
| AQR(IIa) or (IIb) | —F | —I | —OC₃H₇ |
| AQS(IIa) or (IIb) | —F | —I | —CHF₂ |
| AQT(IIa) or (IIb) | —F | —I | —CF₃ |
| AQU(IIa) or (IIb) | —F | —I | —CHCl₂ |
| AQV(IIa) or (IIb) | —F | —I | —CCl₃ |
| AQW(IIa) or (IIb) | —F | —I | —F |
| AQX(IIa) or (IIb) | —F | —I | —Cl |
| AQY(IIa) or (IIb) | —F | —I | —Br |
| AQZ(IIa) or (IIb) | —F | —I | —I |
| ARA(IIa) or (IIb) | —F | —NO₂ | —H |
| ARB(IIa) or (IIb) | —F | —NO₂ | —CH₃ |
| ARC(IIa) or (IIb) | —F | —NO₂ | -n-propyl |
| ARD(IIa) or (IIb) | —F | —NO₂ | -n-butyl |
| ARE(IIa) or (IIb) | —F | —NO₂ | -t-butyl |
| ARF(IIa) or (IIb) | —F | —NO₂ | -iso-butyl |
| ARG(IIa) or (IIb) | —F | —NO₂ | —OCH₃ |
| ARH(IIa) or (IIb) | —F | —NO₂ | —OC₂H₅ |
| ARI(IIa) or (IIb) | —F | —NO₂ | —OC₃H₇ |
| ARJ(IIa) or (IIb) | —F | —NO₂ | —CHF₂ |
| ARK(IIa) or (IIb) | —F | —NO₂ | —CF₃ |
| ARL(IIa) or (IIb) | —F | —NO₂ | —CHCl₂ |
| ARM(IIa) or (IIb) | —F | —NO₂ | —CCl₃ |
| ARN(IIa) or (IIb) | —F | —NO₂ | —F |
| ARO(IIa) or (IIb) | —F | —NO₂ | —Cl |
| ARP(IIa) or (IIb) | —F | —NO₂ | —Br |
| ARQ(IIa) or (IIb) | —F | —NO₂ | —I |
| ARR(IIa) or (IIb) | —F | —CN | —H |
| ARS(IIa) or (IIb) | —F | —CN | —CH₃ |
| ART(IIa) or (IIb) | —F | —CN | -n-propyl |
| ARU(IIa) or (IIb) | —F | —CN | -n-butyl |
| ARV(IIa) or (IIb) | —F | —CN | -t-butyl |
| ARW(IIa) or (IIb) | —F | —CN | -iso-butyl |
| ARX(IIa) or (IIb) | —F | —CN | —OCH₃ |
| ARY(IIa) or (IIb) | —F | —CN | —OC₂H₅ |
| ARZ(IIa) or (IIb) | —F | —CN | —OC₃H₇ |
| ASA(IIa) or (IIb) | —F | —CN | —CHF₂ |
| ASB(IIa) or (IIb) | —F | —CN | —CF₃ |
| ASC(IIa) or (IIb) | —F | —CN | —CHCl₂ |
| ASD(IIa) or (IIb) | —F | —CN | —CCl₃ |
| ASE(IIa) or (IIb) | —F | —CN | —F |
| ASF(IIa) or (IIb) | —F | —CN | —Cl |
| ASG(IIa) or (IIb) | —F | —CN | —Br |
| ASH(IIa) or (IIb) | —F | —CN | —I |
| ASI(IIa) or (IIb) | —F | —NH₂ | —H |
| ASJ(IIa) or (IIb) | —F | —NH₂ | —CH₃ |
| ASK(IIa) or (IIb) | —F | —NH₂ | -n-propyl |
| ASL(IIa) or (IIb) | —F | —NH₂ | -n-butyl |
| ASM(IIa) or (IIb) | —F | —NH₂ | -t-butyl |
| ASN(IIa) or (IIb) | —F | —NH₂ | -iso-butyl |
| ASO(IIa) or (IIb) | —F | —NH₂ | —OCH₃ |
| ASP(IIa) or (IIb) | —F | —NH₂ | —OC₂H₅ |
| ASQ(IIa) or (IIb) | —F | —NH₂ | —OC₃H₇ |
| ASR(IIa) or (IIb) | —F | —NH₂ | —CHF₂ |
| ASS(IIa) or (IIb) | —F | —NH₂ | —CF₃ |
| AST(IIa) or (IIb) | —F | —NH₂ | —CHCl₂ |
| ASU(IIa) or (IIb) | —F | —NH₂ | —CCl₃ |
| ASV(IIa) or (IIb) | —F | —NH₂ | —F |
| ASW(IIa) or (IIb) | —F | —NH₂ | —Cl |
| ASX(IIa) or (IIb) | —F | —NH₂ | —Br |
| ASY(IIa) or (IIb) | —F | —NH₂ | —I |
| ASZ(IIa) or (IIb) | —F | —CH₃ | —H |
| ATA(IIa) or (IIb) | —F | —CH₃ | —CH₃ |
| ATB(IIa) or (IIb) | —F | —CH₃ | -n-propyl |
| ATC(IIa) or (IIb) | —F | —CH₃ | -n-butyl |
| ATD(IIa) or (IIb) | —F | —CH₃ | -t-butyl |
| ATE(IIa) or (IIb) | —F | —CH₃ | -iso-butyl |
| ATF(IIa) or (IIb) | —F | —CH₃ | —OCH₃ |
| ATG(IIa) or (IIb) | —F | —CH₃ | —OC₂H₅ |
| ATH(IIa) or (IIb) | —F | —CH₃ | —OC₃H₇ |
| ATI(IIa) or (IIb) | —F | —CH₃ | —CHF₂ |
| ATJ(IIa) or (IIb) | —F | —CH₃ | —CF₃ |
| ATK(IIa) or (IIb) | —F | —CH₃ | —CHCl₂ |
| ATL(IIa) or (IIb) | —F | —CH₃ | —CCl₃ |
| ATM(IIa) or (IIb) | —F | —CH₃ | —F |
| ATN(IIa) or (IIb) | —F | —CH₃ | —Cl |
| ATO(IIa) or (IIb) | —F | —CH₃ | —Br |
| ATP(IIa) or (IIb) | —F | —CH₃ | —I |
| ATQ(IIa) | —Cl | —H | —H |
| ATR(IIa) | —Cl | —H | —CH₃ |
| ATS(IIa) | —Cl | —H | -n-propyl |
| ATT(IIa) | —Cl | —H | -n-butyl |
| ATU(IIa) | —Cl | —H | -t-butyl |
| ATV(IIa) | —Cl | —H | -iso-butyl |
| ATW(IIa) | —Cl | —H | —OCH₃ |
| ATX(IIa) | —Cl | —H | —OC₂H₅ |
| ATY(IIa) | —Cl | —H | —OC₃H₇ |
| ATZ(IIa) | —Cl | —H | —CHF₂ |
| AUA(IIa) | —Cl | —H | —CF₃ |
| AUB(IIa) | —Cl | —H | —CHCl₂ |
| AUC(IIa) | —Cl | —H | —CCl₃ |
| AUD(IIa) | —Cl | —H | —F |
| AUE(IIa) | —Cl | —H | —Cl |
| AUF(IIa) | —Cl | —H | —Br |
| AUG(IIa) | —Cl | —H | —I |
| AUH(IIa) or (IIb) | —Cl | —OH | —H |
| AUI(IIa) or (IIb) | —Cl | —OH | —CH₃ |
| AUJ(IIa) or (IIb) | —Cl | —OH | -n-propyl |
| AUK(IIa) or (IIb) | —Cl | —OH | -n-butyl |
| AUL(IIa) or (IIb) | —Cl | —OH | -t-butyl |
| AUM(IIa) or (IIb) | —Cl | —OH | -iso-butyl |
| AUN(IIa) or (IIb) | —Cl | —OH | —OCH₃ |
| AUO(IIa) or (IIb) | —Cl | —OH | —OC₂H₅ |
| AUP(IIa) or (IIb) | —Cl | —OH | —OC₃H₇ |
| AUQ(IIa) or (IIb) | —Cl | —OH | —CHF₂ |
| AUR(IIa) or (IIb) | —Cl | —OH | —CF₃ |
| AUS(IIa) or (IIb) | —Cl | —OH | —CHCl₂ |
| AUT(IIa) or (IIb) | —Cl | —OH | —CCl₃ |
| AUU(IIa) or (IIb) | —Cl | —OH | —F |
| AUV(IIa) or (IIb) | —Cl | —OH | —Cl |
| AUW(IIa) or (IIb) | —Cl | —OH | —Br |
| AUX(IIa) or (IIb) | —Cl | —OH | —I |
| AUY(IIa) or (IIb) | —Cl | —F | —H |
| AUZ(IIa) or (IIb) | —Cl | —F | —CH₃ |
| AVA(IIa) or (IIb) | —Cl | —F | -n-propyl |
| AVB(IIa) or (IIb) | —Cl | —F | -n-butyl |
| AVC(IIa) or (IIb) | —Cl | —F | -t-butyl |
| AVD(IIa) or (IIb) | —Cl | —F | -iso-butyl |
| AVE(IIa) or (IIb) | —Cl | —F | —OCH₃ |
| AVF(IIa) or (IIb) | —Cl | —F | —OC₂H₅ |
| AVG(IIa) or (IIb) | —Cl | —F | —OC₃H₇ |
| AVH(IIa) or (IIb) | —Cl | —F | —CHF₂ |
| AVI(IIa) or (IIb) | —Cl | —F | —CF₃ |
| AVJ(IIa) or (IIb) | —Cl | —F | —CHCl₂ |
| AVK(IIa) or (IIb) | —Cl | —F | —CCl₃ |
| AVL(IIa) or (IIb) | —Cl | —F | —F |
| AVM(IIa) or (IIb) | —Cl | —F | —Cl |
| AVN(IIa) or (IIb) | —Cl | —F | —Br |
| AVO(IIa) or (IIb) | —Cl | —F | —I |
| AVP(IIa) or (IIb) | —Cl | —Cl | —H |
| AVQ(IIa) or (IIb) | —Cl | —Cl | —CH₃ |
| AVR(IIa) or (IIb) | —Cl | —Cl | -n-propyl |
| AVS(IIa) or (IIb) | —Cl | —Cl | -n-butyl |
| AVT(IIa) or (IIb) | —Cl | —Cl | -t-butyl |
| AVU(IIa) or (IIb) | —Cl | —Cl | -iso-butyl |
| AVV(IIa) or (IIb) | —Cl | —Cl | —OCH₃ |
| AVW(IIa) or (IIb) | —Cl | —Cl | —OC₂H₅ |
| AVX(IIa) or (IIb) | —Cl | —Cl | —OC₃H₇ |
| AVY(IIa) or (IIb) | —Cl | —Cl | —CHF₂ |
| AVZ(IIa) or (IIb) | —Cl | —Cl | —CF₃ |
| AWA(IIa) or (IIb) | —Cl | —Cl | —CHCl₂ |
| AWB(IIa) or (IIb) | —Cl | —Cl | —CCl₃ |
| AWC(IIa) or (IIb) | —Cl | —Cl | —F |
| AWD(IIa) or (IIb) | —Cl | —Cl | —Cl |
| AWE(IIa) or (IIb) | —Cl | —Cl | —Br |
| AWF(IIa) or (IIb) | —Cl | —Cl | —I |
| AWG(IIa) or (IIb) | —Cl | —Br | —H |
| AWH(IIa) or (IIb) | —Cl | —Br | —CH₃ |
| AWI(IIa) or (IIb) | —Cl | —Br | -n-propyl |
| AWJ(IIa) or (IIb) | —Cl | —Br | -n-butyl |
| AWK(IIa) or (IIb) | —Cl | —Br | -t-butyl |
| AWL(IIa) or (IIb) | —Cl | —Br | -iso-butyl |
| AWM(IIa) or (IIb) | —Cl | —Br | —OCH₃ |
| AWN(IIa) or (IIb) | —Cl | —Br | —OC₂H₅ |
| AWO(IIa) or (IIb) | —Cl | —Br | —OC₃H₇ |
| AWP(IIa) or (IIb) | —Cl | —Br | —CHF₂ |
| AWQ(IIa) or (IIb) | —Cl | —Br | —CF₃ |
| AWR(IIa) or (IIb) | —Cl | —Br | —CHCl₂ |
| AWS(IIa) or (IIb) | —Cl | —Br | —CCl₃ |
| AWT(IIa) or (IIb) | —Cl | —Br | —F |
| AWU(IIa) or (IIb) | —Cl | —Br | —Cl |
| AWV(IIa) or (IIb) | —Cl | —Br | —Br |
| AWW(IIa) or (IIb) | —Cl | —Br | —I |
| AWX(IIa) or (IIb) | —Cl | —I | —H |
| AWY(IIa) or (IIb) | —Cl | —I | —CH₃ |
| AWZ(IIa) or (IIb) | —Cl | —I | -n-propyl |
| AXA(IIa) or (IIb) | —Cl | —I | -n-butyl |
| AXB(IIa) or (IIb) | —Cl | —I | -t-butyl |
| AXC(IIa) or (IIb) | —Cl | —I | -iso-butyl |
| AXD(IIa) or (IIb) | —Cl | —I | —OCH₃ |
| AXE(IIa) or (IIb) | —Cl | —I | —OC₂H₅ |
| AXF(IIa) or (IIb) | —Cl | —I | —OC₃H₁₇ |
| AXG(IIa) or (IIb) | —Cl | —I | —CHF₂ |
| AXH(IIa) or (IIb) | —Cl | —I | —CF₃ |
| AXI(IIa) or (IIb) | —Cl | —I | —CHCl₂ |
| AXJ(IIa) or (IIb) | —Cl | —I | —CCl₃ |
| AXK(IIa) or (IIb) | —Cl | —I | —F |
| AXL(IIa) or (IIb) | —Cl | —I | —Cl |
| AXM(IIa) or (IIb) | —Cl | —I | —Br |
| AXN(IIa) or (IIb) | —Cl | —I | —I |
| AXO(IIa) or (IIb) | —Cl | —NO₂ | —H |
| AXP(IIa) or (IIb) | —Cl | —NO₂ | —CH₃ |
| AXQ(IIa) or (IIb) | —Cl | —NO₂ | -n-propyl |
| AXR(IIa) or (IIb) | —Cl | —NO₂ | -n-butyl |
| AXS(IIa) or (IIb) | —Cl | —NO₂ | -t-butyl |
| AXT(IIa) or (IIb) | —Cl | —NO₂ | -iso-butyl |
| AXU(IIa) or (IIb) | —Cl | —NO₂ | —OCH₃ |
| AXV(IIa) or (IIb) | —Cl | —NO₂ | —OC₂H₅ |
| AXW(IIa) or (IIb) | —Cl | —NO₂ | —OC₃H₇ |
| AXX(IIa) or (IIb) | —Cl | —NO₂ | —CHF₂ |
| AXY(IIa) or (IIb) | —Cl | —NO₂ | —CF₃ |
| AXZ(IIa) or (IIb) | —Cl | —NO₂ | —CHCl₂ |
| AYA(IIa) or (IIb) | —Cl | —NO₂ | —CCl₃ |
| AYB(IIa) or (IIb) | —Cl | —NO₂ | —F |
| AYC(IIa) or (IIb) | —Cl | —NO₂ | —Cl |
| AYD(IIa) or (IIb) | —Cl | —NO₂ | —Br |
| AYE(IIa) or (ITh) | —Cl | —NO₂ | —I |
| AYF(IIa) or (IIb) | —Cl | —CN | —H |
| AYG(IIa) or (IIb) | —Cl | —CN | —CH₃ |
| AYH(IIa) or (IIb) | —Cl | —CN | -n-propyl |
| AYI(IIa) or (IIb) | —Cl | —CN | -n-butyl |
| AYJ(IIa) or (IIb) | —Cl | —CN | -t-butyl |
| AYK(IIa) or (IIb) | —Cl | —CN | -iso-butyl |
| AYL(IIa) or (IIb) | —Cl | —CN | —OCH₃ |
| AYM(IIa) or (IIb) | —Cl | —CN | —OC₂H₅ |
| AYN(IIa) or (IIb) | —Cl | —CN | —OC₃H₇ |
| AYO(IIa) or (IIb) | —Cl | —CN | —CHF₂ |
| AYP(IIa) or (IIb) | —Cl | —CN | —CF₃ |
| AYQ(IIa) or (IIb) | —Cl | —CN | —CHCl₂ |
| AYR(IIa) or (IIb) | —Cl | —CN | —CCl₃ |
| AYS(IIa) or (IIb) | —Cl | —CN | —F |
| AYT(IIa) or (IIb) | —Cl | —CN | —Cl |
| AYU(IIa) or (IIb) | —Cl | —CN | —Br |
| AYV(IIa) or (IIb) | —Cl | —CN | —I |
| AYW(IIa) or (IIb) | —Cl | —NH₂ | —H |
| AYX(IIa) or (IIb) | —Cl | —NH₂ | —CH₃ |
| AYY(IIa) or (IIb) | —Cl | —NH₂ | -n-propyl |
| AYZ(IIa) or (IIb) | —Cl | —NH₂ | -n-butyl |
| AZA(IIa) or (IIb) | —Cl | —NH₂ | -t-butyl |
| AZB(IIa) or (IIb) | —Cl | —NH₂ | -iso-butyl |
| AZC(IIa) or (IIb) | —Cl | —NH₂ | —OCH₃ |
| AZD(IIa) or (IIb) | —Cl | —NH₂ | —OC₂H₅ |
| AZE(IIa) or (IIb) | —Cl | —NH₂ | —OC₃H₇ |
| AZF(IIa) or (IIb) | —Cl | —NH₂ | —CHF₂ |
| AZG(IIa) or (IIb) | —Cl | —NH₂ | —CF₃ |
| AZH(IIa) or (IIb) | —Cl | —NH₂ | —CHCl₂ |
| AZI(IIa) or (IIb) | —Cl | —NH₂ | —CCl₃ |
| AZJ(IIa) or (IIb) | —Cl | —NH₂ | —F |
| AZK(IIa) or (IIb) | —Cl | —NH₂ | —Cl |
| AZL(IIa) or (IIb) | —Cl | —NH₂ | —Br |
| AZM(IIa) or (IIb) | —Cl | —NH₂ | —I |
| AZN(IIa) or (IIb) | —Cl | —CH₃ | —H |
| AZO(IIa) or (IIb) | —Cl | —CH₃ | —CH₃ |
| AZP(IIa) or (IIb) | —Cl | —CH₃ | -n-propyl |
| AZQ(IIa) or (IIb) | —Cl | —CH₃ | -n-butyl |
| AZR(IIa) or (IIb) | —Cl | —CH₃ | -t-butyl |
| AZS(IIa) or (IIb) | —Cl | —CH₃ | -iso-butyl |
| AZT(IIa) or (IIb) | —Cl | —CH₃ | —OCH₃ |
| AZU(IIa) or (IIb) | —Cl | —CH₃ | —OC₂H₅ |
| AZV(IIa) or (IIb) | —Cl | —CH₃ | —OC₃H₇ |
| AZW(IIa) or (IIb) | —Cl | —CH₃ | —CHF₂ |
| AZX(IIa) or (IIb) | —Cl | —CH₃ | —CF₃ |
| AZY(IIa) or (IIb) | —Cl | —CH₃ | —CHCl₂ |
| AZZ(IIa) or (IIb) | —Cl | —CH₃ | —CCl₃ |
| BAA(IIa) or (IIb) | —Cl | —CH₃ | —F |
| BAB(IIa) or (IIb) | —Cl | —CH₃ | —Cl |
| BAC(IIa) or (IIb) | —Cl | —CH₃ | —Br |
| BAD(IIa) or (IIb) | —Cl | —CH₃ | —I |
| BAE(IIa) | —CHCl₂ | —H | —H |
| BAF(IIa) | —CHCl₂ | —H | —CH₃ |
| BAG(IIa) | —CHCl₂ | —H | -n-propyl |
| BAH(IIa) | —CHCl₂ | —H | -n-butyl |
| BAI(IIa) | —CHCl₂ | —H | -t-butyl |
| BAJ(IIa) | —CHCl₂ | —H | -iso-butyl |
| BAK(IIa) | —CHCl₂ | —H | —OCH₃ |
| BAL(IIa) | —CHCl₂ | —H | —OC₂H₅ |
| BAM(IIa) | —CHCl₂ | —H | —OC₃H₇ |
| BAN(IIa) | —CHCl₂ | —H | —CHF₂ |
| BAO(IIa) | —CHCl₂ | —H | —CF₃ |
| BAP(IIa) | —CHCl₂ | —H | —CHCl₂ |
| BAQ(IIa) | —CHCl₂ | —H | —CCl₃ |
| BAR(IIa) | —CHCl₂ | —H | —F |
| BAS(IIa) | —CHCl₂ | —H | —Cl |
| BAT(IIa) | —CHCl₂ | —H | —Br |
| BAU(IIa) | —CHCl₂ | —H | —I |
| BAV(IIa) or (IIb) | —CHCl₂ | —OH | —H |
| BAW(IIa) or (IIb) | —CHCl₂ | —OH | —CH₃ |
| BAX(IIa) or (IIb) | —CHCl₂ | —OH | -n-propyl |
| BAY(IIa) or (IIb) | —CHCl₂ | —OH | -n-butyl |
| BAZ(IIa) or (IIb) | —CHCl₂ | —OH | -t-butyl |
| BBA(IIa) or (IIb) | —CHCl₂ | —OH | -iso-butyl |
| BBB(IIa) or (IIb) | —CHCl₂ | —OH | —OCH₃ |
| BBC(IIa) or (IIb) | —CHCl₂ | —OH | —OC₂H₅ |
| BBD(IIa) or (IIb) | —CHCl₂ | —OH | —OC₃H₇ |
| BBE(IIa) or (IIb) | —CHCl₂ | —OH | —CHF₂ |
| BBF(IIa) or (IIb) | —CHCl₂ | —OH | —CF₃ |
| BBG(IIa) or (IIb) | —CHCl₂ | —OH | —CHCl₂ |
| BBH(IIa) or (IIb) | —CHCl₂ | —OH | —CCl₃ |
| BBI(IIa) or (IIb) | —CHCl₂ | —OH | —F |
| BBJ(IIa) or (IIb) | —CHCl₂ | —OH | —Cl |
| BBK(IIa) or (IIb) | —CHCl₂ | —OH | —Br |
| BBL(IIa) or (IIb) | —CHCl₂ | —OH | —I |
| BBM(IIa) or (IIb) | —CHCl₂ | —F | —H |
| BBN(IIa) or (IIb) | —CHCl₂ | —F | —CH₃ |
| BBO(IIa) or (IIb) | —CHCl₂ | —F | -n-propyl |
| BBP(IIa) or (IIb) | —CHCl₂ | —F | -n-butyl |
| BBQ(IIa) or (IIb) | —CHCl₂ | —F | -t-butyl |
| BBR(IIa) or (IIb) | —CHCl₂ | —F | -iso-butyl |
| BBS(IIa) or (IIb) | —CHCl₂ | —F | —OCH₃ |
| BBT(IIa) or (IIb) | —CHCl₂ | —F | —OC₂H₅ |
| BBU(IIa) or (IIb) | —CHCl₂ | —F | —OC₃H₇ |
| BBV(IIa) or (IIb) | —CHCl₂ | —F | —CHF₂ |
| BBW(IIa) or (IIb) | —CHCl₂ | —F | —CF₃ |
| BBX(IIa) or (IIb) | —CHCl₂ | —F | —CHCl₂ |
| BBY(IIa) or (IIb) | —CHCl₂ | —F | —CCl₃ |
| BBZ(IIa) or (IIb) | —CHCl₂ | —F | —F |
| BCA(IIa) or (IIb) | —CHCl₂ | —F | —Cl |
| BCB(IIa) or (IIb) | —CHCl₂ | —F | —Br |
| BCC(IIa) or (IIb) | —CHCl₂ | —F | —I |
| BCD(IIa) or (IIb) | —CHCl₂ | —Cl | —H |
| BCE(IIa) or (IIb) | —CHCl₂ | —Cl | —CH₃ |
| BCF(IIa) or (IIb) | —CHCl₂ | —Cl | -n-propyl |
| BCG(IIa) or (IIb) | —CHCl₂ | —Cl | -n-butyl |
| BCH(IIa) or (IIb) | —CHCl₂ | —Cl | -t-butyl |
| BCI(IIa) or (IIb) | —CHCl₂ | —Cl | -iso-butyl |
| BCJ(IIa) or (IIb) | —CHCl₂ | —Cl | —OCH₃ |
| BCK(IIa) or (IIb) | —CHCl₂ | —Cl | —OC₂H₅ |
| BCL(IIa) or (IIb) | —CHCl₂ | —Cl | —OC₃H₇ |
| BCM(IIa) or (IIb) | —CHCl₂ | —Cl | —CHF₂ |
| BCN(IIa) or (IIb) | —CHCl₂ | —Cl | —CF₃ |
| BCO(IIa) or (IIb) | —CHCl₂ | —Cl | —CHCl₂ |
| BCP(IIa) or (IIb) | —CHCl₂ | —Cl | —CCl₃ |
| BCQ(IIa) or (IIb) | —CHCl₂ | —Cl | —F |
| BCR(IIa) or (IIb) | —CHCl₂ | —Cl | —Cl |
| BCS(IIa) or (IIb) | —CHCl₂ | —Cl | —Br |
| BCT(IIa) or (IIb) | —CHCl₂ | —Cl | —I |
| BCU(IIa) or (IIb) | —CHCl₂ | —Br | —H |
| BCV(IIa) or (IIb) | —CHCl₂ | —Br | —CH₃ |
| BCW(IIa) or (IIb) | —CHCl₂ | —Br | -n-propyl |
| BCX(IIa) or (IIb) | —CHCl₂ | —Br | -n-butyl |
| BCY(IIa) or (IIb) | —CHCl₂ | —Br | -t-butyl |
| BCZ(IIa) or (IIb) | —CHCl₂ | —Br | -iso-butyl |
| BDA(IIa) or (IIb) | —CHCl₂ | —Br | —OCH3 |
| BDB(IIa) or (IIb) | —CHCl₂ | —Br | —OC₂H₅ |
| BDC(IIa) or (IIb) | —CHCl₂ | —Br | —OC₃H₇ |
| BDD(IIa) or (IIb) | —CHCl₂ | —Br | —CHF₂ |
| BDE(IIa) or (IIb) | —CHCl₂ | —Br | —CF₃ |
| BDF(IIa) or (IIb) | —CHCl₂ | —Br | —CHCl₂ |
| BDG(IIa) or (IIb) | —CHCl₂ | —Br | —CCl₃ |
| BDH(IIa) or (IIb) | —CHCl₂ | —Br | —F |
| BDI(IIa) or (IIb) | —CHCl₂ | —Br | —Cl |
| BDJ(IIa) or (IIb) | —CHCl₂ | —Br | —Br |
| BDK(IIa) or (IIb) | —CHCl₂ | —Br | —I |
| BDL(IIa) or (IIb) | —CHCl₂ | —I | —H |
| BDM(IIa) or (IIb) | —CHCl₂ | —I | —CH₃ |
| BDN(IIa) or (IIb) | —CHCl₂ | —I | -n-propyl |
| BDO(IIa) or (IIb) | —CHCl₂ | —I | -n-butyl |
| BDP(IIa) or (IIb) | —CHCl₂ | —I | -t-butyl |
| BDQ(IIa) or (IIb) | —CHCl₂ | —I | -iso-butyl |
| BDR(IIa) or (IIb) | —CHCl₂ | —I | —OCH₃ |
| BDS(IIa) or (IIb) | —CHCl₂ | —I | —OC₂H₅ |
| BDT(IIa) or (IIb) | —CHCl₂ | —I | —OC₃H₇ |
| BDU(IIa) or(IIb) | —CHCl₂ | —I | —CHF₂ |
| BDV(IIa) or (IIb) | —CHCl₂ | —I | —CF₃ |
| BDW(IIa) or (IIb) | —CHCl₂ | —I | —CHCl₂ |
| BDX(IIa) or (IIb) | —CHCl₂ | —I | —CCl₃ |
| BDY(IIa) or (IIb) | —CHCl₂ | —I | —F |
| BDZ(IIa) or (IIb) | —CHCl₂ | —I | —Cl |
| BEA(IIa) or(IIb) | —CHCl₂ | —I | —Br |
| BEB(IIa) or (IIb) | —CHCl₂ | —I | —I |
| BEC(IIa) or (IIb) | —CHCl₂ | —NO₂ | —H |
| BED(IIa) or (IIb) | —CHCl₂ | —NO₂ | —CH₃ |
| BEE(IIa) or (IIb) | —CHCl₂ | —NO₂ | -n-propyl |
| BEF(IIa) or (IIb) | —CHCl₂ | —NO₂ | -n-butyl |
| BEG(IIa) or (IIb) | —CHCl₂ | —NO₂ | -t-butyl |
| BEH(IIa) or (IIb) | —CHCl₂ | —NO₂ | -iso-butyl |
| BEI(IIa) or (IIb) | —CHCl₂ | —NO₂ | —OCH₃ |
| BEJ(IIa) or (IIb) | —CHCl₂ | —NO₂ | —OC₂H₅ |
| BEK(IIa) or (IIb) | —CHCl₂ | —NO₂ | —OC₃H₇ |
| BEL(IIa) or (IIb) | —CHCl₂ | —NO₂ | —CHF₂ |
| BEM(IIa) or (IIb) | —CHCl₂ | —NO₂ | —CF₃ |
| BEN(IIa) or (IIb) | —CHCl₂ | —NO₂ | —CHCl₂ |
| BEO(IIa) or (IIb) | —CHCl₂ | —NO₂ | —CCl₃ |
| BEP(IIa) or (IIb) | —CHCl₂ | —NO₂ | —F |
| BEQ(IIa) or (IIb) | —CHCl₂ | —NO₂ | —Cl |
| BER(IIa) or (IIb) | —CHCl₂ | —NO₂ | —Br |
| BES(IIa) or (IIb) | —CHCl₂ | —NO₂ | —I |
| BET(IIa) or (IIb) | —CHCl₂ | —CN | —H |
| BEU(IIa) or (IIb) | —CHCl₂ | —CN | —CH₃ |
| BEV(IIa) or (IIb) | —CHCl₂ | —CN | -n-propyl |
| BEW(IIa) or (IIb) | —CHCl₂ | —CN | -n-butyl |
| BEX(IIa) or (IIb) | —CHCl₂ | —CN | -t-butyl |
| BEY(IIa) or (IIb) | —CHCl₂ | —CN | -iso-butyl |
| BEZ(IIa) or (IIb) | —CHCl₂ | —CN | —OCH₃ |
| BFA(IIa) or (IIb) | —CHCl₂ | —CN | —OC₂H₅ |
| BFB(IIa) or (IIb) | —CHCl₂ | —CN | —OC₃H₇ |
| BFC(IIa) or (IIb) | —CHCl₂ | —CN | —CHF₂ |
| BFD(IIa) or (IIb) | —CHCl₂ | —CN | —CF₃ |
| BFE(IIa) or (IIb) | —CHCl₂ | —CN | —CHCl₂ |
| BFF(IIa) or (IIb) | —CHCl₂ | —CN | —CCl₃ |
| BFG(IIa) or (IIb) | —CHCl₂ | —CN | —F |
| BFH(IIa) or (IIb) | —CHCl₂ | —CN | —Cl |
| BFI(IIa) or (IIb) | —CHCl₂ | —CN | —Br |
| BFJ(IIa) or (IIb) | —CHCl₂ | —CN | —I |
| BFK(IIa) or (IIb) | —CHCl₂ | —NH₂ | —H |
| BFL(IIa) or (IIb) | —CHCl₂ | —NH₂ | —CH₃ |
| BFM(IIa) or (IIb) | —CHCl₂ | —NH₂ | -n-propyl |
| BFN(IIa) or (IIb) | —CHCl₂ | —NH₂ | -n-butyl |
| BFO(IIa) or (IIb) | —CHCl₂ | —NH₂ | -t-butyl |
| BFP(IIa) or (IIb) | —CHCl₂ | —NH₂ | -iso-butyl |
| BFQ(IIa) or (IIb) | —CHCl₂ | —NH₂ | —OCH₃ |
| BFR(IIa) or (IIb) | —CHCl₂ | —NH₂ | —OC₂H₅ |
| BFS(IIa) or (IIb) | —CHCl₂ | —NH₂ | —OC₃H₇ |
| BFT(IIa) or (IIb) | —CHCl₂ | —NH₂ | —CHF₂ |
| BFU(IIa) or (IIb) | —CHCl₂ | —NH₂ | —CF₃ |
| BFV(IIa) or (IIb) | —CHCl₂ | —NH₂ | —CHCl₂ |
| BFW(IIa) or (IIb) | —CHCl₂ | —NH₂ | —CCl₃ |
| BFX(IIa) or (IIb) | —CHCl₂ | —NH₂ | —F |
| BFY(IIa) or (IIb) | —CHCl₂ | —NH₂ | —Cl |
| BFZ(IIa) or (IIb) | —CHCl₂ | —NH₂ | —Br |
| BGA(IIa) or (IIb) | —CHCl₂ | —NH₂ | —I |
| BGB(IIa) or (IIb) | —CHCl₂ | —CH₃ | —H |
| BGC(IIa) or (IIb) | —CHCl₂ | —CH₃ | —CH₃ |
| BGD(IIa) or (IIb) | —CHCl₂ | —CH₃ | -n-propyl |
| BGE(IIa) or (IIb) | —CHCl₂ | —CH₃ | -n-butyl |
| BGF(IIa) or (IIb) | —CHCl₂ | —CH₃ | -t-butyl |
| BGG(IIa) or (IIb) | —CHCl₂ | —CH₃ | -iso-butyl |
| BGH(IIa) or (IIb) | —CHCl₂ | —CH₃ | —OCH₃ |
| BGI(IIa) or (IIb) | —CHCl₂ | —CH₃ | —OC₂H₅ |
| BGJ(IIa) or (IIb) | —CHCl₂ | —CH₃ | —OC₃H₇ |
| BGK(IIa) or (IIb) | —CHCl₂ | —CH₃ | —CHF₂ |
| BGL(IIa) or (IIb) | —CHCl₂ | —CH₃ | —CF₃ |
| BGM(IIa) or (IIb) | —CHCl₂ | —CH₃ | —CHCl₂ |
| BGN(IIa) or (IIb) | —CHCl₂ | —CH₃ | —CCl₃ |
| BGO(IIa) or (IIb) | —CHCl₂ | —CH₃ | —F |
| BGP(IIa) or (IIb) | —CHCl₂ | —CH₃ | —Cl |
| BGQ(IIa) or (IIb) | —CHCl₂ | —CH₃ | —Br |
| BGR(IIa) or (IIb) | —CHCl₂ | —CH₃ | —I |
| BGS(IIa) | —CF₃ | —H | —H |
| BGT(IIa) | —CF₃ | —H | —CH₃ |
| BGU(IIa) | —CF₃ | —H | -n-propyl |
| BGV(IIa) | —CF₃ | —H | -n-butyl |
| BGW(IIa) | —CF₃ | —H | -t-butyl |
| BGX(IIa) | —CF₃ | —H | -iso-butyl |
| BGY(IIa) | —CF₃ | —H | —OCH₃ |
| BGZ(IIa) | —CF₃ | —H | —OC₂H₅ |
| BHA(IIa) | —CF₃ | —H | —OC₃H₇ |
| BHB(IIa) | —CF₃ | —H | —CHF₂ |
| BHC(IIa) | —CF₃ | —H | —CF₃ |
| BHD(IIa) | —CF₃ | —H | —CHCl₂ |
| BHE(IIa) | —CF₃ | —H | —CCl₃ |
| BHF(IIa) | —CF₃ | —H | —F |
| BHG(IIa) | —CF₃ | —H | —Cl |
| BHH(IIa) | —CF₃ | —H | —Br |
| BHI(IIa) | —CF₃ | —H | —I |
| BHJ(IIa) or (IIb) | —CF₃ | —OH | —H |
| BHK(IIa) or (IIb) | —CF₃ | —OH | —CH₃ |
| BHL(IIa) or (IIb) | —CF₃ | —OH | -n-propyl |
| BHM(IIa) or (IIb) | —CF₃ | —OH | -n-butyl |
| BHN(IIa) or (IIb) | —CF₃ | —OH | -t-butyl |
| BHO(IIa) or (IIb) | —CF₃ | —OH | -iso-butyl |
| BHP(IIa) or (IIb) | —CF₃ | —OH | —OCH₃ |
| BHQ(IIa) or (IIb) | —CF₃ | —OH | —OC₂H₅ |
| BHR(IIa) or (IIb) | —CF₃ | —OH | —OC₃H₇ |
| BHS(IIa) or (IIb) | —CF₃ | —OH | —CHF₂ |
| BHT(IIa) or (IIb) | —CF₃ | —OH | —CF₃ |
| BHU(IIa) or (IIb) | —CF₃ | —OH | —CHCl₂ |
| BHV(IIa) or (IIb) | —CF₃ | —OH | —CCl₃ |
| BHW(IIa) or (IIb) | —CF₃ | —OH | —F |
| BHX(IIa) or (IIb) | —CF₃ | —OH | —Cl |
| BHY(IIa) or (IIb) | —CF₃ | —OH | —Br |
| BHZ(IIa) or (IIb) | —CF₃ | —OH | —I |
| BIA(IIa) or (IIb) | —CF₃ | —F | —H |
| BIB(IIa) or (IIb) | —CF₃ | —F | —CH₃ |
| BIC(IIa) or (IIb) | —CF₃ | —F | -n-propyl |
| BID(IIa) or (IIb) | —CF₃ | —F | -n-butyl |
| BIE(IIa) or (IIb) | —CF₃ | —F | -t-butyl |
| BIF(IIa) or (IIb) | —CF₃ | —F | -iso-butyl |
| BIG(IIa) or (IIb) | —CF₃ | —F | —OCH₃ |
| BIH(IIa) or (IIb) | —CF₃ | —F | —OC₂H₅ |
| BII(IIa) or (IIb) | —CF₃ | —F | —OC₃H₇ |
| BIJ(IIa) or (IIb) | —CF₃ | —F | —CHF₂ |
| BIK(IIa) or (IIb) | —CF₃ | —F | —CF₃ |
| BIL(IIa) or (IIb) | —CF₃ | —F | —CHCl₂ |
| BIM(IIa) or (IIb) | —CF₃ | —F | —CCl₃ |
| BIN(IIa) or (IIb) | —CF₃ | —F | —F |
| BIO(IIa) or (IIb) | —CF₃ | —F | —Cl |
| BIP(IIa) or (IIb) | —CF₃ | —F | —Br |
| BIQ(IIa) or (IIb) | —CF₃ | —F | —I |
| BIR(IIa) or (IIb) | —CF₃ | —Cl | —H |
| BIS(IIa) or (IIb) | —CF₃ | —Cl | —CH₃ |
| BIT(IIa) or (IIb) | —CF₃ | —Cl | -n-propyl |
| BIU(IIa) or (IIb) | —CF₃ | —Cl | -n-butyl |
| BIV(IIa) or (IIb) | —CF₃ | —Cl | -t-butyl |
| BIW(IIa) or (IIb) | —CF₃ | —Cl | -iso-butyl |
| BIX(IIa) or (IIb) | —CF₃ | —Cl | —OCH₃ |
| BIY(IIa) or (IIb) | —CF₃ | —Cl | —OC₂H₅ |
| BIZ(IIa) or (IIb) | —CF₃ | —Cl | —OC₃H₇ |
| BJA(IIa) or (IIb) | —CF₃ | —Cl | —CHF₂ |
| BJB(IIa) or (IIb) | —CF₃ | —Cl | —CF₃ |
| BJC(IIa) or (IIb) | —CF₃ | —Cl | —CHCl₂ |
| BJD(IIa) or (IIb) | —CF₃ | —Cl | —CCl₃ |
| BJE(IIa) or (IIb) | —CF₃ | —Cl | —F |
| BJF(IIa) or (IIb) | —CF₃ | —Cl | —Cl |
| BJG(IIa) or (IIb) | —CF₃ | —Cl | —Br |
| BJH(IIa) or (IIb) | —CF₃ | —Cl | —I |
| BJI(IIa) or (IIb) | —CF₃ | —Br | —H |
| BJJ(IIa) or (IIb) | —CF₃ | —Br | —CH₃ |
| BJK(IIa) or (IIb) | —CF₃ | —Br | -n-propyl |
| BJL(IIa) or (IIb) | —CF₃ | —Br | -n-butyl |
| BJM(IIa) or (IIb) | —CF₃ | —Br | -t-butyl |
| BJN(IIa) or (IIb) | —CF₃ | —Br | -iso-butyl |
| BJO(IIa) or (IIb) | —CF₃ | —Br | —OCH₃ |
| BJP(IIa) or (IIb) | —CF₃ | —Br | —OC₂H₅ |
| BJQ(IIa) or (IIb) | —CF₃ | —Br | —OC₃H₇ |
| BJR(IIa) or (IIb) | —CF₃ | —Br | —CHF₂ |
| BJS(IIa) or (IIb) | —CF₃ | —Br | —CF₃ |
| BJT(IIa) or (IIb) | —CF₃ | —Br | —CHCl₂ |
| BJU(IIa) or (IIb) | —CF₃ | —Br | —CCl₃ |
| BJV(IIa) or (IIb) | —CF₃ | —Br | —F |
| BJW(IIa) or (IIb) | —CF₃ | —Br | —Cl |
| BJX(IIa) or (IIb) | —CF₃ | —Br | —Br |
| BJY(IIa) or (IIb) | —CF₃ | —Br | —I |
| BJZ(IIa) or (IIb) | —CF₃ | —I | —H |
| BKA(IIa) or (IIb) | —CF₃ | —I | —CH₃ |
| BKB(IIa) or (IIb) | —CF₃ | —I | -n-propyl |
| BKC(IIa) or (IIb) | —CF₃ | —I | -n-butyl |
| BKD(IIa) or (IIb) | —CF₃ | —I | -t-butyl |
| BKE(IIa) or (IIb) | —CF₃ | —I | -iso-butyl |
| BKF(IIa) or (IIb) | —CF₃ | —I | —OCH₃ |
| BKG(IIa) or (IIb) | —CF₃ | —I | —OC₂H₅ |
| BKH(IIa) or (IIb) | —CF₃ | —I | —OC₃H₇ |
| BKI(IIa) or (IIb) | —CF₃ | —I | —CHF₂ |
| BKJ(IIa) or (IIb) | —CF₃ | —I | —CF₃ |
| BKK(IIa) or (IIb) | —CF₃ | —I | —CHCl₂ |
| BKL(IIa) or (IIb) | —CF₃ | —I | —CCl₃ |
| BKM(IIa) or (IIb) | —CF₃ | —I | —F |
| BKN(IIa) or (IIb) | —CF₃ | —I | —Cl |
| BKO(IIa) or (IIb) | —CF₃ | —I | —Br |
| BKP(IIa) or (IIb) | —CF₃ | —I | —I |
| BKQ(IIa) or (IIb) | —CF₃ | —NO₂ | —H |
| BKR(IIa) or (IIb) | —CF₃ | —NO₂ | —CH₃ |
| BKS(IIa) or (IIb) | —CF₃ | —NO₂ | -n-propyl |
| BKT(IIa) or (IIb) | —CF₃ | —NO₂ | -n-butyl |
| BKU(IIa) or (IIb) | —CF₃ | —NO₂ | -t-butyl |
| BKV(IIa) or (IIb) | —CF₃ | —NO₂ | -iso-butyl |
| BKW(IIa) or (IIb) | —CF₃ | —NO₂ | —OCH₃ |
| BKX(IIa) or (IIb) | —CF₃ | —NO₂ | —OC₂H₅ |
| BKY(IIa) or (IIb) | —CF₃ | —NO₂ | —OC₃H₇ |
| BKZ(IIa) or (IIb) | —CF₃ | —NO₂ | —CHF₂ |
| BLA(IIa) or (IIb) | —CF₃ | —NO₂ | —CF₃ |
| BLB(IIa) or (IIb) | —CF₃ | —NO₂ | —CHCl₂ |
| BLC(IIa) or (IIb) | —CF₃ | —NO₂ | —CCl₃ |
| BLD(IIa) or (IIb) | —CF₃ | —NO₂ | —F |
| BLE(IIa) or (IIb) | —CF₃ | —NO₂ | —Cl |
| BLF(IIa) or (IIb) | —CF₃ | —NO₂ | —Br |
| BLG(IIa) or (IIb) | —CF₃ | —NO₂ | —I |
| BLH(IIa) or (IIb) | —CF₃ | —CN | —H |
| BLI(IIa) or (IIb) | —CF₃ | —CN | —CH₃ |
| BLJ(IIa) or (IIb) | —CF₃ | —CN | -n-propyl |
| BLK(IIa) or (IIb) | —CF₃ | —CN | -n-butyl |
| BLL(IIa) or (IIb) | —CF₃ | —CN | -t-butyl |
| BLM(IIa) or (IIb) | —CF₃ | —CN | -iso-butyl |
| BLN(IIa) or (IIb) | —CF₃ | —CN | —OCH₃ |
| BLO(IIa) or (IIb) | —CF₃ | —CN | —OC₂H₅ |
| BLP(IIa) or (IIb) | —CF₃ | —CN | —OC₃H₇ |
| BLQ(IIa) or (IIb) | —CF₃ | —CN | —CHF₂ |
| BLR(IIa) or (IIb) | —CF₃ | —CN | —CF₃ |
| BLS(IIa) or (IIb) | —CF₃ | —CN | —CHCl₂ |
| BLT(IIa) or (IIb) | —CF₃ | —CN | —CCl₃ |
| BLU(IIa) or (IIb) | —CF₃ | —CN | —F |
| BLV(IIa) or (IIb) | —CF₃ | —CN | —Cl |
| BLW(IIa) or (IIb) | —CF₃ | —CN | —Br |
| BLX(IIa) or (IIb) | —CF₃ | —CN | —I |
| BLY(IIa) or (IIb) | —CF₃ | —NH₂ | —H |
| BLZ(IIa) or (IIb) | —CF₃ | —NH₂ | —CH₃ |
| BMA(IIa) or (IIb) | —CF₃ | —NH₂ | -n-propyl |
| BMB(IIa) or (IIb) | —CF₃ | —NH₂ | -n-butyl |
| BMC(IIa) or (IIb) | —CF₃ | —NH₂ | -t-butyl |
| BMD(IIa) or (IIb) | —CF₃ | —NH₂ | -iso-butyl |
| BME(IIa) or (IIb) | —CF₃ | —NH₂ | —OCH₃ |
| BMF(IIa) or (IIb) | —CF₃ | —NH₂ | —OC₂H₅ |
| BMG(IIa) or (IIb) | —CF₃ | —NH₂ | —OC₃H₇ |
| BMH(IIa) or (IIb) | —CF₃ | —NH₂ | —CHF₂ |
| BMI(IIa) or (IIb) | —CF₃ | —NH₂ | —CF₃ |
| BMJ(IIa) or (IIb) | —CF₃ | —NH₂ | —CHCl₂ |
| BMK(IIa) or (IIb) | —CF₃ | —NH₂ | —CCl₃ |
| BML(IIa) or (IIb) | —CF₃ | —NH₂ | —F |
| BMM(IIa) or (IIb) | —CF₃ | —NH₂ | —Cl |
| BMN(IIa) or (IIb) | —CF₃ | —NH₂ | —Br |
| BMO(IIa) or (IIb) | —CF₃ | —NH₂ | —I |
| BMP(IIa) or (IIb) | —CF₃ | —CH₃ | —H |
| BMQ(IIa) or (IIb) | —CF₃ | —CH₃ | —CH₃ |
| BMR(IIa) or (IIb) | —CF₃ | —CH₃ | -n-propyl |
| BMS(IIa) or (IIb) | —CF₃ | —CH₃ | -n-butyl |
| BMT(IIa) or (IIb) | —CF₃ | —CH₃ | -t-butyl |
| BMU(IIa) or (IIb) | —CF₃ | —CH₃ | -iso-butyl |
| BMV(IIa) or (IIb) | —CF₃ | —CH₃ | —OCH₃ |
| BMW(IIa) or (IIb) | —CF₃ | —CH₃ | —OC₂H₅ |
| BMX(IIa) or (IIb) | —CF₃ | —CH₃ | —OC₃H₇ |
| BMY(IIa) or (IIb) | —CF₃ | —CH₃ | —CHF₂ |
| BMZ(IIa) or (IIb) | —CF₃ | —CH₃ | —CF₃ |
| BNA(IIa) or (IIb) | —CF₃ | —CH₃ | —CHCl₂ |
| BNB(IIa) or (IIb) | —CF₃ | —CH₃ | —CCl₃ |
| BNC(IIa) or (IIb) | —CF₃ | —CH₃ | —F |
| BND(IIa) or (IIb) | —CF₃ | —CH₃ | —Cl |
| BNE(IIa) or (IIb) | —CF₃ | —CH₃ | —Br |
| BNF(IIa) or (IIb) | —CF₃ | —CH₃ | —I |
| BNG(IIa) | —NO₂ | —H | —H |
| BNH(IIa) | —NO₂ | —H | —CH₃ |
| BNI(IIa) | —NO₂ | —H | -n-propyl |
| BNJ(IIa) | —NO₂ | —H | -n-butyl |
| BNK(IIa) | —NO₂ | —H | -t-butyl |
| BNL(IIa) | —NO₂ | —H | -iso-butyl |
| BNM(IIa) | —NO₂ | —H | —OCH₃ |
| BNN(IIa) | —NO₂ | —H | —OC₂H₅ |
| BNO(IIa) | —NO₂ | —H | —OC₃H₇ |
| BNP(IIa) | —NO₂ | —H | —CHF₂ |
| BNQ(IIa) | —NO₂ | —H | —CF₃ |
| BNR(IIa) | —NO₂ | —H | —CHCl₂ |
| BNS(IIa) | —NO₂ | —H | —CCl₃ |
| BNT(IIa) | —NO₂ | —H | —F |
| BNU(IIa) | —NO₂ | —H | —Cl |
| BNV(IIa) | —NO₂ | —H | —Br |
| BNW(IIa) | —NO₂ | —H | —I |
| BNX(IIa) or (IIb) | —NO₂ | —OH | —H |
| BNY(IIa) or (IIb) | —NO₂ | —OH | —CH₃ |
| BNZ(IIa) or (IIb) | —NO₂ | —OH | -n-propyl |
| BOA(IIa) or (IIb) | —NO₂ | —OH | -n-butyl |
| BOB(IIa) or (IIb) | —NO₂ | —OH | -t-butyl |
| BOC(IIa) or (IIb) | —NO₂ | —OH | -iso-butyl |
| BOD(IIa) or (IIb) | —NO₂ | —OH | —OCH₃ |
| BOE(IIa) or (IIb) | —NO₂ | —OH | —OC₂H₅ |
| BOF(IIa) or (IIb) | —NO₂ | —OH | —OC₃H₇ |
| BOG(IIa) or (IIb) | —NO₂ | —OH | —CHF₂ |
| BOH(IIa) or (IIb) | —NO₂ | —OH | —CF₃ |
| BOI(IIa) or (IIb) | —NO₂ | —OH | —CHCl₂ |
| BOJ(IIa) or (IIb) | —NO₂ | —OH | —CCl₃ |
| BOK(IIa) or (IIb) | —NO₂ | —OH | —F |
| BOL(IIa) or (IIb) | —NO₂ | —OH | —Cl |
| BOM(IIa) or (IIb) | —NO₂ | —OH | —Br |
| BON(IIa) or (IIb) | —NO₂ | —OH | —I |
| BOO(IIa) or (IIb) | —NO₂ | —F | —H |
| BOP(IIa) or (IIb) | —NO₂ | —F | —CH₃ |
| BOQ(IIa) or (IIb) | —NO₂ | —F | -n-propyl |
| BOR(IIa) or (IIb) | —NO₂ | —F | -n-butyl |
| BOS(IIa) or (IIb) | —NO₂ | —F | -t-butyl |
| BOT(IIa) or (IIb) | —NO₂ | —F | -iso-butyl |
| BOU(IIa) or (IIb) | —NO₂ | —F | —OCH₃ |
| BOV(IIa) or (IIb) | —NO₂ | —F | —OC₂H₅ |
| BOW(IIa) or (IIb) | —NO₂ | —F | —OC₃H₇ |
| BOX(IIa) or (IIb) | —NO₂ | —F | —CHF₂ |
| BOY(IIa) or (IIb) | —NO₂ | —F | —CF₃ |
| BOZ(IIa) or (IIb) | —NO₂ | —F | —CHCl₂ |
| BPA(IIa) or (IIb) | —NO₂ | —F | —CCl₃ |
| BPB(IIa) or (IIb) | —NO₂ | —F | —F |
| BPC(IIa) or (IIb) | —NO₂ | —F | —Cl |
| BPD(IIa) or (IIb) | —NO₂ | —F | —Br |
| BPE(IIa) or (IIb) | —NO₂ | —F | —I |
| BPF(IIa) or (IIb) | —NO₂ | —Cl | —H |
| BPG(IIa) or (IIb) | —NO₂ | —Cl | —CH₃ |
| BPH(IIa) or (IIb) | —NO₂ | —Cl | -n-propyl |
| BPI(IIa) or (IIb) | —NO₂ | —Cl | -n-butyl |
| BPJ(IIa) or (IIb) | —NO₂ | —Cl | -t-butyl |
| BPK(IIa) or (IIb) | —NO₂ | —Cl | -iso-butyl |
| BPL(IIa) or (IIb) | —NO₂ | —Cl | —OCH₃ |
| BPM(IIa) or (IIb) | —NO₂ | —Cl | —OC₂H₅ |
| BPN(IIa) or (IIb) | —NO₂ | —Cl | —OC₃H₇ |
| BPO(IIa) or (IIb) | —NO₂ | —Cl | —CHF2 |
| BPP(IIa) or (IIb) | —NO₂ | —Cl | —CF₃ |
| BPQ(IIa) or (IIb) | —NO₂ | —Cl | —CHCl₂ |
| BPR(IIa) or (IIb) | —NO₂ | —Cl | —CCl₃ |
| BPS(IIa) or (IIb) | —NO₂ | —Cl | —F |
| BPT(IIa) or (IIb) | —NO₂ | —Cl | —Cl |
| BPU(IIa) or (IIb) | —NO₂ | —Cl | —Br |
| BPV(IIa) or (IIb) | —NO₂ | —Cl | —I |
| BPW(IIa) or (IIb) | —NO₂ | —Br | —H |
| BPX(IIa) or (IIb) | —NO₂ | —Br | —CH₃ |
| BPY(IIa) or (IIb) | —NO₂ | —Br | -n-propyl |
| BPZ(IIa) or (IIb) | —NO₂ | —Br | -n-butyl |
| BQA(IIa) or (IIb) | —NO₂ | —Br | -t-butyl |
| BQB(IIa) or (IIb) | —NO₂ | —Br | -iso-butyl |
| BQC(IIa) or (IIb) | —NO₂ | —Br | —OCH₃ |
| BQD(IIa) or (IIb) | —NO₂ | —Br | —OC₂H₅ |
| BQE(IIa) or (IIb) | —NO₂ | —Br | —OC₃H₇ |
| BQF(IIa) or (IIb) | —NO₂ | —Br | —CHF₂ |
| BQG(IIa) or (IIb) | —NO₂ | —Br | —CF₃ |
| BQH(IIa) or (IIb) | —NO₂ | —Br | —CHCl₂ |
| BQI(IIa) or (IIb) | —NO₂ | —Br | —CCl₃ |
| BQJ(IIa) or (IIb) | —NO₂ | —Br | —F |
| BQK(IIa) or (IIb) | —NO₂ | —Br | —Cl |
| BQL(IIa) or (IIb) | —NO₂ | —Br | —Br |
| BQM(IIa) or (IIb) | —NO₂ | —Br | —I |
| BQN(IIa) or (IIb) | —NO₂ | —I | —H |
| BQO(IIa) or (IIb) | —NO₂ | —I | —CH₃ |
| BQP(IIa) or (IIb) | —NO₂ | —I | -n-propyl |
| BQQ(IIa) or (IIb) | —NO₂ | —I | -n-butyl |
| BQR(IIa) or (IIb) | —NO₂ | —I | -t-butyl |
| BQS(IIa) or (IIb) | —NO₂ | —I | -iso-butyl |
| BQT(IIa) or (IIb) | —NO₂ | —I | —OCH₃ |
| BQU(IIa) or (IIb) | —NO₂ | —I | —OC₂H₅ |
| BQV(IIa) or (IIb) | —NO₂ | —I | —OC₃H₇ |
| BQW(IIa) or (IIb) | —NO₂ | —I | —CHF₂ |
| BQX(IIa) or (IIb) | —NO₂ | —I | —CF₃ |
| BQY(IIa) or (IIb) | —NO₂ | —I | —CHCl₂ |
| BQZ(IIa) or (IIb) | —NO₂ | —I | —CCl₃ |
| BRA(IIa) or (IIb) | —NO₂ | —I | —F |
| BRB(IIa) or (IIb) | —NO₂ | —I | —Cl |
| BRC(IIa) or (IIb) | —NO₂ | —I | —Br |
| BRD(IIa) or (IIb) | —NO₂ | —I | —I |
| BRE(IIa) or (IIb) | —NO₂ | —NO₂ | —H |
| BRF(IIa) or (IIb) | —NO₂ | —NO₂ | —CH₃ |
| BRG(IIa) or (IIb) | —NO₂ | —NO₂ | -n-propyl |
| BRH(IIa) or (IIb) | —NO₂ | —NO₂ | -n-butyl |
| BRI(IIa) or (IIb) | —NO₂ | —NO₂ | -t-butyl |
| BRJ(IIa) or (IIb) | —NO₂ | —NO₂ | -iso-butyl |
| BRK(IIa) or (IIb) | —NO₂ | —NO₂ | —OCH₃ |
| BRL(IIa) or (IIb) | —NO₂ | —NO₂ | —OC₂H₅ |
| BRM(IIa) or (IIb) | —NO₂ | —NO₂ | —OC₃H₇ |
| BRN(IIa) or (IIb) | —NO₂ | —NO₂ | —CHF₂ |
| BRO(IIa) or (IIb) | —NO₂ | —NO₂ | —CF₃ |
| BRP(IIa) or (IIb) | —NO₂ | —NO₂ | —CHCl₂ |
| BRQ(IIa) or (IIb) | —NO₂ | —NO₂ | —CCl₃ |
| BRR(IIa) or (IIb) | —NO₂ | —NO₂ | —F |
| BRS(IIa) or (IIb) | —NO₂ | —NO₂ | —Cl |
| BRT(IIa) or (IIb) | —NO₂ | —NO₂ | —Br |
| BRU(IIa) or (IIb) | —NO₂ | —NO₂ | —I |
| BRV(IIa) or (IIb) | —NO₂ | —CN | —H |
| BRW(IIa) or (IIb) | —NO₂ | —CN | —CH₃ |
| BRX(IIa) or (IIb) | —NO₂ | —CN | -n-propyl |
| BRY(IIa) or (IIb) | —NO₂ | —CN | -n-butyl |
| BRZ(IIa) or (IIb) | —NO₂ | —CN | -t-butyl |
| BSA(IIa) or (IIb) | —NO₂ | —CN | -iso-butyl |
| BSB(IIa) or (IIb) | —NO₂ | —CN | —OCH₃ |
| BSC(IIa) or (IIb) | —NO₂ | —CN | —OC₂H₅ |
| BSD(IIa) or (IIb) | —NO₂ | —CN | —OC₃H₇ |
| BSE(IIa) or (IIb) | —NO₂ | —CN | —CHF₂ |
| BSF(IIa) or (IIb) | —NO₂ | —CN | —CF₃ |
| BSG(IIa) or (IIb) | —NO₂ | —CN | —CHCl₂ |
| BSH(IIa) or (IIb) | —NO₂ | —CN | —CCl₃ |
| BSI(IIa) or (IIb) | —NO₂ | —CN | —F |
| BSJ(IIa) or (IIb) | —NO₂ | —CN | —Cl |
| BSK(IIa) or (IIb) | —NO₂ | —CN | —Br |
| BSL(IIa) or (IIb) | —NO₂ | —CN | —I |
| BSM(IIa) or (IIb) | —NO₂ | —NH₂ | —H |
| BSN(IIa) or (IIb) | —NO₂ | —NH₂ | —CH₃ |
| BSO(IIa) or (IIb) | —NO₂ | —NH₂ | -n-propyl |
| BSP(IIa) or (IIb) | —NO₂ | —NH₂ | -n-butyl |
| BSQ(IIa) or (IIb) | —NO₂ | —NH₂ | -t-butyl |
| BSR(IIa) or (IIb) | —NO₂ | —NH₂ | -iso-butyl |
| BSS(IIa) or (IIb) | —NO₂ | —NH₂ | —OCH₃ |
| BST(IIa) or (IIb) | —NO₂ | —NH₂ | —OC₂H₅ |
| BSU(IIa) or (IIb) | —NO₂ | —NH₂ | —OC₃H₇ |
| BSV(IIa) or (IIb) | —NO₂ | —NH₂ | —CHF₂ |
| BSW(IIa) or (IIb) | —NO₂ | —NH₂ | —CF₃ |
| BSX(IIa) or (IIb) | —NO₂ | —NH₂ | —CHCl₂ |
| BSY(IIa) or (IIb) | —NO₂ | —NH₂ | —CCl₃ |
| BSZ(IIa) or (IIb) | —NO₂ | —NH₂ | —F |
| BTA(IIa) or (IIb) | —NO₂ | —NH₂ | —Cl |
| BTB(IIa) or (IIb) | —NO₂ | —NH₂ | —Br |
| BTC(IIa) or (IIb) | —NO₂ | —NH₂ | —I |
| BTD(IIa) or (IIb) | —NO₂ | —CH₃ | —H |
| BTE(IIa) or (IIb) | —NO₂ | —CH₃ | —CH₃ |
| BTF(IIa) or (IIb) | —NO₂ | —CH₃ | -n-propyl |
| BTG(IIa) or (IIb) | —NO₂ | —CH₃ | -n-butyl |
| BTH(IIa) or (IIb) | —NO₂ | —CH₃ | -t-butyl |
| BTI(IIa) or (IIb) | —NO₂ | —CH₃ | -iso-butyl |
| BTJ(IIa) or (IIb) | —NO₂ | —CH₃ | —OCH₃ |
| BTK(IIa) or (IIb) | —NO₂ | —CH₃ | —OC₂H₅ |
| BTL(IIa) or (IIb) | —NO₂ | —CH₃ | —OC₃H₇ |
| BTM(IIa) or (IIb) | —NO₂ | —CH₃ | —CHF₂ |
| BTN(IIa) or (IIb) | —NO₂ | —CH₃ | —CF₃ |
| BTO(IIa) or (IIb) | —NO₂ | —CH₃ | —CHCl₂ |
| BTP(IIa) or (IIb) | —NO₂ | —CH₃ | —CCl₃ |
| BTQ(IIa) or (IIb) | —NO₂ | —CH₃ | —F |
| BTR(IIa) or (IIb) | —NO₂ | —CH₃ | —Cl |
| BTS(IIa) or (IIb) | —NO₂ | —CH₃ | —Br |
| BTT(IIa) or (IIb) | —NO₂ | —CH₃ | —I |
| BTU(IIa) | —CN | —H | —H |
| BTV(IIa) | —CN | —H | —CH₃ |
| BTW(IIa) | —CN | —H | -n-propyl |
| BTX(IIa) | —CN | —H | -n-butyl |
| BTY(IIa) | —CN | —H | -t-butyl |
| BTZ(IIa) | —CN | —H | -iso-butyl |
| BUA(IIa) | —CN | —H | —OCH₃ |
| BUB(IIa) | —CN | —H | —OC₂H₅ |
| BUC(IIa) | —CN | —H | —OC₃H₇ |
| BUD(IIa) | —CN | —H | —CHF₂ |
| BUE(IIa) | —CN | —H | —CF₃ |
| BUF(IIa) | —CN | —H | —CHCl₂ |
| BUG(IIa) | —CN | —H | —CCl₃ |
| BUH(IIa) | —CN | —H | —F |
| BUI(IIa) | —CN | —H | —Cl |
| BUJ(IIa) | —CN | —H | —Br |
| BUK(IIa) | —CN | —H | —I |
| BUL(IIa) or (IIb) | —CN | —OH | —H |
| BUM(IIa) or (IIb) | —CN | —OH | —CH₃ |
| BUN(IIa) or (IIb) | —CN | —OH | -n-propyl |
| BUO(IIa) or (IIb) | —CN | —OH | -n-butyl |
| BUP(IIa) or (IIb) | —CN | —OH | -t-butyl |
| BUQ(IIa) or (IIb) | —CN | —OH | -iso-butyl |
| BUR(IIa) or (IIb) | —CN | —OH | —OCH₃ |
| BUS(IIa) or (IIb) | —CN | —OH | —OC₂H₅ |
| BUT(IIa) or (IIb) | —CN | —OH | —OC₃H₇ |
| BUU(IIa) or (IIb) | —CN | —OH | —CHF₂ |
| BUV(IIa) or (IIb) | —CN | —OH | —CF₃ |
| BUW(IIa) or (IIb) | —CN | —OH | —CHCl₂ |
| BUX(IIa) or (IIb) | —CN | —OH | —CCl₃ |
| BUY(IIa) or (IIb) | —CN | —OH | —F |
| BUZ(IIa) or (IIb) | —CN | —OH | —Cl |
| BVA(IIa) or (IIb) | —CN | —OH | —Br |
| BVB(IIa) or (IIb) | —CN | —OH | —I |
| BVC(IIa) or (IIb) | —CN | —F | —H |
| BVD(IIa) or (IIb) | —CN | —F | —CH₃ |
| BVE(IIa) or (IIb) | —CN | —F | -n-propyl |
| BVF(IIa) or (IIb) | —CN | —F | -n-butyl |
| BVG(IIa) or (IIb) | —CN | —F | -t-butyl |
| BVH(IIa) or (IIb) | —CN | —F | -iso-butyl |
| BVI(IIa) or (IIb) | —CN | —F | —OCH₃ |
| BVJ(IIa) or (IIb) | —CN | —F | —OC₂H₅ |
| BVK(IIa) or (IIb) | —CN | —F | —OC₃H₇ |
| BVL(IIa) or (IIb) | —CN | —F | —CHF₂ |
| BVM(IIa) or (IIb) | —CN | —F | —CF₃ |
| BVN(IIa) or (IIb) | —CN | —F | —CHCl₂ |
| BVO(IIa) or (IIb) | —CN | —F | —CCl₃ |
| BVP(IIa) or (IIb) | —CN | —F | —F |
| BVQ(IIa) or (IIb) | —CN | —F | —Cl |
| BVR(IIa) or (IIb) | —CN | —F | —Br |
| BVS(IIa) or (IIb) | —CN | —F | —I |
| BVT(IIa) or (IIb) | —CN | —Cl | —H |
| BVU(IIa) or (IIb) | —CN | —Cl | —CH₃ |
| BVV(IIa) or (IIb) | —CN | —Cl | -n-propyl |
| BVW(IIa) or (IIb) | —CN | —Cl | -n-butyl |
| BVX(IIa) or (IIb) | —CN | —Cl | -t-butyl |
| BVY(IIa) or (IIb) | —CN | —Cl | -iso-butyl |
| BVZ(IIa) or (IIb) | —CN | —Cl | —OCH₃ |
| BWA(IIa) or (IIb) | —CN | —Cl | —OC₂H₅ |
| BWB(IIa) or (IIb) | —CN | —Cl | —OC₃H₇ |
| BWC(IIa) or (IIb) | —CN | —Cl | —CHF₂ |
| BWD(IIa) or (IIb) | —CN | —Cl | —CF₃ |
| BWE(IIa) or (IIb) | —CN | —Cl | —CHCl₂ |
| BWF(IIa) or (IIb) | —CN | —Cl | —CCl₃ |
| BWG(IIa) or (IIb) | —CN | —Cl | —F |
| BWH(IIa) or (IIb) | —CN | —Cl | —Cl |
| BWI(IIa) or (IIb) | —CN | —Cl | —Br |
| BWJ(IIa) or (IIb) | —CN | —Cl | —I |
| BWK(IIa) or (IIb) | —CN | —Br | —H |
| BWL(IIa) or (IIb) | —CN | —Br | —CH₃ |
| BWM(IIa) or (IIb) | —CN | —Br | -n-propyl |
| BWN(IIa) or (IIb) | —CN | —Br | -n-butyl |
| BWO(IIa) or (IIb) | —CN | —Br | -t-butyl |
| BWP(IIa) or (IIb) | —CN | —Br | -iso-butyl |
| BWQ(IIa) or (IIb) | —CN | —Br | —OCH₃ |
| BWR(IIa) or (IIb) | —CN | —Br | —OC₂H₅ |
| BWS(IIa) or (IIb) | —CN | —Br | —OC₃H₇ |
| BWT(IIa) or (IIb) | —CN | —Br | —CHF₂ |
| BWU(IIa) or (IIb) | —CN | —Br | —CF₃ |
| BWV(IIa) or (IIb) | —CN | —Br | —CHCl₂ |
| BWW(IIa) or (IIb) | —CN | —Br | —CCl₃ |
| BWX(IIa) or (IIb) | —CN | —Br | —F |
| BWY(IIa) or (IIb) | —CN | —Br | —Cl |
| BWZ(IIa) or (IIb) | —CN | —Br | —Br |
| BXA(IIa) or (IIb) | —CN | —Br | —I |
| BXB(IIa) or (IIb) | —CN | —I | —H |
| BXC(IIa) or (IIb) | —CN | —I | —CH₃ |
| BXD(IIa) or (IIb) | —CN | —I | -n-propyl |
| BXE(IIa) or (IIb) | —CN | —I | -n-butyl |
| BXF(IIa) or (IIb) | —CN | —I | -t-butyl |
| BXG(IIa) or (IIb) | —CN | —I | -iso-butyl |
| BXH(IIa) or (IIb) | —CN | —I | —OCH₃ |
| BXI(IIa) or (IIb) | —CN | —I | —OC₂H₅ |
| BXI(IIa) or (IIb) | —CN | —I | —OC₃H₇ |
| BXK(IIa) or (IIb) | —CN | —I | —CHF₂ |
| BXL(IIa) or (IIb) | —CN | —I | —CF₃ |
| BXM(IIa) or (IIb) | —CN | —I | —CHCl₂ |
| BXN(IIa) or (IIb) | —CN | —I | —CCl₃ |
| BXO(IIa) or (IIb) | —CN | —I | —F |
| BXP(IIa) or (IIb) | —CN | —I | —Cl |
| BXQ(IIa) or (IIb) | —CN | —I | —Br |
| BXR(IIa) or (IIb) | —CN | —I | —I |
| BXS(IIa) or (IIb) | —CN | —NO₂ | —H |
| BXT(IIa) or (IIb) | —CN | —NO₂ | —CH₃ |
| BXU(IIa) or (IIb) | —CN | —NO₂ | -n-propyl |
| BXV(IIa) or (IIb) | —CN | —NO₂ | -n-butyl |
| BXW(Ha) or (IIb) | —CN | —NO₂ | -t-butyl |
| BXX(IIa) or (IIb) | —CN | —NO₂ | -iso-butyl |
| BXY(IIa) or (IIb) | —CN | —NO₂ | —OCH₃ |
| BXZ(IIa) or (IIb) | —CN | —NO₂ | —OC₂H₅ |
| BYA(IIa) or (IIb) | —CN | —NO₂ | —OC₃H₇ |
| BYB(IIa) or (IIb) | —CN | —NO₂ | —CHF₂ |
| BYC(IIa) or (IIb) | —CN | —NO₂ | —CF₃ |
| BYD(IIa) or (IIb) | —CN | —NO₂ | —CHCl₂ |
| BYE(IIa) or (IIb) | —CN | —NO₂ | —CCl₃ |
| BYF(IIa) or (IIb) | —CN | —NO₂ | —F |
| BYG(IIa) or (IIb) | —CN | —NO₂ | —Cl |
| BYH(IIa) or (IIb) | —CN | —NO₂ | —Br |
| BYI(IIa) or (IIb) | —CN | —NO₂ | —I |
| BYJ(IIa) or (IIb) | —CN | —CN | —H |
| BYK(IIa) or (IIb) | —CN | —CN | —CH₃ |
| BYL(IIa) or (IIb) | —CN | —CN | -n-propyl |
| BYM(IIa) or (IIb) | —CN | —CN | -n-butyl |
| BYN(IIa) or (IIb) | —CN | —CN | -t-butyl |
| BYO(IIa) or (IIb) | —CN | —CN | -iso-butyl |
| BYP(IIa) or (IIb) | —CN | —CN | —OCH₃ |
| BYQ(IIa) or (IIb) | —CN | —CN | —OC₂H₅ |
| BYR(IIa) or (IIb) | —CN | —CN | —OC₃H₇ |
| BYS(IIa) or (IIb) | —CN | —CN | —CHF₂ |
| BYT(IIa) or (IIb) | —CN | —CN | —CF₃ |
| BYU(IIa) or (IIb) | —CN | —CN | —CHCl₂ |
| BYV(IIa) or (IIb) | —CN | —CN | —CCl₃ |
| BYW(IIa) or (IIb) | —CN | —CN | —F |
| BYX(IIa) or (IIb) | —CN | —CN | —Cl |
| BYY(IIa) or (IIb) | —CN | —CN | —Br |
| BYZ(IIa) or (IIb) | —CN | —CN | —I |
| BZA(IIa) or (IIb) | —CN | —NH₂ | —H |
| BZB(IIa) or (IIb) | —CN | —NH₂ | —CH₃ |
| BZC(IIa) or (IIb) | —CN | —NH₂ | -n-propyl |
| BZD(IIa) or (IIb) | —CN | —NH₂ | -n-butyl |
| BZE(IIa) or (IIb) | —CN | —NH₂ | -t-butyl |
| BZF(IIa) or (IIb) | —CN | —NH₂ | -iso-butyl |
| BZG(IIa) or (IIb) | —CN | —NH₂ | —OCH₃ |
| BZH(IIa) or (IIb) | —CN | —NH₂ | —OC₂H₅ |
| BZI(IIa) or (IIb) | —CN | —NH₂ | —OC₃H₇ |
| BZJ(IIa) or (IIb) | —CN | —NH₂ | —CHF₂ |
| BZK(IIa) or (IIb) | —CN | —NH₂ | —CF₃ |
| BZL(IIa) or (IIb) | —CN | —NH₂ | —CHCl₂ |
| BZM(IIa) or (IIb) | —CN | —NH₂ | —CCl₃ |
| BZN(IIa) or (IIb) | —CN | —NH₂ | —F |
| BZO(IIa) or (IIb) | —CN | —NH₂ | —Cl |
| BZP(IIa) or (IIb) | —CN | —NH₂ | —Br |
| BZQ(IIa) or (IIb) | —CN | —NH₂ | —I |
| BZR(IIa) or (IIb) | —CN | —CH₃ | —H |
| BZS(IIa) or (IIb) | —CN | —CH₃ | —CH₃ |
| BZT(IIa) or (IIb) | —CN | —CH₃ | -n-propyl |
| BZU(IIa) or (IIb) | —CN | —CH₃ | -n-butyl |
| BZV(IIa) or (IIb) | —CN | —CH₃ | -t-butyl |
| BZW(IIa) or (IIb) | —CN | —CH₃ | -iso-butyl |
| BZX(IIa) or (IIb) | —CN | —CH₃ | —OCH₃ |
| BZY(IIa) or (IIb) | —CN | —CH₃ | —OC₂H₅ |
| BZZ(IIa) or (IIb) | —CN | —CH₃ | —OC₃H₇ |
| CAA(IIa) or (IIb) | —CN | —CH₃ | —CHF₂ |
| CAB(IIa) or (IIb) | —CN | —CH₃ | —CF₃ |
| CAC(IIa) or (IIb) | —CN | —CH₃ | —CHCl₂ |
| CAD(IIa) or (IIb) | —CN | —CH₃ | —CCl₃ |
| CAE(IIa) or (IIb) | —CN | —CH₃ | —F |
| CAF(IIa) or (IIb) | —CN | —CH₃ | —Cl |
| CAG(IIa) or (IIb) | —CN | —CH₃ | —Br |
| CAH(IIa) or (IIb) | —CN | —CH₃ | —I |
| CAI(IIa) | —CH₃ | —H | —H |
| CAJ(IIa) | —CH₃ | —H | —CH₃ |
| CAK(IIa) | —CH₃ | —H | -n-propyl |
| CAL(IIa) | —CH₃ | —H | -n-butyl |
| CAM(IIa) | —CH₃ | —H | -t-butyl |
| CAN(IIa) | —CH₃ | —H | -iso-butyl |
| CAO(IIa) | —CH₃ | —H | —OCH₃ |
| CAP(IIa) | —CH₃ | —H | —OC₂H₅ |
| CAQ(IIa) | —CH₃ | —H | —OC₃H₇ |
| CAR(IIa) | —CH₃ | —H | —CHF₂ |
| CAS(IIa) | —CH₃ | —H | —CF₃ |
| CAT(IIa) | —CH₃ | —H | —CHCl₂ |
| CAU(IIa) | —CH₃ | —H | —CCl₃ |
| CAV(IIa) | —CH₃ | —H | —F |
| CAW(IIa) | —CH₃ | —H | —Cl |
| CAX(IIa) | —CH₃ | —H | —Br |
| CAY(IIa) | —CH₃ | —H | —I |
| CAZ(IIa) or (IIb) | —CH₃ | —OH | —H |
| CBA(IIa) or (IIb) | —CH₃ | —OH | —CH₃ |
| CBB(IIa) or (IIb) | —CH₃ | —OH | -n-propyl |
| CBC(IIa) or (IIb) | —CH₃ | —OH | -n-butyl |
| CBD(IIa) or (IIb) | —CH₃ | —OH | -t-butyl |
| CBE(IIa) or (IIb) | —CH₃ | —OH | -iso-butyl |
| CBF(IIa) or (IIb) | —CH₃ | —OH | —OCH₃ |
| CBG(IIa) or (IIb) | —CH₃ | —OH | —OC₂H₅ |
| CBH(IIa) or (IIb) | —CH₃ | —OH | —OC₃H₇ |
| CBI(IIa) or (IIb) | —CH₃ | —OH | —CHF₂ |
| CBJ(IIa) or (IIb) | —CH₃ | —OH | —CF₃ |
| CBK(IIa) or (IIb) | —CH₃ | —OH | —CHCl₂ |
| CBL(IIa) or (IIb) | —CH₃ | —OH | —CCl₃ |
| CBM(IIa) or (IIb) | —CH₃ | —OH | —F |
| CBN(IIa) or (IIb) | —CH₃ | —OH | —Cl |
| CBO(IIa) or (IIb) | —CH₃ | —OH | —Br |
| CBP(IIa) or (IIb) | —CH₃ | —OH | —I |
| CBQ(IIa) or (IIb) | —CH₃ | —F | —H |
| CBR(IIa) or (IIb) | —CH₃ | —F | —CH₃ |
| CBS(IIa) or (IIb) | —CH₃ | —F | -n-propyl |
| CBT(IIa) or (IIb) | —CH₃ | —F | -n-butyl |
| CBU(IIa) or (IIb) | —CH₃ | —F | -t-butyl |
| CBV(IIa) or (IIb) | —CH₃ | —F | -iso-butyl |
| CBW(IIa) or (IIb) | —CH₃ | —F | —OCH₃ |
| CBX(IIa) or (IIb) | —CH₃ | —F | —OC₂H₅ |
| CBY(IIa) or (IIb) | —CH₃ | —F | —OC₃H₇ |
| CBZ(IIa) or (IIb) | —CH₃ | —F | —CHF₂ |
| CCA(IIa) or (IIb) | —CH₃ | —F | —CF₃ |
| CCB(IIa) or (IIb) | —CH₃ | —F | —CHCl₂ |
| CCC(IIa) or (IIb) | —CH₃ | —F | —CCl₃ |
| CCD(IIa) or (IIb) | —CH₃ | —F | —F |
| CCE(IIa) or (IIb) | —CH₃ | —F | —Cl |
| CCF(IIa) or (IIb) | —CH₃ | —F | —Br |
| CCG(IIa) or (IIb) | —CH₃ | —F | —I |
| CCH(IIa) or (IIb) | —CH₃ | —Cl | —H |
| CCI(IIa) or (IIb) | —CH₃ | —Cl | —CH₃ |
| CCJ(IIa) or (IIb) | —CU3 | —Cl | -n-propyl |
| CCK(IIa) or (IIb) | —CH₃ | —Cl | -n-butyl |
| CCL(IIa) or (IIb) | —CU3 | —Cl | -t-butyl |
| CCM(IIa) or (IIb) | —CU3 | —Cl | -iso-butyl |
| CCN(IIa) or (IIb) | —CH₃ | —Cl | —OCH₃ |
| CCO(IIa) or (IIb) | —CH₃ | —Cl | —OC₂H₅ |
| CCP(IIa) or (IIb) | —CH₃ | —Cl | —OC₃H₇ |
| CCQ(IIa) or (IIb) | —CH₃ | —Cl | —CHF₂ |
| CCR(IIa) or (IIb) | —CH₃ | —Cl | —CF₃ |
| CCS(IIa) or (IIb) | —CH₃ | —Cl | —CHCl₂ |
| CCT(IIa) or (IIb) | —CH₃ | —Cl | —CCl₃ |
| CCU(IIa) or (IIb) | —CH₃ | —Cl | —F |
| CCV(IIa) or (IIb) | —CH₃ | —Cl | —Cl |
| CCW(IIa) or (IIb) | —CH₃ | —Cl | —Br |
| CCX(IIa) or (IIb) | —CH₃ | —Cl | —I |
| CCY(IIa) or (IIb) | —CH₃ | —Br | —H |
| CCZ(IIa) or (IIb) | —CH₃ | —Br | —CH₃ |
| CDA(IIa) or (IIb) | —CH₃ | —Br | -n-propyl |
| CDB(IIa) or (IIb) | —CH₃ | —Br | -n-butyl |
| CDC(IIa) or (IIb) | —CH₃ | —Br | -t-butyl |
| CDD(IIa) or (IIb) | —CH₃ | —Br | -iso-butyl |
| CDE(IIa) or (IIb) | —CH₃ | —Br | —OCH₃ |
| CDF(IIa) or (IIb) | —CH₃ | —Br | —OC₂H₅ |
| CDG(IIa) or (IIb) | —CH₃ | —Br | —OC₃H₇ |
| CDH(IIa) or (IIb) | —CH₃ | —Br | —CHF₂ |
| CDI(IIa) or (IIb) | —CH₃ | —Br | —CF₃ |
| CDI(IIa) or (IIb) | —CH₃ | —Br | —CHCl₂ |
| CDK(IIa) or (IIb) | —CH₃ | —Br | —CCl₃ |
| CDL(IIa) or (IIb) | —CH₃ | —Br | —F |
| CDM(IIa) or (IIb) | —CH₃ | —Br | —Cl |
| CDN(IIa) or (IIb) | —CH₃ | —Br | —Br |
| CDO(IIa) or (IIb) | —CH₃ | —Br | —I |
| CDP(IIa) or (IIb) | —CH₃ | —I | —H |
| CDQ(IIa) or (IIb) | —CH₃ | —I | —CH₃ |
| CDR(IIa) or (IIb) | —CH₃ | —I | -n-propyl |
| CDS(IIa) or (IIb) | —CH₃ | —I | -n-butyl |
| CDT(IIa) or (IIb) | —CH₃ | —I | -t-butyl |
| CDU(IIa) or (IIb) | —CH₃ | —I | -iso-butyl |
| CDV(IIa) or (IIb) | —CH₃ | —I | —OCH₃ |
| CDW(IIa) or (IIb) | —CH₃ | —I | —OC₂H₅ |
| CDX(IIa) or (IIb) | —CH₃ | —I | —OC₃H₇ |
| CDY(IIa) or (IIb) | —CH₃ | —I | —CHF₂ |
| CDZ(IIa) or (IIb) | —CH₃ | —I | —CF₃ |
| CEA(IIa) or (IIb) | —CH₃ | —I | —CHCl₂ |
| CEB(IIa) or (IIb) | —CH₃ | —I | —CCl₃ |
| CEC(IIa) or (IIb) | —CH₃ | —I | —F |
| CED(IIa) or (IIb) | —CH₃ | —I | —Cl |
| CEE(IIa) or (IIb) | —CH₃ | —I | —Br |
| CEF(IIa) or (IIb) | —CH₃ | —I | —I |
| CEG(IIa) or (IIb) | —CH₃ | —NO₂ | —H |
| CEH(IIa) or (IIb) | —CH₃ | —NO₂ | —CH₃ |
| CEI(IIa) or (IIb) | —CH₃ | —NO₂ | -n-propyl |
| CEJ(IIa) or (IIb) | —CH₃ | —NO₂ | -n-butyl |
| CEK(IIa) or (IIb) | —CH₃ | —NO₂ | -t-butyl |
| CEL(IIa) or (IIb) | —CH₃ | —NO₂ | -iso-butyl |
| CEM(IIa) or (IIb) | —CH₃ | —NO₂ | —OCH₃ |
| CEN(IIa) or (IIb) | —CH₃ | —NO₂ | —OC₂H₅ |
| CEO(IIa) or (IIb) | —CH₃ | —NO₂ | —OC₃H₇ |
| CEP(IIa) or (IIb) | —CH₃ | —NO₂ | —CHF₂ |
| CEQ(IIa) or (IIb) | —CH₃ | —NO₂ | —CF₃ |
| CER(IIa) or (IIb) | —CH₃ | —NO₂ | —CHCl₂ |
| CES(IIa) or (IIb) | —CH₃ | —NO₂ | —CCl₃ |
| CET(IIa) or (IIb) | —CH₃ | —NO₂ | —F |
| CEU(IIa) or (IIb) | —CH₃ | —NO₂ | —Cl |
| CEV(IIa) or (IIb) | —CH₃ | —NO₂ | —Br |
| CEW(IIa) or (IIb) | —CH₃ | —NO₂ | —I |
| CEX(IIa) or (IIb) | —CH₃ | —CN | —H |
| CEY(IIa) or (IIb) | —CH₃ | —CN | —CH₃ |
| CEZ(IIa) or (IIb) | —CH₃ | —CN | -n-propyl |
| CFA(IIa) or (IIb) | —CH₃ | —CN | -n-butyl |
| CFB(IIa) or (IIb) | —CH₃ | —CN | -t-butyl |
| CFC(IIa) or (IIb) | —CH₃ | —CN | -iso-butyl |
| CFD(IIa) or (IIb) | —CH₃ | —CN | —OCH₃ |
| CFE(IIa) or (IIb) | —CH₃ | —CN | —OC₂H₅ |
| CFF(IIa) or (IIb) | —CH₃ | —CN | —OC₃H₇ |
| CFG(IIa) or (IIb) | —CH₃ | —CN | —CHF₂ |
| CFH(IIa) or (IIb) | —CH₃ | —CN | —CF₃ |
| CFI(IIa) or (IIb) | —CH₃ | —CN | —CHCl₂ |
| CFJ(IIa) or (IIb) | —CH₃ | —CN | —CCl₃ |
| CFK(IIa) or (IIb) | —CH₃ | —CN | —F |
| CFL(IIa) or (IIb) | —CH₃ | —CN | —Cl |
| CFM(IIa) or (IIb) | —CH₃ | —CN | —Br |
| CFN(IIa) or (IIb) | —CH₃ | —CN | —I |
| CFO(IIa) or (IIb) | —CH₃ | —NH₂ | —H |
| CFP(IIa) or (IIb) | —CH₃ | —NH2 | —CH₃ |
| CFQ(IIa) or (IIb) | —CH₃ | —NH₂ | -n-propyl |
| CFR(IIa) or (IIb) | —CH₃ | —NH₂ | -n-butyl |
| CFS(IIa) or (IIb) | —CH₃ | —NH₂ | -t-butyl |
| CFT(IIa) or (IIb) | —CH₃ | —NH₂ | -iso-butyl |
| CFU(IIa) or (IIb) | —CH₃ | —NH₂ | —OCH₃ |
| CFV(IIa) or (IIb) | —CH₃ | —NH₂ | —OC₂H₅ |
| CFW(IIa) or (IIb) | —CH₃ | —NH₂ | —OC₃H₇ |
| CFX(IIa) or (IIb) | —CH₃ | —NH₂ | —CHF₂ |
| CFY(IIa) or (IIb) | —CH₃ | —NH₂ | —CF₃ |
| CFZ(IIa) or (IIb) | —CH₃ | —NH₂ | —CHCl₂ |
| CGA(IIa) or (IIb) | —CH₃ | —NH₂ | —CCl₃ |
| CGB(IIa) or (IIb) | —CH₃ | —NH₂ | —F |
| CGC(IIa) or (IIb) | —CH₃ | —NH₂ | —Cl |
| CGD(IIa) or (IIb) | —CH₃ | —NH₂ | —Br |
| CGE(IIa) or (IIb) | —CH₃ | —NH₂ | —I |
| CGF(IIa) or (IIb) | —CH₃ | —CH₃ | —H |
| CGG(IIa) or (IIb) | —CH₃ | —CH₃ | —CH₃ |
| CGH(IIa) or (IIb) | —CH₃ | —CH₃ | -n-propyl |
| CGI(IIa) or (IIb) | —CH₃ | —CH₃ | -n-butyl |
| CGJ(IIa) or (IIb) | —CH₃ | —CH₃ | -t-butyl |
| CGK(IIa) or (IIb) | —CH₃ | —CH₃ | -iso-butyl |
| CGL(IIa) or (IIb) | —CH₃ | —CH₃ | —OCH₃ |
| CGM(IIa) or (IIb) | —CH₃ | —CH₃ | —OC₂H₅ |
| CGN(IIa) or (IIb) | —CH₃ | —CH₃ | —OC₃H₇ |
| CGO(IIa) or (IIb) | —CH₃ | —CH₃ | —CHF₂ |
| CGP(IIa) or (IIb) | —CH₃ | —CH₃ | —CF₃ |
| CGQ(IIa) or (IIb) | —CH₃ | —CH₃ | —CHCl₂ |
| CGR(IIa) or (IIb) | —CH₃ | —CH₃ | —CCl₃ |
| CGS(IIa) or (IIb) | —CH₃ | —CH₃ | —F |
| CGT(IIa) or (IIb) | —CH₃ | —CH₃ | —Cl |
| CGU(IIa) or (IIb) | —CH₃ | —CH₃ | —Br |
| CGV(IIa) or (IIb) | —CH₃ | —CH₃ | —I |

TABLE 2

| | |
|---|---|
| | (IIIa) |
| 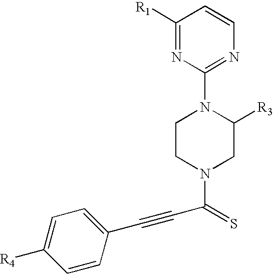 | |
| | (IIIb) |
| 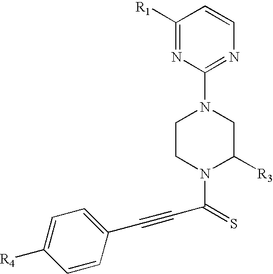 | |

and pharmaceutically acceptable salts thereof, where:

| Compound | R₁ | R₃ | R₄ |
|---|---|---|---|
| CGW(IIIa) | —H | —H | —H |
| CGX(IIIa) | —H | —H | —CH₃ |
| CGY(IIIa) | —H | —H | -n-propyl |
| CGZ(IIIa) | —H | —H | -n-butyl |
| CHA(IIIa) | —H | —H | -t-butyl |
| CHB(IIIa) | —H | —H | -iso-butyl |
| CHC(IIIa) | —H | —H | —OCH₃ |
| CHD(IIIa) | —H | —H | —OC₂H₅ |
| CHE(IIIa) | —H | —H | —OC₃H₇ |
| CHF(IIIa) | —H | —H | —CHF₂ |
| CHG(IIIa) | —H | —H | —CF₃ |
| CHH(IIIa) | —H | —H | —CHCl₂ |
| CHI(IIIa) | —H | —H | —CCl₃ |
| CHJ(IIIa) | —H | —H | —F |
| CHK(IIIa) | —H | —H | —Cl |
| CHL(IIIa) | —H | —H | —Br |
| CHM(IIIa) | —H | —H | —I |
| CHN(IIIa) or (IIIb) | —H | —OH | —H |
| CHO(IIIa) or (IIIb) | —H | —OH | —CH₃ |
| CHP(IIIa) or (IIIb) | —H | —OH | -n-propyl |
| CHQ(IIIa) or (IIIb) | —H | —OH | -n-butyl |
| CHR(IIIa) or (IIIb) | —H | —OH | -t-butyl |
| CHS(IIIa) or (IIIb) | —H | —OH | -iso-butyl |
| CHT(IIIa) or (IIIb) | —H | —OH | —OCH₃ |
| CHU(IIIa) or (IIIb) | —H | —OH | —OC₂H₅ |
| CHV(IIIa) or (IIIb) | —H | —OH | —OC₃H₇ |
| CHW(IIIa) or (IIIb) | —H | —OH | —CHF₂ |
| CWX(IIIa) or (IIIb) | —H | —OH | —CF₃ |
| CHY(IIIa) or (IIIb) | —H | —OH | —CHCl₂ |
| CHZ(IIIa) or (IIIb) | —H | —OH | —CCl₃ |
| CIA(IIIa) or (IIIb) | —H | —OH | —F |
| CIB(IIIa) or (IIIb) | —H | —OH | —Cl |
| CIC(IIIa) or (IIIb) | —H | —OH | —Br |
| CID(IIIa) or (IIIb) | —H | —OH | —I |
| CIE(IIIa) or (IIIb) | —H | —F | —H |
| CIF(IIIa) or (IIIb) | —H | —F | —CH₃ |
| CIG(IIIa) or (IIIb) | —H | —F | -n-propyl |
| CIH(IIIa) or (IIIb) | —H | —F | -n-butyl |
| CII(IIIa) or (IIIb) | —H | —F | -t-butyl |
| CIJ(IIIa) or (IIIb) | —H | —F | -iso-butyl |
| CIK(IIIa) or (IIIb) | —H | —F | —OCH₃ |
| CIL(IIIa) or (IIIb) | —H | —F | —OC₂H₅ |
| CIM(IIIa) or (IIIb) | —H | —F | —OC₃H₇ |
| CIN(IIIa) or (IIIb) | —H | —F | —CHF₂ |
| CIO(IIIa) or (IIIb) | —H | —F | —CF₃ |
| CIP(IIIa) or (IIIb) | —H | —F | —CHCl₂ |
| CIQ(IIIa) or (IIIb) | —H | —F | —CCl₃ |
| CIR(IIIa) or (IIIb) | —H | —F | —F |
| CIS(IIIa) or (IIIb) | —H | —F | —Cl |
| CIT(IIIa) or (IIIb) | —H | —F | —Br |
| CIU(IIIa) or (IIIb) | —H | —F | —I |
| CIV(IIIa) or (IIIb) | —H | —Cl | —H |
| CIW(IIIa) or (IIIb) | —H | —Cl | —CH₃ |
| CIX(IIIa) or (IIIb) | —H | —Cl | -n-propyl |
| CIY(IIIa) or (IIIb) | —H | —Cl | -n-butyl |
| CIZ(IIIa) or (IIIb) | —H | —Cl | -t-butyl |
| CJA(IIIa) or (IIIb) | —H | —Cl | -iso-butyl |
| CJB(IIIa) or (IIIb) | —H | —Cl | —OCH₃ |
| CJC(IIIa) or (IIIb) | —H | —Cl | —OC₂H₅ |
| CJD(IIIa) or (IIIb) | —H | —Cl | —OC₃H₇ |
| CJE(IIIa) or (IIIb) | —H | —Cl | —CHF₂ |
| CJF(IIIa) or (IIIb) | —H | —Cl | —CF₃ |
| CJG(IIIa) or (IIIb) | —H | —Cl | —CHCl₂ |
| CJH(IIIa) or (IIIb) | —H | —Cl | —CCl₃ |
| CJI(IIIa) or (IIIb) | —H | —Cl | —F |
| CJJ(IIIa) or (IIIb) | —H | —Cl | —Cl |
| CJK(IIIa) or (IIIb) | —H | —Cl | —Br |
| CJL(IIIa) or (IIIb) | —H | —Cl | —I |
| CJM(IIIa) or (IIIb) | —H | —Br | —H |
| CJN(IIIa) or (IIIb) | —H | —Br | —CH₃ |
| CJO(IIIa) or (IIIb) | —H | —Br | -n-propyl |
| CJP(IIIa) or (IIIb) | —H | —Br | -n-butyl |
| CJQ(IIIa) or (IIIb) | —H | —Br | -t-butyl |
| CJR(IIIa) or (IIIb) | —H | —Br | -iso-butyl |
| CJS(IIIa) or (IIIb) | —H | —Br | —OCH₃ |
| CJT(IIIa) or (IIIb) | —H | —Br | —OC₂H₅ |
| CJU(IIIa) or (IIIb) | —H | —Br | —OC₃H₇ |
| CJV(IIIa) or (IIIb) | —H | —Br | —CHF₂ |
| CJW(IIIa) or (IIIb) | —H | —Br | —CF₃ |
| CJX(IIIa) or (IIIb) | —H | —Br | —CHCl₂ |
| CJY(IIIa) or (IIIb) | —H | —Br | —CCl₃ |
| CJZ(IIIa) or (IIIb) | —H | —Br | —F |
| CKA(IIIa) or (IIIb) | —H | —Br | —Cl |
| CKB(IIIa) or (IIIb) | —H | —Br | —Br |
| CKC(IIIa) or (IIIb) | —H | —Br | —I |
| CKD(IIIa) or (IIIb) | —H | —I | —H |
| CKE(IIIa) or (IIIb) | —H | —J | —CH₃ |
| CKF(IIIa) or (IIIb) | —H | —I | -n-propyl |
| CKG(IIIa) or (IIIb) | —H | —I | -n-butyl |
| CKH(IIIa) or (IIIb) | —H | —I | -t-butyl |
| CKI(IIIa) or (IIIb) | —H | —I | -iso-butyl |
| CKJ(IIIa) or (IIIb) | —H | —I | —OCH₃ |
| CKK(IIIa) or (IIIb) | —H | —I | —OC₂H₅ |
| CKL(IIIa) or (IIIb) | —H | —I | —OC₃H₇ |
| CKM(IIIa) or (IIIb) | —H | —I | —CHF₂ |
| CKN(IIIa) or (IIIb) | —H | —I | —CF₃ |
| CKO(IIIa) or (IIIb) | —H | —I | —CHCl₂ |
| CKP(IIIa) or (IIIb) | —H | —I | —CCl₃ |
| CKQ(IIIa) or (IIIb) | —H | —I | —F |
| CKR(IIIa) or (IIIb) | —H | —I | —C |
| CKS(IIIa) or (IIIb) | —H | —I | —Br |
| CKT(IIIa) or (IIIb) | —H | —I | —I |
| CKU(IIIa) or (IIIb) | —H | —NO₂ | —H |
| CKV(IIIa) or (IIIb) | —H | —NO₂ | —CH₃ |
| CKW(IIIa) or (IIIb) | —H | —NO₂ | -n-propyl |
| CKX(IIIa) or (IIIb) | —H | —NO₂ | -n-butyl |
| CKY(IIIa) or (IIIb) | —H | —NO₂ | -t-butyl |
| CKZ(IIIa) or (IIIb) | —H | —NO₂ | -iso-butyl |
| CLA(IIIa) or (IIIb) | —H | —NO₂ | —OCH₃ |
| CLB(IIIa) or (IIIb) | —H | —NO₂ | —OC₂H₅ |
| CLC(IIIa) or (IIIb) | —H | —NO₂ | —OC₃H₇ |
| CLD(IIIa) or (IIIb) | —H | —NO₂ | —CHF₂ |
| CLE(IIIa) or (IIIb) | —H | —NO₂ | —CF₃ |
| CLF(IIIa) or (IIIb) | —H | —NO₂ | —CHCl₂ |
| CLG(IIIa) or (IIIb) | —H | —NO₂ | —CCl₃ |
| CLH(IIIa) or (IIIb) | —H | —NO₂ | —F |
| CLI(IIIa) or (IIIb) | —H | —NO₂ | —Cl |
| CLJ(IIIa) or (IIIb) | —H | —NO₂ | —Br |
| CLK(IIIa) or (IIIb) | —H | —NO₂ | —I |
| CLL(IIIa) or (IIIb) | —H | —CN | —H |
| CLM(IIIa) or (IIIb) | —H | —CN | —CH₃ |
| CLN(IIIa) or (IIIb) | —H | —CN | -n-propyl |
| CLO(IIIa) or (IIIb) | —H | —CN | -n-butyl |
| CLP(IIIa) or (IIIb) | —H | —CN | -t-butyl |
| CLQ(IIIa) or (IIIb) | —H | —CN | -iso-butyl |
| CLR(IIIa) or (IIIb) | —H | —CN | —OCH₃ |
| CLS(IIIa) or (IIIb) | —H | —CN | —OC₂H₅ |
| CLT(IIIa) or (IIIb) | —H | —CN | —OC₃H₇ |
| CLU(IIIa) or (IIIb) | —H | —CN | —CHF₂ |
| CLV(IIIa) or (IIIb) | —H | —CN | —CF₃ |
| CLW(IIIa) or (IIIb) | —H | —CN | —CHCl₂ |
| CLX(IIIa) or (IIIb) | —H | —CN | —CCl₃ |
| CLY(IIIa) or (IIIb) | —H | —CN | —F |
| CLZ(IIIa) or (IIIb) | —H | —CN | —Cl |
| CMA(IIIa) or (IIIb) | —H | —CN | —Br |
| CMB(IIIa) or (IIIb) | —H | —CN | —I |
| CMC(IIIa) or (IIIb) | —H | —NH₂ | —H |
| CMD(IIIa) or (IIIb) | —H | —NH₂ | —CH₃ |
| CME(IIIa) or (IIIb) | —H | —NH₂ | -n-propyl |
| CMF(IIIa) or (IIIb) | —H | —NH₂ | -n-butyl |
| CMG(IIIa) or (IIIb) | —H | —NH₂ | -t-butyl |
| CMH(IIIa) or (IIIb) | —H | —NH₂ | -iso-butyl |
| CMI(IIIa) or (IIIb) | —H | —NH₂ | —OCH₃ |
| CMJ(IIIa) or (IIIb) | —H | —NH₂ | —OC₂H₅ |
| CMK(IIla) or (IIIb) | —H | —NH₂ | —OC₃H₇ |
| CML(IIIa) or (IIIb) | —H | —NH₂ | —CHF₂ |
| CMM(IIIa) or (IIIb) | —H | —NH₂ | —CF₃ |
| CMN(IIIa) or (IIIb) | —H | —NH₂ | —CHCl₂ |
| CMO(IIIa) or (IIIb) | —H | —NH₂ | CCl₃ |
| CMP(IIIa) or (IIIb) | —H | —NH₂ | —F |
| CMQ(IIIa) or (IIIb) | —H | —NH₂ | —Cl |
| CMR(IIIa) or (IIIb) | —H | —NH₂ | —Br |
| CMS(IIIa) or (IIIb) | —H | —NH₂ | —I |
| CMT(IIIa) or (IIIb) | —H | —CH₃ | —H |
| CMU(IIIa) or (IIIb) | —H | —CH₃ | —CH₃ |
| CMV(IIIa) or (IIIb) | —H | —CH₃ | -n-propyl |
| CMW(IIIa) or (IIIb) | —H | —CH₃ | -n-butyl |
| CMX(IIIa) or (IIIb) | —H | —CH₃ | -t-butyl |
| CMY(IIIa) or (IIIb) | —H | —CH₃ | -iso-butyl |
| CMZ(IIIa) or (IIIb) | —H | —CH₃ | —OCH₃ |
| CNA(IIIa) or (IIIb) | —H | —CH₃ | —OC₂H₅ |
| CNB(IIIa) or (IIIb) | —H | —CH₃ | —OC₃H₇ |
| CNC(IIIa) or (IIIb) | —H | —CH₃ | —CHF₂ |
| CND(IIIa) or (IIIb) | —H | —CH₃ | —CF₃ |
| CNE(IIIa) or (IIIb) | —H | —CH₃ | —CHCl₂ |
| CNF(IIIa) or (IIIb) | —H | —CH₃ | —CCl₃ |
| CNG(IIIa) or (IIIb) | —H | —CH₃ | —F |
| CNH(IIIa) or (IIIb) | —H | —CH₃ | —Cl |
| CNI(IIIa) or (IIIb) | —H | —CH₃ | —Br |
| CNJ(IIIa) or (IIIb) | —H | —CH₃ | —1 |
| CNK(IIIa) | —OH | —H | —H |
| CNL(IIIa) | —OH | —H | —CH₃ |
| CNM(IIIa) | —OH | —H | -n-propyl |
| CNN(IIIa) | —OH | —H | -n-butyl |
| CNO(IIIa) | —OH | —H | -t-butyl |
| CNP(IIIa) | —OH | —H | -iso-butyl |
| CNQ(IIIa) | —OH | —H | —OCH₃ |
| CNR(IlIa) | —OH | —H | —OC₂H₅ |
| CNS(IIIa) | —OH | —H | —OC₃H₇ |
| CNT(IIIa) | —OH | —H | —CHF₂ |
| CNU(IIIa) | —OH | —H | —CF₃ |
| CNV(IIIa) | —OH | —H | —CHCl₂ |
| CNW(IIIa) | —OH | —H | —CCl₃ |
| CNX(IIIa) | —OH | —H | —F |
| CNY(IIIa) | —OH | —H | —Cl |
| CNZ(IIIa) | —OH | —H | —Br |
| COA(IIIa) | —OH | —H | —I |
| COB(IIIa) or (IIIb) | —OH | —OH | —H |
| COC(IIIa) or (IIIb) | —OH | —OH | —CH₃ |
| COD(IIIa) or (IIIb) | —OH | —OH | -n-propyl |
| COE(IIIa) or (IIIb) | —OH | —OH | -n-butyl |
| COF(IIIa) or (IIIb) | —OH | —OH | -t-butyl |
| COG(IIIa) or (IIIb) | —OH | —OH | -iso-butyl |
| COH(IIIa) or (IIIb) | —OH | —OH | —OCH₃ |
| COI(IIIa) or (IIIb) | —OH | —OH | —OC₂H₅ |
| COJ(IIIa) or (IIIb) | —OH | —OH | —OC₃H₇ |
| COK(IIIa) or (IIIb) | —OH | —OH | —CHF₂ |
| COL(IIIa) or (IIIb) | —OH | —OH | —CF₃ |
| COM(IIIa) or (IIIb) | —OH | —OH | —CHCl₂ |
| CON(IIIa) or (IIIb) | —OH | —OH | —CCl₃ |
| COO(IIIa) or (IIIb) | —OH | —OH | —F |
| COP(IIIa) or (IIIb) | —OH | —OH | —Cl |
| COQ(IIIa) or (IIIb) | —OH | —OH | —Br |
| COR(IIIa) or (IIIb) | —OH | —OH | —I |
| COS(IIIa) or (IIIb) | —OH | —F | —H |
| COT(IIIa) or (IIIb) | —OH | —F | —CH₃ |
| COU(IIIa) or (IIIb) | —OH | —F | -n-propyl |
| COV(IIIa) or (IIIb) | —OH | —F | -n-butyl |
| COW(IIIa) or (IIIb) | —OH | —F | -t-butyl |
| COX(IIIa) or (IIIb) | —OH | —F | -iso-butyl |
| COY(IIIa) or (IIIb) | —OH | —F | —OCH₃ |
| COZ(IIIa) or (IIIb) | —OH | —F | —OC₂H₅ |
| CPA(IIIa) or (IIIb) | —OH | —F | —OC₃H₇ |
| CPB(IIIa) or (IIIb) | —OH | —F | —CHF₂ |
| CPC(IIIa) or (IIIb) | —OH | —F | —CF₃ |
| CPD(IIIa) or (IIIb) | —OH | —F | —CHCl₂ |
| CPE(IIIa) or (IIIb) | —OH | —F | —CCl₃ |
| CPF(IIIa) or (IIIb) | —OH | —F | —F |
| CPG(IIIa) or (IIIb) | —OH | —F | —Cl |
| CPH(IIIa) or (IIIb) | —OH | —F | —Br |
| CPI(IIIa) or (IIIb) | —OH | —F | —I |
| CPJ(IIIa) or (IIIb) | —OH | —Cl | —H |
| CPK(IIIa) or (IIIb) | —OH | —Cl | —CH₃ |
| CPL(IIIa) or (IIIb) | —OH | —Cl | -n-propyl |
| CPM(IIIa) or (IIIb) | —OH | —Cl | -n-butyl |
| CPN(IIIa) or (IIIb) | —OH | —Cl | -t-butyl |
| CPO(IIIa) or (IIIb) | —OH | —Cl | -iso-butyl |
| CPP(IIIa) or (IIIb) | —OH | —Cl | —OCH₃ |
| CPQ(IIIa) or (IIIb) | —OH | —Cl | —OC₂H₅ |
| CPR(IIIa) or (IIIb) | —OH | —Cl | —OC₃H₇ |
| CPS(IIIa) or (IIIb) | —OH | —Cl | —CHF₂ |
| CPT(IIIa) or (IIIb) | —OH | —Cl | —CF₃ |
| CPU(IIIa) or (IIIb) | —OH | —Cl | —CHCl₂ |
| CPV(IIIa) or (IIIb) | —OH | —Cl | —CCl₃ |
| CPW(IIIa) or (IIIb) | —OH | —Cl | —F |
| CPX(IIIa) or (IIIb) | —OH | —Cl | —Cl |
| CPY(IIIa) or (IIIb) | —OH | —Cl | —Br |
| CPZ(IIIa) or (IIIb) | —OH | —Cl | —I |
| CQA(IIIa) or (IIIb) | —OH | —Br | —H |
| CQB(IIIa) or (IIIb) | —OH | —Br | —CH₃ |
| CQC(IIIa) or (IIIb) | —OH | —Br | -n-propyl |
| CQD(IIIa) or (IIIb) | —OH | —Br | -n-butyl |
| CQE(IIIa) or (IIIb) | —OH | —Br | -t-butyl |
| CQF(IIIa) or (IIIb) | —OH | —Br | -iso-butyl |
| CQG(IIIa) or (IIIb) | —OH | —Br | —OCH₃ |
| CQH(IIla) or (IIIb) | —OH | —Br | —OC₂H₅ |
| CQI(IIIa) or (IIIb) | —OH | —Br | —OC₃H₇ |
| CQJ(IIIa) or (IIIb) | —OH | —Br | —CHF₂ |
| CQK(IIIa) or (IIIb) | —OH | —Br | —CF₃ |
| CQL(IIIa) or (IIIb) | —OH | —Br | —CHCl₂ |
| CQM(IIIa) or (IIIb) | —OH | —Br | —CCl₃ |
| CQN(IIIa) or (IIIb) | —OH | —Br | —F |
| CQO(IIIa) or (IIIb) | —OH | —Br | —Cl |
| CQP(IIIa) or (IIIb) | —OH | —Br | —Br |
| CQQ(IIIa) or (IIIb) | —OH | —Br | —I |
| CQR(IIIa) or (IIIb) | —OH | —I | —H |
| CQS(IIIa) or (IIIb) | —OH | —I | —CH₃ |
| CQT(IIIa) or (IIIb) | —OH | —I | -n-propyl |
| CQU(IIIa) or (IIIb) | —OH | —I | -n-butyl |
| CQV(IIIa) or (IIIb) | —OH | —I | -t-butyl |
| CQW(IIIa) or (IIIb) | —OH | —I | -iso-butyl |
| CQX(IIIa) or (IIIb) | —OH | —I | —OCH₃ |
| CQY(IIIa) or (IIIb) | —OH | —L | —OC₂H₅ |
| CQZ(IIIa) or (IIIb) | —OH | —I | —OC₃H₇ |
| CRA(IIIa) or (IIIb) | —OH | —I | —CHF₂ |
| CRB(IIIa) or (IIIb) | —OH | —I | —CF₃ |
| CRC(IIIa) or (IIIb) | —OH | —I | —CHCl₂ |
| CRD(IIIa) or (IIIb) | —OH | —I | —CCl₃ |
| CRE(IIIa) or (IIIb) | —OH | —I | —F |
| CRF(IIIa) or (IIIb) | —OH | —I | —Cl |
| CRG(IIIa) or (IIIb) | —OH | —I | —Br |
| CRH(IIIa) or (IIIb) | —OH | —I | —I |
| CRI(IIIa) or (IIIb) | —OH | —NO₂ | —H |
| CRJ(IIIa) or (IIIb) | —OH | —NO₂ | —CH₃ |
| CRK(IIIa) or (IIIb) | —OH | —NO₂ | -n-propyl |
| CRL(IIIa) or (IIIb) | —OH | —NO₂ | -n-butyl |
| CRM(IIIa) or (IIIb) | —OH | —NO₂ | -t-butyl |
| CRN(IIIa) or (IIIb) | —OH | —NO₂ | -iso-butyl |
| CRO(IIIa) or (IIIb) | —OH | —NO₂ | —OCH₃ |
| CRP(IIIa) or (IIIb) | —OH | —NO₂ | —OC₂H₅ |
| CRQ(IIIa) or (IIIb) | —OH | —NO₂ | —OC₃H₇ |
| CRR(IIIa) or (IIIb) | —OH | —NO₂ | —CHF₂ |
| CRS(IIIa) or (IIIb) | —OH | —NO₂ | —CF₃ |
| CRT(IIIa) or (IIIb) | —OH | —NO₂ | —CHCl₂ |
| CRU(IIIa) or (IIIb) | —OH | —NO₂ | —CCl₃ |
| CRV(IIIa) or (IIIb) | —OH | —NO₂ | —F |
| CRW(IIIa) or (IIIb) | —OH | —NO₂ | —Cl |
| CRX(IIIa) or (IIIb) | —OH | —NO₂ | —Br |
| CRY(IIIa) or (IIIb) | —OH | —NO₂ | —I |
| CRZ(IIIa) or (IIIb) | —OH | —CN | —H |
| CSA(IIIa) or (IIIb) | —OH | —CN | —CH₃ |
| CSB(IIIa) or (IIIb) | —OH | —CN | -n-propyl |
| CSC(IIIa) or (IIIb) | —OH | —CN | -n-butyl |
| CSD(IIIa) or (IIIb) | —OH | —CN | -t-butyl |
| CSE(IIIa) or (IIIb) | —OH | —CN | -iso-butyl |
| CSF(IIIa) or (IIIb) | —OH | —CN | —OCH₃ |
| CSG(IIIa) or (IIIb) | —OH | —CN | —OC₂H₅ |
| CSH(IIIa) or (IIIb) | —OH | —CN | —OC₃H₇ |
| CSI(IIIa) or (IIIb) | —OH | —CN | —CHF₂ |
| CSJ(IIIa) or (IIIb) | —OH | —CN | —CF₃ |
| CSK(IIIa) or (IIIb) | —OH | —CHCl₂ | |
| CSL(IIIa) or (IIIb) | —OH | —CN | —CCl₃ |
| CSM(IIIa) or (IIIb) | —OH | —CN | —F |
| CSN(IIIa) or (IIIb) | —OH | —CN | —Cl |
| CSO(IIIa) or (IIIb) | —OH | —CN | —Br |
| CSP(IIIa) or (IIIb) | —OH | —CN | —I |
| CSQ(IIIa) or (IIIb) | —OH | —NH₂ | —H |
| CSR(IIIa) or (IIIb) | —OH | —NH₂ | —CH₃ |
| CSS(IIIa) or (IIIb) | —OH | —NH₂ | -n-propyl |
| CST(IIIa) or (IIIb) | —OH | -n-butyl | |
| CSU(IIIa) or (IIIb) | —OH | —NH₂ | -t-butyl |
| CSV(IIIa) or (IIIb) | —OH | —NH₂ | -iso-butyl |
| CSW(IIIa) or (IIIb) | —OH | —NH₂ | —OCH₃ |
| CSX(IIIa) or (IIIb) | —OH | —NH₂ | —OC₂H₅ |
| CSY(IIIa) or (IIIb) | —OH | —NH₂ | —OC₃H₇ |
| CSZ(IIIa) or (IIIb) | —OH | —NH₂ | —CHF₂ |
| CTA(IIIa) or (IIIb) | —OH | —NH₂ | —CF₃ |
| CTB(IIIa) or (IIIb) | —OH | —NH₂ | —CHCl₂ |
| CTC(IIIa) or (IIIb) | —OH | —NH₂ | —CCl₃ |
| CTD(IIIa) or (IIIb) | —OH | —NH₂ | —F |
| CTE(IIIa) or (IIIb) | —OH | —NH₂ | —Cl |
| CTF(IIIa) or (IIIb) | —OH | —NH₂ | —Br |
| CTG(IIIa) or (IIIb) | —OH | —NH₂ | —I |
| CTH(IIIa) or (IIIb) | —OH | —CH₃ | —H |
| CTI(IIIa) or (IIIb) | —OH | —CH₃ | —CH₃ |
| CTJ(IIIa) or (IIIb) | —OH | —CH₃ | -n-propyl |
| CTK(IIIa) or (IIIb) | —OH | —CH₃ | -n-butyl |
| CTL(IIIa) or (IIIb) | —OH | —CH₃ | -t-butyl |
| CTM(IIIa) or (IIIb) | —OH | —CH₃ | -iso-butyl |
| CTN(IIIa) or (IIIb) | —OH | —CH₃ | —OCH₃ |
| CTO(IIIa) or (IIIb) | —OH | —CH₃ | —OC₂II₅ |
| CTP(IIIa) or (IIIb) | —OH | —CH₃ | —OC₃II₇ |
| CTQ(IIIa) or (IIIb) | —OH | —CH₃ | —CHF₂ |
| CTR(IIIa) or (IIIb) | —OH | —CH₃ | —CF₃ |
| CTS(IIIa) or (IIIb) | —OH | —CH₃ | —CHCl₂ |
| CTT(IIIa) or (IIIb) | —OH | —CH₃ | —CCl₃ |
| CTU(IIIa) or (IIIb) | —OH | —CH₃ | —F |
| CTV(IIIa) or (IIIb) | —OH | —CH₃ | —Cl |
| CTW(IIIa) or (IIIb) | —OH | —CH₃ | —Br |
| CTX(IIIa) or (IIIb) | —OH | —CH₃ | —I |
| CTY(IIIa) | —F | —H | —H |
| CTZ(IIIa) | —F | —H | —CH₃ |
| CUA(IIIa) | —F | —H | -n-propyl |
| CUB(IIIa) | —F | —H | -n-butyl |
| CUC(IIIa) | —F | —H | -t-butyl |
| CUD(IIIa) | —F | —H | -iso-butyl |
| CUE(IIIa) | —F | —H | —OCH₃ |
| CUF(IIIa) | —F | —H | —OC₂H₅ |
| CUG(IIIa) | —F | —H | —OC₃H₇ |
| CUH(IIIa) | —F | —H | —CHF₂ |
| CUI(IIIa) | —F | —H | —CF₃ |
| CUJ(IIIa) | —F | —H | —CHCl₂ |
| CUK(IIIa) | —F | —H | —CCl₃ |
| CUL(IIIa) | —F | —H | —F |
| CUM(IIIa) | —F | —H | —Cl |
| CUN(IIIa) | —F | —H | —Br |
| CUO(IIIa) | —F | —H | —I |
| CUP(IIIa) or (IIIb) | —F | —OH | —H |
| CUQ(IIIa) or (IIIb) | —F | —OH | —CH₃ |
| CUR(IIIa) or (IIIb) | —F | —OH | -n-propyl |
| CUS(IIIa) or (IIIb) | —F | —OH | -n-butyl |
| CUT(IIIa) or (IIIb) | —F | —OH | -t-butyl |
| CUU(IIIa) or (IIIb) | —F | —OH | -iso-butyl |
| CUV(IIIa) or (IIIb) | —F | —OH | —OCH₃ |
| CUW(IIIa) or (IIIb) | —F | —OH | —OC₂H₅ |
| CUX(IIIa) or (IIIb) | —F | —OH | —OC₃H₇ |
| CUY(IIIa) or (IIIb) | —F | —OH | —CHF₂ |
| CUZ(IIIa) or (IIIb) | —F | —OH | —CF₃ |
| CVA(IIIa) or (IIIb) | —F | —OH | —CHCl₂ |
| CVB(IIIa) or (IIIb) | —F | —OH | —CCl₃ |
| CVC(IIIa) or (IIIb) | —F | —OH | —F |
| CVD(LIIa) or (IIIb) | —F | —OH | —Cl |
| CVE(IIIa) or (IIIb) | —F | —OH | —Br |
| CVF(IIIa) or (IIIb) | —F | —OH | —I |
| CVG(IIIa) or (IIIb) | —F | —F | —H |
| CVH(IIIa) or (IIIb) | —F | —F | —CH₃ |
| CVI(IIIa) or (IIIb) | —F | —F | -n-propyl |
| CVJ(IIIa) or (IIIb) | —F | —F | -n-butyl |
| CVK(IIIa) or (IIIb) | —F | —F | -t-butyl |
| CVL(IIIa) or (IIIb) | —F | —F | -iso-butyl |
| CVM(IIIa) or (IIIb) | —F | —F | —OCH₃ |
| CVN(IIIa) or (IIIb) | —F | —F | —OC₂H₅ |
| CVO(IIIa) or (IIIb) | —F | —F | —OC₃H₇ |
| CVP(IIIa) or (IIIb) | —F | —F | —CHF₂ |
| CVQ(IIIa) or (IIIb) | —F | —F | —CF₃ |
| CVR(IIIa) or (IIIb) | —F | —F | —CHCl₂ |
| CVS(IIIa) or (IIIb) | —F | —F | —CCl₃ |
| CVT(IIIa) or (IIIb) | —F | —F | —F |
| CVU(IIIa) or (IIIb) | —F | —F | —Cl |
| CVV(IIIa) or (IIIb) | —F | —F | —Br |
| CVW(IIIa) or (IIIb) | —F | —F | —I |
| CVX(IIIa) or (IIIb) | —F | —Cl | —H |
| CVY(IIIa) or (IIIb) | —F | —Cl | —CH₃ |
| CVZ(IIIa) or (IIIb) | —F | —Cl | -n-propyl |
| CWA(IIIa) or (IIIb) | —F | —Cl | -n-butyl |
| CWB(IIIa) or (IIIb) | —F | —Cl | -t-butyl |
| CWC(IIIa) or (IIIb) | —F | —Cl | -iso-butyl |
| CWD(IIIa) or (IIIb) | —F | —Cl | —OCH₃ |
| CWE(IIIa) or (IIIb) | —F | —Cl | —OC₂H₅ |
| CWF(IIIa) or (IIIb) | —F | —Cl | —OC₃H₇ |
| CWG(IIIa) or (IIIh) | —F | —Cl | —CHF₂ |
| CWH(IIIa) or (IIIb) | —F | —Cl | —CF₃ |
| CWI(IIIa) or (IIIb) | —F | —Cl | —CHCl₂ |
| CWJ(IIIa) or (IIIb) | —F | —Cl | —CCl₃ |
| CWK(IIIa) or (IIIb) | —F | —Cl | —F |
| CWL(IIIa) or (IIIb) | —F | —Cl | —Cl |
| CWM(IIIa) or (IIIb) | —F | —Cl | —Br |
| CWN(IIIa) or (IIIb) | —F | —Cl | —I |
| CWO(IIIa) or (IIIb) | —F | —Br | —H |
| CWP(IIIa) or (IIIb) | —F | —Br | —CH₃ |
| CWQ(IIIa) or (IIIb) | —F | —Br | -n-propyl |
| CWR(IIIa) or (IIIb) | —F | —Br | -n-butyl |
| CWS(IIIa) or (IIIb) | —F | —Br | -t-butyl |
| CWT(IIIa) or (IIIb) | —F | —Br | -iso-butyl |
| CWU(IIIa) or (IIIb) | —F | —Br | —OCH₃ |
| CWV(IIIa) or (IIIb) | —F | —Br | —OC₂H₅ |
| CWW(IIIa) or (IIIb) | —F | —Br | —OC₃H₇ |
| CWX(IIIa) or (IIIb) | —F | —Br | —CHF₂ |
| CWY(IIIa) or (IIIb) | —F | —Br | —CF₃ |
| CWZ(IIIa) or (IIIb) | —F | —Br | —CHCl₂ |
| CXA(IIIa) or (IIIb) | —F | —Br | —CCl₃ |
| CXB(IIIa) or (IIIb) | —F | —Br | —F |
| CXC(IIIa) or (IIIb) | —F | —Br | —Cl |
| CXD(IIIa) or (IIIb) | —F | —Br | —Br |
| CXE(IIIa) or (IIIb) | —F | —Br | —I |
| CXF(IIIa) or (IIIb) | —F | —I | —H |
| CXG(IIIa) or (IIIb) | —F | —I | —CH₃ |
| CXH(IIIa) or (IIIb) | —F | —I | -n-propyl |
| CXI(IIIa) or (IIIb) | —F | —I | -n-butyl |
| CXJ(IIIa) or (IIIh) | —F | —I | -t-butyl |
| CXK(IIIa) or (IIIb) | —F | —I | -iso-butyl |
| CXL(IIIa) or (IIIb) | —F | —I | —OCH₃ |
| CXM(IIIa) or (IIIb) | —F | —I | —OC₂H₅ |
| CXN(IIIa) or (IIIb) | —F | —I | —OC₃H₇ |
| CXO(IIIa) or (IIIb) | —F | —I | —CHF₂ |
| CXP(IIIa) or (IIIb) | —F | —I | —CF₃ |
| CXQ(IIIa) or (IIIb) | —F | —I | —CHCl₂ |
| CXR(IIIa) or (IIIb) | —F | —I | —CCl₃ |
| CXS(IIIa) or (IIIb) | —F | —I | —F |
| CXT(IIIa) or (IIIb) | —F | —I | —Cl |
| CXU(IIIa) or (IIIb) | —F | —I | —Br |
| CXV(IIIa) or (IIIb) | —F | —I | —I |
| CXW(IIIa) or (IIIb) | —F | —NO₂ | —H |
| CXX(IIIa) or (IIIb) | —F | —NO₂ | —CH₃ |
| CXY(IIIa) or (IIIb) | —F | —NO₂ | -n-propyl |
| CXZ(IIIa) or (IIIb) | —F | —NO₂ | -n-butyl |
| CYA(IIIa) or (IIIb) | —F | —NO₂ | -t-butyl |
| CYB(IIIa) or (IIIb) | —F | —NO₂ | -iso-butyl |
| CYC(IIIa) or (IIIb) | —F | —NO₂ | —OCH₃ |
| CYD(IIIa) or (IIIb) | —F | —NO₂ | —OC₂H₅ |
| CYE(IIIa) or (IIIb) | —F | —NO₂ | —OC₃H₇ |
| CYF(IIIa) or (IIIb) | —F | —NO₂ | —CHF₂ |
| CYG(IIIa) or (IIIb) | —F | —NO₂ | —CF₃ |
| CYH(IIIa) or (IIIb) | —F | —NO₂ | —CHCl₂ |
| CYI(IIIa) or (IIIb) | —F | —NO₂ | —CCl₃ |
| CYJ(IIIa) or (IIIb) | —F | —NO₂ | —F |
| CYK(IIIa) or (IIIb) | —F | —NO₂ | —Cl |
| CYL(IIIa) or (IIIb) | —F | —NO₂ | —Br |
| CYM(IIIa) or (IIIb) | —F | —NO₂ | —I |
| CYN(IIIa) or (IIIb) | —F | —CN | —H |
| CYO(IIIa) or (IIIb) | —F | —CN | —CH₃ |
| CYP(IIIa) or (IIIb) | —F | —CN | -n-propyl |
| CYQ(IIIa) or (IIIb) | —F | —CN | -n-butyl |
| CYR(IIIa) or (IIIb) | —F | —CN | -t-butyl |
| CYS(IIIa) or (IIIb) | —F | —CN | -iso-butyl |
| CYT(IIIa) or (IIIb) | —F | —CN | —OCH₃ |
| CYU(IIIa) or (IIIb) | —F | —CN | —OC₃H₅ |
| CYV(IIIa) or (IIIb) | —F | —CN | —OC₃H₇ |
| CYW(IIIa) or (IIIb) | —F | —CN | —CHF₃ |
| CYX(IIIa) or (IIIb) | —F | —CN | —CF₃ |
| CYY(IIIa) or (IIIb) | —F | —CN | —CHCl₂ |
| CYZ(IIIa) or (IIIb) | —F | —CN | —CCl₃ |
| CZA(IIIa) or (IIIb) | —F | —CN | —F |
| CZB(IIIa) or (IIIb) | —F | —CN | —Cl |
| CZC(IIIa) or (IIIb) | —F | —CN | —Br |
| CZD(IIIa) or (IIIb) | —F | —CN | —I |
| CZE(IIIa) or (IIIb) | —F | —NH₂ | —H |
| CZF(IIIa) or (IIIb) | —F | —NH₂ | —CH₃ |
| CZG(IIIa) or (IIIb) | —F | —NH₂ | -n-propyl |
| CZH(IIIa) or (IIIb) | —F | —NH₂ | -n-butyl |
| CZI(IIIa) or (IIIb) | —F | —NH₂ | -t-butyl |
| CZJ(IIIa) or (IIIb) | —F | —NH₂ | -iso-butyl |
| CZK(IIIa) or (IIIb) | —F | —NH₂ | —OCH₃ |
| CZL(IIIa) or (IIIb) | —F | —NH₂ | —OC₂H₅ |
| CZM(IIIa) or (IIIb) | —F | —NH₂ | —OC₃H₇ |
| CZN(IIIa) or (IIIb) | —F | —NH₂ | —CHF₂ |
| CZO(IIIa) or (IIIb) | —F | —NH₂ | —CF₃ |
| CZP(IIIa) or (IIIb) | —F | —NH₂ | —CHCl₂ |
| CZQ(IIIa) or (IIIb) | —F | —NH₂ | —CCl₃ |
| CZR(IIIa) or (IIIb) | —F | —NH₂ | —F |
| CZS(IIIa) or (IIIb) | —F | —NH₂ | —Cl |
| CZT(IIIa) or (IIIb) | —F | —NH₂ | —Br |
| CZU(IIIa) or (IIIb) | —F | —NH₂ | —I |
| CZV(IIIa) or (IIIb) | —F | —CH₃ | —H |
| CZW(IIIa) or (IIIb) | —F | —CH₃ | —CH₃ |
| CZX(IIIa) or (IIIb) | —F | —CH₃ | -n-propyl |
| CZY(IIIa) or (IIIb) | —F | —CH₃ | -n-butyl |
| CZZ(IIIa) or (IIIb) | —F | —CH₃ | -t-butyl |
| DAA(IIIa) or (IIIb) | —F | —CH₃ | -iso-butyl |
| DAB(IIIa) or (IIIb) | —F | —CH₃ | —OCH₃ |
| DAC(IIIa) or (IIIb) | —F | —CH₃ | —OC₂H₅ |
| DAD(IIIa) or (IIIb) | —F | —CH₃ | —OC₃H₇ |
| DAE(IIIa) or (IIIb) | —F | —CH₃ | —CHF₂ |
| DAF(IIIa) or (IIIb) | —F | —CH₃ | —CF₃ |
| DAG(IIIa) or (IIIb) | —F | —CH₃ | —CHCl₂ |
| DAH(IIIa) or (IIIb) | —F | —CH₃ | —CCl₃ |
| DAI(IIIa) or (IIIb) | —F | —CH₃ | —F |
| DAJ(IIIa) or (IIIb) | —F | —CH₃ | —Cl |
| DAK(IIIa) or (IIIb) | —F | —CH₃ | —Br |
| DAL(IIIa) or (IIIb) | —F | —CH₃ | —I |
| DAM(IIIa) | —Cl | —H | —H |
| DAN(IIIa) | —Cl | —H | —CH₃ |
| DAO(IIIa) | —Cl | —H | -n-propyl |
| DAP(IIIa) | —Cl | —H | -n-butyl |
| DAQ(IIIa) | —Cl | —H | -t-butyl |
| DAR(IIIa) | —Cl | —H | -iso-butyl |
| DAS(IIIa) | —Cl | —H | —OCH₃ |
| DAT(IIIa) | —Cl | —H | —OC₂H₅ |
| DAU(IIIa) | —Cl | —H | —OC₃H₇ |
| DAV(IIIa) | —Cl | —H | —CHF₂ |
| DAW(IIIa) | —Cl | —H | —CF₃ |
| DAX(IIIa) | —Cl | —H | —CHCl₂ |
| DAY(IIIa) | —Cl | —H | —CCl₃ |
| DAZ(IIIa) | —Cl | —H | —F |
| DBA(IIIa) | —Cl | —H | —Cl |
| DBB(IIIa) | —Cl | —H | —Br |
| DBC(IIIa) | —Cl | —H | —I |
| DBD(IIIa) or (IIIb) | —Cl | —OH | —H |
| DBE(IIIa) or (IIIb) | —Cl | —OH | —CH₃ |
| DBF(IIIa) or (IIIb) | —Cl | —OH | -n-propyl |
| DBG(IIIa) or (IIIb) | —Cl | —OH | -n-butyl |
| DBH(IIIa) or (IIIb) | —Cl | —OH | -t-butyl |
| DBI(IIIa) or (IIIb) | —Cl | —OH | -iso-butyl |
| DBJ(IIIa) or (IIIb) | —Cl | —OH | —OCHhd 3 |
| DBK(IIIa) or (IIIb) | —Cl | —OH | —OC₂H₅ |
| DBL(IIIa) or (IIIb) | —Cl | —OH | —OC₃H₇ |
| DBM(IIIa) or (IIIb) | —Cl | —OH | —CHF₂ |
| DBN(IIIa) or (IIIb) | —Cl | —OH | —CF₃ |
| DBO(IIIa) or (IIIb) | —Cl | —OH | —CHCl₂ |
| DBP(IIIa) or (IIIb) | —Cl | —OH | —CCl₃ |
| DBQ(IIIa) or (IIIb) | —Cl | —OH | —F |
| DBR(IIIa) or (IIIb) | —Cl | —OH | —Cl |
| DBS(IIIa) or (IIIb) | —Cl | —OH | —Br |
| DBT(IIIa) or (IIIb) | —Cl | —OH | —I |
| DBU(IIIa) or (IIIb) | —Cl | —F | —H |
| DBV(IIIa) or (IIIb) | —Cl | —F | —CH₃ |
| DBW(IIIa) or (IIIb) | —Cl | —F | -n-propyl |
| DBX(IIIa) or (IIIb) | —Cl | —F | -n-butyl |
| DBY(IIIa) or (IIIb) | —Cl | —F | -t-butyl |
| DBZ(IIIa) or (IIIb) | —Cl | —F | -iso-butyl |
| DCA(IIIa) or (IIIb) | —Cl | —F | —OCH₃ |
| DCB(IIIa) or (IIIb) | —Cl | —F | —OC₂H₅ |
| DCC(IIIa) or (IIIb) | —Cl | —F | —OC₃H₇ |
| DCD(IIIa) or (IIIb) | —Cl | —F | —CHF₂ |
| DCE(IIIa) or (IIIb) | —Cl | —F | —CF₃ |
| DCF(IIIa) or (IIIb) | —Cl | —F | —CHCl₂ |
| DCG(IIIa) or (IIIb) | —Cl | —F | —CCl₃ |
| DCH(IIIa) or (IIIb) | —Cl | —F | —F |
| DCI(IIIa) or (IIIb) | —Cl | —F | —Cl |
| DCJ(IIIa) or (IIIb) | —Cl | —F | —Br |
| DCK(IIIa) or (IIIb) | —Cl | —F | —I |
| DCL(IIIa) or (IIIb) | —Cl | —Cl | —H |
| DCM(IIIa) or (IIIb) | —Cl | —Cl | —CH₃ |
| DCN(IIIa) or (IIIb) | —Cl | —Cl | -n-propyl |
| DCO(IIIa) or (IIIb) | —Cl | —Cl | -n-butyl |
| DCP(IIIa) or (IIIb) | —Cl | —Cl | -t-butyl |
| DCQ(IIIa) or (IIIb) | —Cl | —Cl | -iso-butyl |
| DCR(IIIa) or (IIIb) | —Cl | —Cl | —OCH₃ |
| DCS(IIIa) or (IIIb) | —Cl | —Cl | —OC₂H₅ |
| DCT(IIIa) or (IIIb) | —Cl | —Cl | —OC₃H₇ |
| DCU(IIIa) or (IIIb) | —Cl | —Cl | —CHF₂ |
| DCV(IIIa) or (IIIb) | —Cl | —Cl | —CF₃ |
| DCW(IIIa) or (IIIb) | —Cl | —Cl | —CHCl₂ |
| DCX(IIIa) or (IIIb) | —Cl | —Cl | —CCl₃ |
| DCY(IIIa) or (IIIb) | —Cl | —Cl | —F |
| DCZ(IIIa) or (IIIb) | —Cl | —Cl | —Cl |
| DDA(IIIa) or (IIIb) | —Cl | —Cl | —Br |
| DDB(IIIa) or (IIIb) | —Cl | —Cl | —I |
| DDC(IIIa) or (IIIb) | —Cl | —Br | —H |
| DDD(IIIa) or (IIIb) | —Cl | —Br | —CH₃ |
| DDE(IIIa) or (IIIb) | —Cl | —Br | -n-propyl |
| DDF(IIIa) or (IIIb) | —Cl | —Br | -n-butyl |
| DDG(IIIa) or (IIIb) | —Cl | —Br | -t-butyl |
| DDH(IIIa) or (IIIb) | —Cl | —Br | -iso-butyl |
| DDI(IIIa) or (IIIb) | —Cl | —Br | —OCH₃ |
| DDJ(IIIa) or (IIIb) | —Cl | —Br | —OC₂H₅ |
| DDK(IIIa) or (IIIb) | —Cl | —Br | —OC₃H₇ |
| DDL(IIIa) or (IIIb) | —Cl | —Br | —CHF₂ |
| DDM(IIIa) or (IIIb) | —Cl | —Br | —CF₃ |
| DDN(IIIa) or (IIIb) | —Cl | —Br | —CHCl₂ |
| DDO(IIIa) or (IIIb) | —Cl | —Br | —CCl₃ |
| DDP(IIIa) or (IIIb) | —Cl | —Br | —F |
| DDQ(IIIa) or (IIIb) | —Cl | —Br | —Cl |
| DDR(IIIa) or (IIIb) | —Cl | —Br | —Br |
| DDS(IIIa) or (IIIb) | —Cl | —Br | —I |
| DDT(IIIa) or (IIIb) | —Cl | —I | —H |
| DDU(IIIa) or (IIIb) | —Cl | —I | —CH₃ |
| DDV(IIIa) or (IIIb) | —Cl | —I | -n-propyl |
| DDW(IIIa) or (IIIb) | —Cl | —I | -n-butyl |
| DDX(IIIa) or (IIIb) | —Cl | —I | -t-butyl |
| DDY(IIIa) or (IIIb) | —Cl | —I | -iso-butyl |
| DDZ(IIla) or (IIIb) | —Cl | —I | —OCH₃ |
| DEA(IIIa) or (IIIb) | —Cl | —I | —OC₂H₅ |
| DEB(IIIa) or (IIb) | —Cl | —I | —OC₃H₇ |
| DEC(IIIa) or (IIIb) | —Cl | —I | —CHF₂ |
| DED(IIIa) or (IIIb) | —Cl | —I | —CF₃ |
| DEE(IIIa) or (IIIb) | —Cl | —I | —CHCl₂ |
| DEF(IIIa) or (IIIb) | —Cl | —I | —CCl₃ |
| DEG(IIIa) or (IIIb) | —Cl | —I | —F |
| DEH(IIIa) or (IIIb) | —Cl | —I | —Cl |
| DEI(IIIa) or (IIIb) | —Cl | —I | —Br |
| DEJ(IIIa) or (IIIb) | —Cl | —I | —I |
| DEK(IIIa) or (IIIb) | —Cl | —NO₂ | —H |
| DEL(IIIa) or (IIIb) | —Cl | —NO₂ | —CH₃ |
| DEM(IIIa) or (IIIb) | —Cl | —NO₂ | -n-propyl |
| DEN(IIIa) or (IIIb) | —Cl | —NO₂ | -n-butyl |
| DEO(IIIa) or (IIb) | —Cl | —NO₂ | -t-butyl |
| DEP(IIIa) or (IIIb) | —Cl | —NO₂ | -iso-butyl |
| DEQ(IIIa) or (IIIb) | —Cl | —NO₂ | —OCH₃ |
| DER(IIIa) or (IIIb) | —Cl | —NO₂ | —OC₂H₅ |
| DES(IIIa) or (IIIb) | —Cl | —NO₂ | —OC₃H₇ |
| DET(IIIa) or (IIIb) | —Cl | —NO₂ | —CHF₂ |
| DEU(IIIa) or (IIIb) | —Cl | —NO₂ | —CF₃ |
| DEV(IIIa) or (IIIb) | —Cl | —NO₂ | —CHCl₂ |
| DEW(IIIa) or (IIIb) | —Cl | —NO₂ | —CCl₃ |
| DEX(IIIa) or (IIIb) | —Cl | —NO₂ | —F |
| DEY(IIIa) or (IIIb) | —Cl | —NO₂ | —Cl |
| DEZ(IIIa) or (IIIb) | —Cl | —NO₂ | —Br |
| DFA(IIIa) or (IIIb) | —Cl | —NO₂ | —I |
| DFB(IIIa) or (IIIb) | —Cl | —CN | —H |
| DFC(IIIa) or (IIIb) | —Cl | —CN | —CH₃ |
| DFD(IIIa) or (IIIb) | —Cl | —CN | -n-propyl |
| DFE(IIIa) or (IIIb) | —Cl | —CN | -n-butyl |
| DFF(IIIa) or (IIIb) | —Cl | —CN | -t-butyl |
| DFG(IIIa) or (IIIb) | —Cl | —CN | -iso-butyl |
| DFH(IIIa) or (IIIb) | —Cl | —CN | —OCH₃ |
| DFI(IIIa) or (IIIb) | —Cl | —CN | —OC₂H₅ |
| DFJ(IIIa) or (IIIb) | —Cl | —CN | —OC₃H₇ |
| DFK(IIIa) or (IIIb) | —Cl | —CN | —CHF₂ |
| DFL(IIIa) or (lIIb) | —Cl | —CN | —CF₃ |
| DFM(IIIa) or (IIIb) | —Cl | —CN | —CHCl₂ |
| DFN(IIIa) or (IIIb) | —Cl | —CN | —CCl₃ |
| DFO(IIIa) or (IIIb) | —Cl | —CN | —F |
| DFP(IIIa) or (IIIb) | —Cl | —CN | —Cl |
| DFQ(IIIa) or (IIIb) | —Cl | —CN | —Br |
| DFR(IIIa) or (IIIb) | —Cl | —CN | —I |
| DFS(IIIa) or (IIIb) | —Cl | —NH₂ | |
| DFT(IIIa) or (IIIb) | —Cl | —NH₂ | —CH₃ |
| DFU(IIIa) or (IIIb) | —Cl | —NH₂ | -n-propyl |
| DFV(IIIa) or (IIIb) | —Cl | —NH₂ | -n-butyl |
| DFW(IIIa) or (IIIb) | —Cl | —NH₂ | -t-butyl |
| DFX(IIIa) or (IIIb) | —Cl | —NH₂ | -iso-butyl |
| DFY(lIIIa) or (IIIb) | —Cl | —NH₂ | —OCH₃ |
| DFZ(IIIa) or (IIIb) | —Cl | —NH₂ | —OC₂H₅ |
| DGA(IIIa) or (IIIb) | —Cl | —NH₂ | —OC₃H₇ |
| DGB(IIIa) or (IIIb) | —Cl | —NH₂ | —CHF₂ |
| DGC(IIIa) or (IIIb) | —Cl | —NH₂ | —CF₃ |
| DGD(IIIa) or (IIIb) | —Cl | —NH₂ | —CHCl₂ |
| DGE(IIIa) or (IIIb) | —Cl | —NH₂ | —CCl₃ |
| DGF(IIIa) or (IIIb) | —Cl | —NH₂ | —F |
| DGG(IIIa) or (IIIb) | —Cl | —NH₂ | —Cl |
| DGH(IIIa) or (IIIb) | —Cl | —NH₂ | —Br |
| DGI(IIIa) or (IIIb) | —Cl | —NH₂ | —I |
| DGJ(IIIa) or (IIIb) | —Cl | —CH₃ | —H |
| DGK(IIIa) or (IIIb) | —Cl | —CH₃ | —CH₃ |
| DGL(IIIa) or (IIIb) | —Cl | —CH₃ | -n-propyl |
| DGM(IIIa) or (IIIb) | —Cl | —CH | -n-butyl |
| DGN(IIIa) or (IIIb) | —Cl | —CH₃ | -t-butyl |
| DGO(IIIa) or (IIIb) | —Cl | —CH₃ | -iso-butyl |
| DGP(IIIa) or (IIIb) | —Cl | —CH₃ | —OCH₃ |
| DGQ(IIIa) or (IIIb) | —Cl | —CH₃ | —OC₂H₅ |
| DGR(IIIa) or (IIIb) | —Cl | —CH₃ | —OC₃H₇ |
| DGS(IIIa) or (IIIb) | —Cl | —CH₃ | —CHF₂ |
| DGT(IIla) or (IIIb) | —Cl | —CH₃ | —CF₃ |
| DGU(IIIa) or (IIIb) | —Cl | —CH₃ | —CHCl₂ |
| DGV(IIla) or (IIIb) | —Cl | —CH₃ | —CCl₃ |
| DGW(IIIa) or (IIIb) | —Cl | —CH₃ | —F |
| DGX(IIIa) or (IIIb) | —Cl | —CH₃ | —Cl |
| DGY(IIIa) or (IIIb) | —Cl | —CH₃ | —Br |
| DGZ(IIIa) or (IIIb) | —Cl | —CH₃ | —I |
| DHA(IIIa) | —CHCl₂ | —H | —H |
| DHB(IIIa) | —CHCl₂ | —H | —CH₃ |
| DHC(IIIa) | —CHCl₂ | —H | -n-propyl |
| DHD(IIIa) | —CHCl₂ | —H | -n-butyl |
| DHE(IIIa) | —CHCl₂ | —H | -t-butyl |
| DHF(IIIa) | —CHCl₂ | —H | -iso-butyl |
| DHG(IIIa) | —CHCl₂ | —H | —OCH₃ |
| DHH(IIIa) | —CHCl₂ | —H | —OC₂H₅ |
| DHI(IIIa) | —CHCl₂ | —H | —OC₃H₇ |
| DHJ(IIIa) | —CHCl₂ | —H | —CHF₂ |
| DHK(IIIa) | —CHCl₂ | —H | —CF₃ |
| DHL(IIIa) | —CHCl₂ | —H | —CHCl₂ |
| DHM(IIIa) | —CHCl₂ | —H | —CCl₃ |
| DHN(IIIa) | —CHCl₂ | —H | —F |
| DHO(IIIa) | —CHCl₂ | —H | —Cl |
| DHP(IIIa) | —CHCl₂ | —H | —Br |
| DHQ(IIIa) | —CHCl₂ | —H | —I |
| DHR(IIIa) or (IIIb) | —CHCl₂ | —OH | —H |
| DHS(IIIa) or (IIIb) | —CHCl₂ | —OH | —CH₃ |
| DHT(IIIa) or (IIIb) | —CHCl₂ | —OH | -n-propyl |
| DHU(IIIa) or (IIIb) | —CHCl₂ | —OH | -n-butyl |
| DHV(IIIa) or (IIIb) | —CHCl₂ | —OH | -t-butyl |
| DHW(IIIa) or (IIIb) | —CHCl₂ | —OH | -iso-butyl |
| DHX(IIIa) or (IIIb) | —CHCl₂ | —OH | —OCH₃ |
| DHY(IIIa) or (IIIb) | —CHCl₂ | —OH | —OC₂H₅ |
| DHZ(IIIa) or (IIIb) | —CHCl₂ | —OH | —OC₃H₇ |
| DIA(IIIa) or (IIIb) | —CHCl₂ | —OH | —CHF₂ |
| DIB(IIIa) or (IIIb) | —CHCl₂ | —OH | —CF₃ |
| DIC(IIIa) or (IIIb) | —CHCl₂ | —OH | —CHCl₂ |
| DID(IIIa) or (IIIb) | —CHCl₂ | —OH | —CCl₃ |
| DIE(IIIa) or (IIIb) | —CHCl₂ | —OH | —F |
| DIF(IIIa) or (IIIb) | —CHCl₂ | —OH | —Cl |
| DIG(IIIa) or (IIIb) | —CHCl₂ | —OH | —Br |
| DIH(IIIa) or (IIIb) | —CHCl₂ | —OH | —I |
| DII(IIIa) or (IIIb) | —CHCl₂ | —F | —H |
| DIJ(IIIa) or (IIIb) | —CHCl₂ | —F | —CH₃ |
| DIK(IIIa) or (IIIb) | —CHCl₂ | —F | -n-propyl |
| DIL(IIIa) or (IIIb) | —CHCl₂ | —F | -n-butyl |
| DIM(IIIa) or (IIIb) | —CHCl₂ | —F | -t-butyl |
| DIN(IIIa) or (IIIb) | —CHCl₂ | —F | -iso-butyl |
| DIO(IIIa) or (IIIb) | —CHCl₂ | —F | —OCH₃ |
| DIP(IIIa) or (IIIb) | —CHCl₂ | —F | —OC₂H₅ |
| DIQ(IIIa) or (IIIb) | —CHCl₂ | —F | —OC₃H₇ |
| DIR(IIIa) or (IIIb) | —CHCl₂ | —F | —CHF₂ |
| DIS(IIIa) or (IIIb) | —CHCl₂ | —F | —CF₃ |
| DIT(IIIa) or (IIIb) | —CHCl₂ | —F | —CHCl₂ |
| DIU(IIIa) or (IIIb) | —CHCl₂ | —F | —CCl₃ |
| DIV(IIIa) or (IIIb) | —CHCl₂ | —F | —F |
| DIW(IIla) or (IIIb) | —CHCL2 | —F | —Cl |
| DLX(IIIa) or (IIIb) | —CHCl₂ | —F | —Br |
| DIY(IIIa) or (IIIb) | —CHCl₂ | —F | —I |
| DIZ(IIIa) or (IIb) | —CHCl₂ | —Cl | —H |
| DIA(IIIa) or (IIIb) | —CHCl₂ | —Cl | —CH₃ |
| DIB(IIIa) or (IIIb) | —CHCl₂ | —Cl | -n-propyl |
| DIC(IIIa) or (IIIb) | —CHCl₂ | —Cl | -n-butyl |
| DID(IIIa) or (IIIb) | —CFICl2 | —Cl | -t-butyl |
| DIE(IIIa) or (IIIb) | —CHCl₂ | —Cl | -iso-butyl |
| DIF(lIla) or (IIIb) | —CHCl₂ | —Cl | —OCH₃ |
| DIG(IIIa) or (IIIb) | —CHCl₂ | —Cl | —OC₂H₅ |
| DIH(IIIa) or (IIIb) | —CHCl₂ | —Cl | —OC₃H₇ |
| DII(IIIa) or (IIIb) | —CHCl₂ | —Cl | —CHF₂ |
| DIJ(IIIa) or (IlIb) | —CHCl₂ | —Cl | —CF₃ |
| DIK(IIIa) or (IIIb) | —CHCl₂ | —Cl | —CHCl₂ |
| DIL(IIIa) or (IIIb) | —CHCl₂ | —Cl | —Cd3 |
| DIM(IIIa) or (IIIb) | —CHCl₂ | —Cl | —F |
| DIN(IIIa) or (IIIb) | —CHCl₂ | —Cl | —Cl |
| DIO(IIIa) or (IIIb) | —CHCl₂ | —Cl | —Br |
| DIP(IIIa) or (IIIb) | —CHCL2 | —Cl | —I |
| DIQ(IIIa) or (IIIb) | —CHCl₂ | —Br | —H |
| DIR(IIIa) or (IIIb) | —CHCl₂ | —Br | —CH₃ |
| DIS(IIIa) or (IIIb) | —CHCi2 | —Br | -n-propyl |
| DIT(IIIa) or (IIIb) | —CHCl₂ | —Br | -n-butyl |
| DIU(IIIa) or (IIIb) | —CHCl₂ | —Br | -t-butyl |
| DIV(IIIa) or (IIIb) | —CHCl₂ | —Br | -iso-butyl |
| DIW(IIIa) or (IIIb) | —CHCl₂ | —Br | —OCH₃ |
| DIX(IIIa) or (IIIb) | —CHCl₂ | —Br | —OC₂H₅ |
| DIY(IIIa) or (IIIb) | —CHCl₂ | —Br | —OC₃H₇ |
| DIZ(IIIa) or (IIIb) | —CHCl₂ | —Br | —CHF₂ |
| DJA(IIIa) or (IIIb) | —CHCl₂ | —Cl | —CH₃ |
| DJB(IIIa) or (IIIb) | —CHCl₂ | —Cl | -n-propyl |
| DJC(IIIa) or (IIIb) | —CHCl₂ | —Cl | -n-butyl |
| DJD(IIIa) or (IIIb) | —CHCl₂ | —Cl | -t-butyl |
| DJE(IIIa) or (IIIb) | —CHCl₂ | —Cl | -iso-butyl |
| DJF(IIIa) or (IIIb) | —CHCl₂ | —Cl | —OCH₃ |
| DJG(IIIa) or (IIIb) | —CHCl₂ | —Cl | —OC₂H₅ |
| DJH(IIIa) or (IIIb) | —CHCl₂ | —Cl | —OC₃H₇ |
| DJI(IIIa) or (IIIb) | —CHCl₂ | —Cl | —CHF₂ |
| DJK(IIIa) or (IIIb) | —CHCl₂ | —Cl | —CHCl₂ |
| DJL(IIIa) or (IIIb) | —CHCl₂ | —Cl | —CCl₃ |
| DJM(IIIa) or (IIIb) | —CHCl₂ | —Cl | —F |
| DJN(IIIa) or (IIIb) | —CHCl₂ | —Cl | —Cl |
| DJO(IIIa) or (IIIb) | —CHCl₂ | —Cl | —Br |
| DJP(IIIa) or (IIIb) | —CHCl₂ | —Cl | —I |
| DJQ(IIIa) or (IIIb) | —CHCl₂ | —Br | —H |
| DJR(IIIa) or (IIIb) | —CHCl₂ | —Br | —CH₃ |
| DJS(IIIa) or (IIIb) | —CHCl₂ | —Br | -n-propyl |
| DJT(IIIa) or (IIIb) | —CHCl₂ | —Br | -n-butyl |
| DJU(IIIa) or (IIIb) | —CHCl₂ | —Br | -t-butyl |
| DJV(IIIa) or (IIIb) | —CHCl₂ | —Br | -iso-butyl |
| DJW(IIIa) or (IIIb) | —CHCl₂ | —Br | —OCH₃ |
| DJX(IIIa) or (IIIb) | —CHCl₂ | —Br | —OC₂H₅ |
| DJY(IIIa) or (IIIb) | —CHCl₂ | —Br | —OC₃H₇ |
| DKA(IIIa) or (IIIb) | —CHCl₂ | —Br | —CF₃ |
| DKB(IIIa) or (IIIb) | —CHCl₂ | —Br | —CHCl₂ |
| DKC(IIIa) or (IIIb) | —CHCl₂ | —Br | —CCl₃ |
| DKD(IIIa) or (IIIb) | —CHCl₂ | —Br | —F |
| DKE(IIIa) or (IIIb) | —CHCl₂ | —Br | —Cl |
| DKF(IIIa) or (IIIb) | —CHCl₂ | —Br | —Br |
| DKG(IIIa) or (IIIb) | —CHCl₂ | —Br | —I |
| DKH(IIla) or (IIIb) | —CHCl₂ | —I | —H |
| DKI(IIIa) or (IIIb) | —CHCl₂ | —I | —CH₃ |
| DKJ(IIIa) or (IIIb) | —CHCl₂ | —I | -n-propyl |
| DKK(IIIa) or (IIIb) | —CHCl₂ | —I | -n-butyl |
| DKL(IIIa) or (IIIb) | —CHCl₂ | —I | -t-butyl |
| DKM(IIIa) or (IIIb) | —CHCl₂ | —I | -iso-butyl |
| DKN(IIIa) or (IIIb) | —CHCl₂ | —I | —OCH₃ |
| DKO(IIIa) or (IIIb) | —CHCl₂ | —I | —OC₂H₅ |
| DKP(IIIa) or (Ilib) | —CHCl₂ | —I | —OC₃H₇ |
| DKQ(IIIa) or (IIIb) | —CHCl₂ | —I | —CHF₂ |
| DKR(IIIa) or (IIIb) | —CHCl₂ | —I | —CF₃ |
| DKS(IIIa) or (IIIb) | —CHCl₂ | —I | —CHCl₂ |
| DKT(IIIa) or (IIIb) | —CHCl₂ | —I | —CCl₃ |
| DKU(IIIa) or (IIIb) | —CHCl₂ | —I | —F |
| DKV(IIIa) or (IIIb) | —CHCl₂ | —I | —Cl |
| DKW(IIIa) or (IIIb) | —CHCl₂ | —I | —Br |
| DKX(IIIa) or (IIIb) | —CHCl₂ | —I | —I |
| DKY(IIIa) or (IIIb) | —CHCl₂ | —NO₂ | —H |
| DKZ(IIIa) or (IIIb) | —CHCl₂ | —NO₂ | —CH₃ |
| DLA(IIIa) or (IIIb) | —CHCl₂ | —NO₂ | -n-propyl |
| DLB(IIIa) or (IIIb) | —CHCl₂ | —NO₂ | -n-butyl |
| DLC(IIIa) or (IIIb) | —CHCl₂ | —NO₂ | -t-butyl |
| DLD(IIIa) or (IIIb) | —CHCl₂ | —NO₂ | -iso-butyl |
| DLE(IIIa) or (IIIb) | —CHCl₂ | —NO₂ | —OCH₃ |
| DLF(IIIa) or (IIIb) | —CHCl₂ | —NO₂ | —OC₂H₅ |
| DLG(IIIa) or (IlIb) | —CHCl₂ | —NO₂ | —OC₃H₇ |
| DLH(IIIa) or (IIIb) | —CHCl₂ | —NO₂ | —CHF₂ |
| DLI(IIIa) or (IIIb) | —CHCl₂ | —NO₂ | —CF₃ |
| DLJ(IIIa) or (IIIb) | —CHCl₂ | —NO₂ | —CHCl₂ |
| DLK(IIIa) or (IIIb) | —CHCl₂ | —NO₂ | —CCl₃ |
| DLL(IIIa) or (IIIb) | —CHCl₂ | —NO₂ | —F |
| DLM(IIIa) or (IIIb) | —CHCl₂ | —NO₂ | —Cl |
| DLN(IIIa) or (IIIb) | —CHCl₂ | —NO₂ | —Br |
| DLO(IIIa) or (IIIb) | —CHCl₂ | —NO₂ | —I |
| DLP(IIIa) or (lIIb) | —CHCl₂ | —CN | —H |
| DLQ(IIIa) or (IIIb) | —CHCl₂ | —CN | —CH₃ |
| DLR(IIIa) or (IIIb) | —CHCl₂ | —CN | -n-propyl |
| DLS(IIIa) or (IIIb) | —CHCl₂ | —CN | -n-butyl |
| DLT(IIIa) or (IIIb) | —CHCl₂ | —CN | -t-butyl |
| DLU(IIIa) or (IIIb) | —CHCl₂ | —CN | -iso-butyl |
| DLV(IIIa) or (IIIb) | —CHCl₂ | —CN | —OCU3 |
| DLW(IIIa) or (IIIb) | —CHCl₂ | —CN | —OC₂H₅ |
| DLX(IIIa) or (IIIb) | —CHCl₂ | —CN | —OC₃H₇ |
| DLY(IIIa) or (IIIb) | —CHCl₂ | —CN | —CHF₂ |
| DLZ(IIIa) or (IIIb) | —CHCl₂ | —CN | —CF₃ |
| DMA(IIIa) or (IIIb) | —CHCl₂ | —CN | —CHCl₂ |
| DMB(IIIa) or (IIIb) | —CHCl₂ | —CN | —CCl₃ |
| DMC(IIIa) or (IIIb) | —CHCl₂ | —CN | —F |
| DMD(IIIa) or (IIIb) | —CHCl₂ | —CN | —Cl |
| DME(IIIa) or (IIIb) | —CHCl₂ | —CN | —Br |
| DMF(IIIa) or (IIIb) | —CHCl₂ | —CN | —I |
| DMG(IIIa) or (IIIb) | —CHCl₂ | —NH₂ | —H |
| DMH(IIIa) or (IIIb) | —CHCl₂ | —NH₂ | CH₃ |
| DMI(IIIa) or (IIIb) | —CHCl₂ | —NH₂ | -n-propyl |
| DMJ(IIIa) or (IIIb) | —CHCl₂ | —NH₂ | -n-butyl |
| DMK(IIIa) or (IIIb) | —CHCl₂ | —NH₂ | -t-butyl |
| DML(IIIa) or (IIIb) | —CHCl₂ | —NH₂ | -iso-butyl |
| DMM(IIIa) or (IIIb) | —CHCl₂ | —NH₂ | OCH₃ |
| DMN(IIIa) or (IIIb) | —CHCl₂ | —NH₂ | —OC₂H₅ |
| DMO(IIIa) or (IIIb) | —CHCl₂ | —NH₂ | —OC₃H₇ |
| DMP(IIIa) or (IIIb) | —CHCl₂ | —NH₂ | —CHF₂ |
| DMQ(IIIa) or (IIIb) | —CHCl₂ | —NH₂ | —CF₃ |
| DMR(IIIa) or (IIIb) | —CHCl₂ | —NH₂ | —CHCl₂ |
| DMS(IIIa) or (IIIb) | —CHCl₂ | —NH₂ | —CCl₃ |
| DMT(IIIa) or (IIIb) | —CHCl₂ | —NH₂ | —F |
| DMU(IIIa) or (IIIb) | —CHCl₂ | —NH₂ | —Cl |
| DMV(IIIa) or (IIIb) | —CHCl₂ | —NH₂ | —Br |
| DMW(IIIa) or (IIIb) | —CHCl₂ | —NH₂ | —I |
| DMX(IIla) or (IIIb) | —CHCl₂ | —CH₃ | —H |
| DMY(IIIa) or (IIIb) | —CHCl₂ | —CH₃ | —CH₃ |
| DMZ(IIIa) or (IIIb) | —CHCl₂ | —CH₃ | -n-propyl |
| DNA(IIIa) or (IIIb) | —CHCl₂ | —CH₃ | -n-butyl |
| DNB(IIIa) or (IIIb) | —CHCl₂ | —CH₃ | -t-butyl |
| DNC(IIIa) or (IIIb) | —CHCl₂ | —CH₃ | -iso-butyl |
| DND(IIIa) or (IIIb) | —CHCl₂ | —CH₃ | —OCH₃ |
| DNE(IIIa) or (IIIb) | —CHCl₂ | —CH₃ | —OC₂H₅ |
| DNF(IIIa) or (IIIb) | —CHCl₂ | —CH₃ | —OC₃H₇ |
| DNG(IIIa) or (IIIb) | —CHCl₂ | —CH₃ | —CHF₂ |
| DNH(IIIa) or (IIIb) | —CHCl₂ | —CH₃ | —CF₃ |
| DNI(IIIa) or (IIIb) | —CHCl₂ | —CH₃ | —CHCl₂ |
| DNJ(IIIa) or (IIIb) | —CHCl | —CH₃ | —CCl₃ |
| DNK(IIIa) or (IIIb) | —CHCl₂ | —CH₃ | —F |
| DNL(IIIa) or (IIIb) | —CHCl₂ | —CH₃ | —Cl |
| DNM(IIIa) or (IIIb) | —CHCl₂ | —CH₃ | —Br |
| DNN(IIIa) or (IIIb) | —CHCl₂ | —CH₃ | —I |
| DNO(IIIa) | —CF₃ | —H | —H |
| DNP(IIIa) | —CF₃ | —H | —CH₃ |
| DNQ(IIIa) | —CF₃ | —H | -n-propyl |
| DNR(IIIa) | —CF₃ | —H | -n-butyl |
| DNS(IIIa) | —CF₃ | —H | -t-butyl |
| DNT(IIIa) | —CF₃ | —H | -iso-butyl |
| DNU(IIIa) | —CF₃ | —H | —OCH₃ |
| DNV(IIIa) | —CF₃ | —H | —OC₂H₅ |
| DNW(IIIa) | —CF₃ | —H | —OC₃H₇ |
| DNX(IIIa) | —CF₃ | —H | —CHF₂ |
| DNY(IIIa) | —CF₃ | —H | —CF₃ |
| DNZ(IIIa) | —CF₃ | —H | —CHCl₂ |
| DOA(IIIa) | —CF₃ | —H | —CCl₃ |
| DOB(IIIa) | —CF₃ | —H | —F |
| DOC(IIIa) | —CF₃ | —H | —Cl |
| DOD(IIla) | —CF₃ | —H | —Br |
| DOE(IIIa) | —CF₃ | —H | —I |
| DOF(IIIa) or (IIIb) | —CF₃ | —OH | —H |
| DOG(IIIa) or (IIIb) | —CF₃ | —OH | —CH₃ |
| DOH(IIIa) or (IIIb) | —CF₃ | —OH | -n-propyl |
| DOI(IIIa) or (IIIb) | —CF₃ | —OH | -n-butyl |
| DOJ(IIIa) or (IIIb) | —CF₃ | —OH | -t-butyl |
| DOK(IIIa) or (IIIb) | —CF₃ | —OH | -iso-butyl |
| DOL(IIIa) or (IIIb) | —CF₃ | —OH | —OCH₃ |
| DOM(IIIa) or (IIIb) | —CF₃ | —OH | —OC₂H₅ |
| DON(IIIa) or (IIIb) | —CF₃ | —OH | —OC₃H₇ |
| DOO(IIIa) or (IIIb) | —CF₃ | —OH | —CHF₂ |
| DOP(IIIa) or (IIIb) | —CF₃ | —OH | —CF₃ |
| DOQ(IIIa) or (IIIb) | —CF₃ | —OH | —CHCl₂ |
| DOR(IIIa) or (IIIb) | —CF₃ | —OH | —CCl₃ |
| DOS(IIIa) or (IIIb) | —CF₃ | —OH | —F |
| DOT(IIIa) or (IIIb) | —CF₃ | —OH | —Cl |
| DOU(IIla) or (IIIb) | —CF₃ | —OH | —Br |
| DOV(IIIa) or (IIIb) | —CF₃ | —OH | —I |
| DOW(IIIa) or (IIIb) | —CF₃ | —F | —H |
| DOX(IIIa) or (IIIb) | —CF₃ | —F | —CH₃ |
| DOY(IIIa) or (IIIb) | —CF₃ | —F | -n-propyl |
| DOZ(IIIa) or (IIIb) | —CF₃ | —F | -n-butyl |
| DPA(IIIa) or (IIIb) | —CF₃ | —F | -t-butyl |
| DPB(IIIa) or (IIIb) | —CF₃ | —F | -iso-butyl |
| DPC(IIIa) or (IIIb) | —CF₃ | —F | —OCH₃ |
| DPD(IIIa) or (IIIb) | —CF₃ | —F | —OC₂H₅ |
| DPE(IIIa) or (IIIb) | —CF₃ | —F | —OC₃H₇ |
| DPF(IIIa) or (IIIb) | —CF₃ | —F | —CHF₂ |
| DPG(IIIa) or (IIIb) | —CF₃ | —F | —CF₃ |
| DPH(IIIa) or (IIIb) | —CF₃ | —F | —CHCl₂ |
| DPI(IIIa) or (IIIb) | —CF₃ | —F | —CCl₃ |
| DPJ(IIIa) or (IIIb) | —CF₃ | —F | —F |
| DPK(IIIa) or (IIIb) | —CF₃ | —F | —Cl |
| DPL(IIIa) or (IIIb) | —CF₃ | —F | —Br |
| DPM(IIIa) or (IIIb) | —CF₃ | —F | —I |
| DPN(IIIa) or (IIIb) | —CF₃ | —Cl | —H |
| DPO(IIIa) or (IIIb) | —CF₃ | —Cl | —CH₃ |
| DPP(IIIa) or (IIIb) | —CF₃ | —Cl | -n-propyl |
| DPQ(IIIa) or (IIIb) | —CF₃ | —Cl | -n-butyl |
| DPR(IIIa) or (IIIb) | —CF₃ | —Cl | -t-butyl |
| DPS(IIIa) or (IIIb) | —CF₃ | —Cl | -iso-butyl |
| DPT(IIIa) or (IIIb) | —CF₃ | —Cl | —OCH₃ |
| DPU(IIIa) or (IIIb) | —CF₃ | —Cl | —OC₂H₅ |
| DPV(IIIa) or (IIIb) | —CF₃ | —Cl | —OC₃H₇ |
| DPW(IIIa) or (IIIb) | —CF₃ | —Cl | —CHF₂ |
| DPX(IIIa) or (IIIb) | —CF₃ | —Cl | —CF₃ |
| DPY(IIIa) or (IIIb) | —CF₃ | —Cl | —CHCl₂ |
| DPZ(IIIa) or (IIIb) | —CF₃ | —Cl | —CCl₃ |
| DQA(IIIa) or (IIIb) | —CF₃ | —Cl | —F |
| DQB(IIIa) or (IIIb) | —CF₃ | —Cl | —Cl |
| DQC(IIIa) or (IIIb) | —CF₃ | —Cl | —Br |
| DQD(IIIa) or (IIIb) | —CF₃ | —Cl | —I |
| DQE(IIIa) or (IIIb) | —CF₃ | —Br | —H |
| DQF(IIIa) or (IIIb) | —CF₃ | —Br | —CH₃ |
| DQG(IIIa) or (IIIb) | —CF₃ | —Br | -n-propyl |
| DQH(IIIa) or (IIIb) | —CF₃ | —Br | -n-butyl |
| DQI(IIIa) or (IIIb) | —CF₃ | —Br | -t-butyl |
| DQJ(IIIa) or (IIIb) | —CF₃ | —Br | -iso-butyl |
| DQK(IIIa) or (IIIb) | —CF₃ | —Br | —OCH₃ |
| DQL(IIIa) or (IIIb) | —CF₃ | —Br | —OC₂H₅ |
| DQM(IIIa) or (IIIb) | —CF₃ | —Br | —OC₃H₇ |
| DQN(IIIa) or (IIIb) | —CF₃ | —Br | —CHF₂ |
| DQO(IIIa) or (IIIb) | —CF₃ | —Br | —CF₃ |
| DQP(IIIa) or (IIIb) | —CF₃ | —Br | —CHCl₂ |
| DQQ(IIIa) or (IIIb) | —CF₃ | —Br | —CCl₃ |
| DQR(IIIa) or (IIIb) | —CF₃ | —Br | —F |
| DQS(IIIa) or (IIIb) | —CF₃ | —Br | —Cl |
| DQT(IIIa) or (IIIb) | —CF₃ | —Br | —Br |
| DQU(IIIa) or (IIIb) | —CF₃ | —Br | —I |
| DQV(IIIa) or (IIIb) | —CF₃ | —I | —H |
| DQW(IIIa) or (IIIb) | —CF₃ | —I | —CH₃ |
| DQX(IIIa) or (IIIb) | —CF₃ | —I | -n-propyl |
| DQY(IIIa) or (IIIb) | —CF₃ | —I | -n-butyl |
| DQZ(IIIa) or (IIIb) | —CF₃ | —I | -t-butyl |
| DRA(IIIa) or (IIIb) | —CF₃ | —I | -iso-butyl |
| DRB(IIIa) or (IIIb) | —CF₃ | —I | —OCH₃ |
| DRC(IIIa) or (IIIb) | —CF₃ | —I | —OC₂H₅ |
| DRD(IIIa) or (IIIb) | —CF₃ | —I | —OC₃H₇ |
| DRE(IIIa) or (IIIb) | —CF₃ | —I | —CHF₂ |
| DRF(IIIa) or (IIIb) | —CF₃ | —L | —CF₃ |
| DRG(IIIa) or (IIIb) | —CF₃ | —I | —CHCl₂ |
| DRH(IIIa) or (IIIb) | —CF₃ | —I | —Cd3 |
| DRI(IIIa) or (IIIb) | —CF₃ | —I | —F |
| DRI(IIIa) or (IIIb) | —CF₃ | —I | —Cl |
| DRK(IIIa) or (IIIb) | —CF₃ | —I | —Br |
| DRL(IIIa) or (IIIb) | —CF₃ | —I | —I |
| DRM(IIIa) or (IIIb) | —CF₃ | —NO₂ | —H |
| DRN(IIIa) or (IIIb) | —CF₃ | —NO₂ | —CH₃ |
| DRO(IIIa) or (IIIb) | —CF₃ | —NO₂ | -n-propyl |
| DRP(IIIa) or (IIIb) | —CF₃ | —NO₂ | -n-butyl |
| DRQ(IIIa) or (IIIb) | —CF₃ | —NO₂ | -t-butyl |
| DRR(IIIa) or (IIIb) | —CF₃ | —NO₂ | -iso-butyl |
| DRS(IIIa) or (IIIb) | —CF₃ | —NO₂ | —OCH₃ |
| DRT(IIIa) or (IIIb) | —CF₃ | —NO₂ | —OC₂H₅ |
| DRU(IIIa) or (IIIb) | —CF₃ | —NO₂ | —OC₃H₇ |
| DRV(IIIa) or (IIIb) | —CF₃ | —NO₂ | —CHF₂ |
| DRW(IIIa) or (IIIb) | —CF₃ | —NO₂ | —CF₃ |
| DRX(IIIa) or (IIIb) | —CF₃ | —NO₂ | —CHCl₂ |
| DRY(IIIa) or (IIIb) | —CF₃ | —NO₂ | —CCl₃ |
| DRZ(IIIa) or (IIIb) | —CF₃ | —NO₂ | —F |
| DSA(IIIa) or (IIIb) | —CF₃ | —NO₂ | —Cl |
| DSB(IIIa) or (IIIb) | —CF₃ | —NO₂ | —Br |
| DSC(IIIa) or (IIIb) | —CF₃ | —NO₂ | —I |
| DSD(IIIa) or (IIIb) | —CF₃ | —CN | —H |
| DSE(IIIa) or (IIIb) | —CF₃ | —CN | —CH₃ |
| DSF(IIIa) or (IIIb) | —CF₃ | —CN | -n-propyl |
| DSG(IIIa) or (IIIb) | —CF₃ | —CN | -n-butyl |
| DSH(IIIa) or (IIIb) | —CF₃ | —CN | -t-butyl |
| DSI(IIIa) or (IIIb) | —CF₃ | —CN | -iso-butyl |
| DSJ(IIIa) or (IIIb) | —CF₃ | —CN | —OCH₃ |
| DSK(IIIa) or (IIIb) | —CF₃ | —CN | —OC₂H₅ |
| DSL(IIIa) or (IIIb) | —CF₃ | —CN | —OC₃H₇ |
| DSM(IIIa) or (IIIb) | —CF₃ | —CN | —CHF₂ |
| DSN(IIIa) or (IIIb) | —CF₃ | —CN | —CF₃ |
| DSO(IIIa) or (IIIb) | —CF₃ | —CN | —CHCl₂ |
| DSP(IIIa) or (IIIb) | —CF₃ | —CN | —CCl₃ |
| DSQ(IIIa) or (IIIb) | —CF₃ | —CN | —F |
| DSR(IIIa) or (IIIb) | —CF₃ | —CN | —Cl |
| DSS(IIIa) or (IIIb) | —CF₃ | —CN | —Br |
| DST(IIla) or (IIIb) | —CF₃ | —CN | —I |
| DSU(IIIa) or (IIIb) | —CF₃ | —NH₂ | —H |
| DSV(IIIa) or (IIIb) | —CF₃ | —NH₂ | —CH₃ |
| DSW(IIIa) or (IIIb) | —CF₃ | —NH₂ | -n-propyl |
| DSX(IIIa) or (IIIb) | —CF₃ | —NH₂ | -n-butyl |
| DSY(IIIa) or (IIIb) | —CF₃ | —NH₂ | -t-butyl |
| DSZ(IIIa) or (IIIb) | —CF₃ | —NH₂ | -iso-butyl |
| DTA(IIIa) or (IIIb) | —CF₃ | —NH₂ | —OCH₃ |
| DTB(IIIa) or (IIIb) | —CF₃ | —NH₂ | —OC₂H₅ |
| DTC(IIIa) or (IIIb) | —CF₃ | —NH₂ | —OC₃H₇ |
| DTD(IIIa) or (IIIb) | —CF₃ | —NH₂ | —CHF₂ |
| DTE(IIIa) or (IIIb) | —CF₃ | —NH₂ | —CF₃ |
| DTF(IIIa) or (IIIb) | —CF₃ | —NH₂ | —CHCl₂ |
| DTG(IIIa) or (IIIb) | —CF₃ | —NH₂ | —CCl₃ |
| DTH(IIIa) or (IIIb) | —CF₃ | —NH₂ | —F |
| DTI(IIIa) or (IIIb) | —CF₃ | —NH₂ | —Cl |
| DTJ(IIIa) or (IIIb) | —CF₃ | —NH₂ | —Br |
| DTK(IIIa) or (IIIb) | —CF₃ | —NH₂ | —I |
| DTL(IIIa) or (IIIb) | —CF₃ | —CH₃ | —H |
| DTM(IIIa) or (IIIb) | —CF₃ | —CH₃ | —CH₃ |
| DTN(IIIa) or (IIIb) | —CF₃ | —CH₃ | -n-propyl |
| DTO(IIIa) or (IIIb) | —CF₃ | —CH₃ | -n-butyl |
| DTP(IIIa) or (IIIb) | —CF₃ | —CH₃ | -t-butyl |
| DTQ(IIIa) or (IIIb) | —CF₃ | —CH₃ | -iso-butyl |
| DTR(IIIa) or (IIIb) | —CF₃ | —CH₃ | —OCH₃ |
| DTS(IIIa) or (IIIb) | —CF₃ | —CH₃ | —OC₂H₅ |
| DTT(IIIa) or (IIIb) | —CF₃ | —CH₃ | —OC₃H₇ |
| DTU(IIIa) or (IIIb) | —CF₃ | —CH₃ | —CHF₂ |
| DTV(IIIa) or (IIIb) | —CF₃ | —CH₃ | —CF₃ |
| DTW(IIIa) or (IIIb) | —CF₃ | —CH₃ | —CHCl₂ |
| DTX(IIIa) or (IIIb) | —CF₃ | —CH₃ | —CCl₃ |
| DTY(IIIa) or (IIIb) | —CF₃ | —CH₃ | —F |
| DTZ(IIIa) or (IIIb) | —CF₃ | —CH₃ | —Cl |
| DUA(IIIa) or (IIIb) | —CF₃ | —CH₃ | —Br |
| DUB(IIIa) or (IIIb) | —CF₃ | —CH₃ | —I |
| DUC(IIIa) | —NO₂ | —H | —H |
| DUD(IIIa) | —NO₂ | —H | —CH₃ |
| DUE(IIIa) | —NO₂ | —H | -n-propyl |
| DUF(IIIa) | —NO₂ | —H | -n-butyl |
| DUG(IIIa) | —NO₂ | —H | -t-butyl |
| DUH(IIIa) | —NO₂ | —H | -iso-butyl |
| DUI(IIIa) | —NO₂ | —H | —OCH₃ |
| DUJ(IIIa) | —NO₂ | —H | —OC₂H₅ |
| DUK(IIIa) | —NO₂ | —H | —OC₃H₇ |
| DUL(IIIa) | —NO₂ | —H | —CHF₂ |
| DUM(IIIa) | —NO₂ | —H | —CF₃ |
| DUN(IIIa) | —NO₂ | —H | —CHCl₂ |
| DUO(TIIa) | —NO₂ | —H | —CCI3 |
| DUP(IIIa) | —NO₂ | —H | —F |
| DUQ(IIIa) | —NO₂ | —H | —Cl |
| DUR(IIIa) | —NO₂ | —H | —Br |
| DUS(IIIa) | —NO₂ | —H | —I |
| DUT(IIIa) or (IIIb) | —NO₂ | —OH | —H |
| DUU(IIIa) or (IIIb) | —NO₂ | —OH | —CH₃ |
| DUV(IIIa) or (IIIb) | —NO₂ | —OH | -n-propyl |
| DUW(IIIa) or (IIIb) | —NO₂ | —OH | -n-butyl |
| DUX(IIIa) or (IIIb) | —NO₂ | —OH | -t-butyl |
| DUY(IIIa) or (IIIb) | —NO₂ | —OH | -iso-butyl |
| DUZ(IIIa) or (IIIb) | —NO₂ | —OH | —OCH₃ |
| DVA(IIIa) or (IIIb) | —NO₂ | —OH | —OC₂H₅ |
| DVB(IIIa) or (IIIb) | —NO₂ | —OH | —OC₃H₇ |
| DVC(IIIa) or (IIIb) | —NO₂ | —OH | —CHF₂ |
| DVD(IIIa) or (IIIb) | —NO₂ | —OH | —CF₃ |
| DVE(IIIa) or (IIIb) | —NO₂ | —OH | —CHCl₂ |
| DVF(IIIa) or (IIIb) | —NO₂ | —OH | —CCl₃ |
| DVG(IIIa) or (IIIb) | —NO₂ | —OH | —F |
| DVH(IIIa) or (IIIb) | —NO₂ | —OH | —Cl |
| DVI(IIIa) or (IIIb) | —NO₂ | —OH | —Br |
| DVJ(IIIa) or (IIb) | —NO₂ | —OH | —I |
| DVK(IIIa) or (IIIb) | —NO₂ | —F | —H |
| DVL(IIIa) or (IIIb) | —NO₂ | —F | —CH₃ |
| DVM(IIIa) or (IIIb) | —NO₂ | —F | -n-propyl |
| DVN(IIIa) or (IIIb) | —NO₂ | —F | -n-butyl |
| DVO(IIIa) or (IIIb) | —NO₂ | —F | -t-butyl |
| DVP(IIIa) or (IIIb) | —NO₂ | —F | -iso-butyl |
| DVQ(IIIa) or (IIIb) | —NO₂ | —F | —OCH₃ |
| DVR(IIIa) or (IIIb) | —NO₂ | —F | —OC₂H₅ |
| DVS(IIIa) or (IIIb) | —NO₂ | —F | —OC₃H₇ |
| DVT(IIIa) or (IIIb) | —NO₂ | —F | —CHF₂ |
| DVU(IIIa) or (IIIb) | —NO₂ | —F | —CF₃ |
| DVW(IIIa) or (IIIb) | —NO₂ | —F | —CHCl₂ |
| DVX(IIIa) or (IIIb) | —NO₂ | —F | —CCl₃ |
| DVY(IIIa) or (IIIb) | —NO₂ | —F | —F |
| DVY(IIIa) or (IIIb) | —NO₂ | —F | —Cl |
| DVZ(IIIa) or (IIIb) | —NO₂ | —F | —Br |
| DWA(IIIa) or (IIIb) | —NO₂ | —F | —I |
| DWB(IIIa) or (IIIb) | —NO₂ | —Cl | —H |
| DWC(IIIa) or (IIIb) | —NO₂ | —Cl | —CH₃ |
| DWD(IIIa) or (IIIb) | —NO₂ | —Cl | -n-propyl |
| DWE(IIIa) or (IIIb) | —NO₂ | —Cl | -n-butyl |
| DWF(IIIa) or (IIIb) | —NO₂ | —Cl | -t-butyl |
| DWG(IIIa) or (IIIb) | —NO₂ | —Cl | -iso-butyl |
| DWH(IIIa) or (IIIb) | —NO₂ | —Cl | —OCH₃ |
| DWI(IIla) or (IIIb) | —NO₂ | —Cl | —OC₂H₅ |
| DWJ(IIIa) or (IIIb) | —NO₂ | —Cl | —OC₃H₇ |
| DWK(IIIa) or (IIIb) | —NO₂ | —Cl | —CHF₂ |
| DWL(IIIa) or (IIIb) | —NO₂ | —Cl | —CF₃ |
| DWM(IIIa) or (IIIb) | —NO₂ | —Cl | —CHCl₂ |
| DWN(IIIa) or (IIIb) | —NO₂ | —Cl | —CCl₃ |
| DWO(IIIa) or (IIIb) | —NO₂ | —Cl | —F |
| DWP(IIIa) or (IIIb) | —NO₂ | —Cl | —Cl |
| DWQ(IIIa) or (IIIb) | —NO₂ | —Cl | —Br |
| DWR(IIIa) or (IIIb) | —NO₂ | —Cl | —I |
| DWS(IIIa) or (IIIb) | —NO₂ | —Br | —H |
| DWT(IIIa) or (IIIb) | —NO₂ | —Br | —CH₃ |
| DWU(IIIa) or (IIIb) | —NO₂ | —Br | -n-propyl |
| DWV(IIIa) or (IIIb) | —NO₂ | —Br | -n-butyl |
| DWW(IIIa) or (IIIb) | —NO₂ | —Br | -t-butyl |
| DWX(IIIa) or (IIIb) | —NO₂ | —Br | -iso-butyl |
| DWY(IIIa) or (IIIb) | —NO₂ | —Br | —OCH₃ |
| DWZ(IIIa) or (IIIb) | —NO₂ | —Br | —OC₂H₅ |
| DXA(IIIa) or (IIIb) | —NO₂ | —Br | —OC₃H₇ |
| DXB(IIIa) or (IIIb) | —NO₂ | —Br | —CHF₂ |
| DXC(IIIa) or (IIIb) | —NO₂ | —Br | —CF₃ |
| DXD(IIIa) or (IIIb) | —NO₂ | —Br | —CHCl₂ |
| DXE(IIIa) or (IIIb) | —NO₂ | —Br | —CCl₃ |
| DXF(IIIa) or (IIIb) | —NO₂ | —Br | —F |
| DXG(IIIa) or (IIIb) | —NO₂ | —Br | —Cl |
| DXH(IIIa) or (IIIb) | —NO₂ | —Br | —Br |
| DXI(IIIa) or (IIIb) | —NO₂ | —Br | —I |
| DXJ(IIIa) or (IIIb) | —NO₂ | —I | —H |
| DXK(IIIa) or (IIIb) | —NO₂ | —I | —CH₃ |
| DXL(IIIa) or (IIIb) | —NO₂ | —I | -n-propyl |
| DXM(IIIa) or (IIIb) | —NO₂ | —I | -n-butyl |
| DXN(IIIa) or (IIIb) | —NO₂ | —I | -t-butyl |
| DXO(IIIa) or (IIIb) | —NO₂ | —I | -iso-butyl |
| DXP(IIIa) or (IIIb) | —NO₂ | —I | —OCH₃ |
| DXQ(IIIa) or (IIIb) | —NO₂ | —I | —OC₂H₅ |
| DXR(IIIa) or (IIIb) | —NO₂ | —I | —OC₃H₇ |
| DXS(IIIa) or (IIIb) | —NO₂ | —I | —CHF₂ |
| DXT(IIIa) or (IIIb) | —NO₂ | —I | —CF₃ |
| DXU(IIIa) or (IIIb) | —NO₂ | —I | —CHCl₂ |
| DXV(IIIa) or (IIIb) | —NO₂ | —I | —CCl₃ |
| DXW(IIIa) or (IIIb) | —NO₂ | —I | —F |
| DXX(IIIa) or (IIIb) | —NO₂ | —I | —Cl |
| DXY(IIIa) or (IIIb) | —NO₂ | —I | —Br |
| DXZ(IIIa) or (IIIb) | —NO₂ | —I | —I |
| DYA(IIIa) or (IIIb) | —NO₂ | —NO₂ | —H |
| DYB(IIIa) or (IIIb) | —NO₂ | —NO₂ | —CH₃ |
| DYC(IIIa) or (IIIb) | —NO₂ | —NO₂ | -n-propyl |
| DYD(IIIa) or (IIIb) | —NO₂ | —NO₂ | -n-butyl |
| DYE(IIIa) or (IIIb) | —NO₂ | —NO₂ | -t-butyl |
| DYF(IIIa) or (IIIb) | —NO₂ | —NO₂ | -iso-butyl |
| DYG(IIIa) or (IIIb) | —NO₂ | —NO₂ | —OCH₃ |
| DYH(IIIa) or (IIIb) | —NO₂ | —NO₂ | —OC₂H₅ |
| DYI(IIIa) or (IIIb) | —NO₂ | —NO₂ | —OC₃H₇ |
| DYJ(IIIa) or (IIIb) | —NO₂ | —NO₂ | —CHF₂ |
| DYK(IIIa) or (IIIb) | —NO₂ | —NO₂ | —CF₃ |
| DYL(IIIa) or (IIIb) | —NO₂ | —NO₂ | —CHCl₂ |
| DYM(IIIa) or (IIIb) | —NO₂ | —NO₂ | —CCl₃ |
| DYN(IIIa) or (IIIb) | —NO₂ | —NO₂ | —F |
| DYO(IIIa) or (IIIb) | —NO₂ | —NO₂ | —Cl |
| DYP(IIIa) or (IIIb) | —NO₂ | —NO₂ | —Br |
| DYQ(IIIa) or (IIIb) | —NO₂ | —NO₂ | —I |
| DYR(IIIa) or (IIIb) | —NO₂ | —CN | —H |
| DYS(IIIa) or (IIIb) | —NO₂ | —CN | —CH₃ |
| DYT(IIIa) or (IIIb) | —NO₂ | —CN | -n-propyl |
| DYU(IIIa) or (IIIb) | —NO₂ | —CN | -n-butyl |
| DYV(IIIa) or (IIIb) | —NO₂ | —CN | -t-butyl |
| DYW(IIIa) or (IIIb) | —NO₂ | —CN | -iso-butyl |
| DYX(IIIa) or (IIIb) | —NO₂ | —CN | —OCH₃ |
| DYY(IIIa) or (IIIb) | —NO₂ | —CN | —OC₂H₅ |
| DYZ(IIIa) or (IIIb) | —NO₂ | —CN | —OC₃H₇ |
| DZA(IIIa) or (IIIb) | —NO₂ | —CN | —CHF₂ |
| DZB(IIIa) or (IIIb) | —NO₂ | —CN | —CF₃ |
| DZC(IIIa) or (IIIb) | —NO₂ | —CN | —CHCl₂ |
| DZD(IIIa) or (IIIb) | —NO₂ | —CN | —CCl₃ |
| DZE(IIIa) or (IIIb) | —NO₂ | —CN | —F |
| DZF(IIIa) or (IIIb) | —NO₂ | —CN | —Cl |
| DZG(IIIa) or (IIIb) | —NO₂ | —CN | —Br |
| DZH(IIIa) or (IIIb) | —NO₂ | —CN | —I |
| DZI(IIIa) or (IIIb) | —NO₂ | —NH₂ | —H |
| DZJ(IIIa) or (IIIb) | —NO₂ | —NH₂ | —CH₃ |
| DZK(IIIa) or (IIIb) | —NO₂ | —NH₂ | -n-propyl |
| DZL(IIIa) or (IIIb) | —NO₂ | —NH₂ | -n-butyl |
| DZM(IIIa) or (IIIb) | —NO₂ | —NH₂ | -t-butyl |
| DZN(IIIa) or (IIIb) | —NO₂ | —NH₂ | -iso-butyl |
| DZO(IIIa) or (IIIb) | —NO₂ | —NH₂ | —OCH₃ |
| DZP(IIIa) or (IIIb) | —NO₂ | —NH₂ | —OC₂H₅ |
| DZQ(IIIa) or (IIIb) | —NO₂ | —NH₂ | —OC₃H₇ |
| DZR(IIIa) or (IIIb) | —NO₂ | —NH₂ | —CLIF2 |
| DZS(IIIa) or (IIIb) | —NO₂ | —NH₂ | —CF₃ |
| DZT(IIIa) or (IIIb) | —NO₂ | —NH₂ | —CHCl₂ |
| DZU(IIIa) or (IIIb) | —NO₂ | —NH₂ | —CCl₃ |
| DZV(IIIa) or (IIIb) | —NO₂ | —NH₂ | —F |
| DZW(IIIa) or (IIIb) | —NO₂ | —NH₂ | —Cl |
| DZX(IIIa) or (IIIb) | —NO₂ | —NH₂ | —Br |
| DZY(IIIa) or (IIIb) | —NO₂ | —NH₂ | —I |
| DZZ(IIIa) or (IIIb) | —NO₂ | —CH₃ | —H |
| EAA(IIIa) or (IIIb) | —NO₂ | —CH₃ | —CH₃ |
| EAB(IIIa) or (IIIb) | —NO₂ | —CH₃ | -n-propyl |
| EAC(IIIa) or (IIIb) | —NO₂ | —CH₃ | -n-butyl |
| EAD(IIIa) or (IIIb) | —NO₂ | —CH₃ | -t-butyl |
| EAE(IIIa) or (IIIb) | —NO₂ | —CH₃ | -iso-butyl |
| EAF(IIIa) or (IIIb) | —NO₂ | —CH₃ | —OCH₃ |
| EAG(IIIa) or (IIIb) | —NO₂ | —CH₃ | —OC₂H₅ |
| EAH(IIIa) or (IIIb) | —NO₂ | —CH₃ | —OC₃H₇ |
| EAI(IIIa) or (IIIb) | —NO₂ | —CH₃ | —CHF₂ |
| EAJ(IIIa) or (IIIb) | —NO₂ | —CH₃ | —CF₃ |
| EAK(IIIa) or (IIIb) | —NO₂ | —CH₃ | —CHCl₂ |
| EAL(IIIa) or (IIIb) | —NO₂ | —CH₃ | —CCI3 |
| EAM(IIIa) or (IIIb) | —NO₂ | —CH₃ | —F |
| EAN(IIIa) or (IIIb) | —NO₂ | —Cl3 | —Cl |
| EAO(IIIa) or (IIIb) | —NO₂ | —CH₃ | —Br |
| EAP(IIIa) or (IIIb) | —NO₂ | —CH₃ | —I |
| EAQ(IIIa) | —CN | —H | —H |
| EAR(IIIa) | —CN | —H | —CH₃ |
| EAS(IIIa) | —CN | —H | -n-propyl |
| EAT(IIIa) | —CN | —H | -n-butyl |
| EAU(IIIa) | —CN | —H | -t-butyl |
| EAV(IIIa) | —CN | —H | -iso-butyl |
| EAW(IIIa) | —CN | —H | —OCH₃ |
| EAX(IIIa) | —CN | —H | —OC₂H₅ |
| EAY(IIIa) | —CN | —H | —OC₃H₇ |
| EAZ(IIIa) | —CN | —H | —CHF₂ |
| EBA(IIIa) | —CN | —H | —CF₃ |
| EBB(IIIa) | —CN | —H | —CHCl₂ |
| EBC(IIIa) | —CN | —H | —CCl₃ |
| EBD(IIIa) | —CN | —H | —F |
| EBE(IIIa) | —CN | —H | —Cl |
| EBF(IIIa) | —CN | —H | —Br |
| EBG(IIIa) | —CN | —H | —I |
| EBH(IIIa) or (IIIb) | —CN | —OH | —H |
| EBI(IIIa) or (IIIb) | —CN | —OH | —CH₃ |
| EBJ(IIIa) or (IIIb) | —CN | —OH | -n-propyl |
| EBK(IIIa) or (IIIb) | —CN | —OH | -n-butyl |
| EBL(IIIa) or (IIIb) | —CN | —OH | -t-butyl |
| EBM(IIIa) or (IIIb) | —CN | —OH | -iso-butyl |
| EBN(IIIa) or (IIIb) | —CN | —OH | —OCH₃ |
| EBO(IIIa) or (IIIb) | —CN | —OH | —OC₂H₅ |
| EBP(IIIa) or (IIIb) | —CN | —OH | —OC₃H₇ |
| EBQ(IIIa) or (IIIb) | —CN | —OH | —CHF₂ |
| EBR(IIIa) or (IIIb) | —CN | —OH | —CF₃ |
| EBS(IIIa) or (IIIb) | —CN | —OH | —CHCl₂ |
| EBT(IIIa) or (IIIb) | —CN | —OH | —CCl₃ |
| EBU(IIIa) or (IIIb) | —CN | —OH | —F |
| EBV(IIIa) or (IIIb) | —CN | —OH | —Cl |
| EBW(IIIa) or (IIIb) | —CN | —OH | —Br |
| EBX(IIIa) or (IIIb) | —CN | —OH | —I |
| EBY(IIIa) or (IIIb) | —CN | —F | —H |
| EBZ(IIIa) or (IIIb) | —CN | —F | —CH₃ |
| ECA(IIIa) or (IIIb) | —CN | —F | -n-propyl |
| ECB(IIIa) or (IIIb) | —CN | —F | -n-butyl |
| ECC(IIIa) or (IIIb) | —CN | —F | -t-butyl |
| ECD(IIIa) or (IIIb) | —CN | —F | -iso-butyl |
| ECE(IIIa) or (IIIb) | —CN | —F | —OCH₃ |
| ECF(IIIa) or (IIIb) | —CN | —F | —OC₂H₅ |
| ECG(IIIa) or (IIIb) | —CN | —F | —OC₃H₇ |
| ECH(IIIa) or (IIIb) | —CN | —F | —CHF₂ |
| ECI(IIIa) or (IIIb) | —CN | —F | —CF₃ |
| ECJ(IIIa) or (IIIb) | —CN | —F | —CHCl₂ |
| ECK(IIIa) or (IIIb) | —CN | —F | —CCl₃ |
| ECL(IIIa) or (IIIb) | —CN | —F | —F |
| ECM(IIIa) or (IIIb) | —CN | —F | —Cl |
| ECN(IIIa) or (IIIb) | —CN | —F | —Br |
| ECO(IIIa) or (IIIb) | —CN | —F | —I |
| ECP(IIIa) or (IIIb) | —CN | —Cl | —H |
| ECQ(IIIa) or (IIIb) | —CN | —Cl | —CH₃ |
| ECR(IIIa) or (IIIb) | —CN | —Cl | -n-propyl |
| ECS(IIIa) or (IIIb) | —CN | —Cl | -n-butyl |
| ECT(IIIa) or (IIIb) | —CN | —Cl | -t-butyl |
| ECU(IIIa) or (IIIb) | —CN | —Cl | -iso-butyl |
| ECV(IIIa) or (IIIb) | —CN | —Cl | —OCH₃ |
| ECW(IIIa) or (IIIb) | —CN | —Cl | —OC₂H₅ |
| ECX(IIIa) or (IIIb) | —CN | —Cl | —OC₃H₇ |
| ECY(IIIa) or (IIIb) | —CN | —Cl | —CHF₂ |
| ECZ(IIIa) or (IIIb) | —CN | —Cl | —CF₃ |
| EDA(IIIa) or (IIIb) | —CN | —Cl | —CHCl₂ |
| EDB(IIIa) or (IIIb) | —CN | —Cl | —CCl₃ |
| EDC(IIIa) or (IIIb) | —CN | —Cl | —F |
| EDD(IIIa) or (IIIb) | —CN | —Cl | —Cl |
| EDE(IIIa) or (IIIb) | —CN | —Cl | —Br |
| EDF(IIIa) or (IIIb) | —CN | —Cl | —I |
| EDG(IIIa) or (IIIb) | —CN | —Br | —H |
| EDH(IIIa) or (IIIb) | —CN | —Br | —CH₃ |
| EDI(IIIa) or (IIIb) | —CN | —Br | -n-propyl |
| EDJ(IIIa) or (IIIb) | —CN | —Br | -n-butyl |
| EDK(IIIa) or (IIIb) | —CN | —Br | -t-butyl |
| EDL(IIIa) or (IIIb) | —CN | —Br | -iso-butyl |
| EDM(IIIa) or (IIIb) | —CN | —Br | —OCH₃ |
| EDN(IIIa) or (IIIb) | —CN | —Br | —OC₂H₅ |
| EDO(IIIa) or (IIIb) | —CN | —Br | —OC₃H₇ |
| EDP(IIIa) or (IIIb) | —CN | —Br | —CHF₂ |
| EDQ(IIIa) or (IIIb) | —CN | —Br | —CF₃ |
| EDR(IIIa) or (IIIb) | —CN | —Br | —CHCl₂ |
| EDS(IIIa) or (IIIb) | —CN | —Br | —CCl₃ |
| EDT(IIIa) or (IIIb) | —CN | —Br | —F |
| EDU(IIIa) or (IIIb) | —CN | —Br | —Cl |
| EDV(IIIa) or (IIIb) | —CN | —Br | —Br |
| EDW(IIIa) or (IIIb) | —CN | —Br | —I |
| EDX(IIIa) or (IIIb) | —CN | —I | —H |
| EDY(IIIa) or (IIlb) | —CN | —I | —CH₃ |
| EDZ(IIIa) or (IIIb) | —CN | —I | -n-propyl |
| EEA(IIIa) or (IIIb) | —CN | —I | -n-butyl |
| EEB(IIIa) or (IIIb) | —CN | —I | -t-butyl |
| EEC(IIIa) or (IIIb) | —CN | —I | -iso-butyl |
| EED(IIIa) or (IIIb) | —CN | —I | —OCH₃ |
| EEE(IIIa) or (IIIb) | —CN | —I | —OC₂H₅ |
| EEF(IIIa) or (IIIb) | —CN | —1 | —OC₃H₇ |
| EEG(IIIa) or (IIIb) | —CN | —I | —CHF₂ |
| EEH(IIIa) or (IIIb) | —CN | —I | —CF₃ |
| EEI(IIIa) or (IIIb) | —CN | —I | —CHCl₂ |
| EEJ(IIIa) or (IIIb) | —CN | —I | —CCL₃ |
| EEK(IIIa) or (IIIb) | —CN | —I | —F |
| EEL(IIIa) or (IIIb) | —CN | —I | —Cl |
| EEM(IIIa) or (IIIb) | —CN | —I | —Br |
| EEN(IIIa) or (IIIb) | —CN | —I | —I |
| EEO(IIIa) or (IIIb) | —CN | —NO₂ | —H |
| EEP(IIIa) or (IIIb) | —CN | —NO₂ | —CH₃ |
| EEQ(IIIa) or (IIIb) | —CN | —NO₂ | -n-propyl |
| EER(IIIa) or (IIIb) | —CN | —NO₂ | -n-butyl |
| EES(IIIa) or (IIIb) | —CN | —NO₂ | -t-butyl |
| EET(IIIa) or (IIIb) | —CN | —NO₂ | -iso-butyl |
| EEU(IIIa) or (IIIb) | —CN | —NO₂ | —OCH₃ |
| EEV(IIIa) or (IIIb) | —CN | —NO₂ | —OC₂H₅ |
| EEW(IIIa) or (IIIb) | —CN | —NO₂ | —OC₃H₇ |
| EEX(IIIa) or (IIIb) | —CN | —NO₂ | —CHF₂ |
| EEY(IIIa) or (IIIb) | —CN | —NO₂ | —CF₃ |
| EEZ(IIIa) or (IIIb) | —CN | —NO₂ | —CHCl₂ |
| EFA(IIIa) or (IIIb) | —CN | —NO₂ | —CCL₃ |
| EFB(IIIa) or (IIIb) | —CN | —NO₂ | —F |
| EFC(IIIa) or (IIIb) | —CN | —NO₂ | —Cl |
| EFD(IIIa) or (IIIb) | —CN | —NO₂ | —Br |
| EFE(IIIa) or (IIIb) | —CN | —NO₂ | —I |
| EFF(IIIa) or (IIIb) | —CN | —CN | —H |
| EFG(IIIa) or (IIIb) | —CN | —CN | —CH₃ |
| EFH(IIIa) or (IIIb) | —CN | —CN | -n-propyl |
| EFI(IIIa) or (IIIb) | —CN | —CN | -n-butyl |
| EFJ(IIIa) or (IIIb) | —CN | —CN | -t-butyl |
| EFK(IIIa) or (IIIb) | —CN | —CN | -iso-butyl |
| EFL(IIIa) or (IIIb) | —CN | —CN | —OCH₃ |
| EFM(IIIa) or (IIIb) | —CN | —CN | —OC₂h₅ |
| EFN(IIIa) or (IIIb) | —CN | —CN | —OC₃H₇ |
| EFO(IIIa) or (IIIb) | —CN | —CN | —CHF₂ |
| EFP(IIIa) or (IIIb) | —CN | —CN | —CF₃ |
| EFQ(IIIa) or (IIIb) | —CN | —CN | —CHCl₂ |
| EFR(IIIa) or (IIIb) | —CN | —CN | —CCl₃ |
| EFS(IIIa) or (IIIb) | —CN | —CN | —F |
| EFT(IIIa) or (IIIb) | —CN | —CN | —Cl |
| EFU(IIIa) or (IIIb) | —CN | —CN | —Br |
| EFV(IIIa) or (IIIb) | —CN | —CN | —I |
| EFW(IIIa) or (IIIb) | —CN | —NH₂ | —H |
| EFX(IIIa) or (IIIb) | —CN | —NH₂ | —CH₃ |
| EFY(IIIa) or (IIIb) | —CN | —NH₂ | -n-propyl |
| EFZ(IIIa) or (IIIb) | —CN | —NH₂ | -n-butyl |
| EGA(IIIa) or (IIIb) | —CN | —NH₂ | -t-butyl |
| EGB(IIIa) or (IIIb) | —CN | —NH₂ | -iso-butyl |
| EGC(IIIa) or (IIIb) | —CN | —NH₂ | OCH₃ |
| EGD(IIIa) or (IIIb) | —CN | —NH₂ | —OC₂H₅ |
| EGE(IIIa) or (IIIb) | —CN | —NH₂ | —OC₃H₇ |
| EGF(IIIa) or (IIIb) | —CN | —NH₂ | —CHF₂ |
| EGG(IIIa) or (IIIb) | —CN | —NH₂ | —CF₃ |
| EGH(IIIa) or (IIIb) | —CN | —NH₂ | —CHCl₂ |
| EGI(IIIa) or (IIIb) | —CN | —NH₂ | —CCl₃ |
| EGJ(IIIa) or (IIIb) | —CN | —NH₂ | —F |
| EGK(IIIa) or (IIIb) | —CN | —NH₂ | —Cl |
| EGL(IIIa) or (IIIb) | —CN | —NH₂ | —Br |
| EGM(IIIa) or (IIIb) | —CN | —NH₂ | —I |
| EGN(IIIa) or (IIIb) | —CN | —CH₃ | —H |
| EGO(IIIa) or (IIIb) | —CN | —CH₃ | —CH₃ |
| EGP(IIIa) or (IIIb) | —CN | —CH₃ | -n-propyl |
| EGQ(IIIa) or (IIIb) | —CN | —CH₃ | -n-butyl |
| EGR(IIIa) or (IIIb) | —CN | —CH₃ | -t-butyl |
| EGS(IIIa) or (IIIb) | —CN | —CH₃ | -iso-butyl |
| EGT(IIIa) or (IIIb) | —CN | —CH₃ | —OCH₃ |
| EGU(IIIa) or (IIIb) | —CN | —CH₃ | —OC₂H₅ |
| EGV(IIIa) or (IIIb) | —CN | —CH₃ | —OC₃H₇ |
| EGW(IIIa) or (IIIb) | —CN | —CH₃ | —CHF₂ |
| EGX(IIIa) or (IIIb) | —CN | —CH₃ | —CF₃ |
| EGY(IIIa) or (IIIb) | —CN | —CH₃ | —CHCl₂ |
| EGZ(IIIa) or (IIIb) | —CN | —CH₃ | —CCl₃ |
| EHA(IIIa) or (IIIb) | —CN | —CH₃ | —F |
| EHB(IIIa) or (IIIb) | —CN | —CH₃ | —Cl |
| EHC(IIIa) or (IIIb) | —CN | —CH₃ | —Br |
| EHD(IIIa) or (IIIb) | —CN | —CH₃ | —I |
| EHE(TIIa) | —CH₃ | —H | —H |
| EHF(IIIa) | —CH₃ | —H | —CH₃ |
| EHG(IIIa) | —CH₃ | —H | -n-propyl |
| EHH(IIIa) | —CH₃ | —H | -n-butyl |
| EHI(IIIa) | —CH₃ | —H | -t-butyl |
| EHI(IIIa) | —CH₃ | —H | -iso-butyl |
| EHK(IIIa) | —CH₃ | —H | —OCH₃ |
| EHL(IIIa) | —CH₃ | —H | —OC₂H₅ |
| EHM(IIIa) | —CH₃ | —H | —OC₃H₇ |
| EHN(IIIa) | —CH₃ | —H | —CHF₂ |
| EHO(IIIa) | —CH₃ | —H | —CF₃ |
| EHP(IIIa) | —CH₃ | —H | —CHCl₂ |
| EHQ(IIIa) | —CH₃ | —H | —CCl₃ |
| EHR(IIIa) | —CH₃ | —H | —F |
| EHS(IIIa) | —CH₃ | —H | —Cl |
| EHT(IIIa) | —CH₃ | —H | —Br |
| EHU(IIIa) | —CH₃ | —H | —I |
| EHV(IIIa) or (IIIb) | —CH₃ | —OH | —H |
| EHW(IIIa) or (IIIb) | —CH₃ | —OH | —CH₃ |
| EHX(IIIa) or (IIIb) | —CH₃ | —OH | -n-propyl |
| EHY(IIIa) or (IIIb) | —CH₃ | —OH | -n-butyl |
| EHZ(IIIa) or (IIIb) | —CH₃ | —OH | -t-butyl |
| EIA(IIIa) or (IIIb) | —CH₃ | —OH | -iso-butyl |
| EIB(IIIa) or (IIIb) | —CH₃ | —OH | —OCH₃ |
| EIC(IIIa) or (IIIb) | —CH₃ | —OH | —OC₂H₅ |
| EID(IIIa) or (IIIb) | —CH₃ | —OH | —OC₃H₇ |
| EIE(IIIa) or (IIIb) | —CH₃ | —OH | —CHF₂ |
| EIF(IIIa) or (IIIb) | —CH₃ | —OH | —CF₃ |
| EIG(IIIa) or (IIIb) | —CH₃ | —OH | —CHCl₂ |
| EIH(IIIa) or (IIIb) | —CH₃ | —OH | —CCI3 |
| EII(IIIa) or (IIIb) | —CH₃ | —OH | —F |
| EIJ(IIIa) or (IIIb) | —CH₃ | —OH | —CI |
| EIK(IIIa) or (IIIb) | —CH₃ | —OH | —Br |
| EIL(IIIa) or (IIIb) | —CH₃ | —OH | —I |
| EIM(IIIa) or (IIIb) | —CH₃ | —F | —H |
| EIN(IIIa) or (IIIb) | —CH₃ | —F | —CH₃ |
| EIO(IIIa) or (IIIb) | —CH₃ | —F | -n-propyl |
| EIP(IIIa) or (IIIb) | —CH₃ | —F | -n-butyl |
| EIQ(IIIa) or (IIIb) | —CH₃ | —F | -t-butyl |
| EIR(IIIa) or (IIIb) | —CH₃ | —F | -iso-butyl |
| EIS(IIIa) or (IIIb) | —CH₃ | —F | —OCH₃ |
| EIT(IIIa) or (IIIb) | —CH₃ | —F | —OC₂H₅ |
| EIU(IIIa) or (IIIb) | —CH₃ | —F | —OC₃H₇ |
| EIV(IIIa) or (IIIb) | —CH₃ | —F | —CHF₂ |
| EIW(IIIa) or (IIIb) | —CH₃ | —F | —CF₃ |
| EIX(IIIa) or (IIIb) | —CH₃ | —F | —CHCl₂ |
| EIY(IIIa) or (IIIb) | —CH₃ | —F | —CCl₃ |
| EIZ(IIIa) or (IIIb) | —CH₃ | —F | —F |
| EJA(IIIa) or (IIIb) | —CH₃ | —F | —Cl |
| EJB(IIIa) or (IIIb) | —CH, | —F | —Br |
| EJC(IIIa) or (IIIb) | —CH₃ | —F | —I |
| EJD(IIIa) or (IIIb) | —CH₃ | —Cl | —H |
| EJE(IIIa) or (IIIb) | —CH₃ | —Cl | —CH₃ |
| EJF(IIIa) or (IIIb) | —CH₃ | —Cl | -n-propyl |
| EJG(IIIa) or (IIIb) | —CH₃ | —Cl | -n-butyl |
| EJH(IIIa) or (IIIb) | —CH₃ | —Cl | -t-butyl |
| EJI(IIIa) or (IIIb) | —CH₃ | —Cl | -iso-butyl |
| EJJ(IIIa) or (IIIb) | —CH₃ | —Cl | —OCH, |
| EJK(IIIa) or (IIIb) | —CH₃ | —Cl | —OC₂H₅ |
| EJL(IIIa) or (IIIb) | —CH₃ | —Cl | —OC₃H₇ |
| EJM(IIIa) or (IIIb) | —CH₃ | —Cl | —CHF₂ |
| EJN(IIIa) or (IIIb) | —CH₃ | —Cl | —CF₃ |
| EJO(IIIa) or (IIIb) | —CH₃ | —Cl | —CHCL2 |
| EJP(IIIa) or (IIIb) | —CH₃ | —Cl | —CCI3 |
| EJQ(IIIa) or (IIIb) | —CH₃ | —Cl | —F |
| EJR(IIIa) or (IIIb) | —CH₃ | —Cl | —Cl |
| EJS(IIIa) or (IIIb) | —CH₃ | —Cl | —Br |
| EJT(IIIa) or (IIIb) | —CH₃ | —Cl | —I |
| EJU(IIIa) or (IIIb) | —CH₃ | —Br | —H |
| EJV(IIIa) or (IIIb) | —CH₃ | —Br | —CH₃ |
| EJW(IIIa) or (IIIb) | —CH₃ | —Br | -n-propyl |
| EJX(IIIa) or (IIIb) | —CH₃ | —Br | -n-butyl |
| EJY(IIIa) or (IIIb) | —CH₃ | —Br | -t-butyl |
| EJZ(IIIa) or (IIIb) | —CH₃ | —Br | -iso-butyl |
| EKA(IIIa) or (IIIb) | —CH₃ | —Br | —OCH₃ |
| EKB(IIIa) or (IIIb) | —CH₃ | —Br | —OC₂H₅ |
| EKC(IIIa) or (IIIb) | —CH₃ | —Br | —OC₃H₇ |
| EKD(IIIa) or (IIIb) | —CH₃ | —Br | —CHF₂ |
| EKE(IIIa) or (IIIb) | —CH₃ | —Br | —CF₃ |
| EKF(IIIa) or (IIIb) | —CH₃ | —Br | —CHCl₂ |
| EKG(IIIa) or (IIIb) | —CH₃ | —Br | —CCl₃ |
| EKH(IIIa) or (IIIb) | —CH₃ | —Br | —F |
| EKI(IIIa) or (IIIb) | —CH₃ | —Br | —Cl |
| EKI(IIIa) or (IIIb) | —CH₃ | —Br | —Br |
| EKK(IIIa) or (IIIb) | —CH₃ | —Br | —l |
| EKL(IIIa) or (IIIb) | —CH₃ | —I | —H |
| EKM(IIIa) or (IIIb) | —CH₃ | —I | —CH₃ |
| EKN(IIIa) or (IIIb) | —CH₃ | —I | -n-propyl |
| EKO(IIIa) or (IIIb) | —CR3 | —I | -n-butyl |
| EKP(IIIa) or (IIIb) | —CH₃ | —I | -t-butyl |
| EKQ(IIIa) or (IIIb) | —CH₃ | —I | -iso-butyl |
| EKR(IIIa) or (IIIb) | —CH₃ | —I | —OCH₃ |
| EKS(IIIa) or (IIIb) | —CH₃ | —I | —OC₂H₅ |
| EKT(IIIa) or (IIIb) | —CH₃ | —I | —OC₃h₇ |
| EKU(IIIa) or (IIIb) | —CH₃ | —I | —CHF₂ |
| EKV(IIIa) or (IIIb) | —CH₃ | —I | —CF₃ |
| EKW(IIIa) or (IIIb) | —CH₃ | —I | —CHCl₂ |
| EKX(IIIa) or (IIIb) | —CH₃ | —I | —CCl₃ |
| EKY(IIIa) or (IIIb) | —CH₃ | —I | —F |
| EKZ(IIIa) or (IIIb) | —CH₃ | —I | —Cl |
| ELA(IIIa) or (IIIb) | —CH₃ | —I | —Br |
| ELB(IIIa) or (IIIb) | —CH₃ | —I | —I |
| ELC(IIIa) or (IIIb) | —CH₃ | —NO₂ | —H |
| ELD(IIIa) or (IIIb) | —CH₃ | —NO₂ | —CH₃ |
| ELE(IIIa) or (IIIb) | —CH₃ | —NO₂ | -n-propyl |
| ELF(IIIa) or (IIIb) | —CH₃ | —NO₂ | -n-butyl |
| ELG(IIIa) or (IIIb) | —CH₃ | —NO₂ | -t-butyl |
| ELH(IIIa) or (IIIb) | —CH₃ | —NO₂ | -iso-butyl |
| ELI(IIIa) or (IIIb) | —CH₃ | —NO₂ | —OCH₃ |
| ELJ(IIIa) or (IIIb) | —CH₃ | —NO₂ | —OC₂H₅ |
| ELK(IIIa) or (IIIb) | —CH₃ | —NO₂ | —OC₃H₇ |
| ELL(IIIa) or (IIIb) | —CH₃ | —NO₂ | —CHF₂ |
| ELM(IIIa) or (IIIb) | —CH₃ | —NO₂ | —CF₃ |
| ELN(IIIa) or (IIIb) | —CH₃ | —NO₂ | —CHCl₂ |
| ELO(IIla) or (IIIb) | —CH₃ | —NO₂ | —CCl₃ |
| ELP(IIla) or (IIIb) | —CH₃ | —NO₂ | —F |
| ELQ(IIIa) or (IIIb) | —CH₃ | —NO₂ | —Cl |
| ELR(IIIa) or (IIIb) | —CH₃ | —NO₂ | —Br |
| ELS(IIIa) or (IIIb) | —CH₃ | —NO₂ | —I |
| ELT(IIIa) or (IIIb) | —CH₃ | —CN | —H |
| ELU(IIIa) or (IIIb) | —CH₃ | —CN | —CH₃ |
| ELV(IIIa) or (IIIb) | —CH₃ | —CN | -n-propyl |
| ELW(IIIa) or (IIIb) | —CH₃ | —CN | -n-butyl |
| ELX(IIIa) or (IIIb) | —CH₃ | —CN | -t-butyl |
| ELY(IIIa) or (IIIb) | —CH₃ | —CN | -iso-butyl |
| ELZ(IIIa) or (IIIb) | —CH₃ | —CN | —OCH₃ |
| EMA(IIIa) or (IIIb) | —CH₃ | —CN | —OC₂H₅ |
| EMB(IIIa) or (IIIb) | —CH₃ | —CN | —OC₃H₇ |
| EMC(IIIa) or (IIIb) | —CH₃ | —CN | —CHF₂ |
| EMD(IIIa) or (IIIb) | —CH₃ | —CN | —CF₃ |
| EME(IIIa) or (IIIb) | —CH₃ | —CN | —CHCl₂ |
| EMF(IIIa) or (IIIb) | —CH₃ | —CN | —CCI3 |
| EMG(IIIa) or (IIIb) | —CH₃ | —CN | —F |
| EMH(IIIa) or (IIIb) | —CH₃ | —CN | —Cl |
| EMI(IIIa) or (IIIb) | —CH₃ | —CN | —Br |
| EMJ(IIIa) or (IIIb) | —CH₃ | —CN | —I |
| EMK(IIIa) or (IIIb) | —CH₃ | —NH₂ | —H |
| EML(IIIa) or (IIIb) | —CH₃ | —NH₂ | —CH₃ |
| EMM(IIIa) or (IIIb) | —CH₃ | —NH₂ | -n-propyl |
| EMN(IIIa) or (IIIb) | —CH₃ | —NH₂ | -n-butyl |
| EMO(IIIa) or (IIIb) | —CH₃ | —NH₂ | -t-butyl |
| EMP(IIla) or (IIIb) | —CH₃ | —NH₂ | -iso-butyl |
| EMQ(IIIa) or (IIIb) | —CH₃ | —NH₂ | —OCH₃ |
| EMR(IIIa) or (IIIb) | —CH₃ | —NH₂ | —OC₂H₅ |
| EMS(IIIa) or (IIIb) | —CH₃ | —NH₂ | —OC317 |
| EMT(IIIa) or (IIIb) | —CH₃ | —NH₂ | —CHF₂ |
| EMU(IIIa) or (IIIb) | —CH₃ | —NH₂ | —CF₃ |
| EMV(IIIa) or (IIIb) | —CH₃ | —NH₂ | —CHCl₂ |
| EMW(IIIa) or (IIIb) | —CH₃ | —NH₂ | —CCl₃ |
| EMX(IIIa) or (IIIb) | —CH₃ | —NH₂ | —F |
| EMY(IIIa) or (IIIb) | —CH₃ | —NH₂ | —Cl |
| EMZ(IIIa) or (IIIb) | —CH₃ | —NH₂ | —Br |
| ENA(IIIa) or (IIIb) | —CH₃ | —NH₂ | —I |
| ENB(IIIa) or (IIIb) | —CH₃ | —Cl3 | —H |
| ENC(IIIa) or (IIIb) | —CH₃ | —CH₃ | —CH₃ |
| END(IlIa) or (IIIb) | —CH₃ | —CH₃ | -n-propyl |
| ENE(IIIa) or (IIIb) | —CH₃ | —CH₃ | -n-butyl |
| ENF(IIIa) or (IIIb) | —CH₃ | —CH₃ | -t-butyl |
| ENG(IIIa) or (IIIb) | —CH₃ | —CH₃ | -iso-butyl |
| ENH(IIIa) or (IIIb) | —CH₃ | —CH₃ | —OCH₃ |
| ENI(IIIa) or (IIIb) | —CH₃ | —CH₃ | —OC₂H₅ |
| ENJ(IIIa) or (IIIb) | —CH₃ | —CH₃ | —OC₃H₇ |
| ENK(IIIa) or (IIIb) | —CH₃ | —CH₃ | —CHF₂ |
| ENL(IIIa) or (IIIb) | —CH₃ | —CH₃ | —CF₃ |
| ENM(IIIa) or (IIIb) | —CH₃ | —CH₃ | —CHCl₂ |
| ENN(IIIa) or (IIIb) | —CH₃ | —CH₃ | —CCl₃ |
| ENO(IIIa) or (IIIb) | —CH₃ | —CH₃ | —F |
| ENP(IIIa) or (IIIb) | —CE3 | —CH₃ | —Cl |
| ENQ(IIIa) or (IIIb) | —CH₃ | —CH₃ | —Br |
| ENR(IIIa) or (IIIb) | —CH₃ | —CH₃ | —I |

TABLE 3

| | |
|---|---|
| | (IVa) |
| 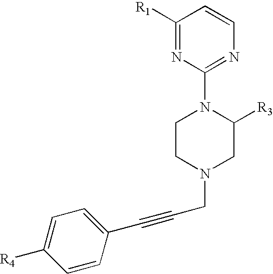 | |
| | (IVb) |
| 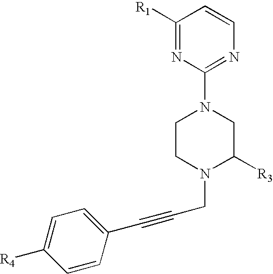 | |

and pharmaceutically acceptable salts thereof, where:

| | | | |
|---|---|---|---|
| Compound | R₁ | R₃ | R₄ |

| | | | |
|---|---|---|---|
| ENS(IVa) | —H | —H | —H |
| ENT(IVa) | —H | —H | —CH₃ |
| ENU(IVa) | —H | —H | -n-propyl |
| ENV(IVa) | —H | —H | -n-butyl |
| ENW(IVa) | —H | —H | -t-butyl |
| ENX(IVa) | —H | —H | -iso-butyl |
| ENY(IVa) | —H | —H | —OCH₃ |
| ENZ(IVa) | —H | —H | —OC₂H₅ |
| EOA(IVa) | —H | —H | —OC₃H₇ |
| EOB(IVa) | —H | —H | —CHF₂ |
| EOC(IVa) | —H | —H | —CF₃ |
| EOD(IVa) | —H | —H | —CHCl₂ |
| EOE(IVa) | —H | —H | —CCl₃ |
| EOF(IVa) | —H | —H | —F |
| EOG(IVa) | —H | —H | —Cl |
| EOH(IVa) | —H | —H | —Br |
| EOI(IVa) | —H | —H | —I |
| EOJ(IVa) or (IVb) | —H | —OH | —H |
| EOK(IVa) or (IVb) | —H | —OH | —CH₃ |
| EOL(IVa) or (IVb) | —H | —OH | -n-propyl |
| EOM(IVa) or (IVb) | —H | —OH | -n-butyl |
| EON(IVa) or (IVb) | —H | —OH | -t-butyl |
| EOO(IVa) or (IVb) | —H | —OH | -iso-butyl |
| EOP(IVa) or (IVb) | —H | —OH | —OCH₃ |
| EOQ(IVa) or (IVb) | —H | —OH | —OC₂H₅ |
| EOR(IVa) or (IVb) | —H | —OH | —OC₃H₇ |
| EOS(IVa) or (IVb) | —H | —OH | —CHF₂ |
| EOT(IVa) or (IVb) | —H | —OH | —CF₃ |
| EOU(IVa) or (IVb) | —H | —OH | —CHCl₂ |
| EOV(IVa) or (IVb) | —H | —OH | —CCl₃ |
| EOW(lVa) or (IVb) | —H | —OH | —F |
| EOX(IVa) or (IVb) | —H | —OH | —Cl |
| EOY(IVa) or (IVb) | —H | —OH | —Br |
| EOZ(IVa) or (IVb) | —H | —OH | —I |
| EPA(IVa) or (IVb) | —H | —F | —H |
| EPB(lVa) or (IVb) | —H | —F | —CH₃ |
| EPC(IVa) or (IVb) | —H | —F | -n-propyl |
| EPD(IVa) or (IVb) | —H | —F | -n-butyl |
| EPE(IVa) or (IVb) | —H | —F | -t-butyl |
| EPF(IVa) or (IVb) | —H | —F | -iso-butyl |
| EPG(IVa) or (IVb) | —H | —F | —OCH₃ |
| EPH(IVa) or (IVb) | —H | —F | —OC₂H₅ |
| EPI(IVa) or (IVb) | —H | —F | —OC₃H₇ |
| EPJ(IVa) or (IVb) | —H | —F | —CHF₂ |
| EPK(IVa) or (IVb) | —H | —F | —CF₃ |
| EPL(IVa) or (IVb) | —H | —F | —CHCl₂ |
| EPM(IVa) or (IVb) | —H | —F | —CCl₃ |
| EPN(IVa) or (IVb) | —H | —F | —F |
| EPO(IVa) or (IVb) | —H | —F | —Cl |
| EPP(IVa) or (IVb) | —H | —F | —Br |
| EPQ(IVa) or (IVb) | —H | —F | —I |
| EPR(IVa) or (IVb) | —H | —Cl | —H |
| EPS(IVa) or (IVb) | —H | —Cl | —CH₃ |
| EPT(IVa) or (IVb) | —H | —Cl | -n-propyl |
| EPU(IVa) or (IVb) | —H | —Cl | -n-butyl |
| EPV(IVa) or (IVb) | —H | —Cl | -t-butyl |
| EPW(IVa) or (IVb) | —H | —Cl | -iso-butyl |
| EPX(IVa) or (IVb) | —H | —Cl | —OCH₃ |
| EPY(IVa) or (IVb) | —H | —Cl | —OC₂H₅ |
| EPZ(IVa) or (IVb) | —H | —Cl | —OC₃H₇ |
| EQA(IVa) or (IVb) | —H | —Cl | —CHF₂ |
| EQB(IVa) or (IVb) | —H | —Cl | —CF₃ |
| EQC(IVa) or (IVb) | —H | —Cl | —CHCl₂ |
| EQD(IVa) or (IVb) | —H | —Cl | —CCl₃ |
| EQE(IVa) or (IVb) | —H | —Cl | —F |
| EQF(IVa) or (IVb) | —H | —Cl | —Cl |
| EQG(IVa) or (IVb) | —H | —Cl | —Br |
| EQH(IVa) or (IVb) | —H | —Cl | —I |
| EQI(IVa) or (IVb) | —H | —Br | —H |
| EQJ(IVa) or (IVb) | —H | —Br | —CH₃ |
| EQK(IVa) or (IVb) | —H | —Br | -n-propyl |
| EQL(IVa) or (IVb) | —H | —Br | -n-butyl |
| EQM(IVa) or (IVb) | —H | —Br | -t-butyl |
| EQN(IVa) or (IVb) | —H | —Br | -iso-butyl |
| EQO(IVa) or (IVb) | —H | —Br | —OCH₃ |
| EQP(IVa) or (IVb) | —H | —Br | —OC₂H₅ |
| EQQ(IVa) or (IVb) | —H | —Br | —OC₃H₇ |
| EQR(IVa) or (IVb) | —H | —Br | —CHF₂ |
| EQS(IVa) or (IVb) | —H | —Br | —CF₃ |
| EQT(IVa) or (IVb) | —H | —Br | —CHCl₂ |
| EQU(IVa) or (IVb) | —H | —Br | —CCl₃ |
| EQV(IVa) or (IVb) | —H | —Br | —F |
| EQW(IVa) or (IVb) | —H | —Br | —Cl |
| EQX(IVa) or (IVb) | —H | —Br | —Br |
| EQY(IVa) or (IVb) | —H | —Br | —I |
| EQZ(IVa) or (IVb) | —H | —I | —H |
| ERA(IVa) or (IVb) | —H | —I | —CH₃ |
| ERB(IVa) or (IVb) | —H | —I | -n-propyl |
| ERC(IVa) or (IVb) | —H | —I | -n-butyl |
| ERD(IVa) or (IVb) | —H | —I | -t-butyl |
| ERE(IVa) or (IVb) | —H | —I | -iso-butyl |
| ERF(IVa) or (IVb) | —H | —I | —OCH₃ |
| ERG(IVa) or (IVb) | —H | —I | —OC₂H₅ |
| ERH(IVa) or (IVb) | —H | —I | —OC₃H₇ |
| ERI(IVa) or (IVb) | —H | —I | —CHF₂ |
| ERJ(IVa) or (IVb) | —H | —I | —CF₃ |
| ERK(IVa) or (IVb) | —H | —I | —CHCl₂ |
| ERL(IVa) or (IVb) | —H | —I | —CCl₃ |
| ERM(IVa) or (IVb) | —H | —I | —F |
| ERN(IVa) or (IVb) | —H | —I | —Cl |
| ERO(IVa) or (IVb) | —H | —I | —Br |
| ERP(IVa) or (IVb) | —H | —I | —I |
| ERQ(IVa) or (IVb) | —H | —NO₂ | —H |
| ERR(IVa) or (IVb) | —H | —NO₂ | —CH₃ |
| ERS(IVa) or (IVb) | —H | —NO₂ | -n-propyl |
| ERT(IVa) or (IVb) | —H | —NO₂ | -n-butyl |
| ERU(IVa) or (IVb) | —H | —NO₂ | -t-butyl |
| ERV(IVa) or (IVb) | —H | —NO₂ | -iso-butyl |
| ERW(IVa) or (IVb) | —H | —NO₂ | —OCH₃ |
| ERX(IVa) or (IVb) | —H | —NO₂ | —OC₂H₅ |
| ERY(IVa) or (IVb) | —H | —NO₂ | —OC₃H₇ |
| ERZ(IVa) or (IVb) | —H | —NO₂ | —CHF₂ |
| ESA(IVa) or (IVb) | —H | —NO₂ | —CF₃ |
| ESB(IVa) or (IVb) | —H | —NO₂ | —CHCl₂ |
| ESC(IVa) or (IVb) | —H | —NO₂ | —CCl₃ |
| ESD(IVa) or (IVb) | —H | —NO₂ | —F |
| ESE(IVa) or (IVb) | —H | —NO₂ | —Cl |
| ESF(IVa) or (IVb) | —H | —NO₂ | —Br |
| ESG(IVa) or (IVb) | —H | —NO₂ | —I |
| ESH(IVa) or (IVb) | —H | —CN | —H |
| ESI(IVa) or (IVb) | —H | —CN | —CH₃ |
| ESJ(IVa) or (IVb) | —H | —CN | -n-propyl |
| ESK(IVa) or (IVb) | —H | —CN | -n-butyl |
| ESL(IVa) or (IVb) | —H | —CN | -t-butyl |
| ESM(IVa) or (IVb) | —H | —CN | -iso-butyl |
| ESN(IVa) or (IVb) | —H | —CN | —OCH₃ |
| ESO(IVa) or (IVb) | —H | —CN | —OC₂H₅ |
| ESP(IVa) or (IVb) | —H | —CN | —OC₃H₇ |
| ESQ(IVa) or (IVb) | —H | —CN | —CHF₂ |
| ESR(IVa) or (IVb) | —H | —CN | —CF₃ |
| ESS(IVa) or (IVb) | —H | —CN | —CHCl₂ |
| EST(IVa) or (IVb) | —H | —CN | —CCl₃ |
| ESU(IVa) or (IVb) | —H | —CN | —F |
| ESX(IVa) or (IVb) | —H | —CN | —Cl |
| ESW(IVa) or (IVb) | —H | —CN | —Br |
| ESX(IVa) or (IVb) | —H | —CN | —I |
| ESY(IVa) or (IVb) | —H | —NH₂ | —H |
| ESZ(IVa) or (IVb) | —H | —NH₂ | CH₃ |
| ETA(IVa) or (IVb) | —H | —NH₂ | -n-propyl |
| ETB(IVa) or (IVb) | —H | —NH₂ | -n-butyl |
| ETC(IVa) or (IVb) | —H | —NH₂ | -t-butyl |
| ETD(IVa) or (IVb) | —H | —NH₂ | -iso-butyl |
| ETE(IVa) or (IVb) | —H | —NH₂ | —OCH₃ |
| ETF(IVa) or (IVb) | —H | —NH₂ | —OC₂H₅ |
| ETG(IVa) or (IVb) | —H | —NH₂ | —OC₃H₇ |
| ETH(IVa) or (IVb) | —H | —NH₂ | —CHF₂ |
| ETI(IVa) or (IVb) | —H | —NH₂ | —CF₃ |
| ETJ(IVa) or (IVb) | —H | —NH₂ | —CHCl₂ |
| ETK(IVa) or (IVb) | —H | —NH₂ | —CCl₃ |
| ETL(IVa) or (IVb) | —H | —NH₂ | —F |
| ETM(IVa) or (IVb) | —H | —NH₂ | —Cl |
| ETN(IVa) or (IVb) | —H | —NH₂ | —Br |
| ETO(IVa) or (IVb) | —H | —NH₂ | —I |
| ETP(IVa) or (IVb) | —H | —CH₃ | —H |
| ETQ(IVa) or (IVb) | —H | —CH₃ | —CH₃ |
| ETR(IVa) or (IVb) | —H | —CH₃ | -n-propyl |
| ETS(IVa) or (IVb) | —H | —CH₃ | -n-butyl |
| ETT(IVa) or (IVb) | —H | —CH₃ | -t-butyl |
| ETU(IVa) or (IVb) | —H | —CH₃ | -iso-butyl |
| ETV(IVa) or (IVb) | —H | —CH₃ | —OCH₃ |
| ETW(IVa) or (IVb) | —H | —CH₃ | —OC₂H₅ |
| ETX(IVa) or (IVb) | —H | —CH₃ | —OC₃H₇ |
| ETY(IVa) or (IVb) | —H | —CH₃ | —CHF₂ |
| ETZ(IVa) or (IVb) | —H | —CH₃ | —CF₃ |
| EUA(IVa) or (IVb) | —H | —CH₃ | —CHCl₂ |
| EUB(IVa) or (IVb) | —H | —CH₃ | —CCl₃ |
| EUC(IVa) or (IVb) | —H | —CH₃ | —F |
| EUD(IVa) or (IVb) | —H | —CH₃ | —Cl |
| EUE(IVa) or (IVb) | —H | —CH₃ | —Br |
| EUF(IVa) or (IVb) | —H | —CH₃ | —I |
| EUG(IVa) | —OH | —H | —H |
| EUI(IVa) | —OH | —H | —CH₃ |
| EUI(IVa) | —OH | —H | -n-propyl |
| EUJ(IVa) | —OH | —H | -n-butyl |
| EUK(IVa) | —OH | —H | -t-butyl |
| EUL(IVa) | —OH | —H | -iso-butyl |
| EUM(IVa) | —OH | —H | —OCH₃ |
| EUN(IVa) | —OH | —H | —OC₂H₅ |
| EUO(IVa) | —OH | —H | —OC₃H₇ |
| EUP(IVa) | —OH | —H | —CHF₂ |
| EUQ(IVa) | —OH | —H | —CF₃ |
| EUR(IVa) | —OH | —H | —CHCl₂ |
| EUS(IVa) | —OH | —H | —CCL₃ |
| EUT(IVa) | —OH | —H | —F |
| EUU(IVa) | —OH | —H | —Cl |
| EUV(IVa) | —OH | —H | —Br |
| EUW(IVa) | —OH | —H | —I |
| EUX(IVa) or (IVb) | —OH | —OH | —H |
| EUY(IVa) or (IVb) | —OH | —OH | —CH₃ |
| EUZ(IVa) or (IVb) | —OH | —OH | -n-propyl |
| EVA(IVa) or (IVb) | —OH | —OH | -n-butyl |
| EVB(IVa) or (IVb) | —OH | —OH | -t-butyl |
| EVC(IVa) or (IVb) | —OH | —OH | -iso-butyl |
| EVD(IVa) or (IVb) | —OH | —OH | —OCH₃ |
| EVE(IVa) or (IVb) | —OH | —OH | —OC₂H₅ |
| EVF(IVa) or (IVb) | —OH | —OH | —OC₃H₇ |
| EVG(IVa) or (IVb) | —OH | —OH | —CHF₂ |
| EVH(IVa) or (IVb) | —OH | —OH | —CF₃ |
| EVI(IVa) or (IVb) | —OH | —OH | —CHCl₂ |
| EVJ(IVa) or (IVb) | —OH | —OH | —CCl₃ |
| EVK(IVa) or (IVb) | —OH | —OH | —F |
| EVL(IVa) or (IVb) | —OH | —OH | —Cl |
| EVM(IVa) or (IVb) | —OH | —OH | —Br |
| EVN(IVa) or (IVb) | —OH | —OH | —I |
| EVO(IVa) or (IVb) | —OH | —F | —H |
| EVP(IVa) or (IVb) | —OH | —F | —CH₃ |
| EVQ(IVa) or (IVb) | —OH | —F | -n-propyl |
| EVR(IVa) or (IVb) | —OH | —F | -n-butyl |
| EVS(IVa) or (IVb) | —OH | —F | -t-butyl |
| EVT(IVa) or (IVb) | —OH | —F | -iso-butyl |
| EVU(IVa) or (IVb) | —OH | —F | —OCH₃ |
| EVV(IVa) or (IVb) | —OH | —F | —OC₂H₅ |
| EVW(IVa) or (IVb) | —OH | —F | —OC₃H₇ |
| EVX(IVa) or (IVb) | —OH | —F | —CHF₂ |
| EVY(IVa) or (IVb) | —OH | —F | —CF₃ |
| EVZ(IVa) or (IVb) | —OH | —F | —CHCl₂ |
| EWA(IVa) or (IVb) | —OH | —F | —CCl₃ |
| EWB(IVa) or (IVb) | —OH | —F | —F |
| EWC(IVa) or (IVb) | —OH | —F | —Cl |
| EWD(IVa) or (IVb) | —OH | —F | —Br |
| EWE(IVa) or (IVb) | —OH | —F | —I |
| EWF(IVa) or (IVb) | —OH | —Cl | —H |
| EWG(IVa) or (IVb) | —OH | —Cl | —CH₃ |
| EWH(IVa) or (IVb) | —OH | —Cl | -n-propyl |
| EWI(IVa) or (IVb) | —OH | —Cl | -n-butyl |
| EWJ(IVa) or (IVb) | —OH | —Cl | -t-butyl |
| EWK(IVa) or (IVb) | —OH | —Cl | -iso-butyl |
| EWL(IVa) or (IVb) | —OH | —Cl | —OCH₃ |
| EWM(IVa) or (IVb) | —OH | —Cl | —OC₂H₅ |
| EWN(LVa) or (IVb) | —OH | —Cl | —OC₃H₇ |
| EWO(IVa) or (IVb) | —OH | —Cl | —CHF₂ |
| EWP(IVa) or (IVb) | —OH | —Cl | —CF₃ |
| EWQ(IVa) or (IVb) | —OH | —Cl | —CHCl₂ |
| EWR(IVa) or (IVb) | —OH | —Cl | —CCl₃ |
| EWS(IVa) or (IVb) | —OH | —Cl | —F |
| EWT(IVa) or (IVb) | —OH | —Cl | —Cl |
| EWU(IVa) or (IVb) | —OH | —Cl | —Br |
| EWV(IVa) or (IVb) | —OH | —Cl | —I |
| EWW(IVa) or (IVb) | —OH | —Br | —H |
| EWX(IVa) or (IVb) | —OH | —Br | —CH₃ |
| EWY(IVa) or (IVb) | —OH | —Br | -n-propyl |
| EWZ(IVa) or (IVb) | —OH | —Br | -n-butyl |
| EXA(IVa) or (IVb) | —OH | —Br | -t-butyl |
| EXB(IVa) or (IVb) | —OH | —Br | -iso-butyl |
| EXC(IVa) or (IVb) | —OH | —Br | —OCH₃ |
| EXD(IVa) or (IVb) | —OH | —Br | —OC₂H₅ |
| EXE(IVa) or (IVb) | —OH | —Br | —OC₃H₇ |
| EXF(IVa) or (IVb) | —OH | —Br | —CHF₂ |
| EXG(IVa) or (IVb) | —OH | —Br | —CF₃ |
| EXH(IVa) or (IVb) | —OH | —Br | —CHCl₂ |
| EXI(IVa) or (IVb) | —OH | —Br | —CCl₃ |
| EXJ(IVa) or (IVb) | —OH | —Br | —F |
| EXK(IVa) or (IVb) | —OH | —Br | —Cl |
| EXL(IVa) or (IVb) | —OH | —Br | —Br |
| EXM(IVa) or (IVb) | —OH | —Br | —L |
| EXN(IVa) or (IVb) | —OH | —I | —H |
| EXO(IVa) or (IVb) | —OH | —I | —CH₃ |
| EXP(IVa) or (IVb) | —OH | —I | -n-propyl |
| EXQ(IVa) or (IVb) | —OH | —I | -n-butyl |
| EXR(IVa) or (IVb) | —OH | —I | -t-butyl |
| EXS(IVa) or (IVb) | —OH | —I | -iso-butyl |
| EXT(IVa) or (IVb) | —OH | —I | —OCH₃ |
| EXU(IVa) or (IVb) | —OH | —I | —OC₂H₅ |
| EXV(IVa) or (IVb) | —OH | —I | —OC₃H₇ |
| EXW(IVa) or (IVb) | —OH | —I | —CHF₂ |
| EXX(IVa) or (IVb) | —OH | —I | —CF₃ |
| EXY(IVa) or (IVb) | —OH | —I | —CHCl₂ |
| EXZ(IVa) or (IVb) | —OH | —I | —CCl₃ |
| EYA(IVa) or (IVb) | —OH | —I | —F |
| EYB(IVa) or (IVb) | —OH | —I | —Cl |
| EYC(IVa) or (IVb) | —OH | —I | —Br |
| EYD(IVa) or (IVb) | —OH | —I | —I |
| EYE(IVa) or (IVb) | —OH | —NO₂ | —H |
| EYF(IVa) or (IVb) | —OH | —NO₂ | —CH₃ |
| EYG(IVa) or (IVb) | —OH | —NO₂ | -n-propyl |
| EYH(LVa) or (IVb) | —OH | —NO₂ | -n-butyl |
| EYI(IVa) or (IVb) | —OH | —NO₂ | -t-butyl |
| EYJ(IVa) or (IVb) | —OH | —NO₂ | -iso-butyl |
| EYK(IVa) or (IVb) | —OH | —NO₂ | —OCH₃ |
| EYL(IVa) or (IVb) | —OH | —NO₂ | —OC₂H₅ |
| EYM(IVa) or (IVb) | —OH | —NO₂ | —OC₃H₇ |
| EYN(IVa) or (IVb) | —OH | —NO₂ | —CHF₂ |
| EYO(IVa) or (IVb) | —OH | —NO₂ | —CF₃ |
| EYP(IVa) or (IVb) | —OH | —NO₂ | —CHCl₂ |
| EYQ(IVa) or (IVb) | —OH | —NO₂ | —CCl₃ |
| EYR(IVa) or (IVb) | —OH | —NO₂ | —F |
| EYS(IVa) or (IVb) | —OH | —NO₂ | —Cl |
| EYT(IVa) or (IVb) | —OH | —NO₂ | —Br |
| EYU(IVa) or (IVb) | —OH | —NO₂ | —I |
| EYV(IVa) or (IVb) | —OH | —CN | —H |
| EYW(IVa) or (IVb) | —OH | —CN | —CH₃ |
| EYX(IVa) or (IVb) | —OH | —CN | -n-propyl |
| EYY(IVa) or (IVb) | —OH | —CN | -n-butyl |
| EYZ(IVa) or (IVb) | —OH | —CN | -t-butyl |
| EZA(IVa) or (IVb) | —OH | —CN | -iso-butyl |
| EZB(IVa) or (IVb) | —OH | —CN | —OCH₃ |
| EZC(IVa) or (IVb) | —OH | —CN | —OC₂H₅ |
| EZD(IVa) or (IVb) | —OH | —CN | —OC₃H₇ |
| EZE(IVa) or (IVb) | —OH | —CN | —CHF₂ |
| EZF(IVa) or (IVb) | —OH | —CN | —CF₃ |
| EZG(IVa) or (IVb) | —OH | —CN | —CHCl₂ |
| EZH(IVa) or (IVb) | —OH | —CN | —CCl₃ |
| EZI(IVa) or (IVb) | —OH | —CN | —F |
| EZJ(IVa) or (IVb) | —OH | —CN | —Cl |
| EZK(IVa) or (IVb) | —OH | —CN | —Br |
| EZL(IVa) or (IVb) | —OH | —CN | —I |
| EZM(IVa) or (IVb) | —OH | —NH₂ | —H |
| EZN(IVa) or (IVb) | —OH | —NH₂ | —CH₃ |
| EZO(IVa) or (IVb) | —OH | —NH₂ | -n-propyl |
| EZP(IVa) or (IVb) | —OH | —NH₂ | -n-butyl |
| EZQ(IVa) or (IVb) | —OH | —NH₂ | -t-butyl |
| EZR(IVa) or (IVb) | —OH | —NH₂ | -iso-butyl |
| EZS(IVa) or (IVb) | —OH | —NH₂ | —OCH₃ |
| EZT(IVa) or (IVb) | —OH | —NH₂ | —OC₂H₅ |
| EZU(IVa) or (IVb) | —OH | —NH₂ | OC₃H₇ |
| EZV(IVa) or (IVb) | —OH | —NH₂ | —CHF₂ |
| EZW(IVa) or (IVb) | —OH | —NH₂ | —CF₃ |
| EZX(IVa) or (IVb) | —OH | —NH₂ | CHCl₂ |
| EZY(IVa) or (IVb) | —OH | —NH₂ | —CCl₃ |
| EZZ(IVa) or (IVb) | —OH | —NH₂ | —F |
| FAA(IVa) or (IVb) | —OH | —NH₂ | —Cl |
| FAB(IVa) or (IVb) | —OH | —NH₂ | —Br |
| FAC(IVa) or (IVb) | —OH | —NH₂ | —I |
| FAD(IVa) or (IVb) | —OH | —CH₃ | —H |
| FAE(IVa) or (IVb) | —OH | —CH₃ | —CH₃ |
| FAF(IVa) or (IVb) | —OH | —CH₃ | -n-propyl |
| FAG(IVa) or (IVb) | —OH | —CH₃ | -n-butyl |
| FAH(IVa) or (IVb) | —OH | —CH₃ | -t-butyl |
| FAI(IVa) or (IVb) | —OH | —CH₃ | -iso-butyl |
| FAJ(IVa) or (IVb) | —OH | —CH₃ | —OCH₃ |
| FAK(IVa) or (IVb) | —OH | —CH₃ | —OC₂H₅ |
| FAL(IVa) or (IVb) | —OH | —CH₃ | —OC₃H₇ |
| FAM(IVa) or (IVb) | —OH | —CH₃ | —CHF₂ |
| FAN(IVa) or (IVb) | —OH | —CH₃ | —CF₃ |
| FAO(IVa) or (IVb) | —OH | —CH₃ | —CHCl₂ |
| FAP(IVa) or (IVb) | —OH | —CH₃ | —CCl₃ |
| FAQ(IVa) or (IVb) | —OH | —CH₃ | —F |
| FAR(IVa) or (IVb) | —OH | —CH₃ | —Cl |
| FAS(IVa) or (IVb) | —OH | —CH₃ | —Br |
| FAT(IVa) or (IVb) | —OH | —CH₃ | —I |
| FAU(IVa) | —F | —H | —H |
| FAV(IVa) | —F | —H | —CH₃ |
| FAW(IVa) | —F | —H | -n-propyl |
| FAX(IVa) | —F | —H | -n-butyl |
| FAY(IVa) | —F | —H | -t-butyl |
| FAZ(IVa) | —F | —H | -iso-butyl |
| FBA(IVa) | —F | —H | —OCH₃ |
| FBB(IVa) | —F | —H | —OC₂H₅ |
| FBC(IVa) | —F | —H | —OC₃H₇ |
| FBD(IVa) | —F | —H | —CHF₂ |
| FBE(IVa) | —F | —H | —CF₃ |
| FBF(IVa) | —F | —H | —CHCl₂ |
| FBG(IVa) | —F | —H | —CCl₃ |
| FBH(IVa) | —F | —H | —F |
| FBI(IVa) | —F | —H | —Cl |
| FBJ(IVa) | —F | —H | —Br |
| FBK(IVa) | —F | —H | —I |
| FBL(IVa) or (IVb) | —F | —OH | —H |
| FBM(IVa) or (IVb) | —F | —OH | —CH₃ |
| FBN(IVa) or (IVb) | ‘3F | —OH | -n-propyl |
| FBO(IVa) or (IVb) | —F | —OH | -n-butyl |
| FBP(IVa) or (IVb) | —F | —OH | -t-butyl |
| FBQ(IVa) or (IVb) | —F | —OH | -iso-butyl |
| FBR(IVa) or (IVb) | —F | —OH | —OCH₃ |
| FBS(IVa) or (IVb) | —F | —OH | —OC₂H₅ |
| FBT(IVa) or (IVb) | —F | —OH | —OC₃H₇ |
| FBU(IVa) or (IVb) | —F | —OH | —CHF₂ |
| FBV(IVa) or (IVb) | —F | —OH | —CF₃ |
| FBW(IVa) or (IVb) | —F | —OH | —CHCl₂ |
| FBX(IVa) or (IVb) | —F | —OH | —CCl₃ |
| FBY(IVa) or (IVb) | —F | —OH | —F |
| FBZ(IVa) or (IVb) | —F | —OH | —Cl |
| FCA(IVa) or (IVb) | —F | —OH | —Br |
| FCB(IVa) or (IVb) | —F | —OH | —I |
| FCC(IVa) or (IVb) | —F | —F | —H |
| FCD(IVa) or (IVb) | —F | —F | —CH₃ |
| FCE(IVa) or (IVb) | —F | —F | -n-propyl |
| FCF(IVa) or (IVb) | —F | —F | -n-butyl |
| FCG(IVa) or (IVb) | —F | —F | -t-butyl |
| FCH(IVa) or (IVb) | —F | —F | -iso-butyl |
| FCI(IVa) or (IVb) | —F | —F | —OCH₃ |
| FCJ(IVa) or (IVb) | —F | —F | —OC₂H₅ |
| FCK(IVa) or (IVb) | —F | —F | —OC₃H₇ |
| FCL(IVa) or (IVb) | —F | —F | —CHF₂ |
| FCM(IVa) or (IVb) | —F | —F | —CF₃ |
| FCN(IVa) or (IVb) | —F | —F | —CHCl₂ |
| FCO(IVa) or (IVb) | —F | —F | —CCl₃ |
| FCP(IVa) or (IVb) | —F | —F | —F |
| FCQ(IVa) or (IVb) | —F | —F | —Cl |
| FCR(IVa) or (IVb) | —F | —F | —Br |
| FCS(IVa) or (IVb) | —F | —F | —I |
| FCT(IVa) or (IVb) | —F | —Cl | —H |
| FCU(IVa) or (IVb) | —F | —Cl | —CH₃ |
| FCV(IVa) or (IVb) | —F | —Cl | -n-propyl |
| FCW(IVa) or (IVb) | —F | —Cl | -n-butyl |
| FCX(IVa) or (IVb) | —F | —Cl | -t-butyl |
| FCY(IVa) or (IVb) | —F | —Cl | -iso-butyl |
| FCZ(IVa) or (IVb) | —F | —Cl | —OCH₃ |
| FDA(IVa) or (IVb) | —F | —Cl | —OC₂H₅ |
| FDB(IVa) or (IVb) | —F | —Cl | —OC₃H₇ |
| FDC(IVa) or (IVb) | —F | —Cl | —CHF₂ |
| FDD(IVa) or (IVb) | —F | —Cl | —CF₃ |
| FDE(IVa) or (IVb) | —F | —Cl | —CHCl₂ |
| FDF(IVa) or (IVb) | —F | —Cl | —CCl₃ |
| FDG(IVa) or (IVb) | —F | —Cl | —F |
| FDH(IVa) or (IVb) | —F | —Cl | —Cl |
| FDI(IVa) or (IVb) | —F | —Cl | —Br |
| FDJ(IVa) or (IVb) | —F | —Cl | —I |
| FDK(IVa) or (IVb) | —F | —Br | —H |
| FDL(IVa) or (IVb) | —F | —Br | —CH₃ |
| FDM(IVa) or (IVb) | —F | —Br | -n-propyl |
| FDN(IVa) or (IVb) | —F | —Br | -n-butyl |
| FDO(IVa) or (IVb) | —F | —Br | -t-butyl |
| FDP(IVa) or (IVb) | —F | —Br | -iso-butyl |
| FDQ(IVa) or (IVb) | —F | —Br | —OCH₃ |
| FDR(IVa) or (IVb) | —F | —Br | —OC₂H₅ |
| FDS(IVa) or (IVb) | —F | —Br | —OC₃H₇ |
| FDT(IVa) or (IVb) | —F | —Br | —CHF₂ |
| FDU(IVa) or (IVb) | —F | —Br | —CF₃ |
| FDV(IVa) or (IVb) | —F | —Br | —CHCl₂ |
| FDW(IVa) or (IVb) | —F | —Br | —CCl₃ |
| FDX(IVa) or (IVb) | —F | —Br | —F |
| FDY(IVa) or (IVb) | —F | —Br | —Cl |
| FDZ(IVa) or (IVb) | —F | —Br | —Br |
| FEA(IVa) or (IVb) | —F | —Br | —I |
| FEB(IVa) or (IVb) | —F | —I | —H |
| FEC(IVa) or (IVb) | —F | —I | —CH₃ |
| FED(IVa) or (IVb) | —F | —I | -n-propyl |
| FEE(IVa) or (IVb) | —F | —I | -n-butyl |
| FEF(IVa) or (IVb) | —F | —I | -t-butyl |
| FEG(IVa) or (IVb) | —F | —I | -iso-butyl |
| FEH(IVa) or (IVb) | —F | —I | —OCH₃ |
| FEI(IVa) or (IVb) | —F | —L | —OC₂H₅ |
| FEJ(IVa) or (IVb) | —F | —I | —OC₃H₇ |
| FEK(IVa) or (IVb) | —F | —I | —CHF₂ |
| FEL(IVa) or (IVb) | —F | —I | —CF₃ |
| FEM(IVa) or (IVb) | —F | —I | —CHCl₂ |
| FEN(IVa) or (IVb) | —F | —I | —CCl₃ |
| FEO(IVa) or (IVb) | —F | —I | —F |
| FEP(IVa) or (IVb) | —F | —I | —Cl |
| FEQ(IVa) or (IVb) | —F | —I | —Br |
| FER(IVa) or (IVb) | —F | —I | —I |
| FES(IVa) or (IVb) | —F | —NO₂ | —H |
| FET(IVa) or (IVb) | —F | —NO₂ | —CH₃ |
| FEU(IVa) or (IVb) | —F | —NO₂ | -n-propyl |
| FEV(IVa) or (IVb) | —F | —NO₂ | -n-butyl |
| FEW(IVa) or (IVb) | —F | —NO₂ | -t-butyl |
| FEX(IVa) or (IVb) | —F | —NO₂ | -iso-butyl |
| FEY(IVa) or (IVb) | —F | —NO₂ | —OCH₃ |
| FEZ(IVa) or (IVb) | —F | —NO₂ | —OC₂H₅ |
| FFA(IVa) or (IVb) | —F | —NO₂ | —OC₃H₇ |
| FFB(IVa) or (IVb) | —F | —NO₂ | —CHF₂ |
| FFC(IVa) or (IVb) | —F | —NO₂ | —CF₃ |
| FFD(IVa) or (IVb) | —F | —NO₂ | —CHCl₂ |
| FFE(IVa) or (IVb) | —F | —NO₂ | —CCl₃ |
| FFF(IVa) or (IVb) | —F | —NO₂ | —F |
| FFG(IVa) or (IVb) | —F | —NO₂ | —Cl |
| FFH(IVa) or (IVb) | —F | —NO₂ | —Br |
| FFI(iVa) or (IVb) | —F | —NO₂ | —I |
| FFJ(IVa) or (IVb) | —F | —CN | —H |
| FFK(IVa) or (IVb) | —F | —CN | —CH₃ |
| FFL(IVa) or (IVb) | —F | —CN | -n-propyl |
| FFM(IVa) or (IVb) | —F | —CN | -n-butyl |
| FFN(IVa) or (IVb) | —F | —CN | -t-butyl |
| FFO(IVa) or (IVb) | —F | —CN | -iso-butyl |
| FFP(IVa) or (IVb) | —F | —CN | —OCH₃ |
| FFQ(IVa) or (IVb) | —F | —CN | —OC₂H₅ |
| FFR(IVa) or (IVb) | —F | —CN | —OC₃H₇ |
| FFS(IVa) or (IVb) | —F | —CN | —CHF₂ |
| FFT(IVa) or (IVb) | —F | —CN | —CF₃ |
| FFU(IVa) or (IVb) | —F | —CN | —CHCl₂ |
| FFV(IVa) or (IVb) | —F | —CN | —CCl₃ |
| FFW(IVa) or (IVb) | —F | —CN | —F |
| FFX(IVa) or (IVb) | —F | —CN | —Cl |
| FFY(IVa) or (IVb) | —F | —CN | —Br |
| FFZ(IVa) or (IVb) | —F | —CN | —I |
| FGA(IVa) or (IVb) | —F | —NH₂ | —H |
| FGB(IVa) or (IVb) | —F | —NH₂ | —CH₃ |
| FGC(IVa) or (IVb) | —F | —NH₂ | -n-propyl |
| FGD(IVa) or (IVb) | —F | —NH₂ | -n-butyl |
| FGE(IVa) or (IVb) | —F | —NH₂ | -t-butyl |
| FGF(IVa) or (IVb) | —F | —NH₂ | -iso-butyl |
| FGG(IVa) or (IVb) | —F | —NH₂ | —OCH₃ |
| FGH(IVa) or (IVb) | —F | —NH₂ | —OC₂H₅ |
| FGI(IVa) or (IVb) | —F | —NH₂ | —OC₃H₇ |
| FGJ(IVa) or (IVb) | —F | —NH₂ | —CHF₂ |
| FGK(IVa) or (IVb) | —F | —NH₂ | —CF₃ |
| FGL(IVa) or (IVb) | —F | —NH₂ | —CHCl₂ |
| FGM(IVa) or (IVb) | —F | —NH₂ | —CCl₃ |
| FGN(IVa) or (IVb) | —F | —NH₂ | —F |
| FGO(IVa) or (IVb) | —F | —NH₂ | —Cl |
| FGP(IVa) or (IVb) | —F | —NH₂ | —Br |
| FGQ(IVa) or (IVb) | —F | —NH₂ | —I |
| FGR(IVa) or (IVb) | —F | —CH₃ | —H |
| FGS(IVa) or (IVb) | —F | —CH₃ | —CH₃ |
| FGT(IVa) or (IVb) | —F | —CH₃ | -n-propyl |
| FGU(IVa) or (IVb) | —F | —CH₃ | -n-butyl |
| FGV(IVa) or (IVb) | —F | —CH₃ | -t-butyl |
| FGW(IVa) or (IVb) | —F | —CH₃ | -iso-butyl |
| FGX(IVa) or (IVb) | —F | —CH₃ | —OCH₃ |
| FGY(IVa) or (IVb) | —F | —CH₃ | —OC₂H₅ |
| FGZ(IVa) or (IVb) | —F | —CH₃ | —OC₃H₇ |
| FHA(IVa) or (IVb) | —F | —CH₃ | —CHF₂ |
| FHB(IVa) or (IVb) | —F | —CH₃ | —CF₃ |
| FHC(IVa) or (IVb) | —F | —CH₃ | —CHCl₂ |
| FHD(IVa) or (IVb) | —F | —CH₃ | —CCl₃ |
| FHE(IVa) or (IVb) | —F | —CH₃ | —F |
| FHF(IVa) or (IVb) | —F | —CH₃ | —Cl |
| FHG(IVa) or (IVb) | —F | —CH₃ | —Br |
| FHH(IVa) or (IVb) | —F | —CH₃ | —I |
| FHI(IVa) | —Cl | —H | —H |
| FHJ(IVa) | —Cl | —H | —CH₃ |
| FHK(IVa) | —Cl | —H | -n-propyl |
| FHL(IVa) | —Cl | —H | -n-butyl |
| FHM(IVa) | —Cl | —H | -t-butyl |
| FHN(IVa) | —Cl | —H | -iso-butyl |
| FHO(IVa) | —Cl | —H | —OCH₃ |
| FHP(IVa) | —Cl | —H | —OC₂H₅ |
| FHQ(IVa) | —Cl | —H | —OC₃H₇ |
| FHR(IVa) | —Cl | —H | —CHF₂ |
| FHS(IVa) | —Cl | —H | —CF₃ |
| FHT(IVa) | —Cl | —H | —CHCl₂ |
| FHU(IVa) | —Cl | —H | —CCl₃ |
| FHV(IVa) | —Cl | —H | —F |
| FHW(IVa) | —Cl | —H | —Cl |
| FHX(IVa) | —Cl | —H | —Br |
| FHY(IVa) | —Cl | —H | —I |
| FHZ(IVa) or (IVb) | —Cl | —OH | —H |
| FIA(IVa) or (IVb) | —Cl | —OH | —CH₃ |
| FIB(IVa) or (IVb) | —Cl | —OH | -n-propyl |
| FIC(IVa) or (IVb) | —Cl | —OH | -n-butyl |
| FID(IVa) or (IVb) | —Cl | —OH | -t-butyl |
| FIE(IVa) or (IVb) | —Cl | —OH | -iso-butyl |
| FIF(IVa) or (IVb) | —Cl | —OH | —OCH₃ |
| FIG(IVa) or (IVb) | —Cl | —OH | —OC₂H₅ |
| FIH(IVa) or (IVb) | —Cl | —OH | —OC₃H₇ |
| FII(IVa) or (IVb) | —Cl | —OH | —CHF₂ |
| FIJ(IVa) or (IVb) | —Cl | —OH | —CF₃ |
| FIK(IVa) or (IVb) | —Cl | —OH | —CHCl₂ |
| FIL(IVa) or (IVb) | —Cl | —OH | —CCl₃ |
| FIM(IVa) or (IVb) | —Cl | —OH | —F |
| FIN(IVa) or (IVb) | —Cl | —OH | —Cl |
| FIO(IVa) or (IVb) | —Cl | —OH | —Br |
| FIP(IVa) or (IVb) | —Cl | —OH | —I |
| FIQ(IVa) or (IVb) | —Cl | —F | —H |
| FIR(IVa) or (IVb) | —Cl | —F | —CH₃ |
| FIS(IVa) or (IVb) | —Cl | —F | -n-propyl |
| FIT(IVa) or (IVb) | —Cl | —F | -n-butyl |
| FIU(IVa) or (IVb) | —Cl | —F | -t-butyl |
| FIV(IVa) or (IVb) | —Cl | —F | -iso-butyl |
| FIW(IVa) or (IVb) | —Cl | —F | —OCH₃ |
| FIX(IVa) or (IVb) | —Cl | —F | —OC₂H₅ |
| FIY(IVa) or (IVb) | —Cl | —F | —OC₃H₇ |
| FIZ(IVa) or (IVb) | —Cl | —F | —CHF₂ |
| FJA(IVa) or (IVb) | —Cl | —F | —CF₃ |
| FJB(IVa) or (IVb) | —Cl | —F | —CHCl₂ |
| FJC(IVa) or (IVb) | —Cl | —F | —CCl₃ |
| FJD(IVa) or (IVb) | —Cl | —F | —F |
| FJE(IVa) or (IVb) | —Cl | —F | —Cl |
| FJF(IVa) or (IVb) | —Cl | —F | —Br |
| FJG(IVa) or (IVb) | —Cl | —F | —I |
| FJH(IVa) or (IVb) | —Cl | —Cl | —H |
| FJI(IVa) or (IVb) | —Cl | —Cl | —CH₃ |
| FJJ(IVa) or (IVb) | —Cl | —Cl | -n-propyl |
| FJK(IVa) or (IVb) | —Cl | —Cl | -n-butyl |
| FJL(IVa) or (IVb) | —Cl | —Cl | -t-butyl |
| FJM(IVa) or (IVb) | —Cl | —Cl | -iso-butyl |
| FJN(IVa) or (IVb) | —Cl | —Cl | —OCH₃ |
| FJO(IVa) or (IVb) | —Cl | —Cl | —OC₂H₅ |
| FJP(IVa) or (IVb) | —Cl | —Cl | —OC₃H₇ |
| FJQ(IVa) or (IVb) | —Cl | —Cl | —CHF₂ |
| FJR(IVa) or (IVb) | —Cl | —Cl | —CF₃ |
| FJS(IVa) or (IVb) | —Cl | —Cl | —CHCl₂ |
| FJT(IVa) or (IVb) | —Cl | —Cl | —CC13 |
| FJU(IVa) or (IVb) | —Cl | —Cl | —F |
| FJV(IVa) or (IVb) | —Cl | —Cl | —Cl |
| FJW(IVa) or (IVb) | —Cl | —Cl | —Br |
| FJX(IVa) or (IVb) | —Cl | —Cl | —I |
| FJY(IVa) or (IVb) | —Cl | —Br | —H |
| FJZ(IVa) or (IVb) | —Cl | —Br | —CH₃ |
| FKA(IVa) or (IVb) | —Cl | —Br | -n-propyl |
| FKB(IVa) or (IVb) | —Cl | —Br | -n-butyl |
| FKC(IVa) or (IVb) | —Cl | —Br | -t-butyl |
| FKD(IVa) or (IVb) | —Cl | —Br | -iso-butyl |
| FKE(IVa) or (IVb) | —Cl | —Br | —OCH₃ |
| FKF(IVa) or (IVb) | —Cl | —Br | —OC₂H₅ |
| FKG(IVa) or (IVb) | —Cl | —Br | —OC₃H₇ |
| FKH(IVa) or (IVb) | —Cl | —Br | —CHF₂ |
| FKJ(IVa) or (IVb) | —Cl | —Br | —CF₃ |
| FKJ(IVa) or (IVb) | —Cl | —Br | —CHCl₂ |
| FKK(IVa) or (IVb) | —Cl | —Br | —CC13 |
| FKL(IVa) or (IVb) | —Cl | —Br | —F |
| FKM(IVa) or (IVb) | —Cl | —Br | —Cl |
| FKN(IVa) or (IVb) | —Cl | —Br | —Br |
| FKO(IVa) or (IVb) | —Cl | —Br | —I |
| FKP(IVa) or (IVb) | —CI | —I | —H |
| FKQ(IVa) or (IVb) | —Cl | —I | —CH₃ |
| FKR(IVa) or (IVb) | —Cl | —I | -n-propyl |
| FKS(IVa) or (IVb) | —Cl | —I | -n-butyl |
| FKT(IVa) or (IVb) | —Cl | —I | -t-butyl |
| FKU(IVa) or (IVb) | —Cl | —I | -iso-butyl |
| FKV(IVa) or (IVb) | —Cl | —I | —OCH₃ |
| FKW(IVa) or (IVb) | —Cl | —I | —OC₂H₅ |
| FKX(IVa) or (IVb) | —Cl | —I | —OC₃H₇ |
| FKY(IVa) or (IVb) | —Cl | —I | —CHF₂ |
| FKZ(IVa) or (IVb) | —Cl | —I | —CF₃ |
| FLA(IVa) or (IVb) | —Cl | —I | —CHCl₂ |
| FLB(IVa) or (IVb) | —Cl | —I | —CCl₃ |
| FLC(IVa) or (IVb) | —Cl | —I | —F |
| FLD(IVa) or (IVb) | —Cl | —I | —Cl |
| FLE(IVa) or (IVb) | —Cl | —I | —Br |
| FLF(IVa) or (IVb) | —Cl | —I | —I |
| FLG(IVa) or (IVb) | —Cl | —NO₂ | —H |
| FLH(IVa) or (IVb) | —Cl | —NO₂ | —CH₃ |
| FLI(IVa) or (IVb) | —Cl | —NO₂ | -n-propyl |
| FLJ(IVa) or (IVb) | —Cl | —NO₂ | -n-butyl |
| FLK(IVa) or (IVb) | —Cl | —NO₂ | -t-butyl |
| FLL(IVa) or (IVb) | —Cl | —NO₂ | -iso-butyl |
| FLM(IVa) or (IVb) | —Cl | —NO₂ | —OCH₃ |
| FLN(IVa) or (IVb) | —Cl | —NO₂ | —OC₂H₅ |
| FLO(IVa) or (IVb) | —Cl | —NO₂ | —OC₃H₇ |
| FLP(IVa) or (IVb) | —Cl | —NO₂ | —CHF₂ |
| FLQ(IVa) or (IVb) | —Cl | —NO₂ | —CF₃ |
| FLR(IVa) or (IVb) | —Cl | —NO₂ | —CHCl₂ |
| FLS(IVa) or (IVb) | —Cl | —NO₂ | —CCl₃ |
| FLT(IVa) or (IVb) | —Cl | —NO₂ | —F |
| FLU(IVa) or (IVb) | —Cl | —NO₂ | —Cl |
| FLV(IVa) or (IVb) | —Cl | —NO₂ | —Br |
| FLW(IVa) or (IVb) | —Cl | —NO₂ | —I |
| FLX(IVa) or (IVb) | —Cl | —CN | —H |
| FLV(IVa) or (IVb) | —Cl | —CN | —CH₃ |
| FLZ(IVa) or (IVb) | —Cl | —CN | -n-propyl |
| FMA(IVa) or (IVb) | —Cl | —CN | -n-butyl |
| FMB(IVa) or (IVb) | —Cl | —CN | -t-butyl |
| FMC(IVa) or (IVb) | —Cl | —CN | -iso-butyl |
| FMD(IVa) or (IVb) | —Cl | —CN | —OCH₃ |
| FME(IVa) or (IVb) | —Cl | —CN | —OC₂H₅ |
| FMF(IVa) or (IVb) | —Cl | —CN | —OC₃H₇ |
| FMG(IVa) or (IVb) | —Cl | —CN | —CHF₂ |
| FMH(IVa) or (IVb) | —Cl | —CN | —CF₃ |
| FMI(IVa) or (IVb) | —Cl | —CN | —CHCl₂ |
| FMJ(IVa) or (IVb) | —Cl | —CN | —CCl₃ |
| FMK(IVa) or (IVb) | —Cl | —CN | —F |
| FML(IVa) or (IVb) | —Cl | —CN | —Cl |
| FMM(IVa) or (IVb) | —Cl | —CN | —Br |
| FMN(IVa) or (IVb) | —Cl | —CN | —I |
| FMO(IVa) or (IVb) | —Cl | —NH₂ | —H |
| FMP(IVa) or (IVb) | —Cl | —NH₂ | CH₃ |
| FMQ(IVa) or (IVb) | —Cl | —NH₂ | -n-propyl |
| FMR(IVa) or (IVb) | —Cl | —NH₂ | -n-butyl |
| FMS(IVa) or (IVb) | —CI | —NH₂ | -t-butyl |
| FMT(IVa) or (IVb) | —Cl | —NH₂ | -iso-butyl |
| FMU(IVa) or (IVb) | —Cl | —NH₂ | —OCH₃ |
| FMV(IVa) or (IVb) | —Cl | —NH₂ | —OC₂H₅ |
| FMW(IVa) or (IVb) | —Cl | —NH₂ | —OC₃H₇ |
| FMX(IVa) or (IVb) | —Cl | —NH₂ | —CHF₂ |
| FMY(IVa) or (IVb) | —CI | —NH₂ | —CF₃ |
| FMZ(IVa) or (IVb) | —Cl | —NH₂ | —CHCl₂ |
| FNA(IVa) or (IVb) | —Cl | —NH₂ | —CCl₃ |
| FNB(IVa) or (IVb) | —Cl | —NH₂ | —F |
| FNC(IVa) or (IVb) | —Cl | —NH₂ | —Cl |
| FND(IVa) or (IVb) | —Cl | —NH₂ | —Br |
| FNE(IVa) or (IVb) | —Cl | —NH₂ | —I |
| FNF(IVa) or (IVb) | —Cl | —CH₃ | —H |
| FNG(IVa) or (IVb) | —Cl | —CH₃ | —CH₃ |
| FNH(IVa) or (IVb) | —Cl | —CH₃ | -n-propyl |
| FNI(IVa) or (IVb) | —Cl | —CH₃ | -n-butyl |
| FNJ(IVa) or (IVb) | —CI | —CH₃ | -t-butyl |
| FNK(IVa) or (IVb) | —Cl | —CH₃ | -iso-butyl |
| FNL(IVa) or (IVb) | —Cl | —CH₃ | —OCH₃ |
| FNM(IVa) or (IVb) | —Cl | —CH₃ | —OC2115 |
| FNN(IVa) or (IVb) | —Cl | —CH₃ | —OC3H1 |
| FNO(IVa) or (IVb) | —Cl | —CH₃ | —CHF₂ |
| FNP(IVa) or (IVb) | —Cl | —CH₃ | —CF₃ |
| FNQ(IVa) or (IVb) | —Cl | —CH₃ | —CHCl₂ |
| FNR(IVa) or (IVb) | —Cl | —CH₃ | —CCl₃ |
| FNS(IVa) or (IVb) | —Cl | —CH₃ | —F |
| FNT(IVa) or (IVb) | —Cl | —CH₃ | —Cl |
| FNU(IVa) or (IVb) | —Cl | —CH₃ | —Br |
| FNV(IVa) or (IVb) | —Cl | —CH₃ | —I |
| FNW(IVa) | —CHCl₂ | —H | —H |
| FNX(IVa) | —CHCl₂ | —H | —CH₃ |
| FNY(IVa) | —CHCl₂ | —H | -n-propyl |
| FNZ(IVa) | —CHCl₂ | —H | -n-butyl |
| FOA(IVa) | —CHCl₂ | —H | -t-butyl |
| FOB(IVa) | —CHCl₂ | —H | -iso-butyl |
| FOC(IVa) | —CHCl₂ | —H | —OCH₃ |
| FOD(IVa) | —CHCl₂ | —H | —OC₂H₅ |
| FOE(IVa) | —CHCl₂ | —H | —OC₃H₇ |
| FOF(IVa) | —CHCl₂ | —H | —CHF₂ |
| FOG(IVa) | —CHCl₂ | —H | —CF₃ |
| FOH(IVa) | —CHCl₂ | —H | —CHCl₂ |
| FOI(IVa) | —CHCl₂ | —H | —CCl₃ |
| FOJ(IVa) | —CHCl₂ | —H | —F |
| FOK(IVa) | —CHCl₂ | —H | —Cl |
| FOL(IVa) | —CHCl₂ | —H | —Br |
| FOM(IVa) | —CHCl₂ | —H | —I |
| FON(IVa) or (IVb) | —CHCl₂ | —OH | —H |
| FOO(IVa) or (IVb) | —CHCL2 | —OH | —CH₃ |
| FOP(IVa) or (IVb) | —CHCl₂ | —OH | -n-propyl |
| FOQ(IVa) or (IVb) | —CHCl₂ | —OH | -n-butyl |
| FOR(IVa) or (IVb) | —CHCl₂ | —OH | -t-butyl |
| FOS(IVa) or (IVb) | —CHCl₂ | —OH | -iso-butyl |
| FOT(IVa) or (IVb) | —CHCl₂ | —OH | —OCH₃ |
| FOU(IVa) or (IVb) | —CHCl₂ | —OH | —OC₂H₅ |
| FOV(IVa) or (IVb) | —CHCl₂ | —OH | —OC₃H₇ |
| FOW(IVa) or (IVb) | —CHCl₂ | —OH | —CHF₂ |
| FOX(IVa) or (IVb) | —CHCl₂ | —OH | —CF₃ |
| FOY(IVa) or (IVb) | —CHCL2 | —OH | —CHCl₂ |
| FOZ(IVa) or (IVb) | —CHCl₂ | —OH | —CCl₃ |
| FPA(IVa) or (IVb) | —CHCl₂ | —OH | —F |
| FPB(IVa) or (IVb) | —CHCl₂ | —OH | —Cl |
| FPC(IVa) or (IVb) | —CHCl₂ | —OH | —Br |
| FPD(IVa) or (IVb) | —CHCl₂ | —OH | —I |
| FPE(IVa) or (IVb) | —CHCl₂ | —F | —H |
| FPF(IVa) or (IVb) | —CHCl₂ | —F | —CH₃ |
| FPG(IVa) or (IVb) | —CHCl₂ | —F | -n-propyl |
| FPH(IVa) or (IVb) | —CHCl₂ | —F | -n-butyl |
| FPI(IVa) or (IVb) | —CHCl₂ | —F | -t-butyl |
| FPJ(IVa) or (IVb) | —CHCl₂ | —F | -iso-butyl |
| FPK(IVa) or (IVb) | —CHCl₂ | —F | —OCH₃ |
| FPL(IVa) or (IVb) | —CHCl₂ | —F | —OC₂H₅ |
| FPM(IVa) or (IVb) | —CHCl₂ | —F | —OC₃H₇ |
| FPN(IVa) or (IVb) | —CHCl₂ | —F | —CHF₂ |
| FPO(IVa) or (IVb) | —CHCl₂ | —F | —CF₃ |
| FPP(IVa) or (IVb) | —CHCl₂ | —F | —CHCl₂ |
| FPQ(IVa) or (IVb) | —CHCl₂ | —F | —CCl₃ |
| FPR(IVa) or (IVb) | —CHCl₂ | —F | —F |
| FPS(IVa) or (IVb) | —CHCl₂ | —F | —Cl |
| FPT(IVa) or (IVb) | —CHCl₂ | —F | —Br |
| FPU(IVa) or (IVb) | —CHCl₂ | —F | —I |
| FPV(IVa) or (IVb) | —CHCl₂ | —Cl | —H |
| FPW(IVa) or (IVb) | —CHCl₂ | —Cl | —CH₃ |
| FPX(IVa) or (IVb) | —CHCl₂ | —Cl | -n-propyl |
| FPY(IVa) or (IVb) | —CHCl₂ | —Cl | -n-butyl |
| FPZ(IVa) or (IVb) | —CHCl₂ | —Cl | -t-butyl |
| FQA(IVa) or (IVb) | —CHCl₂ | —Cl | -iso-butyl |
| FQB(IVa) or (IVb) | —CHCl₂ | —Cl | —OCH₃ |
| FQC(IVa) or (IVb) | —CHCl₂ | —Cl | —OC₂H₅ |
| FQD(IVa) or (IVb) | —CHCl₂ | —Cl | —OC₃H₇ |
| FQE(IVa) or (IVb) | —CHCl₂ | —Cl | —CHF₂ |
| FQF(IVa) or (IVb) | —CHCl₂ | —Cl | —CF₃ |
| FQG(IVa) or (IVb) | —CHCl₂ | —Cl | —CHCl₂ |
| FQH(IVa) or (IVb) | —CHCl₂ | —Cl | —CCl₃ |
| FQI(IVa) or (IVb) | —CHCl₂ | —Cl | —F |
| FQJ(IVa) or (IVb) | —CHCl₂ | —Cl | —Cl |
| FQK(IVa) or (IVb) | —CHCl₂ | —Cl | —Br |
| FQL(IVa) or (IVb) | —CHCl₂ | —Cl | —I |
| FQM(IVa) or (IVb) | —CHCl₂ | —Br | —H |
| FQN(IVa) or (IVb) | —CHCl₂ | —Br | —CH₃ |
| FQO(IVa) or (IVb) | —CHCl₂ | —Br | -n-propyl |
| FQP(IVa) or (IVb) | —CHCl₂ | —Br | -n-butyl |
| FQQ(IVa) or (IVb) | —CHCl₂ | —Br | -t-butyl |
| FQR(IVa) or (IVb) | —CHCl₂ | —Br | -iso-butyl |
| FQS(IVa) or (IVb) | —CHCl₂ | —Br | —OCH₃ |
| FQT(IVa) or (IVb) | —CHCl₂ | —Br | —OC₂H₅ |
| FQU(IVa) or (IVb) | —CHCl₂ | —Br | —OC₃H₇ |
| FQV(IVa) or (IVb) | —CHCl₂ | —Br | —CHF₂ |
| FQW(IVa) or (IVb) | —CHCl₂ | —Br | —CF₃ |
| FQX(IVa) or (IVb) | —CHCl₂ | —Br | —CHCl₂ |
| FQY(IVa) or (IVb) | —CHCl₂ | —Br | —CCl₃ |
| FQZ(IVa) or (IVb) | —CHCl₂ | —Br | —F |
| FRA(IVa) or (IVb) | —CHCl₂ | —Br | —Cl |
| FRB(IVa) or (IVb) | —CHCl₂ | —Br | —Br |
| FRC(IVa) or (IVb) | —CHCl₂ | —Br | —I |
| FRD(IVa) or (IVb) | —CHCl₂ | —J | —H |
| FRE(IVa) or (IVb) | —CHCl₂ | —I | —CH₃ |
| FRF(IVa) or (IVb) | —CHCl₂ | —I | -n-propyl |
| FRG(IVa) or (IVb) | —CHCl₂ | —I | -n-butyl |
| FRH(IVa) or (IVb) | —CHCl₂ | —I | -t-butyl |
| FRI(IVa) or (IVb) | —CHCl₂ | —I | -iso-butyl |
| FRJ(IVa) or (IVb) | —CHCl₂ | —I | —OCH₃ |
| FRK(IVa) or (IVb) | —CHCl₂ | —I | —OC₂H₅ |
| FRL(IVa) or (IVb) | —CHCl₂ | —I | —OC₃H₇ |
| FRM(IVa) or (IVb) | —CHCl₂ | —I | —CHF₂ |
| FRN(IVa) or (IVb) | —CHCl₂ | —I | —CF₃ |
| FRO(IVa) or (IVb) | —CHCl₂ | —I | —CHCl₂ |
| FRP(IVa) or (IVb) | —CHCl₂ | —I | —CCl₃ |
| FRQ(IVa) or (IVb) | —CHCl₂ | —I | —F |
| FRR(IVa) or (IVb) | —CHCl₂ | —I | —Cl |
| FRS(IVa) or (IVb) | —CHCl₂ | —I | —Br |
| FRT(IVa) or (IVb) | —CHCl₂ | —J | —I |
| FRU(IVa) or (IVb) | —CHCl₂ | —NO₂ | —H |
| FRV(IVa) or (IVb) | —CHCl₂ | —NO₂ | —Cl3 |
| FRW(IVa) or (IVb) | —CHCl₂ | —NO₂ | -n-propyl |
| FRX(IVa) or (IVb) | —CHCl₂ | —NO₂ | -n-butyl |
| FRY(IVa) or (IVb) | —CHCl₂ | —NO₂ | -t-butyl |
| FRZ(IVa) or (IVb) | —CHCl₂ | —NO₂ | -iso-butyl |
| FSA(IVa) or (IVb) | —CHCl₂ | —NO₂ | —OCH₃ |
| FSB(IVa) or (IVb) | —CHCl₂ | —NO₂ | —OC₂H₅ |
| FSC(IVa) or (IVb) | —CHCl₂ | —NO₂ | —OC₃H₇ |
| FSD(IVa) or (IVb) | —CHCl₂ | —NO₂ | —CHF₂ |
| FSE(IVa) or (IVb) | —CHCl₂ | —NO₂ | —CF₃ |
| FSF(IVa) or (IVb) | —CHCl₂ | —NO₂ | —CHCl₂ |
| FSG(IVa) or (IVb) | —CHCl₂ | —NO₂ | —CCl₃ |
| FSH(IVa) or (IVb) | —CHCl₂ | —NO₂ | —F |
| FSI(IVa) or (IVb) | —CHCl₂ | —NO₂ | —Cl |
| FSJ(IVa) or (IVb) | —CHCl₂ | —NO₂ | —Br |
| FSK(IVa) or (IVb) | —CHCl₂ | —NO₂ | —I |
| FSL(IVa) or (IVb) | —CHCl₂ | —CN | —H |
| FSM(IVa) or (IVb) | —CHCl₂ | —CN | —CH₃ |
| FSN(IVa) or (IVb) | —CHCl₂ | —CN | -n-propyl |
| FSO(IVa) or (IVb) | —CHCl₂ | —CN | -n-butyl |
| FSP(IVa) or (IVb) | —CHCl₂ | —CN | -t-butyl |
| FSQ(IVa) or (IVb) | —CHCl₂ | —CN | -iso-butyl |
| FSR(IVa) or (IVb) | —CHCl₂ | —CN | —OCH₃ |
| FSS(IVa) or (IVb) | —CHCl₂ | —CN | —OC₂H₅ |
| FST(IVa) or (IVb) | —CHCl₂ | —CN | —OC₃H₇ |
| FSU(IVa) or (IVb) | —CHCl₂ | —CN | —CHF₂ |
| FSV(IVa) or (IVb) | —CHCl₂ | —CN | —CF₃ |
| FSW(IVa) or (IVb) | —CHCl₂ | —CN | —CHCl₂ |
| FSX(IVa) or (IVb) | —CHCl₂ | —CN | —CCl₃ |
| FSY(IVa) or (IVb) | —CHCl₂ | —CN | —F |
| FSZ(IVa) or (IVb) | —CHCl₂ | —CN | —Cl |
| FTA(IVa) or (IVb) | —CHCl₂ | —CN | —Br |
| FTB(IVa) or (IVb) | —CHCl₂ | —CN | —I |
| FTC(IVa) or (IVb) | —CHCl₂ | —NH₂ | —H |
| FTD(IVa) or (IVb) | —CHCl₂ | —NH₂ | —CH₃ |
| FTE(IVa) or (IVb) | —CHCl₂ | —NH₂ | -n-propyl |
| FTF(IVa) or (IVb) | —CHCl₂ | —NH₂ | -n-butyl |
| FTG(IVa) or (IVb) | —CHCl₂ | —NH₂ | -t-butyl |
| FTH(IVa) or (IVb) | —CHCl₂ | —NH₂ | -iso-butyl |
| FTI(IVa) or (IVb) | —CHCl₂ | —NH₂ | —OCH₃ |
| FTJ(IVa) or (IVb) | —CHCl₂ | —NH₂ | —OC₂H₅ |
| FTK(IVa) or (IVb) | —CHCl₂ | —NH₂ | —OC₃H₇ |
| FTL(IVa) or (IVb) | —CHCl₂ | —NH₂ | —CHF₂ |
| FTM(IVa) or (IVb) | —CHCl₂ | —NH₂ | —CF₃ |
| FTN(IVa) or (IVb) | —CHCl₂ | —NH₂ | —CHCl₂ |
| FTO(IVa) or (IVb) | —CHCl₂ | —NH₂ | —CCl₃ |
| FTP(IVa) or (IVb) | —CHCl₂ | —NH₂ | —F |
| FTQ(IVa) or (IVb) | —CHCl₂ | —NH₂ | —Cl |
| FTR(IVa) or (IVb) | —CHCl₂ | —NH₂ | —Br |
| FTS(IVa) or (IVb) | —CHCl₂ | —NH₂ | —I |
| FTT(IVa) or (IVb) | —CHCl₂ | —CH₃ | —H |
| FTU(IVa) or (IVb) | —CHCl₂ | —CH₃ | —CH₃ |
| FTV(IVa) or (IVb) | —CHCl₂ | —CH₃ | -n-propyl |
| FTW(IVa) or (IVb) | —CHCl₂ | —CH₃ | -n-butyl |
| FTX(fVa) or (IVb) | —CHCl₂ | —CH₃ | -t-butyl |
| FTY(IVa) or (IVb) | —CHCl₂ | —CH₃ | -iso-butyl |
| FTZ(IVa) or (IVb) | —CHCl₂ | —CH₃ | —OCH₃ |
| FUA(IVa) or (IVb) | —CHCl₂ | —CH₃ | —OC₂H₅ |
| FUB(IVa) or (IVb) | —CHCl₂ | —CH₃ | —OC₃H₇ |
| FUC(IVa) or (IVb) | —CHCl₂ | —CH₃ | —CHF₂ |
| FUD(IVa) or (IVb) | —CHCl₂ | —CH₃ | —CF₃ |
| FUE(IVa) or (IVb) | —CHCl₂ | —CH₃ | —CHCl₂ |
| FUF(IVa) or (IVb) | —CHCl₂ | —CH₃ | —CCl₃ |
| FUG(IVa) or (IVb) | —CHCl₂ | —CH₃ | —F |
| FUH(IVa) or (IVb) | —CHCl₂ | —CH₃ | —Cl |
| FUI(IVa) or (IVb) | —CHCl₂ | —CH₃ | —Br |
| FUJ(IVa) or (IVb) | —CHCl₂ | —CH₃ | —I |
| FUK(IVa) | —CF₃ | —H | —H |
| FUL(IVa) | —CF₃ | —H | —CH₃ |
| FUM(IVa) | —CF₃ | —H | -n-propyl |
| FUN(IVa) | —CF₃ | —H | -n-butyl |
| FUO(IVa) | —CF₃ | —H | -t-butyl |
| FUP(IVa) | —CF₃ | —H | -iso-butyl |
| FUQ(IVa) | —CF₃ | —H | —OCH₃ |
| FUR(IVa) | —CF₃ | —H | —OC₂H₅ |
| FUS(IVa) | —CF₃ | —H | —OC₃H₇ |
| FUT(IVa) | —CF₃ | —H | —CHF₂ |
| FUU(IVa) | —CF₃ | —H | —CF₃ |
| FUV(IVa) | —CF₃ | —H | —CHCl₂ |
| FUW(IVa) | —CF₃ | —H | —CCl₃ |
| FUX(IVa) | —CF₃ | —H | —F |
| FUY(IVa) | —CF₃ | —H | —Cl |
| FUZ(IVa) | —CF₃ | —H | —Br |
| FVA(IVa) | —CF₃ | —H | —I |
| FVB(IVa) or (IVb) | —CF₃ | —OH | —H |
| FVC(IVa) or (IVb) | —CF₃ | —OH | —CH₃ |
| FVD(IVa) or (IVb) | —CF₃ | —OH | -n-propyl |
| FVE(IVa) or (IVb) | —CF₃ | —OH | -n-butyl |
| FVF(IVa) or (IVb) | —CF₃ | —OH | -t-butyl |
| FVG(IVa) or (IVb) | —CF₃ | —OH | -iso-butyl |
| FVH(IVa) or (IVb) | —CF₃ | —OH | —OCH₃ |
| FVI(IVa) or (IVb) | —CF₃ | —OH | —OC₂H₅ |
| FVJ(IVa) or (IVb) | —CF₃ | —OH | —OC₃H₇ |
| FVK(IVa) or (IVb) | —CF₃ | —OH | —CHF₂ |
| FVL(IVa) or (IVb) | —CF₃ | —OH | —CF₃ |
| FVM(IVa) or (IVb) | —CF₃ | —OH | —CHCl₂ |
| FVN(IVa) or (IVb) | —CF₃ | —OH | —CCl₃ |
| FVO(IVa) or (IVb) | —CF₃ | —OH | —F |
| FVP(IVa) or (IVb) | —CF₃ | —OH | —Cl |
| FVQ(IVa) or (IVb) | —CF₃ | —OH | —Br |
| FVR(IVa) or (IVb) | —CF₃ | —OH | —I |
| FVS(IVa) or (IVb) | —CF₃ | —F | —H |
| FVT(IVa) or (IVb) | —CF₃ | —F | —CH₃ |
| FVU(IVa) or (IVb) | —CF₃ | —F | -n-propyl |
| FVV(IVa) or (IVb) | —CF₃ | —F | -n-butyl |
| FVW(IVa) or (IVb) | —CF₃ | —F | -t-butyl |
| FVX(IVa) or (IVb) | —CF₃ | —F | -iso-butyl |
| FVY(IVa) or (IVb) | —CF₃ | —F | —OCH₃ |
| FVZ(IVa) or (IVb) | —CF₃ | —F | —OC₂H₅ |
| FWA(IVa) or (IVb) | —CF₃ | —F | —OC₃H₇ |
| FWB(IVa) or (IVb) | —CF₃ | —F | —CHF₂ |
| FWC(IVa) or (IVb) | —CF₃ | —F | —CF₃ |
| FWD(IVa) or (IVb) | —CF₃ | —F | —CHCl₂ |
| FWE(IVa) or (IVb) | —CF₃ | —F | —CCl₃ |
| FWF(IVa) or (IVb) | —CF₃ | —F | —F |
| FWG(IVa) or (IVb) | —CF₃ | —F | —Cl |
| FWH(IVa) or (IVb) | —CF₃ | —F | —Br |
| FWJ(IVa) or (IVb) | —CF₃ | —F | —I |
| FWJ(IVa) or (IVb) | —CF₃ | —Cl | —H |
| FWK(IVa) or (IVb) | —CF₃ | —Cl | —CH₃ |
| FWL(IVa) or (IVb) | —CF₃ | —Cl | -n-propyl |
| FWM(IVa) or (IVb) | —CF₃ | —Cl | -n-butyl |
| FWN(IVa) or (IVb) | —CF₃ | —Cl | -t-butyl |
| FWO(IVa) or (IVb) | —CF₃ | —Cl | -iso-butyl |
| FWP(IVa) or (IVb) | —CF₃ | —Cl | —OCH₃ |
| FWQ(IVa) or (IVb) | —CF₃ | —Cl | —OC₂H₅ |
| FWR(IVa) or (IVb) | —CF₃ | —Cl | —OC₃H₇ |
| FWS(IVa) or (IVb) | —CF₃ | —Cl | —CHF₂ |
| FWT(IVa) or (IVb) | —CF₃ | —Cl | —CF₃ |
| FWU(IVa) or (IVb) | —CF₃ | —Cl | —CHCl₂ |
| FWV(IVa) or (IVb) | —CF₃ | —Cl | —CCl₃ |
| FWW(IVa) or (IVb) | —CF₃ | —Cl | —F |
| FWX(IVa) or (IVb) | —CF₃ | —Cl | —Cl |
| FWY(IVa) or (IVb) | —CF₃ | —Cl | —Br |
| FWZ(IVa) or (IVb) | —CF₃ | —Cl | —I |
| FXA(IVa) or (IVb) | —CF₃ | —Br | —H |
| FXB(IVa) or (IVb) | —CF₃ | —Br | —CH₃ |
| FXC(IVa) or (IVb) | —CF₃ | —Br | -n-propyl |
| FXD(IVa) or (IVb) | —CF₃ | —Br | -n-butyl |
| FXE(IVa) or (IVb) | —CF₃ | —Br | -t-butyl |
| FXF(IVa) or (IVb) | —CF₃ | —Br | -iso-butyl |
| FXG(IVa) or (IVb) | —CF₃ | —Br | —OCH₃ |
| FXH(IVa) or (IVb) | —CF₃ | —Br | —OC₂H₅ |
| FXI(IVa) or (IVb) | —CF₃ | —Br | —OC₃H₇ |
| FXJ(IVa) or (IVb) | —CF₃ | —Br | —CHF₂ |
| FXK(IVa) or (IVb) | —CF₃ | —Br | —CF₃ |
| FXL(IVa) or (IVb) | —CF₃ | —Br | —CHCl₂ |
| FXM(IVa) or (IVb) | —CF₃ | —Br | —CCl₃ |
| FXN(IVa) or (IVb) | —CF₃ | —Br | —F |
| FXO(IVa) or (IVb) | —CF₃ | —Br | —Cl |
| FXP(IVa) or (IVb) | —CF₃ | —Br | —Br |
| FXQ(IVa) or (IVb) | —CF₃ | —Br | —I |
| FXR(IVa) or (IVb) | —CF₃ | —I | —H |
| FXS(IVa) or (IVb) | —CF₃ | —I | —CH₃ |
| FXT(IVa) or (IVb) | —CF₃ | —l | -n-propyl |
| FXU(IVa) or (IVb) | —CF₃ | —I | -n-butyl |
| FXV(IVa) or (IVb) | —CF, | —I | -t-butyl |
| FXW(IVa) or (IVb) | —CF₃ | —I | -iso-butyl |
| FXX(IVa) or (IVb) | —CF₃ | —I | —OCH₃ |
| FXY(IVa) or (IVb) | —CF₃ | —I | —OC₂H₅ |
| FXZ(IVa) or (IVb) | —CF₃ | —I | —OC₃H₇ |
| FYA(IVa) or (IVb) | —CF₃ | —I | —CHF₂ |
| FYB(IVa) or (IVb) | —CF₃ | —I | —CF₃ |
| FYC(IVa) or (IVb) | —CF₃ | —I | —CHCl₂ |
| FYD(IVa) or (IVb) | —CF₃ | —I | —CCl₃ |
| FYE(IVa) or (IVb) | —CF₃ | —I | —F |
| FYF(IVa) or (IVb) | —CF₃ | —I | —Cl |
| FYG(IVa) or (IVb) | —CF₃ | —I | —Br |
| FYH(IVa) or (IVb) | —CF₃ | —I | —I |
| FYI(IVa) or (IVb) | —CF₃ | —NO₂ | —H |
| FYJ(IVa) or (IVb) | —CF₃ | —NO₂ | —CH₃ |
| FYK(IVa) or (IVb) | —CF₃ | —NO₂ | -n-propyl |
| FYL(IVa) or (IVb) | —CF₃ | —NO₂ | -n-butyl |
| FYM(IVa) or (IVb) | —CF₃ | —NO₂ | -t-butyl |
| FYN(IVa) or (IVb) | —CF₃ | —NO₂ | -iso-butyl |
| FYO(IVa) or (IVb) | —CF₃ | —NO₂ | —OCH₃ |
| FYP(IVa) or (IVb) | —CF₃ | —NO₂ | —OC₂H₅ |
| FYQ(IVa) or (IVb) | —CF₃ | —NO₂ | —OC₃H₇ |
| FYR(IVa) or (IVb) | —CF₃ | —NO₂ | —CHF₂ |
| FYS(IVa) or (IVb) | —CF₃ | —NO₂ | —CF₃ |
| FYT(IVa) or (IVb) | —CF₃ | —NO₂ | —CHCl₂ |
| FYU(IVa) or (IVb) | —CF₃ | —NO₂ | —CCl₃ |
| FYV(IVa) or (IVb) | —CF₃ | —NO₂ | —F |
| FYW(IVa) or (IVb) | —CF₃ | —NO₂ | —Cl |
| FYX(IVa) or (IVb) | —CF₃ | —NO₂ | —Br |
| FYY(IVa) or (IVb) | —CF₃ | —NO₂ | —I |
| FYZ(IVa) or (IVb) | —CF₃ | —CN | —H |
| FZA(IVa) or (IVb) | —CF₃ | —CN | —CH₃ |
| FZB(IVa) or (IVb) | —CF₃ | —CN | -n-propyl |
| FZC(IVa) or (IVb) | —CF₃ | —CN | -n-butyl |
| FZD(IVa) or (IVb) | —CF₃ | —CN | -t-butyl |
| FZE(IVa) or (IVb) | —CF₃ | —CN | -iso-butyl |
| FZF(IVa) or (IVb) | —CF₃ | —CN | —OCH₃ |
| FZG(IVa) or (IVb) | —CF₃ | —CN | —OC₂H₅ |
| FZH(IVa) or (IVb) | —CF₃ | —CN | —OC₃H₇ |
| FZI(IVa) or (IVb) | —CF₃ | —CN | —CHF₂ |
| FZJ(IVa) or (IVb) | —CF₃ | —CN | —CF₃ |
| FZK(IVa) or (IVb) | —CF₃ | —CN | —CHCl₂ |
| FZL(IVa) or (IVb) | —CF₃ | —CN | —CCl₃ |
| FZM(IVa) or (IVb) | —CF₃ | —CN | —F |
| FZN(IVa) or (IVb) | —CF₃ | —CN | —Cl |
| FZO(IVa) or (IVb) | —CF₃ | —CN | —Br |
| FZP(IVa) or (IVb) | —CF₃ | —CN | —I |
| FZQ(IVa) or (IVb) | —CF₃ | —NH₂ | —H |
| FZR(IVa) or (IVb) | —CF₃ | —NH₂ | CH₃ |
| FZS(IVa) or (IVb) | —CF₃ | —NH₂ | -n-propyl |
| FZT(IVa) or (IVb) | —CF₃ | —NH₂ | -n-butyl |
| FZU(IVa) or (IVb) | —CF₃ | —NH₂ | -t-butyl |
| FZV(IVa) or (IVb) | —CF₃ | —NH₂ | -iso-butyl |
| FZW(IVa) or (IVb) | —CF₃ | —NH₂ | —OCH₃ |
| FZX(IVa) or (IVb) | —CF₃ | —NH₂ | —OC₂H₅ |
| FZY(IVa) or (IVb) | —CF₃ | —NH₂ | —OC₃H₇ |
| FZZ(IVa) or (IVb) | —CF₃ | —NH₂ | —CHF₂ |
| GAA(IVa) or (IVb) | —CF₃ | —NH₂ | —CF₃ |
| GAB(IVa) or (IVb) | —CF₃ | —NH₂ | —CHCl₂ |
| GAC(IVa) or (IVb) | —CF₃ | —NH₂ | —CCl₃ |
| GAD(IVa) or (IVb) | —CF₃ | —NH₂ | —F |
| GAE(IVa) or (IVb) | —CF₃ | —NH₂ | —Cl |
| GAF(IVa) or (IVb) | —CF₃ | —NH₂ | —Br |
| GAG(IVa) or (IVb) | —CF₃ | —NH₂ | —I |
| GAH(IVa) or (IVb) | —CF₃ | —CH₃ | —H |
| GAI(IVa) or (IVb) | —CF₃ | —CH₃ | —CH₃ |
| GAJ(IVa) or (IVb) | —CF₃ | —CH₃ | -n-propyl |
| GAK(IVa) or (IVb) | —CF₃ | —CH₃ | -n-butyl |
| GAL(IVa) or (IVb) | —CF₃ | —CH₃ | -t-butyl |
| GAM(IVa) or (IVb) | —CF₃ | —CH₃ | -iso-butyl |
| GAN(IVa) or (IVb) | —CF₃ | —CU3 | —OCH₃ |
| GAO(IVa) or (IVb) | —CF₃ | —CU3 | —OC₂H₅ |
| GAP(IVa) or (IVb) | —CF₃ | —CH₃ | —OC₃H₇ |
| GAQ(IVa) or (IVb) | —CF₃ | —CH₃ | —CHF₂ |
| GAR(IVa) or (IVb) | —CF₃ | —CU3 | —CF₃ |
| GAS(IVa) or (IVb) | —CF₃ | —CH₃ | —CHCl₂ |
| GAT(IVa) or (IVb) | —CF₃ | —CH₃ | —CCl₃ |
| GAU(IVa) or (IVb) | —CF₃ | —CH₃ | —F |
| GAV(IVa) or (IVb) | —CF₃ | —CH₃ | —Cl |
| GAW(IVa) or (IVb) | —CF₃ | —CH₃ | —Br |
| GAX(IVa) or (IVb) | —CF₃ | —CH₃ | —I |
| GAY(IVa) | —NO₂ | —H | —H |
| GAZ(IVa) | —NO₂ | —H | —CH₃ |
| GBA(IVa) | —NO₂ | —H | -n-propyl |
| GBB(IVa) | —NO₂ | —H | -n-butyl |
| GBC(IVa) | —NO₂ | —H | -t-butyl |
| GBD(IVa) | —NO₂ | —H | -iso-butyl |
| GBE(IVa) | —NO₂ | —U | —OCH₃ |
| GBF(IVa) | —NO₂ | —H | —OC₂H₅ |
| GBG(IVa) | —NO₂ | —U | —OC3U7 |
| GBH(IVa) | —NO₂ | —U | —CHF₂ |
| GBI(IVa) | —NO₂ | —H | —CF₃ |
| GBJ(IVa) | —NO₂ | —H | —CHCl₂ |
| GBK(IVa) | —NO₂ | —H | —CCl₃ |
| GBL(IVa) | —NO₂ | —H | —F |
| GBM(IVa) | —NO₂ | —H | —Cl |
| GBN(IVa) | —NO₂ | —H | —Br |
| GBO(IVa) | —NO₂ | —H | —I |
| GBP(IVa) or (IVb) | —NO₂ | —OH | —H |
| GBQ(IVa) or (IVb) | —NO₂ | —OH | —CH₃ |
| GBR(IVa) or (IVb) | —NO₂ | —OH | -n-propyl |
| GBS(IVa) or (IVb) | —NO₂ | —OH | -n-butyl |
| GBT(IVa) or (IVb) | —NO₂ | —OH | -t-butyl |
| GBU(IVa) or (IVb) | —NO₂ | —OH | -iso-butyl |
| GBV(IVa) or (IVb) | —NO₂ | —OH | —OCH₃ |
| GBW(IVa) or (IVb) | —NO₂ | —OH | —OC₂H₅ |
| GBX(IVa) or (IVb) | —NO₂ | —OH | —OC₃H₇ |
| GBY(IVa) or (IVb) | —NO₂ | —OH | —CHF₂ |
| GBZ(IVa) or (IVb) | —NO₂ | —OH | —CF₃ |
| GCA(IVa) or (IVb) | —NO₂ | —OH | —CHCl₂ |
| GCB(IVa) or (IVb) | —NO₂ | —OH | —CCl₃ |
| GCC(IVa) or (IVb) | —NO₂ | —OH | —F |
| GCD(IVa) or (IVb) | —NO₂ | —OH | —Cl |
| GCE(IVa) or (IVb) | —NO₂ | —OH | —Br |
| GCF(IVa) or (IVb) | —NO₂ | —OH | —I |
| GCG(IVa) or (IVb) | —NO₂ | —F | —H |
| GCH(IVa) or (IVb) | —NO₂ | —F | —CH₃ |
| GCI(IVa) or (IVb) | —NO₂ | —F | -n-propyl |
| GCJ(IVa) or (IVb) | —NO₂ | —F | -n-butyl |
| GCK(IVa) or (IVb) | —NO₂ | —F | -t-butyl |
| GCL(IVa) or (IVb) | —NO₂ | —F | -iso-butyl |
| GCM(IVa) or (IVb) | —NO₂ | —F | —OCH₃ |
| GCN(IVa) or (IVb) | —NO₂ | —F | —OC₂H₅ |
| GCO(IVa) or (IVb) | —NO₂ | —F | —OC₃H₇ |
| GCP(IVa) or (IVb) | —NO₂ | —F | —CHF₂ |
| GCQ(IVa) or (IVb) | —NO₂ | —F | —CF₃ |
| GCR(IVa) or (IVb) | —NO₂ | —F | —CHCl₂ |
| GCS(IVa) or (IVb) | —NO₂ | —F | —CCl₃ |
| GCT(IVa) or (IVb) | —NO₂ | —F | —F |
| GCU(IVa) or (IVb) | —NO₂ | —F | —Cl |
| GCV(IVa) or (IVb) | —NO₂ | —F | —Br |
| GCW(IVa) or (IVb) | —NO₂ | —F | —I |
| GCX(IVa) or (IVb) | —NO₂ | —Cl | —H |
| GCY(IVa) or (IVb) | —NO₂ | —Cl | —CH₃ |
| GCZ(IVa) or (IVb) | —NO₂ | —Cl | -n-propyl |
| GDA(IVa) or (IVb) | —NO₂ | —Cl | -n-butyl |
| GDB(IVa) or (IVb) | —NO₂ | —Cl | -t-butyl |
| GDC(IVa) or (IVb) | —NO₂ | —Cl | -iso-butyl |
| GDD(IVa) or (IVb) | —NO₂ | —Cl | —OCH₃ |
| GDE(IVa) or (IVb) | —NO₂ | —Cl | —OC₂H₅ |
| GDF(IVa) or (IVb) | —NO₂ | —Cl | —OC₃H₇ |
| GDG(IVa) or (IVb) | —NO₂ | —Cl | —CH₂ |
| GDH(IVa) or (IVb) | —NO₂ | —Cl | —CF₃ |
| GDI(IVa) or (IVb) | —NO₂ | —Cl | —CHCl₂ |
| GDJ(IVa) or (IVb) | —NO₂ | —Cl | —CCl₃ |
| GDK(IVa) or (IVb) | —NO₂ | —Cl | —F |
| GDL(IVa) or (IVb) | —NO₂ | —Cl | —Cl |
| GDM(IVa) or (IVb) | —NO₂ | —Cl | —Br |
| GDN(IVa) or (IVb) | —NO₂ | —Cl | —I |
| GDO(IVa) or (IVb) | —NO₂ | —Br | —H |
| GDP(IVa) or (IVb) | —NO₂ | —Br | —CH₃ |
| GDQ(IVa) or (IVb) | —NO₂ | —Br | -n-propyl |
| GDR(IVa) or (IVb) | —NO₂ | —Br | -n-butyl |
| GDS(IVa) or (IVb) | —NO₂ | —Br | -t-butyl |
| GDT(IVa) or (IVb) | —NO₂ | —Br | -iso-butyl |
| GDU(IVa) or (IVb) | —NO₂ | —Br | —OCH₃ |
| GDV(IVa) or (IVb) | —NO₂ | —Br | —OC₂H₅ |
| GDW(IVa) or (IVb) | —NO₂ | —Br | —OC₃H₇ |
| GDX(IVa) or (IVb) | —NO₂ | —Br | —CHF₂ |
| GDY(IVa) or (IVb) | —NO₂ | —Br | —CF₃ |
| GDZ(IVa) or (IVb) | —NO₂ | —Br | —CHCJ2 |
| GEA(IVa) or (IVb) | —NO₂ | —Br | —CCl₃ |
| GEB(IVa) or (IVb) | —NO₂ | —Br | —F |
| GEC(IVa) or (IVb) | —NO₂ | —Br | —Cl |
| GED(IVa) or (IVb) | —NO₂ | —Br | —Br |
| GEE(IVa) or (IVb) | —NO₂ | —Br | —I |
| GEF(IVa) or (IVb) | —NO₂ | —I | —H |
| GEG(IVa) or (IVb) | —NO₂ | —I | —CH₃ |
| GEH(IVa) or (IVb) | —NO₂ | —I | -n-propyl |
| GEI(IVa) or (IVb) | —NO₂ | —I | -n-butyl |
| GEJ(IVa) or (IVb) | —NO₂ | —I | -t-butyl |
| GEK(IVa) or (IVb) | —NO₂ | —I | -iso-butyl |
| GEL(IVa) or (IYb) | —NO₂ | —I | —OCH₃ |
| GEM(IVa) or (IVb) | —NO₂ | —I | —OC₂H₅ |
| GEN(IVa) or (IVb) | —NO₂ | —I | —OC₃H₇ |
| GEO(IVa) or (IVb) | —NO₂ | —I | —CHF₂ |
| GEP(IVa) or (IVb) | —NO₂ | —I | —CF₃ |
| GEQ(IVa) or (IVb) | —NO₂ | —I | —CHCl₂ |
| GER(IVa) or (IVb) | —NO₂ | —I | —CCl₃ |
| GES(IVa) or (IVb) | —NO₂ | —I | —F |
| GET(IVa) or (IVb) | —NO₂ | —I | —Cl |
| GEU(IVa) or (IVb) | —NO₂ | —I | —Br |
| GEV(IVa) or (IVb) | —NO₂ | —I | —I |
| GEW(IVa) or (IVb) | —NO₂ | —NO₂ | —H |
| GEX(IVa) or (IVb) | —NO₂ | —NO₂ | —CH₃ |
| GEY(IVa) or (IVb) | —NO₂ | —NO₂ | -n-propyl |
| GEZ(IVa) or (IVb) | —NO₂ | —NO₂ | —n—buty~ |
| GFA(IVa) or (IVb) | —NO₂ | —NO₂ | -t-butyl |
| GFB(IVa) or (IVb) | —NO₂ | —NO₂ | -iso-butyl |
| GFC(IVa) or (IVb) | —NO₂ | —NO₂ | —OCH₃ |
| GFD(IVa) or (IVb) | —NO₂ | —NO₂ | —OC₂H₅ |
| GFE(IVa) or (IVb) | —NO₂ | —NO₂ | —OC₃H₇ |
| GFF(IVa) or (IVb) | —NO₂ | —NO₂ | —CHF₂ |
| GFG(IVa) or (IVb) | —NO₂ | —NO₂ | —CF₃ |
| GFH(IVa) or (IVb) | —NO₂ | —NO₂ | —CHCl₂ |
| GFI(IVa) or (IVb) | —NO₂ | —NO₂ | —CCl₃ |
| GFJ(IVa) or (IVb) | —NO₂ | —NO₂ | —F |
| GFK(IVa) or (IVb) | —NO₂ | —NO₂ | —Cl |
| GFL(IVa) or (IVb) | —NO₂ | —NO₂ | —Br |
| GFM(IVa) or (IVb) | —NO₂ | —NO₂ | —I |
| GFN(IVa) or (IVb) | —NO₂ | —CN | —H |
| GFO(IVa) or (IVb) | —NO₂ | —CN | —CH₃ |
| GFP(IVa) or (IVb) | —NO₂ | —CN | -n-propyl |
| GFQ(IVa) or (IVb) | —NO₂ | —CN | -n-butyl |
| GFR(IVa) or (IVb) | —NO₂ | —CN | -t-butyl |
| GFS(IVa) or (IVb) | —NO₂ | —CN | -iso-butyl |
| GFT(IVa) or (IVb) | —NO₂ | —CN | —OCH₃ |
| GFU(IVa) or (IVb) | —NO₂ | —CN | —OC₂H₅ |
| GFV(IVa) or (IVb) | —NO₂ | —CN | —OC₃H₇ |
| GFW(IVa) or (IVb) | —NO₂ | —CN | —CHF₂ |
| GFX(IVa) or (IVb) | —NO₂ | —CN | —CF₃ |
| GFY(IVa) or (IVb) | —NO₂ | —CN | —CHCl₂ |
| GFZ(IVa) or (IVb) | —NO₂ | —CN | —CCl₃ |
| GGA(IVa) or (IVb) | —NO₂ | —CN | —F |
| GGB(IVa) or (IVb) | —NO₂ | —CN | —Cl |
| GGC(IVa) or (IVb) | —NO₂ | —CN | —Br |
| GGD(IVa) or (IVb) | —NO₂ | —CN | —I |
| GGE(IVa) or (IVb) | —NO₂ | —NH₂ | —H |
| GGF(IVa) or (IVb) | —NO₂ | —NH₂ | —CH₃ |
| GGG(IVa) or (IVb) | —NO₂ | —NH₂ | -n-propyl |
| GGH(IVa) or (IVb) | —NO₂ | —NH₂ | -n-butyl |
| GGI(IVa) or (IVb) | —NO₂ | —NH₂ | -t-butyl |
| GGJ(IVa) or (IVb) | —NO₂ | —NH₂ | -iso-butyl |
| GGK(IVa) or (IVb) | —NO₂ | —NH₂ | —OCH₃ |
| GGL(IVa) or (IVb) | —NO₂ | —NH₂ | —OC₂H₅ |
| GGM(IVa) or (IVb) | —NO₂ | —NH₂ | OC₃H₇ |
| GGN(IVa) or (IVb) | —NO₂ | —NH₂ | —CHF₂ |
| GGO(IVa) or (IVb) | —NO₂ | —NH₂ | —CF₃ |
| GGP(IVa) or (IVb) | —NO₂ | —NH₂ | —CHCl₂ |
| GGQ(IVa) or (IVb) | —NO₂ | —NH₂ | —CCl₃ |
| GGR(IVa) or (IVb) | —NO₂ | —NH₂ | —F |
| GGS(IVa) or (IVb) | —NO₂ | —NH₂ | —Cl |
| GGT(IVa) or (IVb) | —NO₂ | —NH₂ | —Br |
| GGU(IVa) or (IVb) | —NO₂ | —NH₂ | —I |
| GGV(IVa) or (IVb) | —NO₂ | —CH₃ | —H |
| GGW(IVa) or (IVb) | —NO₂ | —CH₃ | —CH₃ |
| GGX(IVa) or (IVb) | —NO₂ | —CH₃ | -n-propyl |
| GGY(IVa) or (IVb) | —NO₂ | —CH₃ | -n-butyl |
| GGZ(IVa) or (IVb) | —NO₂ | —CH₃ | -t-butyl |
| GHA(IVa) or (IVb) | —NO₂ | —CH₃ | -iso-butyl |
| GHB(IVa) or (IVb) | —NO₂ | —CH₃ | —OCH₃ |
| GHC(IVa) or (IVb) | —NO₂ | —CH₃ | —OC₂H₅ |
| GHD(IVa) or (IVb) | —NO₂ | —CH₃ | —OC₃H₇ |
| GHE(IVa) or (IVb) | —NO₂ | —CH₃ | —CHF₂ |
| GHF(IVa) or (IVb) | —NO₂ | —CH₃ | —CF₃ |
| GHG(IVa) or (IVb) | —NO₂ | —CH₃ | —CHCl₂ |
| GHH(IVa) or (IVb) | —NO₂ | —CH₃ | —CCl₃ |
| GHI(IVa) or (IVb) | —NO₂ | —CH₃ | —F |
| GHJ(IVa) or (IVb) | —NO₂ | —CH₃ | —Cl |
| GHK(IVa) or (IVb) | —NO₂ | —CH₃ | —Br |
| GHL(IVa) or (IVb) | —NO₂ | —CH₃ | —I |
| GHM(IVa) | —CN | —H | —H |
| GHN(IVa) | —CN | —H | —CH₃ |
| GHO(IVa) | —CN | —H | -n-propyl |
| GHP(IVa) | —CN | —H | -n-butyl |
| GHQ(IVa) | —CN | —H | -t-butyl |
| GHR(IVa) | —CN | —H | -iso-butyl |
| GHS(IVa) | —CN | —H | —OCH₃ |
| GHT(IVa) | —CN | —H | —OC₂H₅ |
| GHU(IVa) | —CN | —H | —OC₃H₇ |
| GHX(IVa) | —CN | —H | —CHF₂ |
| GHW(IVa) | —CN | —H | —CF₃ |
| GHX(IVa) | —CN | —H | —CHCl₂ |
| GHY(IVa) | —CN | —H | —CCl₃ |
| GHZ(IVa) | —CN | —H | —F |
| GIA(IVa) | —CN | —H | —Cl |
| GIB(IVa) | —CN | —H | —Br |
| GIC(IVa) | —CN | —H | —I |
| GID(IVa) or (IVb) | —CN | —OH | —H |
| GIE(IVa) or (IVb) | —CN | —OH | —CH₃ |
| GIF(IVa) or (IVb) | —CN | —OH | -n-propyl |
| GIG(IVa) or (IVb) | —CN | —OH | -n-butyl |
| GIH(IVa) or (IVb) | —CN | —OH | -t-butyl |
| GII(IVa) or (IVb) | —CN | —OH | -iso-butyl |
| GIJ(IVa) or (IVb) | —CN | —OH | —OCH₃ |
| GIK(IVa) or (IVb) | —CN | —OH | —OC₂H₅ |
| GIL(IVa) or (IVb) | —CN | —OH | —OC₃H₇ |
| GIM(IVa) or (IVb) | —CN | —OH | —CHF₂ |
| GIN(IVa) or (IVb) | —CN | —OH | —CF₃ |
| GIO(IVa) or (IVb) | —CN | —OH | —CHCl₂ |
| GIP(IVa) or (IVb) | —CN | —OH | —CCl₃ |
| GIQ(IVa) or (IVb) | —CN | —OH | —F |
| GIR(IVa) or (IVb) | —CN | —OH | —Cl |
| GIS(IVa) or (IVb) | —CN | —OH | —Br |
| GIT(IVa) or (IVb) | —CN | —OH | —I |
| GIU(IVa) or (IVb) | —CN | —F | —H |
| GIV(IVa) or (IVb) | —CN | —F | —CH₃ |
| GIW(IVa) or (IVb) | —CN | —F | -n-propyl |
| GIX(IVa) or (IVb) | —CN | —F | -n-butyl |
| GIY(IVa) or (IVb) | —CN | —F | -t-butyl |
| GIZ(IVa) or (IVb) | —CN | —F | -iso-butyl |
| GJA(IVa) or (IVb) | —CN | —F | —OCH₃ |
| GJB(IVa) or (IVb) | —CN | —F | —OC₂H₅ |
| GJC(IVa) or (IVb) | —CN | —F | —OC₃H₇ |
| GJD(IVa) or (IVb) | —CN | —F | —CHF₂ |
| GJE(IVa) or (IVb) | —CN | —F | —CF₃ |
| GJF(IVa) or (IVb) | —CN | —F | —CHCl₂ |
| GJG(IVa) or (IVb) | —CN | —F | —CCl₃ |
| GJH(IVa) or (IVb) | —CN | —F | —F |
| GJI(IVa) or (IVb) | —CN | —F | —Cl |
| GJJ(IVa) or (IVb) | —CN | —F | —Br |
| GJK(IVa) or (IVb) | —CN | —F | —I |
| GJL(IVa) or (IVb) | —CN | —Cl | —H |
| GJM(IVa) or (IVb) | —CN | —Cl | —CH₃ |
| GJN(IVa) or (IVb) | —CN | —Cl | -n-propyl |
| GJO(IVa) or (IVb) | —CN | —Cl | -n-butyl |
| GJP(IVa) or (IVb) | —CN | —Cl | -t-butyl |
| GJQ(IVa) or (IVb) | —CN | —Cl | -iso-butyl |
| GJR(IVa) or (IVb) | —CN | —Cl | —OCH₃ |
| GJS(IVa) or (IVb) | —CN | —Cl | —OC₂H₅ |
| GJT(IVa) or (IVb) | —CN | —Cl | —OC₃H₇ |
| GJU(IVa) or (IVb) | —CN | —Cl | —CHF₂ |
| GJV(IVa) or (IVb) | —CN | —Cl | —CF₃ |
| GJW(IVa) or (IVb) | —CN | —Cl | —CHCl₂ |
| GJX(IVa) or (IVb) | —CN | —Cl | —Cd3 |
| GJY(IVa) or (IVb) | —CN | —Cl | —F |
| GJZ(IVa) or (IVb) | —CN | —Cl | —Cl |
| GKA(IVa) or (IVb) | —CN | —Cl | —Br |
| GKB(IVa) or (IVb) | —CN | —Cl | —I |
| GKC(IVa) or (IVb) | —CN | —Br | —H |
| GKD(IVa) or (IVb) | —CN | —Br | —CH₃ |
| GKE(IVa) or (IVb) | —CN | —Br | -n-propyl |
| GKF(IVa) or (IVb) | —CN | —Br | -n-butyl |
| GKG(IVa) or (IVb) | —CN | —Br | -t-butyl |
| GKH(IVa) or (IVb) | —CN | —Br | -iso-butyl |
| GKI(IVa) or (IVb) | —CN | —Br | —OCH₃ |
| GKJ(IVa) or (IVb) | —CN | —Br | —OC₂H₅ |
| GKK(IVa) or (IVb) | —CN | —Br | —OC₃H₇ |
| GKL(IVa) or (IVb) | —CN | —Br | —CHF₂ |
| GKM(IVa) or (IVb) | —CN | —Br | —CF₃ |
| GKN(IVa) or (IVb) | —CN | —Br | —CHCl₂ |
| GKO(IVa) or (IVb) | —CN | —Br | —CCl₃ |
| GKP(IVa) or (IVb) | —CN | —Br | —F |
| GKQ(IVa) or (IVb) | —CN | —Br | —Cl |
| GKR(IVa) or (IVb) | —CN | —Br | —Br |
| GKS(IVa) or (IVb) | —CN | —Br | —I |
| GKT(IVa) or (IVb) | —CN | —I | —H |
| GKU(IVa) or (IVb) | —CN | —I | —CH₃ |
| GKV(IVa) or (IVb) | —CN | —I | -n-propyl |
| GKW(IVa) or (IVb) | —CN | —I | -n-butyl |
| GKX(IVa) or (IVb) | —CN | —I | -t-butyl |
| GKY(IVa) or (IVb) | —CN | —I | -iso-butyl |
| GKZ(IVa) or (IVb) | —CN | —I | —OCH₃ |
| GLA(IVa) or (IVb) | —CN | —I | —OC₂H₅ |
| GLB(IVa) or (IVb) | —CN | —I | —OC₃H₇ |
| GLC(IVa) or (IVb) | —CN | —I | —CHF₂ |
| GLD(IVa) or (IVb) | —CN | —I | —CF₃ |
| GLE(IVa) or (IVb) | —CN | —I | —CHCl₂ |
| GLF(IVa) or (IVb) | —CN | —I | —CCl₃ |
| GLG(IVa) or (IVb) | —CN | —I | —F |
| GLH(IVa) or (IVb) | —CN | —I | —Cl |
| GLI(IVa) or (IVb) | —CN | —I | —Br |
| GLJ(IVa) or (IVb) | —CN | —I | —I |
| GLK(IVa) or (IVb) | —CN | —NO₂ | —H |
| GLL(IVa) or (IVb) | —CN | —NO₂ | —CH₃ |
| GLM(IVa) or (IVb) | —CN | —NO₂ | -n-propyl |
| GLN(IVa) or (IVb) | —CN | —NO₂ | -n-butyl |
| GLO(IVa) or (IVb) | —CN | —NO₂ | -t-butyl |
| GLP(IVa) or (IVb) | —CN | —NO₂ | -iso-butyl |
| GLQ(IVa) or (IVb) | —CN | —NO₂ | —OCH₃ |
| GLR(IVa) or (IVb) | —CN | —NO₂ | —OC₂H₅ |
| GLS(IVa) or (IVb) | —CN | —NO₂ | —OC₃H₇ |
| GLT(IVa) or (IVb) | —CN | —NO₂ | —CHF₂ |
| GLU(IVa) or (IVb) | —CN | —NO₂ | —CF₃ |
| GLV(IVa) or (IVb) | —CN | —NO₂ | —CHCl₂ |
| GLW(IVa) or (IVb) | —CN | —NO₂ | —Cd3 |
| GLX(IVa) or (IVb) | —CN | —NO₂ | —F |
| GLY(IVa) or (IVb) | —CN | —NO₂ | —Cl |
| GLZ(IVa) or (IVb) | —CN | —NO₂ | —Br |
| GMA(IVa) or (IVb) | —CN | —NO₂ | —I |
| GMB(IVa) or (IVb) | —CN | —CN | —H |
| GMC(IVa) or (IVb) | —CN | —CN | —CH₃ |
| GMD(IVa) or (IVb) | —CN | —CN | -n-propyl |
| GME(IVa) or (IVb) | —CN | —CN | -n-butyl |
| GMF(IVa) or (IVb) | —CN | —CN | -t-butyl |
| GMG(IVa) or (IVb) | —CN | —CN | -iso-butyl |
| GMH(IVa) or (IVb) | —CN | —CN | —OCH₃ |
| GMI(IVa) or (IVb) | —CN | —CN | —OC₂H₅ |
| GMJ(IVa) or (IVb) | —CN | —CN | —OC₃H₇ |
| GMK(IVa) or (IVb) | —CN | —CN | —CHF₂ |
| GML(IVa) or (IVb) | —CN | —CN | —CF₃ |
| GMM(IVa) or (IVb) | —CN | —CN | —CHCl₂ |
| GMN(IVa) or (IVb) | —CN | —CN | —CCl₃ |
| GMO(IVa) or (IVb) | —CN | —CN | —F |
| GMP(IVa) or (IVb) | —CN | —CN | —Cl |
| GMQ(IVa) or (IVb) | —CN | —CN | —Br |
| GMR(IVa) or (IVb) | —CN | —CN | —I |
| GMS(IVa) or (IVb) | —CN | —NI—I2 | —H |
| GMT(IVa) or (IVb) | —CN | —NI—I2 | —CH₃ |
| GMU(IVa) or (IVb) | —CN | —NH₂ | -n-propyl |
| GMV(IVa) or (IVb) | —CN | —NH₂ | -n-butyl |
| GMW(IVa) or (IVb) | —CN | —NH₂ | -t-butyl |
| GMX(IVa) or (IVb) | —CN | —NH₂ | -iso-butyl |
| GMY(IVa) or (IVb) | —CN | —NH₂ | —OCH₃ |
| GMZ(IVa) or (IVb) | —CN | —NH₂ | —OC₂H₅ |
| GNA(IVa) or (IVb) | —CN | —NH₂ | —OC₃H₇ |
| GNB(IVa) or (IVb) | —CN | —NH₂ | —CHF₂ |
| GNC(IVa) or (IVb) | —CN | —NH₂ | —CF₃ |
| GND(IVa) or (IVb) | —CN | —NH₂ | —CHCl₂ |
| GNE(IVa) or (IVb) | —CN | —NH₂ | |
| GNF(IVa) or (IVb) | —CN | —NH₂ | —F |
| GNG(IVa) or (IVb) | —CN | —NH₂ | —Cl |
| GNH(IVa) or (IVb) | —CN | —NH₂ | —Br |
| GNI(IVa) or (IVb) | —CN | —NH₂ | —I |
| GNJ(IVa) or (IVb) | —CN | —CH₃ | —H |
| GNK(IVa) or (IVb) | —CN | —CH₃ | —CH₃ |
| GNL(IVa) or (IVb) | —CN | —CH₃ | -n-propyl |
| GNM(IVa) or (IVb) | —CN | —CH₃ | -n-butyl |
| GNN(IVa) or (IVb) | —CN | —CH₃ | -t-butyl |
| GNO(IVa) or (IVb) | —CN | —CH₃ | -iso-butyl |
| GNP(IVa) or (IVb) | —CN | —CH₃ | —OCH₃ |
| GNQ(IVa) or (IVb) | —CN | —CH₃ | —OC2U5 |
| GNR(IVa) or (IVb) | —CN | —CH₃ | —OC₃H₇ |
| GNS(IVa) or (IVb) | —CN | —CH₃ | —CHF₂ |
| GNT(IVa) or (IVb) | —CN | —CH₃ | —CF₃ |
| GNU(IVa) or (IVb) | —CN | —CH₃ | —Cl |
| GNV(IVa) or (IVb) | —CN | —CH₃ | —CCl₃ |
| GNW(IVa) or (IVb) | —CN | —CH₃ | —F |
| GNX(IVa) or (IVb) | —CN | —CH₃ | —Cl |
| GNY(IVa) or (IVb) | —CN | —CH₃ | —Br |
| GNZ(IVa) or (IVb) | —CN | —CH₃ | —I |
| GOA(IVa) | —CH₃ | —H | —H |
| GOB(IVa) | —CH₃ | —H | —CH₃ |
| GOC(IVa) | —CH₃ | —U | -n-propyl |
| GOD(IVa) | —CH₃ | —H | -n-butyl |
| GOE(IVa) | —CH₃ | —H | -t-butyl |
| GOF(IVa) | —CH₃ | —H | -iso-butyl |
| GOG(IVa) | —CH₃ | —U | —OCH₃ |
| GOH(IVa) | —CH₃ | —U | —OC₂H₅ |
| GOI(IVa) | —CH₃ | —H | —OC3₃H₇ |
| GOJ(IVa) | —CH₃ | —U | —CHF₂ |
| GOK(IVa) | —CH₃ | —U | —CF₃ |
| GOL(IVa) | —CH₃ | —U | —CHCl₂ |
| GOM(IVa) | —CH₃ | —U | —CCl₃ |
| GON(IVa) | —CH₃ | —H | —F |
| GOO(IVa) | —CH₃ | —H | —Cl |
| GOP(IVa) | —CH₃ | —H | —Br |
| GOQ(IVa) | —CH₃ | —H | —I |
| GOR(IVa) or (IVb) | —CH₃ | —OH | —H |
| GOS(IVa) or (IVb) | —CH₃ | —OH | —CH₃ |
| GOT(IVa) or (IVb) | —CH₃ | —OH | -n-propyl |
| GOU(IVa) or (IVb) | —CH₃ | —OH | -n-butyl |
| GOV(IVa) or (IVb) | —CH₃ | —OH | -t-butyl |
| GOW(IVa) or (IVb) | —CH₃ | —OH | -iso-butyl |
| GOX(IVa) or (IVb) | —CH₃ | —OH | —OCH₃ |
| GOY(IVa) or (IVb) | —CH₃ | —OH | —OC₂H₅ |
| GOZ(IVa) or (IVb) | —CH₃ | —OH | —OC₃H₇ |
| GPA(IVa) or (IVb) | —CH₃ | —OH | —CHF₂ |
| GPB(IVa) or (IVb) | —CH₃ | —OH | —CF₃ |
| GPC(IVa) or (IVb) | —CH₃ | —OH | —CHCl₂ |
| GPD(IVa) or (IVb) | —CH₃ | —OH | —CCl₃ |
| GPE(IVa) or (IVb) | —CH₃ | —OH | —F |
| GPF(IVa) or (IVb) | —CH₃ | —OH | —Cl |
| GPG(IVa) or (IVb) | —CH₃ | —OH | —Br |
| GPH(IVa) or (IVb) | —CH₃ | —OH | —I |
| GPI(IVa) or (IVb) | —CH₃ | —F | —H |
| GPJ(IVa) or (IVb) | —CH₃ | —F | —CH₃ |
| GPK(IVa) or (IVb) | —CH₃ | —F | -n-propyl |
| GPL(IVa) or (IVb) | —CH₃ | —F | -n-butyl |
| GPM(IVa) or (IVb) | —CH₃ | —F | -t-butyl |
| GPN(IVa) or (IVb) | —CH₃ | —F | -iso-butyl |
| GPO(IVa) or (IVb) | —CH₃ | —F | —OCH₃ |
| GPP(IVa) or (IVb) | —CH₃ | —F | —OC₂H₅ |
| GPQ(IVa) or (IVb) | —CH₃ | —F | —OC₃H₇ |
| GPR(IVa) or (IVb) | —CH₃ | —F | —CHF₂ |
| GPS(IVa) or (IVb) | —CH₃ | —F | —CF₃ |
| GPT(IVa) or (IVb) | —CH₃ | —F | —CHCl₂ |
| GPU(IVa) or (IVb) | —CH₃ | —F | —CCl₃ |
| GPV(IVa) or (IVb) | —CH₃ | —F | —F |
| GPW(IVa) or (IVb) | —CH₃ | —F | —CI |
| GPX(IVa) or (IVb) | —CH₃ | —F | —Br |
| GPY(IVa) or (IVb) | —CH₃ | —F | —I |
| GPZ(IVa) or (IVb) | —CH₃ | —Cl | —H |
| GQA(IVa) or (IVb) | —CH₃ | —Cl | —CH₃ |
| GQB(IVa) or (IVb) | —CH₃ | —Cl | -n-propyl |
| GQC(IVa) or (IVb) | —CH₃ | —Cl | -n-butyl |
| GQD(IVa) or (IVb) | —CH₃ | —Cl | -t-butyl |
| GQE(IVa) or (IVb) | —CH₃ | —Cl | -iso-butyl |
| GQF(IVa) or (IVb) | —CH₃ | —Cl | —OCH₃ |
| GQG(IVa) or (IVb) | —CH₃ | —Cl | —OC₂H₅ |
| GQH(IVa) or (IVb) | —CH₃ | —Cl | —OC₃H₇ |
| GQI(IVa) or (IVb) | —CH₃ | —Cl | —CHF₂ |
| GQJ(IVa) or (IVb) | —CH₃ | —Cl | —CF₃ |
| GQK(IVa) or (IVb) | —CH₃ | —Cl | —CHCl₂ |
| GQL(IVa) or (IVb) | —CH₃ | —Cl | —Cd3 |
| GQM(IVa) or (IVb) | —CH₃ | —Cl | —F |
| GQN(IVa) or (IVb) | —CH₃ | —Cl | —Cl |
| GQO(IVa) or (IVb) | —CH₃ | —Cl | —Br |
| GQP(IVa) or (IVb) | —CH₃ | —Cl | —I |
| GQQ(IVa) or (IVb) | —CH₃ | —Br | —H |
| GQR(IVa) or (IVb) | —CH₃ | —Br | —CH₃ |
| GQS(IVa) or (IVb) | —CH₃ | —Br | -n-propyl |
| GQT(IVa) or (IVb) | —CH₃ | —Br | -n-butyl |
| GQU(IVa) or (IVb) | —CH₃ | —Br | -t-butyl |
| GQV(IVa) or (IVb) | —CH₃ | —Br | -iso-butyl |
| GQW(IVa) or (IVb) | —CH₃ | —Br | —OCH₃ |
| GQX(IVa) or (IVb) | —CH₃ | —Br | —OC₂H₅ |
| GQY(IVa) or (IVb) | —CH₃ | —Br | —OC₃H₇ |
| GQZ(IVa) or (IVb) | —CH₃ | —Br | —CHF₂ |
| GRA(IVa) or (IVb) | —CH₃ | —Br | —CF₃ |
| GRB(IVa) or (IVb) | —CH₃ | —Br | —CHCl₂ |
| GRC(IVa) or (IVb) | —CH₃ | —Br | —CCl₃ |
| GRD(IVa) or (IVb) | —CH₃ | —Br | —F |
| GRE(IVa) or (IVb) | —CH₃ | —Br | —Cl |
| GRF(IVa) or (IVb) | —CH₃ | —Br | —Br |
| GRG(IVa) or (IVb) | —CH₃ | —Br | —I |
| GRH(IVa) or (IVb) | —CH₃ | —I | —H |
| GRI(IVa) or (IVb) | —CH₃ | —I | —CH₃ |
| GRJ(IVa) or (IVb) | —CH₃ | —I | -n-propyl |
| GRK(IVa) or (IVb) | —Cl3 | —I | -n-butyl |
| GRL(IVa) or (IVb) | —CH₃ | —I | -t-butyl |
| GRM(IVa) or (IVb) | —CH₃ | —I | -iso-butyl |
| GRN(IVa) or (IVb) | —Cl3 | —I | —OCH₃ |
| GRO(IVa) or (IVb) | —CH₃ | —I | —OC₂H₅ |
| GRP(IVa) or (IVb) | —Cl3 | —I | —OC₃H₇ |
| GRQ(IVa) or (IVb) | —CH₃ | —I | —CHF₂ |
| GRR(IVa) or (IVb) | —CH₃ | —I | —CF₃ |
| GRS(IVa) or (IVb) | —CH₃ | —I | —CHCl₂ |
| GRT(IVa) or (IVb) | —CH₃ | —I | —CCl₃ |
| GRU(IVa) or (IVb) | —CH₃ | —I | —F |
| GRV(IVa) or (IVb) | —CH₃ | —I | —Cl |
| GRW(IVa) or (IVb) | —CH₃ | —I | —Br |
| GRX(IVa) or (IVb) | —CH₃ | —I | —I |
| GRY(IVa) or (IVb) | —CH₃ | —NO₂ | —H |
| GRZ(IVa) or (IVb) | —CH₃ | —NO₂ | —CH₃ |
| GSA(IVa) or (IVb) | —CH₃ | —NO₂ | -n-propyl |
| GSB(IVa) or (IVb) | —CH₃ | —NO₂ | -n-butyl |
| GSC(IVa) or (IVb) | —CH₃ | —NO₂ | -t-butyl |
| GSD(IVa) or (IVb) | —CH₃ | —NO₂ | -iso-butyl |
| GSE(IVa) or (IVb) | —CH₃ | —NO₂ | —OCH₃ |
| GSF(IVa) or (IVb) | —CH₃ | —NO₂ | —OC₂H₅ |
| GSG(IVa) or (IVb) | —CH₃ | —NO₂ | —OC₃H₇ |
| GSH(IVa) or (IVb) | —CH₃ | —NO₂ | —CHF₂ |
| GSI(IVa) or (IVb) | —CH₃ | —NO₂ | —CF₃ |
| GSJ(IVa) or (IVb) | —CH₃ | —NO₂ | —CHCL2 |
| GSK(IVa) or (IVb) | —CH₃ | —NO₂ | —CCl₃ |
| GSL(IVa) or (IVb) | —CH₃ | —NO₂ | —F |
| GSM(IVa) or (IVb) | —CH₃ | —NO₂ | —Cl |
| GSN(IVa) or (IVb) | —CH₃ | —NO₂ | —Br |
| GSO(IVa) or (IVb) | —CH₃ | —NO₂ | —I |
| GSP(IVa) or (IVb) | —CH₃ | —CN | —H |
| GSQ(IVa) or (IVb) | —CH₃ | —CN | —CH₃ |
| GSR(IVa) or (IVb) | —CH₃ | —CN | -n-propyl |
| GSS(IVa) or (IVb) | —CH₃ | —CN | -n-butyl |
| GST(IVa) or (IVb) | —CH₃ | —CN | -t-butyl |
| GSU(IVa) or (IVb) | —CH₃ | —CN | -iso-butyl |
| GSV(IVa) or (IVb) | —CH₃ | —CN | —OCH₃ |
| GSW(IVa) or (IVb) | —CH₃ | —CN | —OC₂H₅ |
| GSX(IVa) or (IVb) | —CH₃ | —CN | —OC₃H₇ |
| GSY(IVa) or (IVb) | —CH₃ | —CN | —CHF₂ |
| GSZ(IVa) or (IVb) | —CH₃ | —CN | —CF₃ |
| GTA(IVa) or (IVb) | —CH₃ | —CN | —CHCl₂ |
| GTB(IVa) or (IVb) | —CH₃ | —CN | —CCl₃ |
| GTC(IVa) or (IVb) | —CH₃ | —CN | —F |
| GTD(IVa) or (IVb) | —CH₃ | —CN | —Cl |
| GTE(IVa) or (IVb) | —CH₃ | —CN | —Br |
| GTF(IVa) or (IVb) | —CH₃ | —CN | —I |
| GTG(IVa) or (IVb) | —CH₃ | —NH₂ | —H |
| GTH(IVa) or (IVb) | —CH₃ | —NH₂ | —CH₃ |
| GTI(IVa) or (IVb) | —CH₃ | —NH₂ | -n-propyl |
| GTJ(IVa) or (IVb) | —CH₃ | —NH₂ | -n-butyl |
| GTK(IVa) or (IVb) | —CH₃ | —NIH2 | -t-butyl |
| GTL(IVa) or (IVb) | —CH₃ | —NIH2 | -iso-butyl |
| GTM(IVa) or (IVb) | —CH₃ | —NH₂ | —OCH₃ |
| GTN(IVa) or (IVb) | —CH₃ | —NH₂ | —OC₂H₅ |
| GTO(IVa) or (IVb) | —CH₃ | —NH₂ | —OC₃H₇ |
| GTP(IVa) or (IVb) | —CH₃ | —NH₂ | —CHF₂ |
| GTQ(IVa) or (IVb) | —CH₃ | —NH₂ | —CF₃ |
| GTR(IVa) or (IVb) | —CH₃ | —NH₂ | —Cl |
| GTS(IVa) or (IVb) | —CH₃ | —NH₂ | —CCl₃ |
| GTT(IVa) or (IVb) | —CH₃ | —NH₂ | —F |
| GTU(IVa) or (IVb) | —CH₃ | —NH₂ | —Cl |
| GTV(IVa) or (IVb) | —CH₃ | —NH₂ | —Br |
| GTW(IVa) or (IVb) | —CH₃ | —NH₂ | —I |
| GTX(IVa) or (IVb) | —CH₃ | —CH₃ | —H |
| GTY(IVa) or (IVb) | —CH₃ | —CH₃ | —CH₃ |
| GTZ(IVa) or (IVb) | —CH₃ | —CH₃ | -n-propyl |
| GUA(IVa) or (IVb) | —CH₃ | —CH₃ | -n-butyl |
| GUB(IVa) or (IVb) | —CH₃ | —CH₃ | -t-butyl |
| GUC(IVa) or (IVb) | —CH₃ | —CH₃ | -iso-butyl |
| GUD(IVa) or (IVb) | —CH₃ | —CH₃ | —OCH₃ |
| GUE(IVa) or (IVb) | —CH₃ | —CH₃ | —OC₂H₅ |
| GUF(IVa) or (IVb) | —CH₃ | —CH₃ | —OC₃H₇ |
| GUG(IVa) or (IVb) | —CH₃ | —CH₃ | —CHF₂ |
| GUH(IVa) or (IVb) | —CH₃ | —CH₃ | —CF₃ |
| GUI(IVa) or (IVb) | —CH₃ | —CH₃ | —CHCl₂ |
| GUJ(IVa) or (IVb) | —CH₃ | —CH₃ | —CCl₃ |
| GUK(IVa) or (IVb) | —CH₃ | —CH₃ | —F |
| GUL(IVa) or (IVb) | —CH₃ | —CH₃ | —Cl |
| GUM(IVa) or (IVb) | —CH₃ | —CH₃ | —Br |
| GUN(IVa) or (IVb) | —CH₃ | —CH₃ | —I |

TABLE 4

| | |
|---|---|
| | (Va) |
| 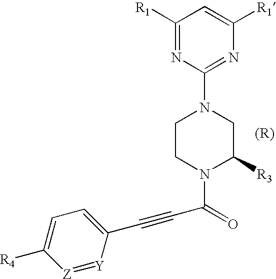 | |
| 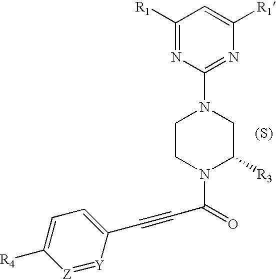 | (Vb) |

and pharmaceutically acceptable salts thereof, where:

| Compound | R₁′ | R₁ | R₃ | R₄ | Y | Z |
|---|---|---|---|---|---|---|
| GUO(Va) or (Vb) | —H | —CH₃ | —CH₃ | —H | —C(H)— | —C(H)— |
| GUP(Va) or (Vb) | —H | —CH₃ | —CH₃ | —H | —C(H)— | —N— |
| GUQ(Va) or (Vb) | —H | —CH₃ | —CH₃ | —H | —N— | —C(H)— |
| GUR(Va) or (Vb) | —H | —CH₃ | —CH₃ | —F | —C(H)— | —C(H)— |
| GUS(Va) or (Vb) | —H | —CH₃ | —CH₃ | —F | —C(H)— | —N— |
| GUT(Va) or (Vb) | —H | —CH₃ | —CH₃ | —F | —N— | —C(H)— |
| GUU(Va) or (Vb) | —H | —CH₃ | —CH₃ | —OCH₃ | —C(H)— | —C(H)— |
| GUV(Va) or (Vb) | —H | —CH₃ | —CH₃ | —OCH₃ | —C(H)— | —N— |
| GUW(Va) or (Vb) | —H | —CH₃ | —CH₃ | —OCH₃ | —N— | —C(H)— |
| GUX(Va) or (Vb) | —H | —CH₃ | —CH₂OH | —H | —C(H)— | —C(H)— |
| GUY(Va) or (Vb) | —H | —CH₃ | —CH₂OH | —H | —C(H)— | —N— |
| GUZ(Va) or (Vb) | —H | —CH₃ | —CH₂OH | —H | —N— | —C(H)— |
| GVA(Va) or (Vb) | —H | —CH₃ | —CH₂OH | —F | —C(H)— | —C(H)— |
| GVB(Va) or (Vb) | —H | —CH₃ | —CH₂OH | —F | —C(H)— | —N— |
| GVC(Va) or (Vb) | —H | —CH₃ | —CH₂OH | —F | —N— | —C(H)— |
| GVD(Va) or (Vb) | —H | —CH₃ | —CH₂OH | —OCH₃ | —C(H)— | —C(H)— |
| GVE(Va) or (Vb) | —H | —CH₃ | —CH₂OH | —OCH₃ | —C(H)— | —N— |
| GVF(Va) or (Vb) | —H | —CH₃ | —CH₂OH | —OCH₃ | —N— | —C(H)— |
| GVG(Va) | —H | —CH₃ | —H | —H | —C(H)— | —C(H)— |
| GVH(Va) | —H | —CH₃ | —H | —H | —C(H)— | —N— |
| GVI(Va) | —H | —CH₃ | —H | —H | —N— | —C(H)— |
| GVJ(Va) | —H | —CH₃ | —H | —F | —C(H)— | —C(H)— |
| GVK(Va) | —H | —CH₃ | —H | —F | —C(H)— | —N— |
| GVL(Va) | —H | —CH₃ | —H | —F | —N— | —C(H)— |
| GVM(Va) | —H | —CH₃ | —H | —OCH₃ | —C(H)— | —C(H)— |
| GVN(Va) | —H | —CH₃ | —H | —OCH₃ | —C(H)— | —N— |
| GVO(Va) | —H | —CH₃ | —H | —OCH₃ | —N— | —C(H)— |
| GVP(Va) or (Vb) | —H | —OCH₃ | —CH₃ | —H | —C(H)— | —C(H)— |
| GVQ(Va) or (Vb) | —H | —OCH₃ | —CH₃ | —H | —C(H)— | —N— |
| GVR(Va) or (Vb) | —H | —OCH₃ | —CH₃ | —H | —N— | —C(H)— |
| GVS(Va) or (Vb) | —H | —OCH₃ | —CH₃ | —F | —C(H)— | —C(H)— |
| GVT(Va) or (Vb) | —H | —OCH₃ | —CH₃ | —F | —C(H)— | —N— |
| GVU(Va) or (Vb) | —H | —OCH₃ | —CH₃ | —F | —N— | —C(H)— |
| GVV(Va) or (Vb) | —H | —OCH₃ | —CH₃ | —OCH₃ | —C(H)— | —C(H)— |
| GVW(Va) or (Vb) | —H | —OCH₃ | —CH₃ | —OCH₃ | —C(H)— | —N— |
| GVX(Va) or (Vb) | —H | —OCH₃ | —CH₃ | —OCH₃ | —N— | —C(H)— |
| GVY(Va) or (Vb) | —H | —OCH₃ | —CH₂OH | —H | —C(H)— | —C(H)— |
| GVZ(Va) or (Vb) | —H | —OCH₃ | —CH₂OH | —H | —C(H)— | —N— |
| GWA(Va) or (Vb) | —H | —OCH₃ | —CH₂OH | —H | —N— | —C(H)— |
| GWB(Va) or (Vb) | —H | —OCH₃ | —CH₂OH | —F | —C(H)— | —C(H)— |
| GWC(Va) or (Vb) | —H | —OCH₃ | —CH₂OH | —F | —C(H)— | —N— |
| GWD(Va) or (Vb) | —H | —OCH₃ | —CH₂OH | —F | —N— | —C(H)— |
| GWE(Va) or (Vb) | —H | —OCH₃ | —CH₂OH | —OCH₃ | —C(H)— | —C(H)— |
| GWF(Va) or (Vb) | —H | —OCH₃ | —CH₂OH | —OCH₃ | —C(H)— | —N— |
| GWG(Va) or (Vb) | —H | —OCH₃ | —CH₂OH | —OCH₃ | —N— | —C(H)— |
| GWH(Va) | —H | —OCH₃ | —H | —H | —C(H)— | —C(H)— |
| GWI(Va) | —H | —OCH₃ | —H | —H | —C(H)— | —N— |
| GWJ(Va) | —H | —OCH₃ | —H | —H | —N— | —C(H)— |
| GWK(Va) | —H | —OCH₃ | —H | —F | —C(H)— | —C(H)— |
| GWL(Va) | —H | —OCH₃ | —H | —F | —C(H)— | —N— |
| GWM(Va) | —H | —OCH₃ | —H | —F | —N— | —C(H)— |
| GWN(Va) | —H | —OCH₃ | —H | —OCH₃ | —C(H)— | —C(H)— |
| GWO(Va) | —H | —OCH₃ | —H | —OCH₃ | —C(H)— | —N— |
| GWP(Va) | —H | —OCH₃ | —H | —OCH₃ | —N— | —C(H)— |
| GWQ(Va) or (Vb) | —H | —Cl | —CH₃ | —H | —C(H)— | —C(H)— |
| GWR(Va) or (Vb) | —H | —Cl | —CH₃ | —H | —C(H)— | —N— |
| GWS(Va) or (Vb) | —H | —Cl | —CH₃ | —H | —N— | —C(H)— |
| GWT(Va) or (Vb) | —H | —Cl | —CH₃ | —F | —C(H)— | —C(H)— |
| GWU(Va) or (Vb) | —H | —Cl | —CH₃ | —F | —C(H)— | —N— |
| GWV(Va) or (Vb) | —H | —Cl | —CH₃ | —F | —N— | —C(H)— |
| GWW(Va) or (Vb) | —H | —Cl | —CH₃ | —OCH₃ | —C(H)— | —C(H)— |
| GWX(Va) or (Vb) | —H | —Cl | —CH₃ | —OCH₃ | —C(H)— | —N— |
| GWY(Va) or (Vb) | —H | —Cl | —CH₃ | —OCH₃ | —N— | —C(H)— |
| GWZ(Va) or (Vb) | —H | —Cl | —CH₂OH | —H | —C(H)— | —C(H)— |
| GXA(Va) or (Vb) | —H | —Cl | —CH₂OH | —H | —C(H)— | —N— |
| GXB(Va) or (Vb) | —H | —Cl | —CH₂OH | —H | —N— | —C(H)— |
| GXC(Va) or (Vb) | —H | —Cl | —CH₂OH | —F | —C(H)— | —C(H)— |
| GXD(Va) or (Vb) | —H | —Cl | —CH₂OH | —F | —C(H)— | —N— |
| GXE(Va) or (Vb) | —H | —Cl | —CH₂OH | —F | —N— | —C(H)— |
| GXF(Va) or (Vb) | —H | —Cl | —CH₂OH | —OCH₃ | —C(H)— | —C(H)— |
| GXG(Va) or (Vb) | —H | —Cl | —CH₂OH | —OCH₃ | —C(H)— | —N— |
| GXH(Va) or (Vb) | —H | —Cl | —CH₂OH | —OCH₃ | —N— | —C(H)— |
| GXI(Va) | —H | —Cl | —H | —H | —C(H)— | —C(H)— |
| GXJ(Va) | —H | —Cl | —H | —H | —C(H)— | —N— |
| GXK(Va) | —H | —Cl | —H | —H | —N— | —C(H)— |
| GXL(Va) | —H | —Cl | —H | —F | —C(H)— | —C(H)— |
| GXM(Va) | —H | —Cl | —H | —F | —C(H)— | —N— |
| GXN(Va) | —H | —Cl | —H | —F | —N— | —C(H)— |
| GXO(Va) | —H | —Cl | —H | —OCH₃ | —C(H)— | —C(H)— |
| GXP(Va) | —H | —Cl | —H | —OCH₃ | —C(H)— | —N— |
| GXQ(Va) | —H | —Cl | —H | —OCH₃ | —N— | —C(H)— |
| GXR(Va) or (Vb) | —H | —H | —CH₃ | —H | —C(H)— | —C(H)— |
| GXS(Va) or (Vb) | —H | —H | —CH₃ | —H | —C(H)— | —N— |
| GXT(Va) or (Vb) | —H | —H | —CH₃ | —H | —N— | —C(H)— |
| GXU(Va) or (Vb) | —H | —H | —CH₃ | —F | —C(H)— | —C(H)— |
| GXV(Va) or (Vb) | —H | —H | —CH₃ | —F | —C(H)— | —N— |
| GXW(Va) or (Vb) | —H | —H | —CH₃ | —F | —N— | —C(H)— |
| GXX(Va) or (Vb) | —H | —H | —CH₃ | —OCH₃ | —C(H)— | —C(H)— |
| GXY(Va) or (Vb) | —H | —H | —CH₃ | —OCH₃ | —C(H)— | —N— |
| GXZ(Va) or (Vb) | —H | —H | —CH₃ | —OCH₃ | —N— | —C(H)— |
| GYA(Va) or (Vb) | —H | —H | —CH₂OH | —H | —C(H)— | —C(H)— |
| GYB(Va) or (Vb) | —H | —H | —CH₂OH | —H | —C(H)— | —N— |
| GYC(Va) or (Vb) | —H | —H | —CH₂OH | —H | —N— | —C(H)— |
| GYD(Va) or (Vb) | —H | —H | —CH₂OH | —F | —C(H)— | —C(H)— |
| GYE(Va) or (Vb) | —H | —H | —CH₂OH | —F | —C(H)— | —N— |
| GYF(Va) or (Vb) | —H | —H | —CH₂OH | —F | —N— | —C(H)— |
| GYG(Va) or (Vb) | —H | —H | —CH₂OH | —OCH₃ | —C(H)— | —C(H)— |
| GYH(Va) or (Vb) | —H | —H | —CH₂OH | —OCH₃ | —C(H)— | —N— |
| GYI(Va) or (Vb) | —H | —H | —CH₂OH | —OCH₃ | —N— | —C(H)— |
| GYJ(Va) | —H | —H | —H | —H | —C(H)— | —C(H)— |
| GYK(Va) | —H | —H | —H | —H | —C(H)— | —N— |
| GYL(Va) | —H | —H | —H | —H | —N— | —C(H)— |
| GYM(Va) | —H | —H | —H | —F | —C(H)— | —C(H)— |
| GYN(Va) | —H | —H | —H | —F | —C(H)— | —N— |
| GYO(Va) | —H | —H | —H | —F | —N— | —C(H)— |
| GYP(Va) | —H | —H | —H | —OCH₃ | —C(H)— | —C(H)— |
| GYQ(Va) | —H | —H | —H | —OCH₃ | —C(H)— | —N— |
| GYR(Va) | —H | —H | —H | —OCH₃ | —N— | —C(H)— |
| GYS(Va) or (Vb) | —CH₃ | —CH₃ | —CH₃ | —H | —C(H)— | —C(H)— |
| GYT(Va) or (Vb) | —CH₃ | —CH₃ | —CH₃ | —H | —C(H)— | —N— |
| GYU(Va) or (Vb) | —CH₃ | —CH₃ | —CH₃ | —H | —N— | —C(H)— |
| GYV(Va) or (Vb) | —CH₃ | —CH₃ | —CH₃ | —F | —C(H)— | —C(H)— |
| GYW(Va) or (Vb) | —CH₃ | —CH₃ | —CH₃ | —F | —C(H)— | —N— |
| GYX(Va) or (Vb) | —CH₃ | —CH₃ | —CH₃ | —F | —N— | —C(H)— |
| GYY(Va) or (Vb) | —CH₃ | —CH₃ | —CH₂OH | —H | —C(H)— | —C(H)— |
| GYZ(Va) or (Vb) | —CH₃ | —CH₃ | —CH₂OH | —H | —C(H)— | —N— |
| GZA(Va) or (Vb) | —CH₃ | —CH₃ | —CH₂OH | —H | —N— | —C(H)— |
| GZB(Va) or (Vb) | —CH₃ | —CH₃ | —CH₂OH | —F | —C(H)— | —C(H)— |
| GZC(Va) or (Vb) | —CH₃ | —CH₃ | —CH₂OH | —F | —C(H)— | —N— |
| GZD(Va) or (Vb) | —CH₃ | —CH₃ | —CH₂OH | —F | —N— | —C(H)— |
| GZE(Va) | —CH₃ | —CH₃ | —H | —H | —C(H)— | —C(H)— |
| GZF(Va) | —CH₃ | —CH₃ | —H | —H | —C(H)— | —N— |
| GZG(Va) | —CH₃ | —CH₃ | —H | —H | —N— | —C(H)— |
| GZH(Va) | —CH₃ | —CH₃ | —H | —F | —C(H)— | —C(H)— |
| GZI(Va) | —CH₃ | —CH₃ | —H | —F | —C(H)— | —N— |
| GZJ(Va) | —CH₃ | —CH₃ | —H | —F | —N— | —C(H)— |
| GZK(Va) or (Vb) | —CH₃ | —OCH₃ | —CH₃ | —H | —C(H)— | —C(H)— |
| GZL(Va) or (Vb) | —CH₃ | —OCH₃ | —CH₃ | —H | —C(H)— | —N— |
| GZM(Va) or (Vb) | —CH₃ | —OCH₃ | —CH₃ | —H | —N— | —C(H)— |
| GZN(Va) or (Vb) | —CH₃ | —OCH₃ | —CH₃ | —F | —C(H)— | —C(H)— |
| GZO(Va) or (Vb) | —CH₃ | —OCH₃ | —CH₃ | —F | —C(H)— | —N— |
| GZP(Va) or (Vb) | —CH₃ | —OCH₃ | —CH₃ | —F | —N— | —C(H)— |
| GZQ(Va) or (Vb) | —CH₃ | —OCH₃ | —CH₃ | —OCH₃ | —C(H)— | —C(H)— |
| GZR(Va) or (Vb) | —CH₃ | —OCH₃ | —CH₃ | —OCH₃ | —C(H)— | —N— |
| GZS(Va) or (Vb) | —CH₃ | —OCH₃ | —CH₃ | —OCH₃ | —N— | —C(H)— |
| GZT(Va) or (Vb) | —CH₃ | —OCH₃ | —CH₂OH | —H | —C(H)— | —C(H)— |
| GZU(Va) or (Vb) | —CH₃ | —OCH₃ | —CH₂OH | —H | —C(H)— | —N— |
| GZV(Va) or (Vb) | —CH₃ | —OCH₃ | —CH₂OH | —H | —N— | —C(H)— |
| GZW(Va) or (Vb) | —CH₃ | —OCH₃ | —CH₂OH | —F | —C(H)— | —C(H)— |
| GZX(Va) or (Vb) | —CH₃ | —OCH₃ | —CH₂OH | —F | —C(H)— | —N— |
| GZY(Va) or (Vb) | —CH₃ | —OCH₃ | —CH₂OH | —F | —N— | —C(H)— |
| GZZ(Va) or (Vb) | —CH₃ | —OCH₃ | —CH₂OH | —OCH₃ | —C(H)— | —C(H)— |
| HAA(Va) or (Vb) | —CH₃ | —OCH₃ | —CH₂OH | —OCH₃ | —C(H)— | —N— |
| HAB(Va) or (Vb) | —CH₃ | —OCH₃ | —CH₂OH | —OCH₃ | —N— | —C(H)— |
| HAC(Va) | —CH₃ | —OCH₃ | —H | —H | —C(H)— | —C(H)— |
| HAD(Va) | —CH₃ | —OCH₃ | —H | —H | —C(H)— | —N— |
| HAE(Va) | —CH₃ | —OCH₃ | —H | —H | —N— | —C(H)— |
| HAF(Va) | —CH₃ | —OCH₃ | —H | —F | —C(H)— | —C(H)— |
| HAG(Va) | —CH₃ | —OCH₃ | —H | —F | —C(H)— | —N— |
| HAH(Va) | —CH₃ | —OCH₃ | —H | —F | —N— | —C(H)— |
| HAI(Va) | —CH₃ | —OCH₃ | —H | —OCH₃ | —C(H)— | —C(H)— |
| HAJ(Va) | —CH₃ | —OCH₃ | —H | —OCH₃ | —C(H)— | —N— |
| HAK(Va) | —CH₃ | —OCH₃ | —H | —OCH₃ | —N— | —C(H)— |
| HAL(Va) or (Vb) | —CH₃ | —Cl | —CH₃ | —H | —C(H)— | —C(H)— |
| HAM(Va) or (Vb) | —CH₃ | —Cl | —CH₃ | —H | —C(H)— | —N— |
| HAN(Va) or (Vb) | —CH₃ | —Cl | —CH₃ | —H | —N— | —C(H) |
| HAO(Va) or (Vb) | —CH₃ | —Cl | —CH₃ | —F | —C(H)— | —C(H)— |
| HAP(Va) or (Vb) | —CH₃ | —Cl | —CH₃ | —F | —C(H)— | —N— |
| HAQ(Va) or (Vb) | —CH₃ | —Cl | —CH₃ | —F | —N— | —C(H)— |
| HAR(Va) or (Vb) | —CH₃ | —Cl | —CH₃ | —OCH₃ | —C(H)— | —C(H)— |
| HAS(Va) or (Vb) | —CH₃ | —Cl | —CH₃ | —OCH₃ | —C(H)— | —N— |
| HAT(Va) or (Vb) | —CH₃ | —Cl | —CH₃ | —OCH₃ | —N— | —C(H)— |
| HAU(Va) or (Vb) | —CH₃ | —Cl | —CH₂OH | —H | —C(H)— | —C(H)— |
| HAV(Va) or (Vb) | —CH₃ | —Cl | —CH₂OH | —H | —C(H)— | —N— |
| HAW(Va) or (Vb) | —CH₃ | —Cl | —CH₂OH | —H | —N— | —C(H)— |
| HAX(Va) or (Vb) | —CH₃ | —Cl | —CH₂OH | —F | —C(H)— | —C(H)— |
| HAY(Va) or (Vb) | —CH₃ | —Cl | —CH₂OH | —F | —C(H)— | —N— |
| HAZ(Va) or (Vb) | —CH₃ | —Cl | —CH₂OH | —F | —N— | —C(H)— |
| HBA(Va) or (Vb) | —CH₃ | —Cl | —CH₂OH | —OCH₃ | —C(H)— | —C(H)— |
| HBB(Va) or (Vb) | —CH₃ | —Cl | —CH₂OH | —OCH₃ | —C(H)— | —N— |
| HBC(Va) or (Vb) | —CH₃ | —Cl | —CH₂OH | —OCH₃ | —N— | —C(H)— |
| HBD(Va) | —CH₃ | —Cl | —H | —H | —C(H)— | —C(H)— |
| HBE(Va) | —CH₃ | —Cl | —H | —H | —C(H)— | —N— |
| HBF(Va) | —CH₃ | —Cl | —H | —H | —N— | —C(H)— |
| HBG(Va) | —CH₃ | —Cl | —H | —F | —C(H)— | —C(H)— |
| HBH(Va) | —CH₃ | —Cl | —H | —F | —C(H)— | —N— |
| HBI(Va) | —CH₃ | —Cl | —H | —F | —N— | —C(H)— |
| HBJ(Va) | —CH₃ | —Cl | —H | —OCH₃ | —C(H)— | —C(H)— |
| HBK(Va) | —CH₃ | —Cl | —H | —OCH₃ | —C(H)— | —N— |
| HBL(Va) | —CH₃ | —Cl | —H | —OCH₃ | —N— | —C(H)— |
| HBM(Va) or (Vb) | —CH₃ | —H | —CH₃ | —H | —C(H)— | —C(H)— |
| HBN(Va) or (Vb) | —CH₃ | —H | —CH₃ | —H | —C(H)— | —N— |
| HBO(Va) or (Vb) | —CH₃ | —H | —CH₃ | —H | —N— | —C(H)— |
| HBP(Va) or (Vb) | —CH₃ | —H | —CH₃ | —F | —C(H)— | —C(H)— |
| HBQ(Va) or (Vb) | —CH₃ | —H | —CH₃ | —F | —C(H)— | —N— |
| HBR(Va) or (Vb) | —CH₃ | —H | —CH₃ | —F | —N— | —C(H)— |
| HBS(Va) or (Vb) | —CH₃ | —H | —CH₃ | —OCH₃ | —C(H)— | —C(H)— |
| HBT(Va) or (Vb) | —CH₃ | —H | —CH₃ | —OCH₃ | —C(H)— | —N— |
| HBU(Va) or (Vb) | —CH₃ | —H | —CH₃ | —OCH₃ | —N— | —C(H)— |
| HBV(Va) or (Vb) | —CH₃ | —H | —CH₂OH | —H | —C(H)— | —C(H)— |
| HBW(Va) or (Vb) | —CH₃ | —H | —CH₂OH | —H | —C(H)— | —N— |
| HBX(Va) or (Vb) | —CH₃ | —H | —CH₂OH | —H | —N— | —C(H)— |
| HBY(Va) or (Vb) | —CH₃ | —H | —CH₂OH | —F | —C(H)— | —C(H)— |
| HBZ(Va) or (Vb) | —CH₃ | —H | —CH₂OH | —F | —C(H)— | —N— |
| HCA(Va) or (Vb) | —CH₃ | —H | —CH₂OH | —F | —N— | —C(H)— |
| HCB(Va) or (Vb) | —CH₃ | —H | —CH₂OH | —OCH₃ | —C(H)— | —C(H)— |
| HCC(Va) or (Vb) | —CH₃ | —H | —CH₂OH | —OCH₃ | —C(H)— | —N— |
| HCD(Va) or (Vb) | —CH₃ | —H | —CH₂OH | —OCH₃ | —N— | —C(H)— |
| HCE(Va) | —CH₃ | —H | —H | —H | —C(H)— | —C(H)— |
| HCF(Va) | —CH₃ | —H | —H | —H | —C(H)— | —N— |
| HCG(Va) | —CH₃ | —H | —H | —H | —N— | —C(H)— |
| HCH(Va) | —CH₃ | —H | —H | —F | —C(H)— | —C(H)— |
| HCI(Va) | —CH₃ | —H | —H | —F | —C(H)— | —N— |
| HCJ(Va) | —CH₃ | —H | —H | —F | —N— | —C(H)— |
| HCK(Va) | —CH₃ | —H | —H | —OCH₃ | —C(H)— | —C(H)— |
| HCL(Va) | —CH₃ | —H | —H | —OCH₃ | —C(H)— | —N— |
| HCM(Va) | —CH₃ | —H | —H | —OCH₃ | —N— | —C(H)— |

### 4.3 Definitions

As used herein, the terms used above having following meaning:

“—(C₁-C₁₀)alkyl” means a saturated straight chain or branched non-cyclic hydrocarbon having from 1 to 10 carbon atoms. Representative saturated straight chain —(C₁-C₁₀)alkyls include -methyl, -ethyl, -n-propyl, -n-butyl, -n-pentyl, -n-hexyl, -n-heptyl, -n-octyl, -n-nonyl, and -n-decyl. Representative saturated branched —(C₁-C₁₀)alkyls include -isopropyl, -sec-butyl, -isobutyl, -tert-butyl, -isopentyl, -2-methylbutyl, -3-methylbutyl, -2,2-dimethylbutyl, -2,3-dimethylbutyl, -2-methylpentyl, -3-methylpentyl, -4-methylpentyl, -2-methylhexyl, -3-methylhexyl, -4-methylhexyl, -5-methylhexyl, -2,3-dimethylbutyl, -2,3-dimethylpentyl, -2,4-dimethylpentyl, -2,3-dimethylhexyl, -2,4-dimethylhexyl, -2,5-dimethylhexyl, -2,2-dimethylpentyl, -2,2-dimethylhexyl, -3,3-dimethylpentyl, -3,3-dimethylhexyl, -4,4-dimethylhexyl, -2-ethylpentyl, -3-ethylpentyl, -2-ethylhexyl, -3-ethylhexyl, -4-ethylhexyl, -2-methyl-2-ethylpentyl, -2-methyl-3-ethylpentyl, -2-methyl-4-ethylpentyl, -2-methyl-2-ethylhexyl, -2-methyl-3-ethylhexyl, -2-methyl-4-ethylhexyl, -2,2-diethylpentyl, -3,3-diethylhexyl, -2,2-diethylhexyl, -3,3-diethylhexyl and the like.

“—(C₁-C₆)alkyl” means a saturated straight chain or branched non-cyclic hydrocarbon having from 1 to 6 carbon atoms. Representative saturated straight chain —(C₁-C₆)alkyls include -methyl, -ethyl, -n-propyl, -n-butyl, -n-pentyl, and -n-hexyl. Representative saturated branched —(C₁-C₆)alkyls include -isopropyl, -sec-butyl, -isobutyl, -tert-butyl, -isopentyl, -2-methylbutyl, -3-methylbutyl, -2,2-dimethylbutyl, -2,3-dimethylbutyl, -2-methylpentyl, -3-methylpentyl, -4-methylpentyl and the like.

“—(C₁-C₄)alkyl” means a saturated straight chain or branched non-cyclic hydrocarbon having from 1 to 4 carbon atoms. Representative saturated straight chain —(C₁-C₄)alkyls include -methyl, -ethyl, -n-propyl, and -n-butyl. Representative saturated branched —(C₁-C₄)alkyls include -isopropyl, -sec-butyl, -isobutyl, and -tert-butyl.

“—(C₁-C₃)alkyl” means a saturated straight chain or branched non-cyclic hydrocarbon having from 1 to 3 carbon atoms. Representative saturated straight chain —(C₁-C₃)alkyls include -methyl, -ethyl, and -n-propyl. A representative saturated branched —(C₁-C₃)alkyl is -isopropyl.

“—(C₂-C₁₀)alkenyl” means a straight chain or branched non-cyclic hydrocarbon having from 2 to 10 carbon atoms and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

“—(C₂-C₆)alkenyl” means a straight chain or branched non-cyclic hydrocarbon having from 2 to 6 carbon atoms and including at least one carbon-carbon double bond. Representative straight chain and branched (C₂-C₆)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl and the like.

“—(C₂-C₁₀)alkynyl” means a straight chain or branched non-cyclic hydrocarbon having from 2 to 10 carbon atoms and including at lease one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl and the like.

“—(C₂-C₆)alkynyl” means a straight chain or branched non-cyclic hydrocarbon having from 2 to 6 carbon atoms and including at lease one carbon-carbon triple bond. Representative straight chain and branched (C₂-C₆)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl and the like.

“—(C₃-C₁₀)cycloalkyl” means a saturated cyclic hydrocarbon having from 3 to 10 carbon atoms. Representative (C₃-C₁₀)cycloalkyls include -cyclopropyl, -cyclobutyl, -cyclopentyl, -cyclohexyl, -cycloheptyl, -cyclooctyl, -cyclononyl, and -cyclodecyl.

“—(C₃-C₈)cycloalkyl” means a saturated cyclic hydrocarbon having from 3 to 8 carbon atoms. Representative (C₃-C₈)cycloalkyls include -cyclopropyl, -cyclobutyl, -cyclopentyl, -cyclohexyl, -cycloheptyl, and -cyclooctyl.

“—(C₈-C₁₄)bicycloalkyl” means a bi-cyclic hydrocarbon ring system having from 8 to 14 carbon atoms and at least one saturated cyclic alkyl ring. Representative —(C₈-C₁₄)bicycloalkyls include -indanyl, -1,2,3,4-tetrahydronaphthyl, -5,6,7,8-tetrahydronaphthyl, -perhydronaphthyl and the like.

“—(C₈-C₁₄)tricycloalkyl” means a tri-cyclic hydrocarbon ring system having from 8 to 14 carbon atoms and at least one saturated cyclic alkyl ring. Representative —(C₈-C₁₄)tricycloalkyls include -pyrenyl, -1,2,3,4-tetrahydroanthracenyl, -perhydroanthracenyl, -aceanthreneyl, -1,2,3,4-tetrahydropenanthrenyl, -5,6,7,8-tetrahydrophenanthrenyl, -perhydrophenanthrenyl and the like.

“—(C₅-C₁₀)cycloalkenyl” means a cyclic non-aromatic hydrocarbon having at least one carbon-carbon double bond in the cyclic system and from 5 to 10 carbon atoms. Representative (C₅-C₁₀)cycloalkenyls include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl, -cyclononenyl, -cyclononadienyl, -cyclodecenyl, -cyclodecadienyl and the like.

“—(C₅-C₈)cycloalkenyl” means a cyclic non-aromatic hydrocarbon having at least one carbon-carbon double bond in the cyclic system and from 5 to 8 carbon atoms. Representative (C₅-C₈)cycloalkenyls include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl and the like.

“—(C₈-C₁₄)bicycloalkenyl” means a bi-cyclic hydrocarbon ring system having at least one carbon-carbon double bond in each ring and from 8 to 14 carbon atoms. Representative —(C₈-C₁₄)bicycloalkenyls include -indenyl, -pentalenyl, -naphthalenyl, -azulenyl, -heptalenyl, -1,2,7,8-tetrahydronaphthalenyl and the like.

“—(C₈-C₁₄)tricycloalkenyl” means a tri-cyclic hydrocarbon ring system having at least one carbon-carbon double bond in each ring and from 8 to 14 carbon atoms. Representative —(C₈-C₁₄)tricycloalkenyls include -anthracenyl, -phenanthrenyl, -phenalenyl, -acenaphthalenyl, -as-indacenyl, -s-indacenyl and the like.

“—(5- to 10-membered)heteroaryl” means an aromatic heterocycle ring of 5 to 10 members, including both mono- and bicyclic ring systems, where at least one carbon atom of one or both of the rings is replaced with a heteroatom independently selected from nitrogen, oxygen, and sulfur. In one embodiment one of the -(5- to 10-membered)heteroaryl's rings contain at least one carbon atom. In another embodiment both of the -(5- to 10-membered)heteroaryl's rings contain at least one carbon atom. Representative (5- to 10-membered)heteroaryls include pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, and quinazolinyl.

“-(3- to 7-membered)heterocycle” or “-(3- to 7-membered)heterocyclo” means a 3- to 7-membered monocyclic heterocyclic ring which is either saturated, unsaturated non-aromatic or aromatic. A 3- or a 4-membered heterocycle can contain up to 3 heteroatoms, a 5-membered heterocycle can contain up to 4 heteroatoms, a 6-membered heterocycle can contain up to 6 heteroatoms, and a 7-membered heterocycle can contain up to 7 heteroatoms. Each heteroatom is independently selected from nitrogen, which can be quaternized; oxygen; and sulfur, including sulfoxide and sulfone. The -(3- to 7-membered)heterocycle can be attached via any heteroatom or carbon atom. Representative -(3- to 7-membered)heterocycles include pyridyl, furyl, thiophenyl, pyrrolyl, oxazolyl, imidazolyl, thiazolyl, isoxazolyl, pyrazolyl, isothiazolyl, pyridazinyl, pyrimidinyl, pyrazinyl, triazinyl, morpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl and the like.

“-(3- to 5-membered)heterocycle” or “-(3- to 5-membered)heterocyclo” means a 3- to 5-membered monocyclic heterocyclic ring which is either saturated, unsaturated non-aromatic or aromatic. A 3- or 4-membered heterocycle can contain up to 3 heteroatoms and a 5-membered heterocycle can contain up to 4 heteroatoms. Each heteroatom is independently selected from nitrogen, which can be quaternized; oxygen; and sulfur, including sulfoxide and sulfone. The -(3- to 5-membered)heterocycle can be attached via any heteroatom or carbon atom. Representative -(3- to 5-membered)heterocycles include furyl, thiophenyl, pyrrolyl, oxazolyl, imidazolyl, thiazolyl, isoxazolyl, pyrazolyl, isothiazolyl, triazinyl, pyrrolidinonyl, pyrrolidinyl, hydantoinyl, oxiranyl, oxetanyl, tetrahydrofuranyl, tetrahydrothiophenyl and the like.

“-(7- to 10-membered)bicycloheterocycle” or “-(7- to 10-membered)bicycloheterocyclo” means a 7- to 10-membered bicyclic, heterocyclic ring which is either saturated, unsaturated non-aromatic or aromatic. A-(7- to 10-membered)bicycloheterocycle contains from 1 to 4 heteroatoms independently selected from nitrogen, which can be quaternized; oxygen; and sulfur, including sulfoxide and sulfone. The (7- to 10-membered)bicycloheterocycle can be attached via any heteroatom or carbon atom. Representative -(7- to 10-membered)bicycloheterocycles include -quinolinyl, -isoquinolinyl, -chromonyl, -coumarinyl, -indolyl, -indolizinyl, -benzo[b]furanyl, -benzo[b]thiophenyl, -indazolyl, -purinyl, -4H-quinolizinyl, -isoquinolyl, -quinolyl, -phthalazinyl, -naphthyridinyl, -carbazolyl, -β-carbolinyl and the like.

“—(C₁₄)aryl” means a 14-membered aromatic carbocyclic moiety such as anthryl and phenanthryl.

“—CH₂(halo)” means a methyl group wherein one of the hydrogens of the methyl group has been replaced with a halogen. Representative —CH₂(halo) groups include —CH₂F, —CH₂Cl, —CH₂Br, and —CH₂I.

“—CH(halo)₂” means a methyl group wherein two of the hydrogens of the methyl group have been replaced with a halogen. Representative —CH(halo)₂ groups include —CHF₂, —CHCl₂, —CHBr₂, CHBrCl, CHClI, and —CH₁₂.

“—C(halo)₃” means a methyl group wherein each of the hydrogens of the methyl group has been replaced with a halogen. Representative —C(halo)₃ groups include —CF₃, —CCl₃, —CBr₃, and —Cl₃.

“-Halogen” or “-Halo” means —F, —Cl, —Br, or —I.

The term “pyrimidinyl ring” means

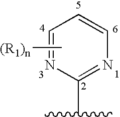

where R₁ and n are defined above for the 2-Pyrimidinylpiperazine Compounds.

The term “animal,” includes, but is not limited to, a cow, monkey, horse, sheep, pig, chicken, turkey, quail, cat, dog, mouse, rat, rabbit, guinea pig, and human.

The phrase “pharmaceutically acceptable salt,” as used herein, is any pharmaceutically acceptable salt that can be prepared from a 2-Pyrimidinylpiperazine Compound, including a salt formed from an acid and a basic functional group, such as a nitrogen group, of one of the 2-Pyrimidinylpiperazine Compounds. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The term “pharmaceutically acceptable salt” also refers to a salt prepared from a 2-Pyrimidinylpiperazine Compound having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia and organic amines, such as unsubstituted or hydroxy-substituted mono-, di- or trialkylamines; dicyclohexylamine; tributyl amine; pyridine; N-methyl-N-ethylamine; diethylamine; triethylamine; mono-, bis- or tris-(2-hydroxy-lower alkyl amines), such as mono-, bis- or tris-(2-hydroxyethyl)amine, 2-hydroxy-tert-butylamine or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)aamine; N-methyl-D-glucamine; and amino acids such as arginine, lysine and the like.

The phrase “effective amount” when used in connection with a 2-Pyrimidinylpiperazine Compound means an amount effective for: (a) treating or preventing a Condition; or (b) inhibiting mGluR5 or mGluR1 function in a cell.

The phrase “effective amount” when used in connection with another therapeutic agent means an amount for providing the therapeutic effect of the other therapeutic agent.

When a first group is “substituted with one or more” second groups, each of one or more of the first group's hydrogen atoms is replaced with a second group.

In one embodiment, a first group is substituted with up to three second groups.

In another embodiment, a first group is substituted with one or two second groups.

In another embodiment, a first group is substituted with only one second group.

The term “UI” means urinary incontinence.

The term “ALS” means amyotrophic lateral sclerosis.

The phrases “treatment of,” “treating” and the like include the amelioration or cessation of a Condition or a symptom thereof.

In one embodiment, treating includes inhibiting, for example, decreasing the overall frequency of episodes of a Condition or a symptom thereof.

The phrases “prevention of,” “preventing” and the like include the avoidance of the onset of a Condition or a symptom thereof.

### 4.4 Methods for Making the 2-Pyrimidinylpiperazine Compounds

The 2-Pyrimidinylpiperazine Compounds can be made using conventional organic synthesis and/or by the following illustrative methods.

The 2-Pyrimidinylpiperazine Compounds of Formula (Ib) where A is —C(O)— or —C(S)— can be made by reacting a compound of formula A with a (C₁-C₁₀)alkyl iodide, or with a (C₂-C₁₀)alkenyl iodide or (C₂-C₁₀)alkynyl iodide in which the iodine atom is bonded to an sp³ carbon atom, at low temperature, e.g., about 0° C. to about −78° C., in the presence of a strong base, e.g., lithium diisopropylamide (“LDA”), optionally in hexamethylphosphoramide (“HMPA”), as shown below in Scheme 1, e.g., for a (C₁-C₁₀)alkyl iodide reactant:

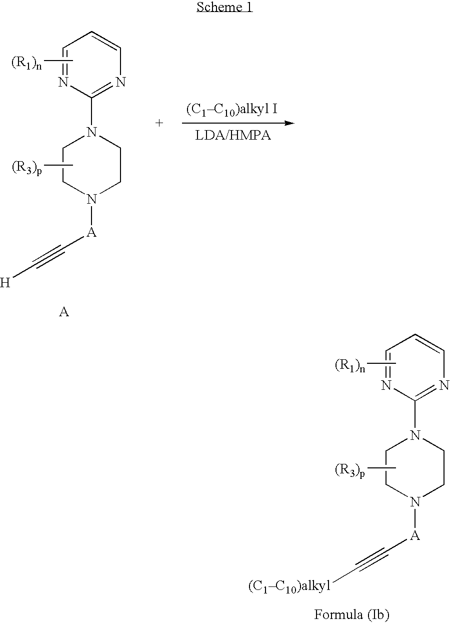

A representative procedure for coupling a terminal acetylene and an alkyl iodide is provided in G. M. Strunz et al., Can. J. Chem. 419-432 (1996).

The 2-Pyrimidinylpiperazine Compounds of Formula (Ic) where A is —C(O)— or —C(S)— can be made by reacting a compound of formula A with an aryl iodide, or with a (C₂-C₁₀)alkenyl iodide or (C₂-C₁₀)alkynyl iodide in which the iodine atom is bonded to an Sp2 or sp carbon atom, at room temperature, e.g., about 25° C., in ethyl acetate (“EtOAc”) in the presence of Pd(Ph₃P)₂(OAc)₂, CuI and triethylamine (“TEA”), as shown below in Scheme 2, e.g., for an aryl iodide reactant:

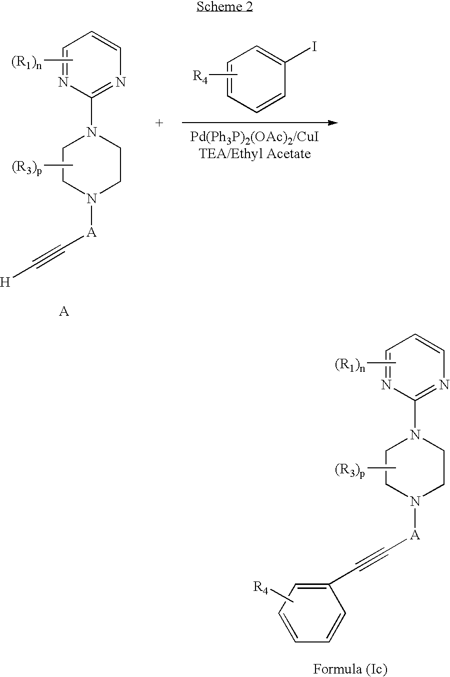

A representative procedure for coupling a terminal acetylene with an aryl iodide is provided in L. A. Hay et al., J. Org. Chem. 5050-5058 (1998).

The compound of formula A where A is —C(O)—, i.e., the compound of formula A′, can be made by reacting a compound of formula B with propynoic acid in the presence of 1-hydroxybenzotriazole hydrate (“HOBtH”) and 1,3-diisopropylcarbodiimide (“DIC”) at room temperature, e.g., about 25° C., as shown below in Scheme 3:

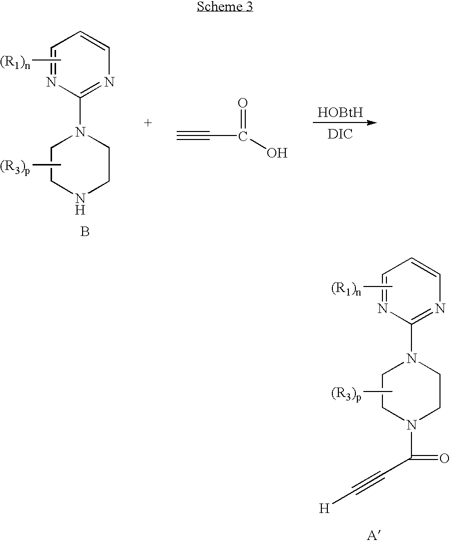

A representative procedure for coupling a carboxylic acid with an amine is provided in F. M. Martin et al., Bioorg. Med. Chem. Lett. 2887-2892 (1999).

The compound of formula A′ can also be made by reacting a compound of formula B with propynoyl chloride in the presence of a tertiary amine, such as TEA, at a temperature about 100° C., as shown below in Scheme 4:

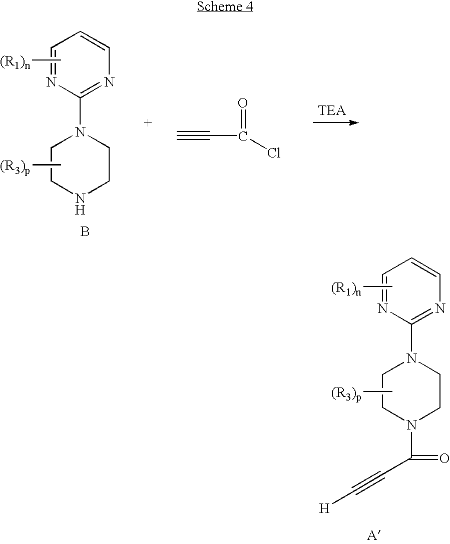

A representative procedure for coupling an acid chloride with an amine is provided in T. R. Herrin et al., J. Med. Chem. 1216-1223 (1975).

The compound of formula A where A is —C(S)—, i.e., the compound of formula A″ below, can be made by, e.g., reacting a compound of formula A′ with Lawesson's reagent at a temperature of about 100° C., as shown below in Scheme 5:

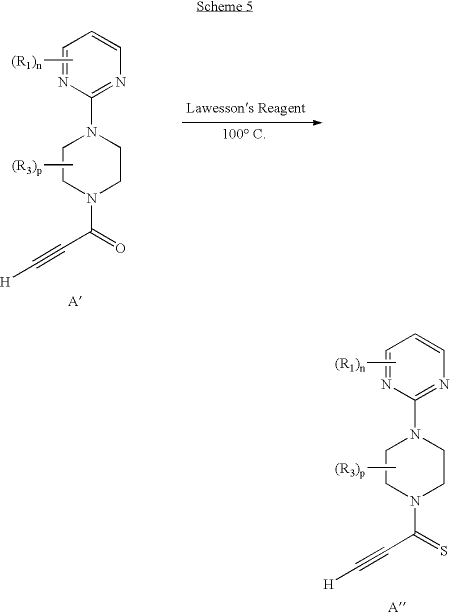

The compound of formula B can be made by reacting a 2-halo-substituted pyrimidine of formula C with an excess of piperazine of formula D in an aprotic organic solvent, e.g., methylene chloride or chloroform, in the presence of a base, e.g., TEA, at a temperature, e.g., of about 50° C., as shown below in Scheme 6:

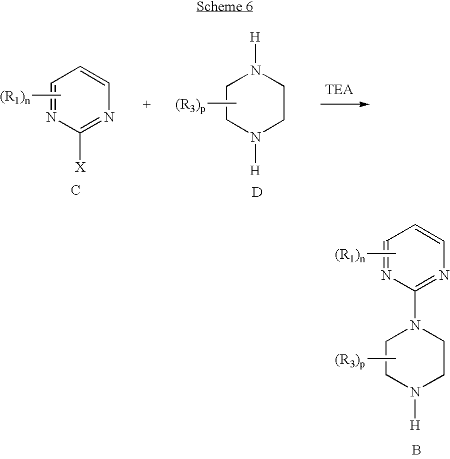

where X is I, Br, Cl or F.

A representative procedure for reacting a 2-halo-pyrimidine with a piperazine is provided in J. A. Tucker et al., J. Med. Chem. 41(19):3727-3735 (1998).

The 2-halo-pyrimidines of formula C and the piperazines of formula D are commercially available or can be made using methods well known to those skilled in the art.

The compound of formula A where A is —CH₂—, —CH(C₁-C₄ alkyl)-, or —C(C₁-C₄ alkyl)(C₁-C₄ alkyl)- can be made by, e.g., reacting Compound B with a halogenated alkyne compound, as shown below in Scheme 7:

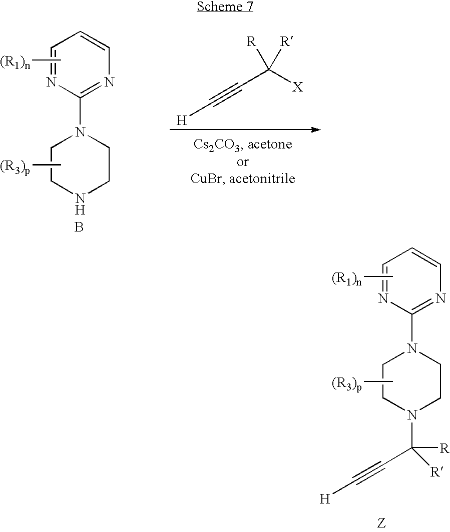

where R and R′ are, independently, H or C₁-C₄ alkyl, and X is Cl, Br, or I. Representative procedures for coupling a halogenated alkyne with an amine are provided in H—R Tsou et al., J. Med. Chem. 2719-2734 (2001) and R. Geri et al., Gazz. Chim. Ital. 241-248 (1994).

Certain 2-Pyrimidinylpiperazine Compounds can have asymmetric centers and therefore exist in different enantiomeric and diastereomeric forms. A 2-Pyrimidinylpiperazine Compound can be in the form of an optical isomer or a diastereomer. Accordingly, the invention encompasses 2-Pyrimidinylpiperazine Compounds and their uses as described herein in the form of their optical isomers, diasteriomers and mixtures thereof, including a racemic mixture. Optical isomers of the 2-Pyrimidinylpiperazine Compounds can be obtained by well known techniques such as chiral chromatography or formation of diastereomeric salts from an optically active acid or base.

In addition, one or more hydrogen, carbon or other atoms of a 2-Pyrimidinylpiperazine Compound can be replaced by an isotope of the hydrogen, carbon or other atoms. Such compounds, which are encompassed by the present invention, are useful as research and diagnostic tools in metabolism pharmacokinetic studies and in binding assays.

### 4.5 Therapeutic Uses of the 2-Pyrimidinylpiperazine Compounds

In accordance with the invention, the 2-Pyrimidinylpiperazine Compounds are administered to an animal in need of treatment or prevention of a Condition.

In one embodiment, an effective amount of a 2-Pyrimidinylpiperazine Compound can be used to treat or prevent any condition treatable or preventable by inhibiting mGluR5. Examples of conditions that are treatable or preventable by inhibiting mGluR5 include, but are not limited to, pain, an addictive disorder, Parkinson's disease, parkinsonism, anxiety, a pruritic condition, and psychosis.

In another embodiment, an effective amount of a 2-Pyrimidinylpiperazine Compound can be used to treat or prevent any condition treatable or preventable by inhibiting mGluR1. Examples of conditions that are treatable or preventable by inhibiting mGluR1 include, but are not limited to, pain, UI, an addictive disorder, Parkinson's disease, parkinsonism, anxiety, epilepsy, a seizure, stroke, a pruritic condition, psychosis, a cognitive disorder, a memory deficit, restricted brain function, Huntington's chorea, ALS, dementia, retinopathy, a muscle spasm, a migraine, vomiting, dyskinesia, and depression.

The 2-Pyrimidinylpiperazine Compounds can be used to treat or prevent acute or chronic pain. Examples of pain treatable or preventable using the 2-Pyrimidinylpiperazine Compounds include, but are not limited to, cancer pain, labor pain, myocardial infarction pain, pancreatic pain, colic pain, post-operative pain, headache pain, muscle pain, arthritic pain, neuropathic pain, and pain associated with a periodontal disease, including gingivitis and periodontitis.

The 2-Pyrimidinylpiperazine Compounds can also be used for treating or preventing pain associated with inflammation or with an inflammatory disease in an animal. Such pain can arise where there is an inflammation of the body tissue which can be a local inflammatory response and/or a systemic inflammation. For example, the 2-Pyrimidinylpiperazine Compounds can be used to treat or prevent pain associated with inflammatory diseases including, but not limited to: organ transplant rejection; reoxygenation injury resulting from organ transplantation (see Grupp et al., J. Mol. Cell Cardiol. 31:297-303 (1999)) including, but not limited to, transplantation of the heart, lung, liver, or kidney; chronic inflammatory diseases of the joints, including arthritis, rheumatoid arthritis, osteoarthritis and bone diseases associated with increased bone resorption; inflammatory lung diseases, such as asthma, adult respiratory distress syndrome, and chronic obstructive airway disease; inflammatory diseases of the eye, including corneal dystrophy, trachoma, onchocerciasis, uveitis, sympathetic ophthalmitis and endophthalmitis; chronic inflammatory diseases of the gum, including gingivitis and periodontitis; tuberculosis; leprosy; inflammatory diseases of the kidney, including uremic complications, glomerulonephritis and nephrosis; inflammatory diseases of the skin, including sclerodermatitis, psoriasis and eczema; inflammatory diseases of the central nervous system, including chronic demyelinating diseases of the nervous system, multiple sclerosis, AIDS-related neurodegeneration and Alzheimer s disease, infectious meningitis, encephalomyelitis, Parkinson's disease, Huntington's disease, amyotrophic lateral sclerosis and viral or autoimmune encephalitis; autoimmune diseases, including Type I and Type II diabetes mellitus; diabetic complications, including, but not limited to, diabetic cataract, glaucoma, retinopathy, nephropathy (such as microaluminuria and progressive diabetic nephropathy), polyneuropathy, mononeuropathies, autonomic neuropathy, gangrene of the feet, atherosclerotic coronary arterial disease, peripheral arterial disease, nonketotic hyperglycemic-hyperosmolar coma, foot ulcers, joint problems, and a skin or mucous membrane complication (such as an infection, a shin spot, a candidal infection or necrobiosis lipoidica diabeticorum); immune-complex vasculitis, and systemic lupus erythematosus (SLE); inflammatory diseases of the heart, such as cardiomyopathy, ischemic heart disease hypercholesterolemia, and atherosclerosis; as well as various other diseases that can have significant inflammatory components, including preeclampsia, chronic liver failure, brain and spinal cord trauma, and cancer. The 2-Pyrimidinylpiperazine Compounds can also be used for treating or preventing pain associated with inflammatory disease that can, for example, be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

The 2-Pyrimidinylpiperazine Compounds can be used to treat or prevent UI. Examples of UI treatable or preventable using the 2-Pyrimidinylpiperazine Compounds include, but are not limited to, urge incontinence, stress incontinence, overflow incontinence, eurogenic incontinence, and total incontinence.

The 2-Pyrimidinylpiperazine Compounds can be used to treat or prevent an addictive disorder, including but not limited to, an eating disorder, an impulse-control disorder, an alcohol-related disorder, a nicotine-related disorder, an amphetamine-related disorder, a cannabis-related disorder, a cocaine-related disorder, an hallucinogen-related disorder, an inhalant-related disorders, and an opioid-related disorder, all of which are further sub-classified as listed below.

Eating disorders include, but are not limited to, Bulimia Nervosa, Nonpurging Type; Bulimia Nervosa, Purging Type; Anorexia; and Eating Disorder not otherwise specified (NOS).

Impulse control disorders include, but are not limited to, Intermittent Explosive Disorder, Kleptomania, Pyromania, Pathological Gambling, Trichotillomania, and Impulse Control Disorder not otherwise specified (NOS).

Alcohol-related disorders include, but are not limited to, Alcohol-Induced Psychotic Disorder with delusions, Alcohol Abuse, Alcohol Intoxication, Alcohol Withdrawal, Alcohol Intoxication Delirium, Alcohol Withdrawal Delirium, Alcohol-Induced Persisting Dementia, Alcohol-Induced Persisting Amnestic Disorder, Alcohol Dependence, Alcohol-Induced Psychotic Disorder with hallucinations, Alcohol-Induced Mood Disorder, Alcohol-Induced Anxiety Disorder, Alcohol-Induced Sexual Dysfunction, Alcohol-Induced Sleep Disorder, and Alcohol-Related Disorder not otherwise specified (NOS).

Nicotine-related disorders include, but are not limited to, Nicotine Dependence, Nicotine Withdrawal, and Nicotine-Related Disorder not otherwise specified (NOS).

Amphetamine-related disorders include, but are not limited to, Amphetamine Dependence, Amphetamine Abuse, Amphetamine Intoxication, Amphetamine Withdrawal, Amphetamine Intoxication Delirium, Amphetamine-Induced Psychotic Disorder with delusions, Amphetamine-Induced Psychotic Disorders with hallucinations, Amphetamine-Induced Mood Disorder, Amphetamine-Induced Anxiety Disorder, Amphetamine-Induced Sexual Dysfunction, Amphetamine-Induced Sleep Disorder, and Amphetamine Related Disorder not otherwise specified (NOS).

Cannabis-related disorders include, but are not limited to, Cannabis Dependence, Cannabis Abuse, Cannabis Intoxication, Cannabis Intoxication Delirium, Cannabis-Induced Psychotic Disorder with delusions, Cannabis-Induced Psychotic Disorder with hallucinations, Cannabis-Induced Anxiety Disorder, and Cannabis Related Disorder not otherwise specified (NOS).

Cocaine-related disorders include, but are not limited to, Cocaine Dependence, Cocaine Abuse, Cocaine Intoxication, Cocaine Withdrawal, Cocaine Intoxication Delirium, Cocaine-Induced Psychotic Disorder with delusions, Cocaine-Induced Psychotic Disorders with hallucinations, Cocaine-Induced Mood Disorder, Cocaine-Induced Anxiety Disorder, Cocaine-Induced Sexual Dysfunction, Cocaine-Induced Sleep Disorder, and Cocaine Related Disorder not otherwise specified (NOS).

Hallucinogen-related disorders include, but are not limited to, Hallucinogen Dependence, Hallucinogen Abuse, Hallucinogen Intoxication, Hallucinogen Withdrawal, Hallucinogen Intoxication Delirium, Hallucinogen-Induced Psychotic Disorder with delusions, Hallucinogen-Induced Psychotic Disorders with hallucinations, Hallucinogen-Induced Mood Disorder, Hallucinogen-Induced Anxiety Disorder, Hallucinogen-Induced Sexual Dysfunction, Hallucinogen-Induced Sleep Disorder, Hallucinogen Persisting Perception Disorder (Flashbacks), and Hallucinogen Related Disorder not otherwise specified (NOS).

Inhalant-related disorders include, but are not limited to, Inhalant Dependence, Inhalant Abuse, Inhalant Intoxication, Inhalant Intoxication Delirium, Inhalant-Induced Psychotic Disorder with delusions, Inhalant-Induced Psychotic Disorder with hallucinations, Inhalant-Induced Anxiety Disorder, and Inhalant Related Disorder not otherwise specified (NOS).

Opioid-related disorders include, but are not limited to, Opioid Dependence, Opioid Abuse, Opioid Intoxication, Opioid Intoxication Delirium, Opioid-Induced Psychotic Disorder with delusions, Opioid-Induced Psychotic Disorder with hallucinations, Opioid-Induced Anxiety Disorder, Opioid Withdrawal, and Opioid Related Disorder not otherwise specified (NOS).

The 2-Pyrimidinylpiperazine Compounds can be used to treat or prevent Parkinson's disease and parkinsonism and the symptoms associated with Parkinson's disease and parkinsonism, including but not limited to, bradykinesia, muscular rigidity, resting tremor, and impairment of postural balance.

The 2-Pyrimidinylpiperazine Compounds can be used to treat or prevent generalized anxiety or severe anxiety and the symptoms associated with anxiety, including but not limited to, restlessness, tension, tachycardia, dyspnea, depression including chronic “neurotic” depression, panic disorder, agoraphobia and other specific phobias, eating disorders, and personality disorders.

The 2-Pyrimidinylpiperazine Compounds can be used to treat or prevent epilepsy, including but not limited to, partial epilepsy, generalized epilepsy, and the symptoms associated with epilepsy, including but not limited to, simple partial seizures, jacksonian seizures, complex partial (psychomotor) seizures, convulsive seizures (grand mal or tonic-clonic seizures), petit mal (absence) seizures, and status epilepticus.

The 2-Pyrimidinylpiperazine Compounds can be used to treat or prevent a seizure, including but not limited to, infantile spasms, febrile seizures, and epileptic seizures.

The 2-Pyrimidinylpiperazine Compounds can be used to treat or prevent strokes, including but not limited to, ischemic strokes and hemorrhagic strokes.

The 2-Pyrimidinylpiperazine Compounds can be used to treat or prevent a pruritic condition, including but not limited to, pruritus caused by dry skin, scabies, dermatitis, herpetiformis, atopic dermatitis, pruritus vulvae et ani, malaria, insect bites, pediculosis, contact dermatitis, drug reactions, urticaria, urticarial eruptions of pregnancy, psoriasis, lichen planus, lichen simplex chronicus, exfoliative dermatitis, folliculitis, bullous pemphigoid, and fiberglass dermatitis.

The 2-Pyrimidinylpiperazine Compounds can be used to treat or prevent psychosis, including but not limited to, schizophrenia, including paranoid schizophrenia, hebephrenic or disorganized schizophrenia, catatonic schizophrenia, undifferentiated schizophrenia, negative or deficit subtype schizophrenia, and non-deficit schizophrenia; a delusional disorder, including erotomanic subtype delusional disorder, grandiose subtype delusional disorder, jealous subtype delusional disorder, persecutory subtype delusional disorder, and somatic subtype delusional disorder; and brief psychosis.

The 2-Pyrimidinylpiperazine Compounds can be used to treat or prevent a cognitive disorder, including but not limited to, delirium and dementia such as multi-infarct dementia, dementia pugilistica, dementia caused by AIDS, and dementia caused by Alzheimer's disease.

The 2-Pyrimidinylpiperazine Compounds can be used to treat or prevent a memory deficiency, including but not limited to, dissociative amnesia and dissociative fugue.

The 2-Pyrimidinylpiperazine Compounds can be used to treat or prevent restricted brain function, including but not limited to, that caused by surgery or an organ transplant, restricted blood supply to the brain, a spinal cord injury, a head injury, hypoxia, cardiac arrest, and hypoglycemia.

The 2-Pyrimidinylpiperazine Compounds can be used to treat or prevent Huntington's chorea.

The 2-Pyrimidinylpiperazine Compounds can be used to treat or prevent ALS.

The 2-Pyrimidinylpiperazine Compounds can be used to treat or prevent retinopathy, including but not limited to, arteriosclerotic retinopathy, diabetic arteriosclerotic retinopathy, hypertensive retinopathy, non-proliferative retinopathy, and proliferative retinopathy.

The 2-Pyrimidinylpiperazine Compounds can be used to treat or prevent a muscle spasm.

The 2-Pyrimidinylpiperazine Compounds can be used to treat or prevent a migraine.

The 2-Pyrimidinylpiperazine Compounds can be used to treat or prevent vomiting, including but not limited to, nausea vomiting, dry vomiting (retching), and regurgitation.

The 2-Pyrimidinylpiperazine Compounds can be used to treat or prevent dyskinesia, including but not limited to, tardive dyskinesia and biliary dyskinesia.

The 2-Pyrimidinylpiperazine Compounds can be used to treat or prevent depression, including but not limited to, major depression and bipolar disorder.

Without wishing to be bound by theory, Applicants believe that the 2-Pyrimidinylpiperazine Compounds are antagonists for mGluR5.

The invention relates to methods for inhibiting mGluR5 function in a cell comprising contacting a cell capable of expressing mGluR5 with an amount of a 2-Pyrimidinylpiperazine Compound effective to inhibit mGluR5 function in the cell. This method can be used in vitro, for example, as an assay to select cells that express mGluR5 and, accordingly, are useful as part of an assay to select compounds useful for treating or preventing pain, an addictive disorder, Parkinson's disease, parkinsonism, anxiety, a pruritic condition, or psychosis. The method is also useful for inhibiting mGluR5 function in a cell in vivo, in an animal, a human in one embodiment, by contacting a cell in an animal with an amount of a 2-Pyrimidinylpiperazine Compound effective to inhibit mGluR5 function in the cell. In one embodiment, the method is useful for treating or preventing pain in an animal in need thereof. In another embodiment, the method is useful for treating or preventing an addictive disorder in an animal in need thereof. In another embodiment, the method is useful for treating or preventing Parkinson's disease in an animal in need thereof. In another embodiment, the method is useful for treating or preventing parkinsonism in an animal in need thereof. In another embodiment, the method is useful for treating or preventing anxiety in an animal in need thereof. In another embodiment, the method is useful for treating or preventing a pruritic condition in an animal in need thereof. In another embodiment, the method is useful for treating or preventing psychosis in an animal in need thereof.

Examples of cells capable of expressing mGluR5 are neuronal and glial cells of the central nervous system, particularly the brain, especially in the nucleus accumbens. Methods for assaying cells that express mGluR5 are known in the art.

Without wishing to be bound by theory, Applicants believe that the 2-Pyrimidinylpiperazine Compounds are antagonists for mGluR1.

The invention relates to methods for inhibiting mGluR1 function in a cell comprising contacting a cell capable of expressing mGluR1 with an amount of a 2-Pyrimidinylpiperazine Compound effective to inhibit mGluR1 function in the cell. This method can be used in vitro, for example, as an assay to select cells that express mGluR1 and, accordingly, are useful as part of an assay to select compounds useful for treating or preventing a Condition. The method is also useful for inhibiting mGluR1 function in a cell in vivo, in an animal, a human in one embodiment, by contacting a cell, in an animal, with an amount of a 2-Pyrimidinylpiperazine Compound effective to inhibit mGluR1 function in the cell. In one embodiment, the method is useful for treating or preventing pain in an animal in need thereof. In another embodiment, the method is useful for treating or preventing UI in an animal in need thereof. In another embodiment, the method is useful for treating or preventing an addictive disorder in an animal in need thereof. In another embodiment, the method is useful for treating or preventing Parkinson's disease in an animal in need thereof. In another embodiment, the method is useful for treating or preventing parkinsonism in an animal in need thereof. In another embodiment, the method is useful for treating or preventing anxiety in an animal in need thereof. In another embodiment, the method is useful for treating or preventing epilepsy in an animal in need thereof. In another embodiment, the method is useful for treating or preventing a seizure in an animal in need thereof. In another embodiment, the method is useful for treating or preventing stroke in an animal in need thereof. In another embodiment, the method is useful for treating or preventing a pruritic condition in an animal in need thereof. In another embodiment, the method is useful for treating or preventing psychosis in an animal in need thereof. In another embodiment, the method is useful for treating or preventing a cognitive disorder in an animal in need thereof. In another embodiment, the method is useful for treating or preventing a memory deficit in an animal in need thereof. In another embodiment, the method is useful for treating or preventing restricted brain function in an animal in need thereof. In another embodiment, the method is useful for treating or preventing Huntington's chorea in an animal in need thereof. In another embodiment, the method is useful for treating or preventing ALS in an animal in need thereof. In another embodiment, the method is useful for treating or preventing dementia in an animal in need thereof. In another embodiment, the method is useful for treating or preventing retinopathy in an animal in need thereof. In another embodiment, the method is useful for treating or preventing a muscle spasm in an animal in need thereof. In another embodiment, the method is useful for treating or preventing a migraine in an animal in need thereof. In another embodiment, the method is useful for treating or preventing vomiting in an animal in need thereof. In another embodiment, the method is useful for treating or preventing dyskinesia in an animal in need thereof. In another embodiment, the method is useful for treating or preventing depression in an animal in need thereof.

Examples of cells capable of expressing mGluR1 include, but are not limited to, cerebellar Purkinje neuron cells, Purkinje cell bodies (punctate), cells of spine(s) of the cerebellum; neurons and neurophil cells of olfactory-bulb glomeruli; cells of the superficial layer of the cerebral cortex; hippocampus cells; thalamus cells; superior colliculus cells; and spinal trigeminal nucleus cells. Methods for assaying cells that express mGluR1 are known in the art.

### 4.6 Therapeutic/Prophylactic Administration and Compositions of the Invention

Due to their activity, the 2-Pyrimidinylpiperazine Compounds are advantageously useful in veterinary and human medicine. As described above, the 2-Pyrimidinylpiperazine Compounds are useful for treating or preventing a Condition in an animal in need thereof.

When administered to an animal, the 2-Pyrimidinylpiperazine Compounds are administered as a component of a composition that comprises a pharmaceutically acceptable carrier or excipient. The present compositions, which comprise a 2-Pyrimidinylpiperazine Compound, can be administered orally. The 2-Pyrimidinylpiperazine Compounds of the invention can also be administered by any other convenient route, for example, by infusion or bolus injection, by absorption through epithelial or mucocutaneous linings (e.g., oral, rectal, and intestinal mucosa, etc.) and can be administered together with another therapeutically active agent. Administration can be systemic or local. Various delivery systems are known, e.g., encapsulation in liposomes, microparticles, microcapsules, capsules, etc., and can be used to administer the 2-Pyrimidinylpiperazine Compound.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. The mode of administration is left to the discretion of the practitioner. In most instances, administration will result in the release of the 2-Pyrimidinylpiperazine Compounds into the bloodstream.

In specific embodiments, it can be desirable to administer the 2-Pyrimidinylpiperazine Compounds locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce the 2-Pyrimidinylpiperazine Compounds into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the 2-Pyrimidinylpiperazine Compounds can be formulated as a suppository, with traditional binders and excipients such as triglycerides.

In another embodiment, the 2-Pyrimidinylpiperazine Compounds can be delivered in a vesicle, in particular a liposome (see Langer, Science 249:1527-1533 (1990) and Treat et al., Liposomes in the Therapy of Infectious Disease and Cancer 317-327 and 353-365 (1989).

In yet another embodiment, the 2-Pyrimidinylpiperazine Compounds can be delivered in a controlled-release system or sustained-release system (see, e.g., Goodson, in Medical Applications of Controlled Release, supra, vol. 2, pp. 115-138 (1984)). Other controlled- or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance(Smolen and Ball eds., 1984); Ranger and Peppas, J. Macromol. Sci. Rev. Macromol. Chem. 23:61 (1983); Levy et al., Science 228:190 (1985); During et al., Ann. Neurol. 25:351 (1989); and Howard et al., J. Neurosurg. 71:105 (1989)). In yet another embodiment, a controlled- or sustained-release system can be placed in proximity of a target of the 2-Pyrimidinylpiperazine Compounds, e.g., the spinal column, brain, or gastrointestinal tract, thus requiring only a fraction of the systemic dose.

The present compositions can optionally comprise a suitable amount of a pharmaceutically acceptable excipient so as to provide the form for proper administration to the animal.

Such pharmaceutical excipients can be liquids, such as water and oils, including those of petroleum, animal, vegetable, or synthetic origin, such as peanut oil, soybean oil, mineral oil, sesame oil and the like. The pharmaceutical excipients can be saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to an animal. Water, and in one embodiment physiological saline, is a particularly useful excipient when the 2-Pyrimidinylpiperazine Compound is administered intravenously. Saline solutions and aqueous dextrose and glycerol solutions can also be employed as liquid excipients, particularly for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsions, tablets, pills, pellets, capsules, capsules containing liquids, powders, sustained-release formulations, suppositories, aerosols, sprays, suspensions, or any other form suitable for use. In one embodiment, the composition is in the form of a capsule (see e.g., U.S. Pat. No. 5,698,155). Other examples of suitable pharmaceutical excipients are described in Remington 's Pharmaceutical Sciences 1447-1676 (Alfonso R. Gennaro ed., 19th ed. 1995), incorporated herein by reference.

In one embodiment, the 2-Pyrimidinylpiperazine Compounds are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs, for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the 2-Pyrimidinylpiperazine Compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lidocaine to lessen pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the 2-Pyrimidinylpiperazine Compounds are to be administered by infusion, they can be dispensed, for example, with an infusion bottle containing sterile pharmaceutical grade water or saline. Where the 2-Pyrimidinylpiperazine Compounds are administered by injection, an ampule of sterile water for injection or saline can be provided so that the ingredients can be mixed prior to administration.

The 2-Pyrimidinylpiperazine Compounds can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566, each of which is incorporated herein by reference. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

Controlled- or sustained-release pharmaceutical compositions can have a common goal of improving drug therapy over that achieved by their non-controlled or non-sustained counterparts. In one embodiment, a controlled- or sustained-release composition comprises a minimal amount of a 2-Pyrimidinylpiperazine Compound to cure or control the condition in a minimum amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of the 2-Pyrimidinylpiperazine Compound, and can thus reduce the occurrence of adverse side effects.

Controlled- or sustained-release compositions can initially release an amount of a 2-Pyrimidinylpiperazine Compound that promptly produces the desired therapeutic or prophylactic effect, and gradually and continually release other amounts of the 2-Pyrimidinylpiperazine Compound to maintain this level of therapeutic or prophylactic effect over an extended period of time. To maintain a constant level of the 2-Pyrimidinylpiperazine Compound in the body, the 2-Pyrimidinylpiperazine Compound can be released from the dosage form at a rate that will replace the amount of 2-Pyrimidinylpiperazine Compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions or compounds.

The amount of the 2-Pyrimidinylpiperazine Compound that is effective in the treatment or prevention of a Condition and can be determined by standard clinical techniques. In addition, in vitro or in vivo assays can optionally be employed to help identify optimal dosage ranges. The precise dose to be employed will also depend on the route of administration, and the seriousness of the Condition and can be decided according to the judgment of a practitioner and/or each animal's circumstances. Suitable effective dosage amounts, however, range from about 0.01 mg/kg of body weight to about 2500 mg/kg of body weight, although they are typically about 100 mg/kg of body weight or less. In one embodiment, the effective dosage amount ranges from about 0.01 mg/kg of body weight to about 100 mg/kg of body weight of a 2-Pyrimidinylpiperazine Compound, in another embodiment, about 0.02 mg/kg of body weight to about 50 mg/kg of body weight, and in another embodiment, about 0.025 mg/kg of body weight to about 20 mg/kg of body weight. In one embodiment, an effective dosage amount is administered about every 24 h until the Condition is abated. In another embodiment, an effective dosage amount is administered about every 12 h until the Condition is abated. In another embodiment, an effective dosage amount is administered about every 8 h until the Condition is abated. In another embodiment, an effective dosage amount is administered about every 6 h until the Condition is abated. In another embodiment, an effective dosage amount is administered about every 4 h until the Condition is abated. The effective dosage amounts described herein refer to total amounts administered; that is, if more than one 2-Pyrimidinylpiperazine Compound is administered, the effective dosage amounts correspond to the total amount administered.

Where a cell capable of expressing mGluR5 or mGluR1 is contacted with a 2-Pyrimidinylpiperazine Compound in vitro, the amount effective for inhibiting the mGluR5 or mGluR1 receptor function in a cell will typically range from about 0.01 μg/L to about 5 mg/L, in one embodiment, from about 0.01 μg/L to about 2.5 mg/L, in another embodiment, from about 0.01 μg/L to about 0.5 mg/L, and in another embodiment, from about 0.01 μg/L to about 0.25 mg/L of a solution or suspension of a pharmaceutically acceptable carrier or excipient. In one embodiment, the volume of solution or suspension comprising the 2-Pyrimidinylpiperazine Compound is from about 0.01 μL to about 1 mL. In another embodiment, the volume of solution or suspension is about 200 μL.

Where a cell capable of expressing VR1, mGluR5, or mGluR1 is contacted with a 2-Pyrimidinylpiperazine Compound in vivo, the amount effective for inhibiting the receptor function in a cell will typically range from about 0.01 mg/kg of body weight to about 2500 mg/kg of body weight, although it typically ranges from about 100 mg/kg of body weight or less. In one embodiment, the effective dosage amount ranges from about 0.01 mg/kg of body weight to about 100 mg/kg of body weight of a 2-Pyrimidinylpiperazine Compound, in another embodiment, about 0.020 mg/kg of body weight to about 50 mg/kg of body weight, and in another embodiment, about 0.025 mg/kg of body weight to about 20 mg/kg of body weight. In one embodiment, an effective dosage amount is administered about every 24 h. In another embodiment, an effective dosage amount is administered about every 12. In another embodiment, an effective dosage amount is administered about every 8. In another embodiment, an effective dosage amount is administered about every 6 h. In another embodiment, an effective dosage amount is administered about every 4 h.

The 2-Pyrimidinylpiperazine Compounds can be assayed in vitro or in vivo for the desired therapeutic or prophylactic activity prior to use in humans. Animal model systems can be used to demonstrate safety and efficacy.

The present methods for treating or preventing a Condition in an animal in need thereof can further comprise administering another therapeutic agent to the animal being administered a 2-Pyrimidinylpiperazine Compound. In one embodiment, the other therapeutic agent is administered in an effective amount.

The present methods for inhibiting mGluR5 function in a cell capable of expressing mGluR5 can further comprise contacting the cell with an effective amount of another therapeutic agent.

The present methods for inhibiting mGluR1 function in a cell capable of expressing mGluR1 can further comprise contacting the cell with an effective amount of another therapeutic agent.

Effective amounts of the other therapeutic agents are known to those skilled in the art. However, it is well within the skilled artisan's purview to determine the other therapeutic agent's optimal effective-amount range. In one embodiment of the invention, where another therapeutic agent is administered to an animal, the effective amount of the 2-Pyrimidinylpiperazine Compound is less than its effective amount would be where the other therapeutic agent is not administered. In this case, without being bound by theory, it is believed that the 2-Pyrimidinylpiperazine Compounds and the other therapeutic agent act synergistically to treat or prevent a Condition.

The other therapeutic agent can be, but is not limited to, an opioid agonist, a non-opioid analgesic, a non-steroidal anti-inflammatory agent, an antimigraine agent, a Cox-II inhibitor, an antiemetic, a β-adrenergic blocker, an anticonvulsant, an antidepressant, a Ca2+-channel blocker, an anticancer agent, an agent for treating or preventing UI, an agent for treating addictive disorder, an agent for treating Parkinson's disease and parkinsonism, an agent for treating anxiety, an agent for treating epilepsy, an agent for treating a seizure, an agent for treating a stroke, an agent for treating a pruritic condition, an agent for treating psychosis, an agent for treating Huntington's chorea, an agent for treating ALS, an agent for treating a cognitive disorder, an agent for treating a migraine, an agent for treating vomiting, an agent for treating dyskinesia, or an agent for treating depression, and mixtures thereof.

Examples of useful opioid agonists include, but are not limited to, alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, proheptazine, promedol, properidine, propiram, propoxyphene, sufentanil, tilidine, tramadol, pharmaceutically acceptable salts thereof, and mixtures thereof.

In certain embodiments, the opioid agonist is selected from codeine, hydromorphone, hydrocodone, oxycodone, dihydrocodeine, dihydromorphine, morphine, tramadol, oxymorphone, pharmaceutically acceptable salts thereof, and mixtures thereof.

Examples of useful non-opioid analgesics include non-steroidal anti-inflammatory agents, such as aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam, isoxicaam, and pharmaceutically acceptable salts thereof, and mixtures thereof. Other suitable non-opioid analgesics include the following, non-limiting, chemical classes of analgesic, antipyretic, non-steroidal anti-inflammatory drugs: salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid and meclofenamic acid; enolic acids, including oxicams (piroxicam, tenoxicam), and pyrazolidinediones (phenylbutazone, oxyphenthartazone); and alkanones, including nabumetone. For a more detailed description of the NSAIDs, see Paul A. Insel, Analgesic-Antipyretic and Anti-inflammatory Agents and Drugs Employed in the Treatment of Gout, in Goodman & Gilman's The Pharmacological Basis of Therapeutics 617-57 (Perry B. Molinhoff and Raymond W. Ruddon eds., 9^{th} ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

Examples of useful Cox-II inhibitors and 5-lipoxygenase inhibitors, as well as combinations thereof, are described in U.S. Pat. No. 6,136,839, which is hereby incorporated by reference in its entirety. Examples of useful Cox-II inhibitors include, but are not limited to, rofecoxib and celecoxib.

Examples of useful antimigraine agents include, but are not limited to, alpiropride, bromocriptine, dihydroergotamine, dolasetron, ergocornine, ergocorninine, ergocryptine, ergonovine, ergot, ergotamine, flumedroxone acetate, fonazine, ketanserin, lisuride, lomerizine, methylergonovine, methysergide, metoprolol, naratriptan, oxetorone, pizotyline, propranolol, risperidone, rizatriptan, sumatriptan, timolol, trazodone, zolmitriptan, and mixtures thereof.

The other therapeutic agent can alternatively be an agent useful for reducing any potential side effects of a 2-Pyrimidinylpiperazine Compounds. For example, the other therapeutic agent can be an antiemetic agent. Examples of useful antiemetic agents include, but are not limited to, metoclopromide, domperidone, prochlorperazine, promethazine, chlorpromazine, trimethobenzamide, odansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, and mixtures thereof.

Examples of useful β-adrenergic blockers include, but are not limited to, acebutolol, alprenolol, amosulabol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol, indenolol, labetalol, levobunolol, mepindolol, metipranolol, metoprolol, moprolol, nadolol, nadoxolol, nebivalol, nifenalol, nipradilol, oxprenolol, penbutolol, pindolol, practolol, pronethalol, propranolol, sotalol, sulfinalol, talinolol, tertatolol, tilisolol, timolol, toliprolol, and xibenolol.

Examples of useful anticonvulsants include, but are not limited to, acetylpheneturide, albutoin, aloxidone, aminoglutethimide, 4-amino-3-hydroxybutyric acid, atrolactamide, beclamide, buramate, calcium bromide, carbamazepine, cinromide, clomethiazole, clonazepam, decimemide, diethadione, dimethadione, doxenitroin, eterobarb, ethadione, ethosuximide, ethotoin, felbamate, fluoresone, gabapentin, 5-hydroxytryptophan, lamotrigine, magnesium bromide, magnesium sulfate, mephenyloin, mephobarbital, metharbital, methetoin, methsuximide, 5-methyl-5-(3-phenanthryl)-hydantoin, 3-methyl-5-phenylhydantoin, narcobarbital, nimetazepam, nitrazepam, oxcarbazepine, paramethadione, phenacemide, phenetharbital, pheneturide, phenobarbital, phensuximide, phenylmethylbarbituric acid, phenyloin, phethenylate sodium, potassium bromide, pregabaline, primidone, progabide, sodium bromide, solanum, strontium bromide, suclofenide, sulthiame, tetrantoin, tiagabine, topiramate, trimethadione, valproic acid, valpromide, vigabatrin, and zonisamide.

Examples of useful antidepressants include, but are not limited to, binedaline, caroxazone, citalopram, (S)-citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimelidine.

Examples of useful Ca2+-channel blockers include, but are not limited to, bepridil, clentiazem, diltiazem, fendiline, gallopamil, mibefradil, prenylamine, semotiadil, terodiline, verapamil, amlodipine, aranidipine, bamidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, fantofarone, and perhexiline.

Examples of useful anticancer agents include, but are not limited to, acivicin, aclarubicin, acodazole hydrochloride, acronine, adozelesin, aldesleukin, altretamine, ambomycin, ametantrone acetate, aminoglutethimide, amsacrine, anastrozole, anthramycin, asparaginase, asperlin, azacitidine, azetepa, azotomycin, batimastat, benzodepa, bicalutamide, bisantrene hydrochloride, bisnafide dimesylate, bizelesin, bleomycin sulfate, brequinar sodium, bropirimine, busulfan, cactinomycin, calusterone, caracemide, carbetimer, carboplatin, carmustine, carubicin hydrochloride, carzelesin, cedefingol, chlorambucil, cirolemycin, cisplatin, cladribine, crisnatol mesylate, cyclophosphamide, cytarabine, dacarbazine, dactinomycin, daunorubicin hydrochloride, decitabine, dexormaplatin, dezaguanine, dezaguanine mesylate, diaziquone, docetaxel, doxorubicin, doxorubicin hydrochloride, droloxifene, droloxifene citrate, dromostanolone propionate, duazomycin, edatrexate, eflomithine hydrochloride, elsamitrucin, enloplatin, enpromate, epipropidine, epirubicin hydrochloride, erbulozole, esorubicin hydrochloride, estramustine, estramustine phosphate sodium, etanidazole, etoposide, etoposide phosphate, etoprine, fadrozole hydrochloride, fazarabine, fenretinide, floxuridine, fludarabine phosphate, fluorouracil, flurocitabine, fosquidone, fostriecin sodium, gemcitabine, gemcitabine hydrochloride, hydroxyurea, idarubicin hydrochloride, ifosfamide, ilmofosine, interleukin II (including recombinant interleukin II or rIL2), interferon alpha-2a, interferon alpha-2b, interferon alpha-n1, interferon alpha-n3, interferon beta-Ia, interferon gamma-I b, iproplatin, irinotecan hydrochloride, lanreotide acetate, letrozole, leuprolide acetate, liarozole hydrochloride, lometrexol sodium, lomustine, losoxantrone hydrochloride, masoprocol, maytansine, mechlorethamine hydrochloride, megestrol acetate, melengestrol acetate, melphalan, menogaril, mercaptopurine, methotrexate, methotrexate sodium, metoprine, meturedepa, mitindomide, mitocarcin, mitocromin, mitogillin, mitomalcin, mitomycin, mitosper, mitotane, mitoxantrone hydrochloride, mycophenolic acid, nocodazole, nogalamycin, ormaplatin, oxisuran, paclitaxel, pegaspargase, peliomycin, pentamustine, peplomycin sulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, riboprine, rogletimide, safingol, safingol hydrochloride, semustine, simtrazene, sparfosate sodium, sparsomycin, spirogermanium hydrochloride, spiromustine, spiroplatin, streptonigrin, streptozotocin, sulofenur, talisomycin, tecogalan sodium, tegafur, teloxantrone hydrochloride, temoporfin, teniposide, teroxirone, testolactone, thiamiprine, thioguanine, thiotepa, tiazofurin, tirapazamine, toremifene citrate, trestolone acetate, triciribine phosphate, trimetrexate, trimetrexate glucuronate, triptorelin, tubulozole hydrochloride, uracil mustard, uredepa, vapreotide, verteporfin, vinblastine sulfate, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, zorubicin hydrochloride.

Examples of other anti-cancer drugs include, but are not limited to, 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; odansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Examples of useful therapeutic agents for treating or preventing UI include, but are not limited to, propantheline, imipramine, hyoscyamine, oxybutynin, and dicyclomine.

Examples of useful therapeutic agents for treating or preventing an addictive disorder include, but are not limited to, methadone, desipramine, amantadine, fluoxetine, buprenorphine, an opiate agonist, 3-phenoxypyridine, levomethadyl acetate hydrochloride, and serotonin antagonists.

Examples of useful therapeutic agents for treating or preventing Parkinson's disease and parkinsonism include, but are not limited to, carbidopa/levodopa, pergolide, bromocriptine, ropinirole, pramipexole, entacapone, tolcapone, selegiline, amantadine, and trihexyphenidyl hydrochloride.

Examples of useful therapeutic agents for treating or preventing anxiety include, but are not limited to, benzodiazepines, such as alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, and triazolam; non-benzodiazepine agents, such as buspirone, gepirone, ipsapirone, tiospirone, zolpicone, zolpidem, and zaleplon; tranquilizers, such as barbituates, e.g., amobarbital, aprobarbital, butabarbital, butalbital, mephobarbital, methohexital, pentobarbital, phenobarbital, secobarbital, and thiopental; and propanediol carbamates, such as meprobamate and tybamate.

Examples of useful therapeutic agents for treating or preventing epilepsy include, but are not limited to, carbamazepine, ethosuximide, gabapentin, lamotrigine, phenobarbital, phenyloin, primidone, valproic acid, trimethadione, benzodiazepines, gabapentin, lamotrigine, γ-vinyl GABA, acetazolamide, and felbamate.

Examples of useful therapeutic agents for treating or preventing a seizure include, but are not limited to, carbarnazepine, ethosuximide, gabapentin, larnotrigine, phenobarbital, phenyloin, primidone, valproic acid, trimethadione, benzodiazepines, gabapentin, lamotrigine, γ-vinyl GABA, acetazolamide, and felbamate.

Examples of useful therapeutic agents for treating or preventing stroke include, but are not limited to, anticoagulants such as heparin, agents that break up clots such as streptokinase or tissue plasminogen activator, agents that reduce swelling such as mannitol or corticosteroids, and acetylsalicylic acid.

Examples of useful therapeutic agents for treating or preventing a pruritic condition include, but are not limited to, naltrexone; nalmefene; danazol; tricyclics such as amitriptyline, imipramine, and doxepin; antidepressants such as those given below; menthol; camphor; phenol; pramoxine; capsaicin; tar; steroids; and antihistamines.

Examples of useful therapeutic agents for treating or preventing psychosis include, but are not limited to, phenothiazines such as chlorpromazine hydrochloride, mesoridazine besylate, and thoridazine hydrochloride; thioxanthenes such as chloroprothixene and thiothixene hydrochloride; clozapine; risperidone; olanzapine; quetiapine; quetiapine flumarate; haloperidol; haloperidol decanoate; loxapine succinate; molindone hydrochloride; pimozide; and ziprasidone.

Examples of useful therapeutic agents for treating or preventing Huntington's chorea include, but are not limited to, haloperidol and pimozide.

Examples of useful therapeutic agents for treating or preventing ALS include, but are not limited to, baclofen, neurotrophic factors, riluzole, tizanidine, benzodiazepines such as clonazepan and dantrolene.

Examples of useful therapeutic agents for treating or preventing cognitive disorders include, but are not limited to, agents for treating or preventing dementia such as tacrine; donepezil; ibuprofen; antipsychotic drugs such as thioridazine and haloperidol; and antidepressant drugs such as those given below.

Examples of useful therapeutic agents for treating or preventing a migraine include, but are not limited to, sumatriptan; methysergide; ergotamine; caffeine; and beta-blockers such as propranolol, verapamil, and divalproex.

Examples of useful therapeutic agents for treating or preventing vomiting include, but are not limited to, 5-HT₃ receptor antagonists such as odansetron, dolasetron, granisetron, and tropisetron; dopamine receptor antagonists such as prochlorperazine, thiethylperazine, chlorpromazine, metoclopramide, and domperidone; glucocorticoids such as dexamethasone; and benzodiazepines such as lorazepam and alprazolam.

Examples of useful therapeutic agents for treating or preventing dyskinesia include, but are not limited to, reserpine and tetrabenazine.

Examples of useful therapeutic agents for treating or preventing depression include, but are not limited to, tricyclic antidepressants such as amitryptyline, amoxapine, bupropion, clomipramine, desipramine, doxepin, imipramine, maprotiline, nefazadone, nortriptyline, protriptyline, trazodone, trimipramine, and venlafaxine; selective serotonin reuptake inhibitors such as citalopram, (S)-citalopram, fluoxetine, fluvoxamine, paroxetine, and setraline; monoamine oxidase inhibitors such as isocarboxazid, pargyline, phenelzine, and tranylcypromine; and psychostimulants such as dextroamphetamine and methylphenidate.

A 2-Pyrimidinylpiperazine Compound and the other therapeutic agent can act additively or, in one embodiment, synergistically. In one embodiment, a 2-Pyrimidinylpiperazine Compound is administered concurrently with another therapeutic agent; for example, a composition comprising an effective amount of a 2-Pyrimidinylpiperazine Compound, an effective amount of another therapeutic agent can be administered. Alternatively, a composition comprising an effective amount of a 2-Pyrimidinylpiperazine Compound and a different composition comprising an effective amount of another therapeutic agent can be concurrently administered. In another embodiment, an effective amount of a 2-Pyrimidinylpiperazine Compound is administered prior or subsequent to administration of an effective amount of another therapeutic agent. In this embodiment, the 2-Pyrimidinylpiperazine Compound is administered while the other therapeutic agent exerts its therapeutic effect, or the other therapeutic agent is administered while the 2-Pyrimidinylpiperazine Compound exerts its therapeutic effect for treating or preventing a Condition.

A composition of the invention is prepared by a method comprising admixing a 2-Pyrimidinylpiperazine Compound or pharmaceutically acceptable salt and a pharmaceutically acceptable carrier or excipient. Admixing can be accomplished using methods well known for admixing a compound (or salt) and a pharmaceutically acceptable carrier or excipient. In one embodiment the composition is prepared such that the 2-Pyrimidinylpiperazine Compound is present in the composition in an effective amount.

### 4.7 Kits

The invention encompasses kits that can simplify the administration of a 2-Pyrimidinylpiperazine Compound to an animal.

A typical kit of the invention comprises a unit dosage form of a 2-Pyrimidinylpiperazine Compound. In one embodiment, the unit dosage form is a container, which can be sterile, containing an effective amount of a 2-Pyrimidinylpiperazine Compound and a pharmaceutically acceptable carrier or excipient. The kit can further comprise a label or printed instructions instructing the use of the 2-Pyrimidinylpiperazine Compound to treat a Condition. The kit can also further comprise a unit dosage form of another therapeutic agent, for example, a second container containing an effective amount of the other therapeutic agent and a pharmaceutically acceptable carrier or excipient. In another embodiment, the kit comprises a container containing an effective amount of a 2-Pyrimidinylpiperazine Compound, an effective amount of another therapeutic agent and a pharmaceutically acceptable carrier or excipient. Examples of other therapeutic agents include, but are not limited to, those listed above.

Kits of the invention can further comprise a device that is useful for administering the unit dosage forms. Examples of such a device include but are not limited to a syringe, a drip bag, a patch, an inhaler, and an enema bag.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### 5. EXAMPLES

Examples 1-11 relate to the synthesis of illustrative 2-Pyrimidinylpiperazine Compounds.

### 5.1 Example 1

### Synthesis of Compound AAA(IIa)

Compound AAA(IIa) was prepared according to the following scheme:

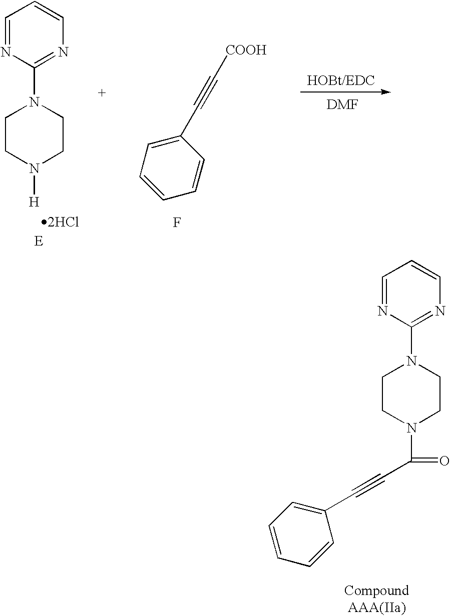

A solution of 1-(2-pyrimidinyl)piperazine dihydrochloride (“Compound E,” 100 mg, 0.42 mmol), 3-phenyl-2-propynoic acid (“Compound F,” 61 mg, 0.42 mmol), 1-hydroxybenzotriazole (“HOBt,” 57 mg, 0.42 mmol), and 1-[3-(dimethylamino)propyl]-3-ethylcarboimide hydrochloride (“EDC,” 97 mg, 0.54 mmol) in 3 mL dimethylformamide (“DMF”) was stirred at room temperature, about 25° C., for 4 hours. After this period, DMF was removed under reduced pressure and the resulting residue was dissolved in ethyl acetate and extracted with brine. The organic layer was dried using Na₂SO₄ and purified using flash chromatography (normal phase silica gel, 35-60 μm particle size (230-400 mesh) with an ethyl acetate/hexane eluent system) to provide 49 mg of Compound AAA(IIa) as a white solid (40% yield).

The structure of Compound AAA(IIa) was confirmed by ¹H NMR and mass spectral (MS) analysis. Compound AAA(IIa): ¹H NMR (CDCl₃) δ 8.37 (bd, J=4.8 Hz, 2H), 7.60 (m, 2H), 7.47 (m, 3H), 6.58 (t, J=8.0, 4.8 Hz, 1H), 3.91 (m, 6H), 3.80 (m, 2H); MS (EI): m/z 315 (M+Na⁺).

### 5.2 Example 2

### Synthesis of Compound AFX(IIb)

Compound AFX(IIb) was prepared according to the following scheme:

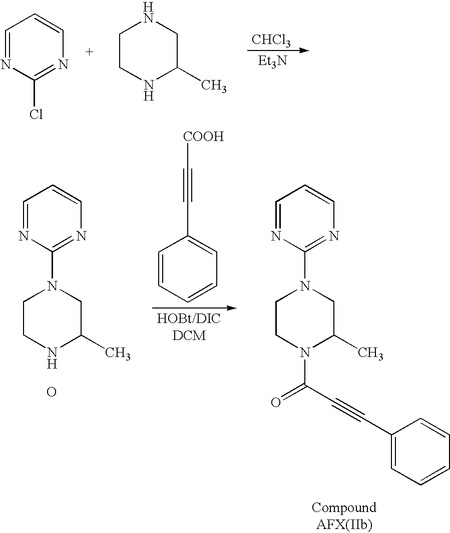

2-Chloropyrimidine (1.14 g, 10.0 mmol), 2-methylpiperazine (1.20 g, 12.0 mmol), and triethylamine (1.52 g, 15 mmol) were dissolved in 10 mL of chloroform and the resulting mixture was stirred at room temperature, about 25° C., for 4 hours. The reaction was quenched with water and the resulting mixture was extracted with chloroform. The organic layer was dried, concentrated, and purified using a silica gel column eluted with gradient elution from ethyl acetate to 2/1 ethyl acetate/methanol to provide Compound O as a yellow oil (95% yield).

A solution of Compound O (178 mg, 1.0 mmol), Compound F (219 mg, 1.5 mmol), HOBt (203 mg, 1.5 mmol), and DIC (189 mg. 1.5 mmol) in 4.5 mL dichloromethane (“DCM”) was stirred at room temperature, about 25° C., for 4 hours. After evaporation, the product was purified using a silica gel column eluted with gradient elution from hexane to 1/1 hexane/ethyl acetate to provide 153 mg of Compound AFX(IIb) as a slight yellowish solid (50% yield).

The structure of Compound AFX(IIb) was confirmed by ¹H NMR and mass spectral (MS) analysis. Compound AFX(IIb): ¹H NMR (CDCl₃) δ 8.35 (d, J=4.7 Hz, 2H), 7.61 (m, 2H), 7.40 (m, 3H), 6.55 (dd, J=4.7, 4.7 Hz, 1H), 4.91 (m, 0.6H), 4.78 (m, 2H), 4.63 (dt, J=1.8, 11.6 Hz, 0.4H), 4.52 (d, J=13.3 Hz, 0.4H), 4.33 (d, J=13.3 Hz, 0.6H), 3.59 (m, 0.6H), 3.20 (m, 2.4H), 1.36 (d, J=6.8 Hz, 1.214), 1.25 (d, J=6.8 Hz, 1.8H); MS (EI): m/z 329 (M+Na⁺).

### 5.3 Example 3

### Synthesis of Compound BGS(IIa)

Compound BGS(IIa) was prepared according to the following scheme:

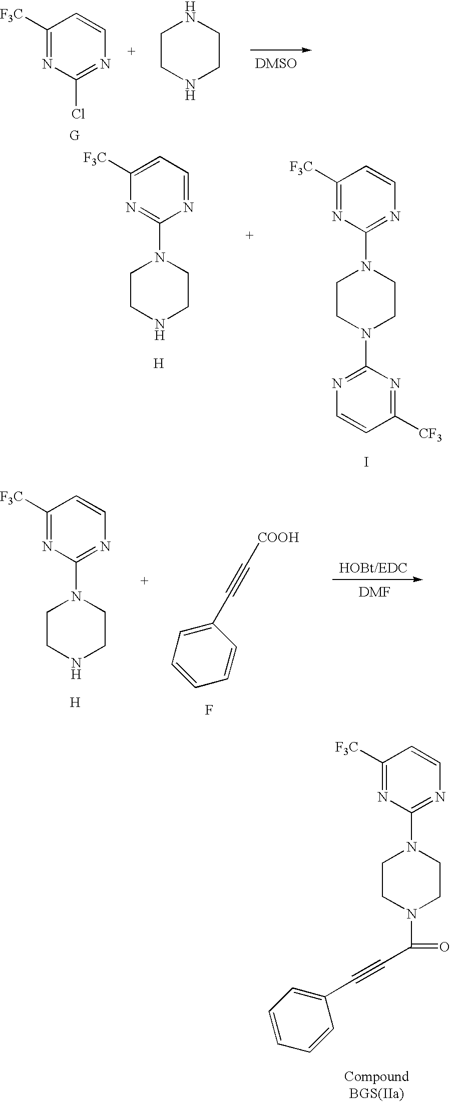

A solution of 2-chloro-4-(trifluoromethyl)pyrimidine (“Compound G.” 400 mg, 2.19 mmol) and piperazine (189 mg, 2.19 mmol) in dimethylsulfoxide (“DMSO,” 4 mL) was placed on a shaker at room temperature, about 25° C., for 5 minutes to provide a mixture of the free-base form of Compound H and Compound I. The resulting mixture of Compound H and Compound I was concentrated and separated using flash chromatography as described in Example 1 to provide 200 mg (39% yield) of Compound H.

A solution of Compound H (200 mg, 0.87 mmol), Compound F (138 mg, 0.95 mmol), HOBt (128 mg, 0.95 mmol) and EDC (182 mg, 0.95 mmol) in 3 mL DMF was stirred at room temperature for 4 hours. After this period, DMF was removed under reduced pressure and the resulting residue was dissolved in ethyl acetate and extracted with brine. The organic layer was dried using Na₂SO₄ and purified using flash chromatography as described in Example 1 to provide 40 mg of Compound BGS(IIa) as a white solid (5% overall yield based on Compound G).

The structure of Compound BGS(IIa) was confirmed by ¹H NMR and mass spectral (MS) analysis. Compound BGS(IIa): ¹H NMR (CDCl₃) δ 8.56 (d, J=4.8 Hz, 1H), 7.62-7.58 (m, 2H), 7.50-7.39 (m, 3H), 6.80 (d, J=4.8 Hz, 1H), 4.05-4.01 (m, 2H), 3.99-3.95 (m, 4H), 3.84-3.80 (m, 2H); MS (EI): m/z 361 (M+Na⁺).

### 5.4 Example 4

### Synthesis of Compound ENS(IVa)

Compound ENS(IVa) was prepared according to the following scheme:

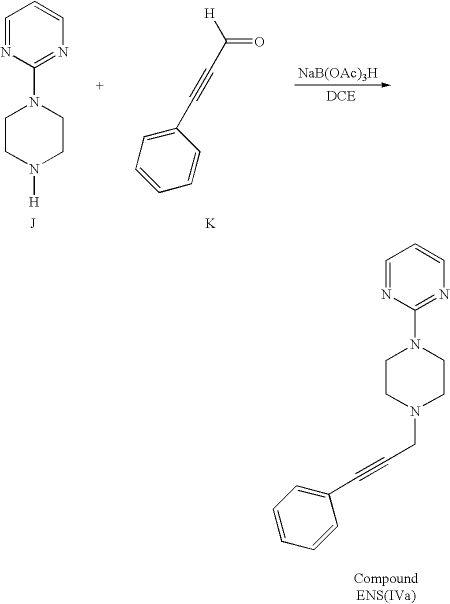

To a solution of 1-(2-pyrimidyl)piperazine (“Compound J,” 200 mg, 1.22 mmol) and 3-phenyl-2-propynal (“Compound K,” 159 mg, 1.22 mmol) in dichloroethane (“DCE,” 10 mL) was added sodium triacetoxyborohydride (“NaB(OAc)₃H,” 1.1 equivalents, 284 mg, 1.34 mmol). The reaction mixture was placed on a shaker at room temperature, about 25° C., for 2 hours. After this period, the decanted solution was purified using flash chromatography as described in Example 1 to provide 100 mg (30% yield) of a brown oil. The brown oil was then dissolved in DCM (1 mL) and 1N HCl (6 drops in 0.5 mL diethyl ether) was added to the resulting mixture to provide 100 mg of Compound ENS(IVa), isolated as its hydrochloride as a white solid (30% overall yield).

The structure of Compound ENS(IVa) was confirmed by ¹H NMR and mass spectral (MS) analysis. Compound ENS(IVa): ¹H NMR (CDCl₃) δ 8.33 (d, J=4.6 Hz, 2H), 7.45-7.44 (m, 2H), 7.33-7.30 (m, 3H), 6.65 (t, J=4.6 Hz, 1H), 3.95-3.91 (m, 4H), 3.60 (s, 2H), 2.75-2.71 (m, 4H); MS (EI): m/z 293 (M+Na⁺).

### 5.5 Example 5

### Synthesis of Compound GZE(Va)

Compound GZE(Va) was prepared according to the following scheme:

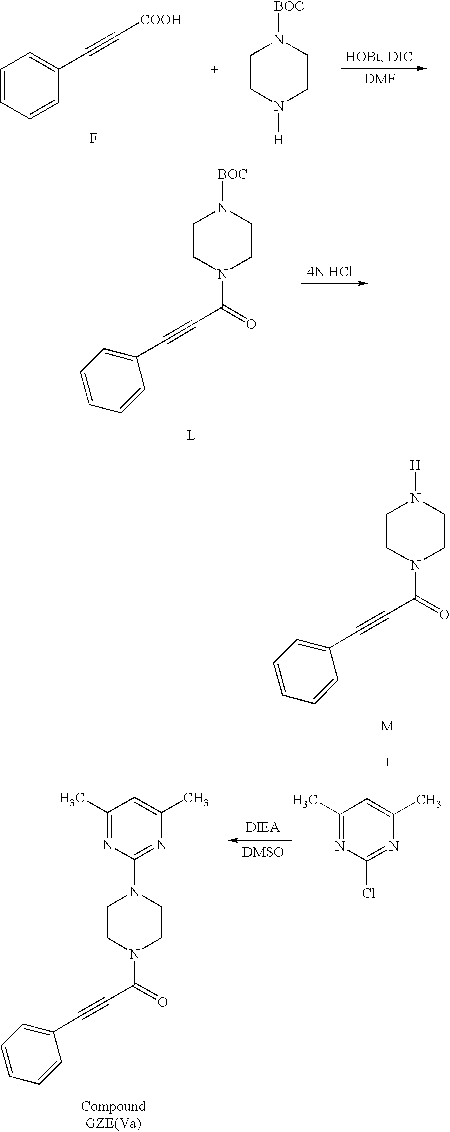

A solution of Compound F (5 g, 34 mmol) and HOBt (0.1 g. 0.74 mmol) in DMF (50 mL) was cooled to 0° C. Piperazine-1-carboxylic acid tert-butyl ester (6.5 g, 34 mmol) was added as a solid in one portion, followed by the addition of DIC (4.2 g, 34 mmol) over 10 minutes. The resulting mixture was kept at 0° C. for 3 hours, then diluted with DCM (300 mL), twice shaken with 50 mL of water, shaken with NaOH (2N aqueous, 40 mL), and shaken with brine (50 mL). After removing the solvent under reduced pressure, the residue was purified on a silica gel column (3/7 ethyl acetate/hexane) to provide 7.0 g of 4-(3-phenyl-propynoyl)-piperazine-1-carboxylic acid tert-butyl ester (“Compound L”) as a white solid (70% yield).

The structure of Compound L was confirmed by ¹H NMR. Compound L: ¹H-NMR (CDCl₃) 7.55-7.58 (m, 2H), 7.36-7.45 (m, 3H), 3.81-3.83 (m, 2H), 3.66-3.69 (m, 2H), 3.52-3.55 (m, 2H), 3.45-3.48 (m, 2H), 1.49 (s, 9H).

A mixture of Compound L (2.0 g) and HCl (4N in 4 mL 1,4-dioxane) in 1,4-dioxane (10 mL) was shaken at about 25° C. for 12 hours. The resulting mixture was diluted with DCM (200 mL) and water (40 mL), then neutralized with NaOH (2N aqueous, 10 mL). The organic layer was separated and the solvent was removed under reduced pressure to provide 1.2 g of 3-phenyl-1-piperazin-1-yl-propynone (“Compound M,” 90% yield).

The structure of Compound M was confirmed by ¹H NMR. Compound M: ¹H-NMR (CDCl₃) 7.55 (dd, J=1.3, 8.1 Hz, 2H), 7.35-7.44 (m, 3H), 3.79-3.84 (m, 2H), 3.68-3.69 (m, 2H), 2.92-2.96 (m, 2H), 2.88-2.89 (m, 2H).

A mixture of Compound M (100 mg, 0.47 mmol), 2-chloro-4,6-dimethyl-pyrimidine (74 mg, 0.47 mmol) and diisopropylethylamine (“DIEA,” 0.5 mL) in DMSO (2 mL) was heated at 70° C. for 18 hours. After cooling to about 25° C., 2 mL of water was added to the reaction mixture. The resulting mixture was shaken at about 25° C. for 1 hour. The solid was collected and purified on a silica gel column (1/1 ethyl acetate/hexane) to provide 100 mg of Compound GZE(Va) as a white solid (70% yield).

The structure of Compound GZE(Va) was confirmed by ¹H NMR and mass spectral (MS) analysis. Compound GZE(Va): ¹H-NMR (CDCl₃) 7.56-7.61 (m, 2H), 7.38-7.47 (m, 3H), 6.35 (s, 1H), 3.95-3.98 (m, 2H), 3.89-3.94 (m, 4H), 3.76-3.79 (m, 2H), 2.32 (s, 6H); MS (EI): m/z 321 (M+H)⁺.

### 5.6 Example 6

### Synthesis of Compound HAC(a)

Compound HAC(Va) was prepared according to a scheme similar to Example 5 except that 0.47 mmol of 2-chloro-4-methyl-6-methoxy-pyrimidine was used in place of 2-chloro-4,6-dimethyl-pyrimidine. After the solid free base of Compound HAC(Va) was collected and purified on a silica gel column (1/1 ethyl acetate/hexane), the free base was dissolved in anhydrous diethyl ether while about 3 equivalents of 1 M HCl in diethyl ether solution was added slowly with stirring. The mixture was sonicated and the top layer was decanted. The remaining solid was washed 3 times with diethyl ether and dried under reduced pressure to provide the hydrochloride salt of Compound HAC(Va).

The structure of Compound HAC(Va) was confirmed by ¹H NMR and mass spectral (MS) analysis. Compound HAC(Va): ¹H-NMR (DMSO-d₆) 7.67 (m, 2H), 7.53 (m, 3H), 6.17 (s, 1H), 3.90 (m, 7H), 3.84 (m, 2H), 3.64 (m, 2H), 2.29 (s, 3H); MS (EI): m/z 337 (M+H)⁺.

### 5.7 Example 7

### Synthesis of Compound HBD(Va)

Compound HBD(Va) was prepared according to a scheme similar to Example 5 except that 0.47 mmol of 2,6-dichloro-4-methyl-pyrimidine was used in place of 2-chloro-4,6-dimethyl-pyrimidine. Compound HBD(Va) was obtained as a white solid.

The structure of Compound HBD(Va) was confirmed by ¹H NMR and mass spectral (MS) analysis. Compound HBD(Va): ¹H-NMR (CDCl₃) 7.57-7.59 (m, 2H), 7.38-7.48 (m, 3H), 6.48 (s, 1H), 3.89-3.98 (m, 6H), 3.76-3.79 (m, 2H), 2.35 (s, 3H); MS (EI): m/z 341 (M+H)⁺.

### 5.8 Example 8

### Synthesis of Compound GZF(Va)

Compound GZF(Va) was prepared according to the following scheme:

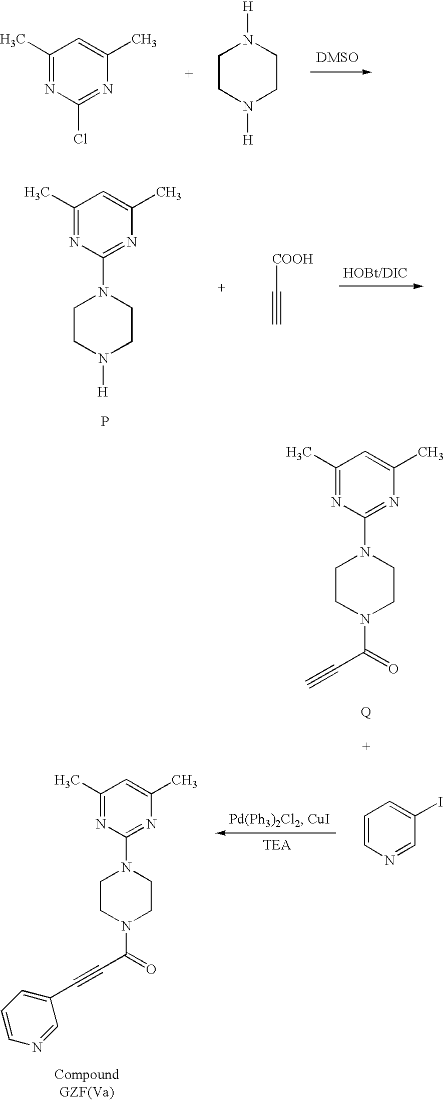

A mixture of 2-chloro-4,6-dimethylpyrimidine (3 g, 21 mmol) and piperazine (9 g, 107 mmol) in 15 mL DMSO was heated in a sealed tube at 100° C. for 16 hours. The solvent was removed and the solid product was purified on a silica gel column, eluting with ethyl acetate followed by 1/9 methanol/ethyl acetate, to provide 3.8 g of 1-(4,6-dimethylpyrimidin-2-yl)-piperazine (“Compound P,” 93% yield).

A mixture of Compound P (3.2 g, 17 mmol), propiolic acid (1.4 g, 20 mmol), HOBt (300 mg, 2 mmol), and DIC (2.6 mL, 17 mmol) in 40 mL DCM was stirred at about 25° C. for 4 hours. The mixture was then shaken with 2N NaOH. The organic layer was separated and dried. After removing the solvent under reduced pressure, the solid product was purified using a silica gel column eluted with gradient elution from 30/70 ethyl acetate/hexane to 70/30 ethyl acetate/hexane to provide 2.2 g of 1-(4-(4,6-dimethylpyrimidin-2-yl)piperazin-1-yl)-2-propyn-1-one (“Compound Q”) as an off-white solid (54% yield).

A mixture of Compound Q (245 mg, 1 mmol), 3-iodo-pyridine (193 mg, 1 mmol), 0.5 mL triethylamine, 30 mg copper(I) iodide and 50 mg dichloro-bis-(triphenylphospine) palladium(II) in 4 mL ethyl acetate was degassed with argon, and heated at 50° C. for 8 hours. The mixture was purified by column chromatography to provide 110 mg of Compound GZF(Va) as a solid (31% yield).

The structure of Compound GZF(Va) was confirmed by ¹H NMR and mass spectral (MS) analysis. Compound GZF(Va): ¹H-NMR (CDCl₃) 8.81 (d, J=1.2 Hz, 1H), 8.67 (m, 1H), 7.88 (m, 1H), 7.36 (m, 1H), 6.36 (s, 1H), 3.98 (m, 2H), 3.91 (m, 4H), 3.78 (m, 2H), 2.33 (s, 6H); MS (EI): m/z 322 (M+H)⁺.

### 5.9 Example 9

### Synthesis of Compound GZG(Va)

Compound GZG(Va) was prepared according to a scheme similar to Example 8 except that 1 mmol of 2-iodo-pyridine was used in place of 3-iodo-pyridine.

The structure of Compound GZG(Va) was confirmed by ¹H NMR and mass spectral (MS) analysis. Compound GZG(Va): ¹H-NMR (CDCl₃) 8.67 (m, 1H), 7.74 (m, 1H), 7.63 (m, 1H), 7.37 (m, 1H), 6.34 (s, 1H), 3.96 (br, 4H), 3.93 (m, 2H), 3.77 (m, 2H), 2.31 (s, 6H); MS (EI): m/z 322 (M+H)⁺.

### 5.10 Example 10

### Synthesis of Compound GZH(Va)

Compound GZH(Va) was prepared according to a scheme similar to Example 8 except that 1 mmol of 1-fluoro-4-iodo-benzene was used in place of 3-iodo-pyridine.

The structure of Compound GZH(Va) was confirmed by ¹H NMR and mass spectral (MS) analysis. Compound GZH(Va): ¹H-NMR (CDCl₃) 7.58 (m, 2H), 7.10 (m, 2H), 6.36 (s, 1H), 3.96 (m, 2H), 3.91 (m, 4H), 3.78 (m, 2H), 2.32 (s, 6H); MS (EI): m/z 339 (M+H)⁺.

### 5.11 Example 11

### Synthesis of Compound GZI(Va)

Compound GZI(Va) was prepared according to a scheme similar to Example 8 except that 1 mmol of 2-fluoro-5-iodo-pyridine was used in place of 3-iodo-pyridine.

The structure of Compound GZI(Va) was confirmed by ¹H NMR and mass spectral (MS) analysis. Compound GZI(Va): ¹H-NMR (CDCl₃) 8.45 (d, J=2.4 Hz, 1H), 7.99 (ddd, J=2.4, 7.3 and 8.4 Hz, 0.5H), 7.96 (ddd, J=2.4, 7.0 and 8.8 Hz, 0.5H), 7.02 (dd, J=0.7, 3.1 Hz, 0.5H), 6.99 (dd, J=0.6, 2.8 Hz, 0.5H), 6.36 (s, 1H), 3.95-4.00 (m, 2H), 3.85-3.94 (m, 4H), 3.75-3.78 (m, 2H), 2.33 (s, 6H); MS (EI): m/z 340 (M+H)⁺.

### 5.12 Example 12

### Binding of an Illustrative 2-Pyrimidinylpiperazine Compound to MGLUR5

The following assay demonstrates that Compound AAA(IIa), an illustrative 2-Pyrimidinylpiperazine Compound, binds to mGluR5.

Cell cultures: Primary glial cultures were prepared from cortices of Sprague-Dawley 18 days old embryos. The cortices were dissected and then dissociated by trituration. The resulting cell homogenate was plated onto poly-D-lysine precoated T175 flasks (BIOCOAT, commercially available from Becton Dickinson and Company Inc. of Franklin Lakes, N.J.) in Dulbecco's Modified Eagle's Medium (“DMEM,” pH 7.4), buffered with 25 mM HEPES, and supplemented with 15% fetal calf serum (“FCS,” commercially available from Hyclone Laboratories Inc. of Omaha, Nebr.), and incubated at 37° C. and 5% CO₂. After 24 hours, FCS supplementation was reduced to 10%. On day six, oligodendrocytes and microglia were removed by strongly tapping the sides of the flasks. One day following this purification step, secondary astrocytes cultures were established by subplating onto 96 poly-D-lysine precoated T175 flasks (BIOCOAT) at a density of 65,000 cells/well in DMEM and 10% FCS. After 24 hours, the astrocytes were washed with serum free medium and then cultured in DMEM, without glutamate, supplemented with 0.5% FCS, 20 mM HEPES, 10 ng/mL epidermal growth factor (“EGF”), 1 mM sodium pyruvate, and 1× penicillin/streptomycin at pH 7.5 for 3 to 5 days at 37° C. and 5% CO₂ The procedure allows the expression of the mGluR5 receptor by astrocytes, as demonstrated by S. Miller et al., J. Neuroscience 15(9):6103-6109 (1995).

Assay Protocol: After 3-5 days incubation with EGF, the astrocytes were washed with 127 mM NaCl, 5 mM KCl, 2 mM MgCl₂, 700 mM NaH₂PO₄, 2 mM CaCl₂, 5 mM NaHCO₃, 8 mM HEPES, 10 mM Glucose at pH 7.4 (“Assay Buffer”) and loaded with the dye Fluo-4 (commercially available from Molecular Probes Inc. of Eugene, Oreg.) using 0.1 mL of Assay Buffer containing Fluo-4 (3 mM final). After 90 minutes of dye loading, the cells were then washed twice with 0.2 mL Assay Buffer and resuspended in 0.1 mL of Assay Buffer. The plates containing the astrocytes were then transferred to a Fluorometric Imaging Plate reader (commercially available from Molecular Devices Corporation of Sunnyvale, Calif.) for the assessment of calcium mobilization flux in the presence of glutamate and in the presence or absence of antagonist. After monitoring fluorescence for 15 seconds to establish a baseline, DMSO solutions containing various concentrations of the 2-Pyrimidinylpiperazine Compounds diluted in Assay Buffer (0.05 mL of 4× dilutions for competition curves) were added to the cell plate and fluorescence was monitored for 2 minutes. 0.05 mL of a 4× glutamate solution (agonist) was then added to each well to provide a final glutamate concentration in each well of 10 mM. Plate fluorescence was then monitored for an additional 60 seconds after agonist addition. The final DMSO concentration in the assay was 1.0%. In each experiment, fluorescence was monitored as a function of time and the data analyzed using Microsoft Excel and GraphPad Prism. Dose-response curves were fit using a non-linear regression to determine IC₅₀ value. Compound AAA(IIa) showed an IC₅₀ value of 554.8±136.8 nM (mean of 5 experiments). In each experiment each data point was determined two times.

### 5.13 Example 13

### Binding of an Illustrative 2-Pyrimidinylpiperazine Compound to MGLUR5

Alternatively, the following assay can be used to demonstrate that a 2-Pyrimidinylpiperazine Compound binds to and modulates the activity of mGluR5.

40,000 CHO-rat mGluR5 cells/well are plated into 96 well plate (Costar 3409, Black, clear bottom, 96 well, tissue culture treated) for an overnight incubation in Dulbecco's Modified Eagle's Medium (DMEM, pH 7.4) and supplemented with glutamine, 10% FBS, 1% Pen/Strep, and 500 ug/mL Geneticin. CHO-rat mGluR5 cells are washed and treated with Optimem medium and incubated for 1-4 hours prior to loading cells. Cell plates are then washed with loading buffer (127 mM NaCl, 5 mM KCl, 2 mM MgCl₂, 700 μM Na H₂PO₄, 2 mM CaCl₂, 5 mM NaHCO₃, 8 mM Hepes, and 10 mM glucose, pH 7.4) and then incubated with 3 μM Fluo 4 (commercially available from Molecular probes Inc. of Eugene, Oreg.) in 0.1 mL of loading buffer. After 90 minutes of dye loading, the cells are then washed twice with 0.2 mL loading buffer and resuspended in 0.1 mL loading buffer.

The plates containing the CHO-rat mGluR5 cells are then transferred to a Fluorometric Imaging Plate Reader (FLIPR) (commercially available from Molecular Devices Corporation of Sunnyvale, Calif.) for the assessment of calcium mobilization flux in the presence of glutamate and in the presence or absence of test compounds. After monitoring fluorescence for 15 seconds to establish a baseline, DMSO solutions containing various concentrations of the test compound diluted in loading buffer (0.05 mL of 4× dilutions for the competition curves) are added to the cell plate and fluorescence is monitored for 2 minutes. 0.05 mL of 4× glutamate solution (agonist) is then added to each well to provide a final glutamate concentration in each well of 10 uM. Plate fluorescence is then monitored for an additional 60 seconds after agonist addition. The final DMSO concentration in the assay is 1.0%. In each experiment, fluorescence is monitored as a function of time and the data analyzed using Microsoft Excel and GraphPad Prism. Dose-response curves are fit using a non-linear regression to determine the IC50 value. In each experiment, each data point is determined two times.

### 5.14 Example 14

### In Vivo Assays for Prevention or Treatment of Pain

Test Animals: Each experiment uses rats weighing between 200-260 g at the start of the experiment. The rats are group-housed and have free access to food and water at all times, except prior to oral administration of a 2-Pyrimidinylpiperazine Compound when food is removed for 16 hours before dosing. A control group acts as a comparison to rats treated with a 2-Pyrimidinylpiperazine Compound. The control group is administered the carrier for the 2-Pyrimidinylpiperazine Compound. The volume of carrier administered to the control group is the same as the volume of carrier and 2-Pyrimidinylpiperazine Compound administered to the test group.

Acute Pain: To assess the actions of the 2-Pyrimidinylpiperazine Compounds for the treatment or prevention of acute pain the rat tail flick test can be used. Rats are gently restrained by hand and the tail exposed to a focused beam of radiant heat at a point 5 cm from the tip using a tail flick unit (Model 7360, commercially available from Ugo Basile of Italy). Tail flick latencies are defined as the interval between the onset of the thermal stimulus and the flick of the tail. Animals not responding within 20 seconds are removed from the tail flick unit and assigned a withdrawal latency of 20 seconds. Tail flick latencies are measured immediately before (pre-treatment) and 1, 3, and 5 hours following administration of a 2-Pyrimidinylpiperazine Compound. Data are expressed as tail flick latency(s) and the percentage of the maximal possible effect (% MPE), i.e., 20 seconds, is calculated as follows:

$$5 \% MPE = \frac{[ ( post administration latency ) - ( per \text{} administration latency ) ]}{( 20 s per \text{} administration latency )} ⨯ 100$$

The rat tail flick test is described in F. E. D'Amour et al., “A Method for Determining Loss of Pain Sensation,” J. Pharmacol. Exp. Ther. 72:74-79 (1941).

Acute pain can also be assessed by measuring the animal's response to noxious mechanical stimuli by determining the paw withdrawal threshold (“PWT”), as described below.

Inflammatory Pain: To assess the actions of the 2-Pyrimidinylpiperazine Compounds for the treatment or prevention of inflammatory pain the Freund's complete adjuvant (“FCA”) model of inflammatory pain is used. FCA-induced inflammation of the rat hind paw is associated with the development of persistent inflammatory mechanical hyperalgesia and provides reliable prediction of the anti-hyperalgesic action of clinically useful analgesic drugs (L. Bartho et al., “Involvement of Capsaicin-sensitive Neurones in Hyperalgesia and Enhanced Opioid Antinociception in Inflammation,” Naunyn-Schmiedeberg's Archives of Pharmacol. 342:666-670 (1990)). The left hind paw of each animal is administered a 50 μL intraplantar injection of 50% FCA. 24 hour post injection, the animal is assessed for response to noxious mechanical stimuli by determining the PWT, as described below. Rats are then administered a single injection of 1, 3, 10 or 30 mg/Kg of either a 2-Pyrimidinylpiperazine Compound; 30 mg/Kg of a control selected from Celebrex, indomethacin or naproxen; or carrier. Responses to noxious mechanical stimuli are then determined 1, 3, 5, and 24 hours post administration. Percentage reversal of hyperalgesia for each animal is defined as:

$$\% Reversal = \frac{[ ( post administration PWT ) - ( per \text{} administration PWT ) ]}{[ ( Baseline PWT ) - ( per \text{} administration PWT ) ]} ⨯ 100$$

Neuropathic Pain: To assess the actions of the 2-Pyrimidinylpiperazine Compounds for the treatment or prevention of neuropathic pain either the Seltzer model or the Chung model can be used.

In the Seltzer model, the partial sciatic nerve ligation model of neuropathic pain is used to produce neuropathic hyperalgesia in rats (Z. Seltzer et al., “A Novel Behavioral Model of Neuropathic Pain Disorders Produced in Rats by Partial Sciatic Nerve Injury,” Pain 43:205-218 (1990)). Partial ligation of the left sciatic nerve is performed under isoflurane/O₂ inhalation anaesthesia. Following induction of anesthesia, the left thigh of the rat is shaved and the sciatic nerve exposed at high thigh level through a small incision and is carefully cleared of surrounding connective tissues at a site near the trocanther just distal to the point at which the posterior biceps semitendinosus nerve branches off of the common sciatic nerve. A 7-0 silk suture is inserted into the nerve with a ⅜ curved, reversed-cutting mini-needle and tightly ligated so that the dorsal ⅓ to ½ of the nerve thickness is held within the ligature. The wound is closed with a single muscle suture (4-0 nylon (Vicryl)) and a Vetbond surgical glue. Following surgery, the wound area is dusted with antibiotic powder. Sham-treated rats undergo an identical surgical procedure except that the sciatic nerve is not manipulated. Following surgery, animals are weighed and placed on a warm pad until they recover from anesthesia. Animals are then returned to their home cages until behavioral testing begins. The animal is assessed for response to noxious mechanical stimuli by determining PWT, as described below, prior to surgery (baseline), then immediately prior to and 1, 3, and 5 hours after drug administration for the left rear paw of the animal. Percentage reversal of neuropathic hyperalgesia is defined as:

$$\% Reversal = \frac{[ ( post administration PWT ) - ( per \text{} administration PWT ) ]}{[ ( Baseline PWT ) - ( per \text{} administration PWT ) ]} ⨯ 100$$

In the Chung model, the spinal nerve ligation model of neuropathic pain is used to produce mechanical hyperalgesia, thermal hyperalgesia and tactile allodynia in rats. Surgery is performed under isoflurane/O₂ inhalation anaesthesia. Following induction of anaesthesia a 3 cm incision is made and the left paraspinal muscles are separated from the spinous process at the L₄-S₂ levels. The L₆ transverse process is carefully removed with a pair of small rongeurs to identify visually the L₄-L₆ spinal nerves. The left L₅ (or L₅ and L₆) spinal nerve(s) is isolated and tightly ligated with silk thread. A complete hemostasis is confirmed and the wound is sutured using non-absorbable sutures, such as nylon sutures or stainless steel staples. Sham-treated rats undergo an identical surgical procedure except that the spinal nerve(s) is not manipulated. Following surgery animals are weighed, administered a subcutaneous (s.c.) injection of saline or ringers lactate, the wound area is dusted with antibiotic powder and they are kept on a warm pad until they recover from the anesthesia. Animals are then returned to their home cages until behavioral testing begins. The animals are assessed for response to noxious mechanical stimuli by determining PWT, as described below, prior to surgery (baseline), then immediately prior to and 1, 3, and 5 hours after being administered a 2-Pyrimidinylpiperazine Compound for the left rear paw of the animal. The animal can also be assessed for response to noxious thermal stimuli or for tactile allodynia, as described below. The Chung model for neuropathic pain is described in S. H. Kim, “An Experimental Model for Peripheral Neuropathy Produced by Segmental Spinal Nerve Ligation in the Rat,” Pain 50(3):355-363 (1992).

Response to Mechanical Stimuli as an Assessment of Mechanical Hyperalzesia: The paw pressure assay can be used to assess mechanical hyperalgesia. For this assay, hind paw withdrawal thresholds (PWT) to a noxious mechanical stimulus are determined using an analgesymeter (Model 7200, commercially available from Ugo Basile of Italy) as described in C. Stein, “Unilateral Inflammation of the Hindpaw in Rats as a Model of Prolonged Noxious Stimulation: Alterations in Behavior and Nociceptive Thresholds,” Pharmacol. Biochem. and Behavior 31:451-455 (1988). The maximum weight that can be applied to the hind paw is set at 250 g and the end point is taken as complete withdrawal of the paw. PWT is determined once for each rat at each time point and only the affected (ipsilateral) paw is tested.

Response to Thermal Stimuli as an Assessment of Thermal Hyperalgesia: The plantar test can be used to assess thermal hyperalgesia. For this test, hind paw withdrawal latencies to a noxious thermal stimulus are determined using a plantar test apparatus (commercially available from Ugo Basile of Italy) following the technique described by K. Hargreaves et al., “A New and Sensitive Method for Measuring Thermal Nociception in Cutaneous Hyperalgesia,” Pain 32(1):77-88 (1988). The maximum exposure time is set at 32 seconds to avoid tissue damage and any directed paw withdrawal from the heat source is taken as the end point. Three latencies are determined at each time point and averaged. Only the affected (ipsilateral) paw is tested.

Assessment of Tactile Allodynia: To assess tactile allodynia, rats are placed in clear, plexiglass compartments with a wire mesh floor and allowed to habituate for a period of at least 15 minutes. After habituation, a series of von Frey monofilaments are presented to the plantar surface of the left (operated) foot of each rat. The series of von Frey monofilaments consists of six monofilaments of increasing diameter, with the smallest diameter fiber presented first. Five trials are conducted with each filament with each trial separated by approximately 2 minutes. Each presentation lasts for a period of 4-8 seconds or until a nociceptive withdrawal behavior is observed. Flinching, paw withdrawal or licking of the paw are considered nociceptive behavioral responses.

### 5.15 Example 15

### In Vivo Assays for Prevention or Treatment of Anxiety

The elevated plus maze test or the shock-probe burying test can be used to assess the anxiolytic activity of 2-Pyrimidinylpiperazine Compounds in rats or mice.

The Elevated Plus Maze Test: The elevated plus maze consists of a platform with 4 arms, two open and two closed (50×10×50 cm enclosed with an open roof). Rats (or mice) are placed in the center of the platform, at the crossroad of the 4 arms, facing one of the closed arms. Time spent in the open arms vs the closed arms and number of open arm entries during the testing period are recorded. This test is conducted prior to drug administration and again after drug administration. Test results are expressed as the mean time spent in open arms and the mean number of entries into open arms. Known anxiolytic drugs increase both the time spent in open arms and number of open arm entries. The elevated plus maze test is described in D. Treit, “Animal Models for the Study of Anti-anxiety Agents: A Review,” Neuroscience & Biobehavioral Reviews 9(2):203-222 (1985).

The Shock-Probe Burying Test: For the shock-probe burying test the testing apparatus consists of a plexiglass box measuring 40×30×40 cm, evenly covered with approximately 5 cm of bedding material (odor absorbent kitty litter) with a small hole in one end through which a shock probe (6.5 cm long and 0.5 cm in diameter) is inserted. The plexiglass shock probe is helically wrapped with two copper wires through which an electric current is administered. The current is set at 2 mA. Rats are habituated to the testing apparatus for 30 min on 4 consecutive days without the shock probe in the box. On test day, rats are placed in one corner of the test chamber following drug administration. The probe is not electrified until the rat touches it with its snout or fore paws, at which point the rat receives a brief 2 mA shock. The 15 min testing period begins once the rat receives its first shock and the probe remains electrified for the remainder of the testing period. The shock elicits burying behavior by the rat. Following the first shock, the duration of time the rat spends spraying bedding material toward or over the probe with its snout or fore paws (burying behavior) is measured as well as the number of contact-induced shocks the rat receives from the probe. Known anxiolytic drugs reduce the amount of burying behavior. In addition, an index of the rat's reactivity to each shock is scored on a 4 point scale. The total time spent immobile during the 15 min testing period is used as an index of general activity. The shock-probe burying test is described in D. Treit, 1985, supra.

### 5.16 Example 16

### In Vivo Assays for Prevention or Treatment of an Addictive Disorder

The conditioned place preference test or drug self-administration test can be used to assess the ability of 2-Pyrimidinylpiperazine Compounds to attenuate the rewarding properties of known drugs of abuse.

The Conditioned Place Preference Test: The apparatus for the conditioned place preference test consists of two large compartments (45×45×30 cm) made of wood with a plexiglass front wall. These two large compartments are distinctly different. Doors at the back of each large compartment lead to a smaller box (36×18×20 cm) box made of wood, painted grey, with a ceiling of wire mesh. The two large compartments differ in terms of shading (white vs black), level of illumination (the plexiglass door of the white compartment is covered with aluminum foil except for a window of 7×7 cm), texture (the white compartment has a 3 cm thick floor board (40×40 cm) with nine equally spaced 5 cm diameter holes and the black has a wire mesh floor), and olfactory cues (saline in the white compartment and 1 mL of 10% acetic acid in the black compartment). On habituation and testing days, the doors to the small box remain open, giving the rat free access to both large compartments.

The first session that a rat is placed in the apparatus is a habituation session and entrances to the smaller grey compartment remain open giving the rat free access to both large compartments. During habituation, rats generally show no preference for either compartment. Following habituation, rats are given 6 conditioning sessions. Rats are divided into 4 groups: carrier pre-treatment+carrier (control group), 2-Pyrimidinylpiperazine Compound pre-treatment+carrier, carrier pre-treatment+morphine, 2-Pyrimidinylpiperazine Compound pre-treatment+morphine. During each conditioning session the rat is injected with one of the drug combinations and confined to one compartment for 30 min. On the following day, the rat receives a carrier+carrier treatment and is confined to the other large compartment. Each rat receives three conditioning sessions consisting of 3 drug combination-compartment and 3 carrier-compartment pairings. The order of injections and the drug/compartment pairings are counterbalanced within groups. On the test day, rats are injected prior to testing (30 min to 1 hour) with either morphine or carrier and the rat is placed in the apparatus, the doors to the grey compartment remain open and the rat is allowed to explore the entire apparatus for 20 min. The time spent in each compartment is recorded. Known drugs of abuse increase the time spent in the drug-paired compartment during the testing session. If the 2-Pyrimidinylpiperazine Compound blocks the acquisition of morphine conditioned place preference (reward), there will be no difference in time spent in each side in rats pre-treated with a 2-Pyrimidinylpiperazine Compound and the group will not be different from the group of rats that was given carrier+carrier in both compartments. Data will be analyzed as time spent in each compartment (drug combination-paired vs carrier-paired). Generally, the experiment is repeated with a minimum of 3 doses of a 2-Pyrimidinylpiperazine Compound.

The Drug Self-Administration Test: The apparatus for the drug self-administration test is a standard commercially available operant conditioning chamber. Before drug trials begin rats are trained to press a lever for a food reward. After stable lever pressing behavior is acquired, rats are tested for acquisition of lever pressing for drug reward. Rats are implanted with chronically indwelling jugular catheters for i.v. administration of compounds and are allowed to recover for 7 days before training begins. Experimental sessions are conducted daily for 5 days in 3 hour sessions. Rats are trained to self-administer a known drug of abuse, such as morphine. Rats are then presented with two levers, an “active” lever and an “inactive” lever. Pressing of the active lever results in drug infusion on a fixed ratio 1 (FR1) schedule (i.e., one lever press gives an infusion) followed by a 20 second time out period (signaled by illumination of a light above the levers). Pressing of the inactive lever results in infusion of excipient. Training continues until the total number of morphine infusions stabilizes to within ±10% per session. Trained rats are then used to evaluate the effect of 2-Pyrimidinylpiperazine Compounds pre-treatment on drug self-administration. On test day, rats are pre-treated with a 2-Pyrimidinylpiperazine Compound or excipient and then are allowed to self-administer drug as usual. If the 2-Pyrimidinylpiperazine Compound blocks the rewarding effects of morphine, rats pre-treated with the 2-Pyrimidinylpiperazine Compound will show a lower rate of responding compared to their previous rate of responding and compared to excipient pre-treated rats. Data is analyzed as the change in number of drug infusions per testing session (number of infusions during test session—number of infusions during training session).

### 5.17 Example 17

### Functional Assay for Characterizing MGLUR1 Antagonistic Properties

Functional assays for the characterization of mGluR 1 antagonistic properties are well known in the art. For example, the following procedure can be used.

A CHO-rat mGluR1 cell line is generated using cDNA encoding rat mGluR1 receptor (M. Masu and S. Nakanishi, Nature 349:760-765 (1991)). The cDNA encoding rat mGluR1 receptor can be obtained from, e.g., Prof. S. Nakanishi (Kyoto, Japan).

40,000 CHO-rat mGluR1 cells/well are plated into a COSTAR 3409, black, clear bottom, 96 well, tissue culture treated plate (commercially available from Fisher Scientific of Chicago, Ill.) and are incubated in Dulbecco's Modified Eagle's Medium (DMEM, pH 7.4) supplemented with glutamine, 10% FBS, 1% Pen/Strep, and 500 μg/mL Geneticin for about 12 h. The CHO-rat mGluR1 cells are then washed and treated with OPTIMEM medium (commercially available from Invitrogen, Carlsbad, Calif.) and incubated for a time period ranging from 1 to 4 hours prior to loading the cells with the dye FLUO-4 (commercially available from Molecular Probes Inc., Eugene, Oreg.). After incubation, the cell plates are washed with loading buffer (127 mM NaCl, 5 mM KCl, 2 mM MgCl₂, 700 μM, NaH₂PO₄, 2 mM CaCl₂, 5 mMNaHCO₃, 8 mM HEPES, and 10 mM glucose, pH 7.4) and incubated with 3 μM FLUO-4 in 0.1 mL loading buffer for 90 min. The cells are then washed twice with 0.2 mL loading buffer, resuspended in 0.1 mL of loading buffer, and transferred to a FLIPR for measurement of calcium mobilization flux in the presence of glutamate and in the presence or absence of a 2-Pyrimidinylpiperazine Compound.

To measure calcium mobilization flux, fluoresence is monitored for about 15 s to establish a baseline and DMSO solutions containing various concentrations of a 2-Pyrimidinylpiperazine Compound ranging from about 50 μM to about 0.8 nM diluted in loading buffer (0.05 mL of a 4× dilution) are added to the cell plate and fluoresence is monitored for about 2 min. 0.05 mL of a 4× glutamate solution (agonist) is then added to each well to provide a final glutamate concentration in each well of 10 μM and fluoresence is monitored for about one additional min. The final DMSO concentration in the assay is 1%. In each experiment fluoresence is monitored as a function of time and the data is analyzed using a non-linear regression to determine the IC₅₀ value. In each experiment each data point is determined twice.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosures of which are incorporated herein by reference.

What is claimed is:

1. A compound of formula (I): 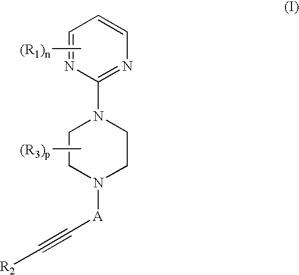
or a pharmaceutically acceptable salt thereof, wherein:
A is —C(O)—, —C(S)—, —CH₂—, —CH(C₁-C₄ alkyl)-, or —C(C₁-C₄ alkyl)(C₁-C₄ alkyl)-;
n is an integer ranging from 0 to 3;
each R₁ is independently —(C₁-C₃)alkyl, —O—(C₁-C₃)alkyl, -halo, —C(halo)₃, —CH(halo)₂, —CH₂(halo), —NO₂, —OH, or —CN;
when A is —CH₂—, —CH(C₁-C₄ alkyl)-, or —C(C₁-C₄ alkyl)(C₁-C₄ alkyl)-, then R₂ is -phenyl, -naphthyl, or -(C₁₄)aryl, each of which is unsubstituted or substituted with one or more R₄ groups, or, when A is —C(O)— or —C(S)—, then R₂ is
(i) —H, —(C₁-C₁₀)alkyl, —(C₂-C₁₀)alkenyl, —(C₂-C₁₀)alkynyl —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which, other than —H, is unsubstituted or substituted with one or more R₅ groups, or
(ii) -phenyl, -naphthyl, —(C₁₄)aryl, or -(5- to 10-membered)heteroaryl, each of which is unsubstituted or substituted with one or more R₄ groups;
p is an integer ranging from 0 to 2;
each R₃ is independently —OH, -halo, —NO₂, —CN, —NH₂, —(C₁-C₃)alkyl, or —CH₂OH;
each R₄ is independently —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, —(C₃-C₅)heterocycle, —C(halo)₃, —CH(halo)₂, —CH₂(halo), —CN, —OH, -halo, —N₃, —NO₂, —N(R₆)₂, —CH═NR₆, —NR₆OH, —COR₆, —C(O)OR₆, —OC(O)R₆, —OC(O)OR₆, —SR₆, —S(O)R₆, or —S(O)₂R₆;
each R₅ is independently —CN, —OH, -halo, —N₃, —NO₂, —N(R₆)₂, —CH═NR₆, —NR₆OH, —COR₆, —C(O)OR₆, —OC(O)R₆, —OC(O)OR₆, —SR₆, —S(O)R₆, or —S(O)₂R₆; and
each R₆ is independently —H, —(C₁-C₆)alkyl, —(C₂-C₆)alkenyl, —(C₂-C₆)alkynyl, —(C₃-C₈)cycloalkyl, —(C₅-C₈)cycloalkenyl, -phenyl, —(C₃-C₅)heterocycle, —C(halo)₃, —CH(halo)₂, or —CH₂(halo); and
each halo is independently —F, —Cl, —Br, or —I.

2. The compound of claim 1, wherein p is 0 or 1.

3. The compound of claim 1, wherein A is —CH₂—.

4. The compound of claim 1, wherein A is —CH(C₁-C₄ alkyl)-.

5. The compound of claim 1, wherein A is —C(C₁-C₄ alkyl)(C₁-C₄ alkyl)-.

6. The compound of claim 1, wherein A is —C(O)—.

7. The compound of claim 6, wherein R₂ is —H, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which is unsubstituted or substituted with one or more R₅ groups.

8. The compound of claim 6, wherein R₂ is -phenyl, -naphthyl, —(C₁₄)aryl, or -(5- to 10-membered)heteroaryl, each of which is unsubstituted or substituted with one or more R₄ groups.

9. The compound of claim 8, wherein R₂ is -phenyl.

10. The compound of claim 9, wherein the phenyl is substituted in its 4-position with an R₄ group.

11. The compound of claim 1, wherein A is —C(S)—.

12. The compound of claim 11, wherein R₂ is —H, —(C₁-C₁₀)alkyl, —(C₂-C₁₀)alkenyl, —(C₂-C₁₀)alkynyl, —(C₃-C₁₀)cycloalkyl, —(C₈-C₁₄)bicycloalkyl, —(C₈-C₁₄)tricycloalkyl, —(C₅-C₁₀)cycloalkenyl, —(C₈-C₁₄)bicycloalkenyl, —(C₈-C₁₄)tricycloalkenyl, -(3- to 7-membered)heterocycle, or -(7- to 10-membered)bicycloheterocycle, each of which is unsubstituted or substituted with one or more R₅ groups.

13. The compound of claim 11, wherein R₂ is -phenyl, -naphthyl, —(C₁₄)aryl, or -(5- to 10-membered)heteroaryl, each of which is unsubstituted or substituted with one or more R₄ groups.

14. The compound of claim 13, wherein R₂ is -phenyl.

15. The compound of claim 14, wherein the phenyl is substituted in its 4-position with an R₄ group.

16. The compound of claim 1 having the formula (Ia): 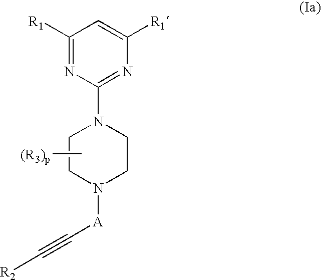
or a pharmaceutically acceptable salt thereof, wherein R₁ and R₁′ are independently —H, —(C₁-C₃)alkyl, —O—(C₁-C₃)alkyl, -halo, —C(halo)₃, —CH(halo)₂, —CH₂(halo), —NO₂, —OH, or —CN.

17. The compound of claim 16, wherein R₁ and R₁₁ are independently —(C₁-C₃)alkyl, —O—(C₁-C₃)alkyl, or -halo.

18. The compound of claim 17, wherein A is —C(O)—.

19. The compound of claim 17, wherein A is —C(S)—.

20. The compound of claim 17, wherein A is —CH₂—.

21. The compound of claim 17, wherein A is —CH(C₁-C₄ alkyl)-.

22. The compound of claim 17, wherein A is —C(C₁-C₄ alkyl)(C₁-C₄ alkyl)-.

23. The compound of claim 17, wherein R₁ is —CH₃ and R₁′ is —Cl.

24. The compound of claim 17, wherein R₁ is —CH₃ and R₁′ is —OCH₃.

25. The compound of claim 16, wherein R₁ and R₁′ are —(C₁-C₃)alkyl.

26. The compound of claim 25, wherein R₁ and R₁′ are —CH₃.

27. A composition comprising an effective amount of a compound or a pharmaceutically acceptable salt of the compound of claim 1 and a pharmaceutically acceptable carrier or excipient.

28. A composition comprising an effective amount of a compound or a pharmaceutically acceptable salt of the compound of claim 16 and a pharmaceutically acceptable carrier or excipient.

29. A method for treating pain, comprising administering to an animal in need thereof an effective amount of a compound or a pharmaceutically acceptable salt of the compound of claim 1.

30. The method of claim 29, further comprising administering to the animal an effective amount of another therapeutic agent.

31. A method for treating pain, comprising administering to an animal in need thereof an effective amount of a compound or a pharmaceutically acceptable salt of the compound of claim 16.

32. The method of claim 31, further comprising administering to the animal an effective amount of another therapeutic agent.

33. A method for treating an addictive disorder, comprising administering to an animal in need thereof an effective amount of a compound or a pharmaceutically acceptable salt of the compound of claim 1.

34. The method of claim 33, further comprising administering to the animal an effective amount of another therapeutic agent.

35. A method for treating an addictive disorder, comprising administering to an animal in need thereof an effective amount of a compound or a pharmaceutically acceptable salt of the compound of claim 16.

36. The method of claim 35, further comprising administering to the animal an effective amount of another therapeutic agent.

37. A method for treating Parkinson's disease, comprising administering to an animal in need thereof an effective amount of a compound or a pharmaceutically acceptable salt of the compound of claim 1.

38. The method of claim 37, further comprising administering to the animal an effective amount of another therapeutic agent.

39. A method for treating Parkinson's disease, comprising administering to an animal in need thereof an effective amount of a compound or a pharmaceutically acceptable salt of the compound of claim 16.

40. The method of claim 39, further comprising administering to the animal an effective amount of another therapeutic agent.

41. A method for treating anxiety, comprising administering to an animal in need thereof an effective amount of a compound or a pharmaceutically acceptable salt of the compound of claim 1.

42. The method of claim 41, further comprising administering to the animal an effective amount of another therapeutic agent.

43. A method for treating anxiety, comprising administering to an animal in need thereof an effective amount of a compound or a pharmaceutically acceptable salt of the compound of claim 16.

44. The method of claim 43, further comprising administering to the animal an effective amount of another therapeutic agent.

45. A method for treating schizophrenia, comprising administering to an animal in need thereof an effective amount of a compound or a pharmaceutically acceptable salt of the compound of claim 1.

46. The method of claim 45, further comprising administering to the animal an effective amount of another therapeutic agent.

47. A method for treating schizophrenia, comprising administering to an animal in need thereof an effective amount of a compound or a pharmaceutically acceptable salt of the compound of claim 16.

48. The method of claim 47, further comprising administering to the animal an effective amount of another therapeutic agent.

49. A method for inhibiting mGluR5-receptor function in a cell, comprising contacting a cell capable of expressing mGluR5 with an effective amount of a compound or a pharmaceutically acceptable salt of the compound of claim 1.

50. The method of claim 49, further comprising contacting the cell with an effective amount of another therapeutic agent.

51. A method for inhibiting mGluR5-receptor function in a cell, comprising contacting a cell capable of expressing mGluR5 with an effective amount of a compound or a pharmaceutically acceptable salt of the compound of claim 16.

52. The method of claim 51, further comprising contacting the cell with an effective amount of another therapeutic agent.

53. A method for preparing a composition, the method comprising admixing a compound or a pharmaceutically acceptable salt of the compound of claim 1 and a pharmaceutically acceptable carrier or excipient.

54. A kit comprising a container containing the composition of claim 27.
